(19) United States
(12) Patent Application Publication
(10) Pub. No.: US 2025/0255088 A1
(43) Pub. Date: Aug. 7, 2025

(54) HETEROCYCLIC COMPOUND, ORGANIC LIGHT-EMITTING ELEMENT COMPRISING SAME, AND COMPOSITION FOR ORGANIC LAYER OF ORGANIC LIGHT-EMITTING ELEMENT

(71) Applicant: LT MATERIALS CO.,LTD., Yongin-si (KR)

(72) Inventors: Eui-Jeong CHOI, Yongin-si (KR); Hyun-Joo LEE, Yongin-si (KR); Young-Beom SEO, Yongin-si (KR); Young-Seok NO, Yongin-si (KR); Dong-Jun KIM, Yongin-si (KR); Dae-Hyuk CHOI, Yongin-si (KR)

(21) Appl. No.: 19/187,890

(22) Filed: Apr. 23, 2025

Related U.S. Application Data
(63) Continuation of application No. PCT/KR2023/015850, filed on Oct. 13, 2023.

(30) Foreign Application Priority Data
Nov. 10, 2022 (KR) 10-2022-0149938

(51) Int. Cl. H10K 50/11; C07D 487/04; C07D 493/00; H10K 50/16; H10K 50/17; H10K 85/60

(52) U.S. Cl. CPC H10K 50/11; C07D 487/04; C07D 493/00; H10K 85/622; H10K 85/654; H10K 85/6572; H10K 85/6574; H10K 85/6576; H10K 50/16; H10K 50/17

(57) ABSTRACT

Provided is a heterocyclic compound, an organic light emitting device including the same, and a composition for an organic material layer of the organic light emitting device. The heterocyclic compound may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a charge generation layer and an electron transport layer of an organic light emitting device.

### TECHNICAL FIELD

The present invention relates to a heterocyclic compound, an organic light emitting device including the same, and a composition for an organic material layer of the organic light emitting device.

This application claims priority to and the benefit of Korean Patent Application No. 10-2022-0149938 filed in the Korean Intellectual Property Office on Nov. 10, 2022, the entire contents of which are incorporated herein by reference.

### BACKGROUND ART

A n electroluminescence device is a kind of self-emitting type display device, and has an advantage in that the viewing angle is wide, the contrast is excellent, and the response speed is fast.

A n organic light emitting device is composed of a structure in which an organic thin film is disposed between two electrodes. When a voltage is applied to an organic light emitting device having such a structure, electrons and holes injected from the two electrodes are combined with each other in the organic thin film to make a pair, and then, the paired electrons and holes emit light while being annihilated. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may serve as a hole injection layer, a hole transport layer, an electron blocking layer, a hole blocking layer, an electron transport layer, an electron injection layer, a charge generation layer, and the like.

In order to improve the performance, efficiency and service life of the organic light emitting device, there is a continuous need for developing a material for an organic thin film.

### DETAILED DESCRIPTION OF THE INVENTION

### Technical Problem

The present invention has been made in an effort to provide a heterocyclic compound, an organic light emitting device including the same, and a composition for an organic material layer of the organic light emitting device.

### Technical Solution

In an exemplary embodiment of the present application, provided is a heterocyclic compound represented by the following Chemical Formula 1.

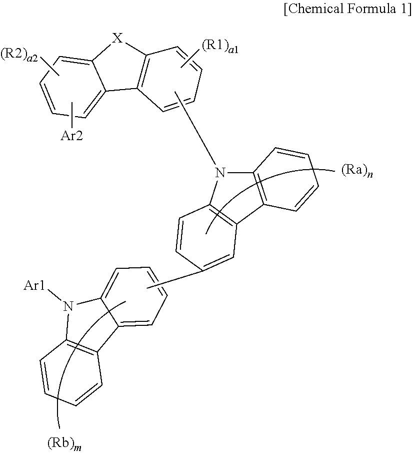

In Chemical Formula 1,
X is O or S,
Ar1 is a substituted or unsubstituted C6 to C60 aryl group,
Ar2 is a C6 to C60 aryl group substituted with at least one deuterium,
R1, R2, Ra, and Rb are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group,
a1 is an integer from 0 to 3, and when a1 is 2 or higher, substituents in the parenthesis are the same as or different from each other,
a2 is an integer from 0 to 3, and when a2 is 2 or higher, substituents in the parenthesis are the same as or different from each other,
m is an integer from 0 to 7, and when m is 2 or higher, substituents in the parenthesis are the same as or different from each other,
n is an integer from 0 to 7, and when n is 2 or higher, substituents in the parenthesis are the same as or different from each other, and
a deuterium substitution rate of

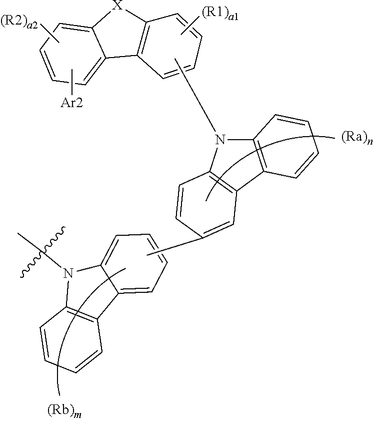

### of Chemical Formula 1 is 60% to 100%.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In addition, in an exemplary embodiment of the present application, provided is an organic light emitting device in which the organic material layer further includes a heterocyclic compound represented by the following Chemical Formula 2.

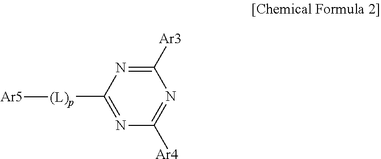

In Chemical Formula 2,
L is a direct bond; a substituted or unsubstituted C6 to C60 arylene group; or a substituted or unsubstituted C2 to C60 heteroarylene group,
p is an integer from 0 to 4, and when p is 2 or higher, substituents in the parenthesis are the same as or different from each other, and
Ar3 to Ar5 are the same as or different from each other, and are each independently a substituted or unsubstituted C6 to C60 aryl group; or a substituted or unsubstituted C2 to C60 heteroaryl group.

Further, in an exemplary embodiment of the present application, provided is a composition for an organic material layer of an organic light emitting device, which includes the heterocyclic compound represented by Chemical Formula 1; and the heterocyclic compound represented by Chemical Formula 2.

### Advantageous Effects

The heterocyclic compound described in the present specification may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a charge generation layer and an electron transport layer of an organic light emitting device.

Specifically, one or two or more of the heterocyclic compounds represented by Chemical Formula 1 can be used, and the heterocyclic compounds represented by Chemical Formula 1 can be used as materials for the charge generation layer and the electron transport layer. In particular, the heterocyclic compound can be used as a material for the charge generation layer and the electron transport layer of an organic light emitting device by introducing various substituents and changing the binding position of the substituent to adjust the bandgap.

The heterocyclic compound of the present invention has an advantage in that the deuterium substitution rate of the remaining portion except for Ar1 in the structure of Chemical Formula 1 is 60% to 100%, thereby accelerating the transfer of holes and electrons.

In particular, when the heterocyclic compound of the present invention is used in the light emitting layer of an organic light emitting device, it is possible to obtain an effect in which the driving voltage of the device is lowered, the efficiency of the device is also increased, and the service life of the device is extended.

### BRIEF DESCRIPTION OF DRAWINGS

FIGS. 1 to 3 are views each schematically illustrating a stacking structure of an organic light emitting device according to an exemplary embodiment of the present application.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

### Best Mode

Hereinafter, the present specification will be described in more detail.

When one part “includes” one constituent element in the present specification, unless otherwise specifically described, this does not mean that another constituent element is excluded, but means that another constituent element may be further included.

In the present specification,

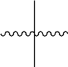

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as

T2/T1×100=T %.

That is, in an example, the deuterium content of 20% in a phenyl group represented by

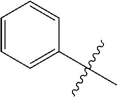 [SMILES: *c1ccccc1]

may be represented by 20% when the total number of substituents that the phenyl group can have is 5 (T1 in the formula) and the number of deuterium atoms among the substituents is 1 (T2 in the formula). That is, a deuterium content of 20% in the phenyl group may be represented by the following structural formula.

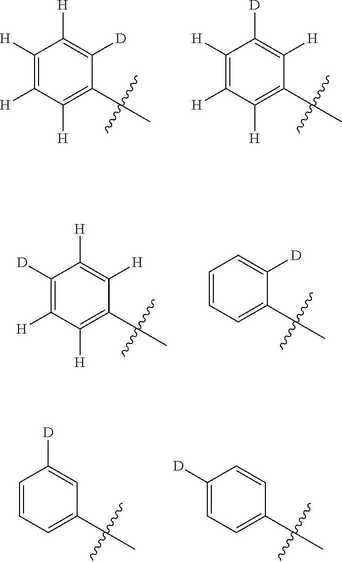 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. H ere, the polycycle means a group in which an aryl group is directly linked to or fused with another cyclic group. H ere, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

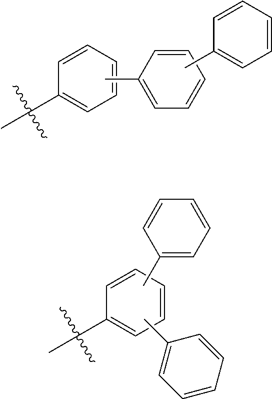

In the present specification, the fluorenyl group may be substituted, and adjacent substituents may be bonded to each other to form a ring.

When the fluorenyl group is substituted, the substituted fluorenyl group may be

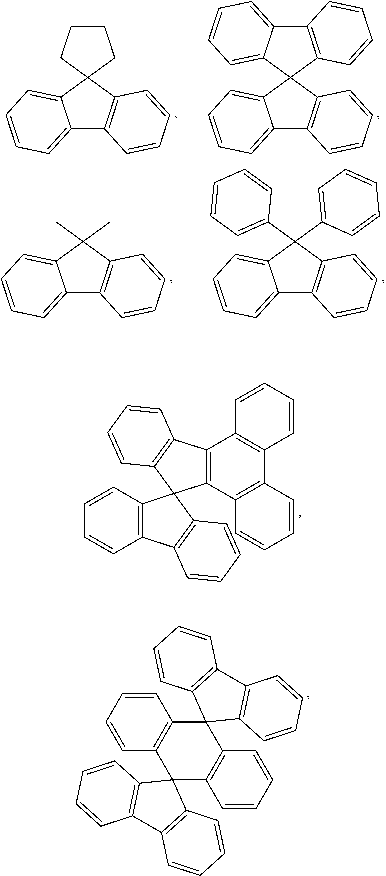

and the like, but is not limited thereto.

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. H ere, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when the substituent is a carbazole group, it means being bonded to nitrogen or carbon of carbazole.

In the present specification, when a carbazole group is substituted, the nitrogen or carbon of the carbazole may be substituted with an additional substituent.

In the present specification, a benzocarbazole group may be any one of the following structures.

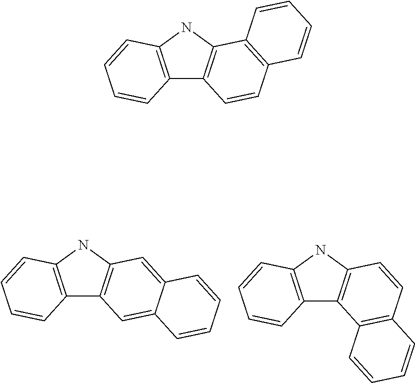

In the present specification, a dibenzocarbazole group may be any one of the following structures.

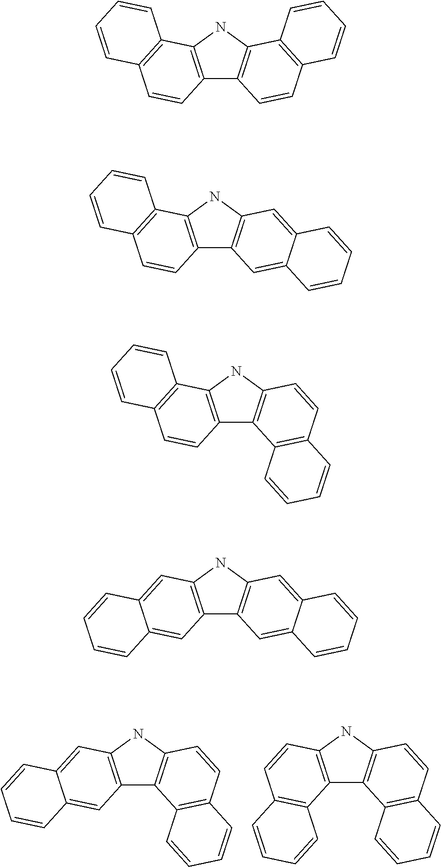

In the present specification, a naphthobenzofuran group may be any one of the following structures.

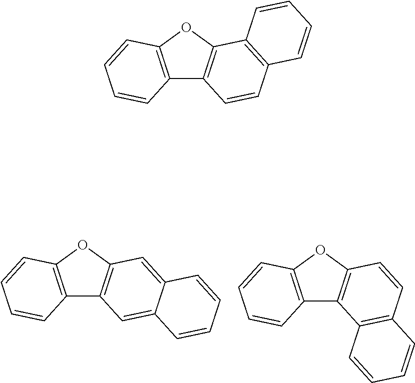

In the present specification, a naphthobenzothiophene group may be any one of the following structures.

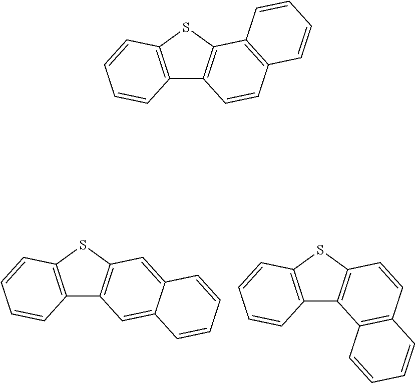

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

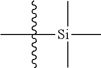 [SMILES: *[Si](C)(C)C]

(a trimethylsilyl group),

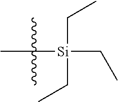 [SMILES: *[Si](CC)(CC)CC]

(a triethylsilyl group),

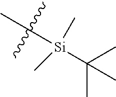 [SMILES: *[Si](C)(C)C(C)(C)C]

(a t-butyldimethylsilyl group),

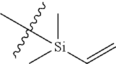 [SMILES: *[Si](C)(C)C=C]

(a vinyldimethylsilyl group),

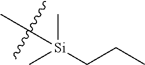 [SMILES: *[Si](C)(C)CCC]

(a propyldimethylsilyl group),

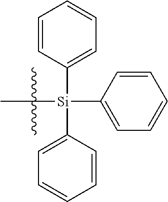 [SMILES: *[Si](c1ccccc1)(c1ccccc1)c1ccccc1]

(a triphenylsilyl group),

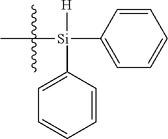 [SMILES: *[SiH](c1ccccc1)c1ccccc1]

(a diphenylsilyl group),

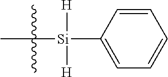 [SMILES: *[SiH2]c1ccccc1]

(a phenylsilyl group) and the like, but are not limited thereto.

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be each applied to the rings, except for those that are not monovalent groups.

In an exemplary embodiment of the present application, provided is the compound represented by Chemical Formula 1.

In an exemplary embodiment of the present application, a group not represented by a substituent; or a group represented by hydrogen may mean being all substitutable with deuterium. That is, it may be shown that hydrogen; or deuterium can be substituted with each other.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having lower vibration energy.

Further, the single bond dissociation energy of carbon and deuterium is higher than the single bond dissociation energy of carbon and hydrogen. Accordingly, the deuterium-substituted structure has an effect of increasing the thermal stability of the molecule and improving the service life of the device using the increased thermal stability.

When a compound is deposited on a silicon wafer, a material including deuterium tends to be packed so that the intermolecular distance is reduced. Further, when the surface of a thin film is observed using an atomic force microscope (AFM), it can be confirmed that the thin film made of a compound including deuterium is deposited with a more uniform surface without any aggregated portion.

There is an advantage in that the deuterium substitution rate of the remaining portion except for Ar1 in the structure of the heterocyclic compound of Chemical Formula 1 of the present application is 60% to 100%, thereby accelerating the transfer of holes and electrons. The deuterium-substituted compound is characterized in that the energy in the ground state is lower than that of the hydrogen-substituted compound, and the shorter the bond length between carbon and deuterium is, the smaller the molecular hardcore volume is. This allows for a stacking structure with shorter intermolecular distances during the manufacture of a device, facilitating smooth electron transport between molecules.

In an exemplary embodiment of the present application, provided is a heterocyclic compound represented by the following Chemical Formula 1.

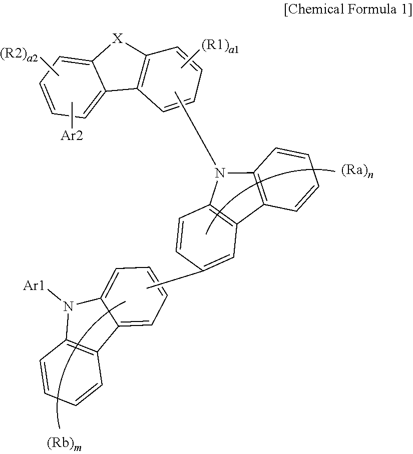

In Chemical Formula 1,
X is O or S,
Ar1 is a substituted or unsubstituted C6 to C60 aryl group,
Ar2 is a C6 to C60 aryl group substituted with at least one deuterium,
R1, R2, Ra, and Rb are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group,
a1 is an integer from 0 to 3, and when a1 is 2 or higher, substituents in the parenthesis are the same as or different from each other,
a2 is an integer from 0 to 3, and when a2 is 2 or higher, substituents in the parenthesis are the same as or different from each other,
m is an integer from 0 to 7, and when m is 2 or higher, substituents in the parenthesis are the same as or different from each other,
n is an integer from 0 to 7, and when n is 2 or higher, substituents in the parenthesis are the same as or different from each other, and
a deuterium substitution rate of

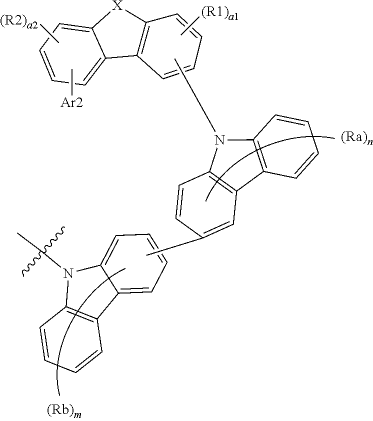

### of Chemical Formula 1 is 60% to 100%.

In an exemplary embodiment of the present application, the deuterium substitution rate

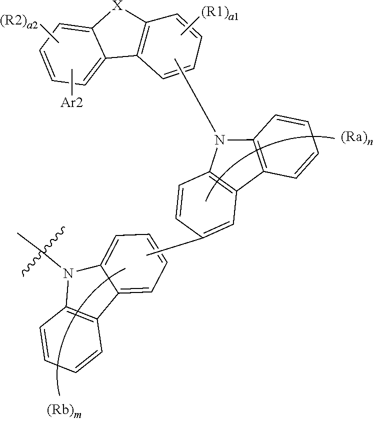

### of Chemical Formula 1 may be 70% to 100%.

In another exemplary embodiment, the deuterium substitution rate of

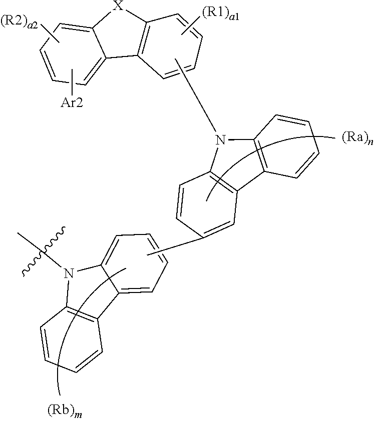

### of Chemical Formula 1 may be 80% to 100%.

In still another exemplary embodiment, the deuterium substitution rate of

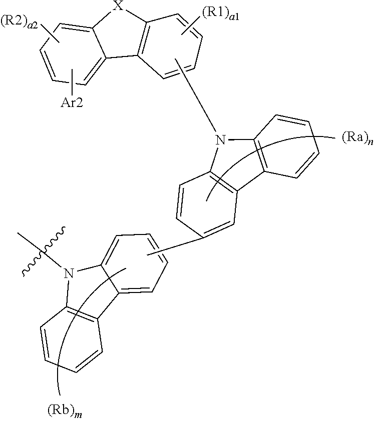

### of Chemical Formula 1 may be 90% to 100%.

In yet another exemplary embodiment, the deuterium substitution rate of

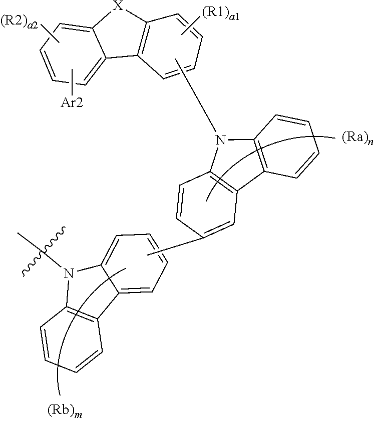

### of Chemical Formula 1 may be 100%.

In an exemplary embodiment of the present application, Chemical Formula 1 may be represented by the following Structural Formulae A to C.

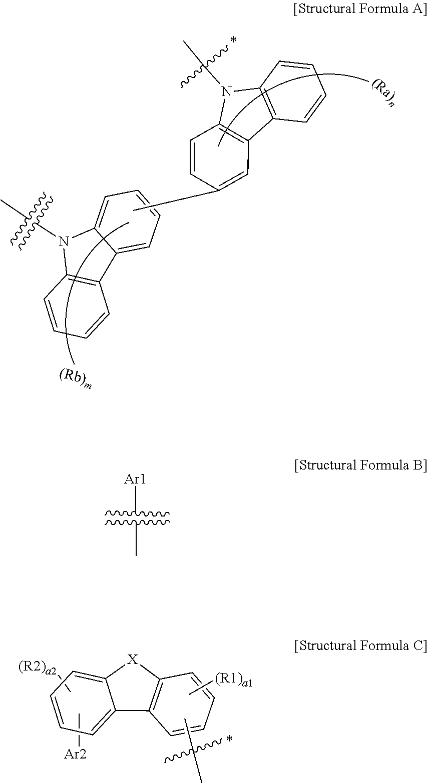

In Structural Formulae A to C,
the definitions of X, Ar1, Ar2, R1, R2, Ra, Rb, a1, a2, m, and n are the same as the definitions in Chemical Formula 1,
the deuterium substitution rate of a structure in which Structural Formula A and Structural Formula C are combined is 60% to 100%, and
the

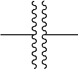

is a position where Structural Formula A and Structural Formula B are linked, and the

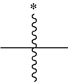

is a position where Structural Formula A and Structural Formula C are linked.

In an exemplary embodiment of the present application, the deuterium substitution rate of a structure in which Structural Formula A and Structural Formula C are combined may be 60% to 100%.

The structure in which Structural Formula A and Structural Formula C are combined means the remaining portion except for Ar1, in

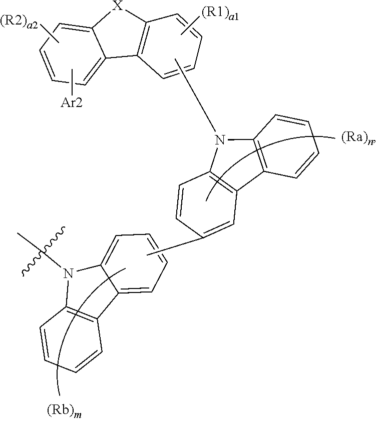

that is, the structure of Chemical Formula 1.

In another exemplary embodiment, the deuterium substitution rate of a structure in which Structural Formula A and Structural Formula C are combined may be 80% to 100%.

In still another exemplary embodiment, the deuterium substitution rate of a structure in which Structural Formula A and Structural Formula C are combined may be 90% to 100%.

In yet another exemplary embodiment, the deuterium substitution rate of a structure in which Structural Formula A and Structural Formula C are combined may be 100%.

In an exemplary embodiment of the present application, the deuterium substitution rate of Ar2, R1, R2, Ra, and Rb may be 70% to 100%.

In another exemplary embodiment, the deuterium substitution rate of Ar2, R1, R2, Ra, and Rb may be 80% to 100%.

In still another exemplary embodiment, the deuterium substitution rate of Ar2, R1, R2, Ra, and Rb may be 90% to 100%.

In yet another exemplary embodiment, the deuterium substitution rate of Ar2, R1, R2, Ra, and Rb may be 100%.

In still yet another exemplary embodiment, the deuterium substitution rate of Ar2, R1, R2, Ra, and Rb may be 70% to 100%.

In an exemplary embodiment of the present application, the deuterium substitution rate of Structural Formula B may be 50% or less.

In another exemplary embodiment, the deuterium substitution rate of Structural Formula B may be 30% or less.

In still another exemplary embodiment, the deuterium substitution rate of Structural Formula B may be 10% or less.

In yet another exemplary embodiment, the deuterium substitution rate of Structural Formula B may be 0%.

In an exemplary embodiment of the present application, the deuterium substitution rate of A r may be 0%.

In an exemplary embodiment of the present application, m+n is 14, and the deuterium substitution rate of Ra and Rb may be 60% or more.

In another exemplary embodiment, m+n is 14, and the deuterium substitution rate of Ra and Rb may be 70% or more and 100% or less.

In still another exemplary embodiment, m+n is 14, and the deuterium substitution rate of Ra and Rb may be 80% or more and 100% or less.

In yet another exemplary embodiment, m+n is 14, and the deuterium substitution rate of Ra and Rb may be 90% or more and 100% or less.

In still yet another exemplary embodiment, m+n is 14, and the deuterium substitution rate of R a and Rb may be 100%.

In the present specification, m+n being 14 means that the total number of R a and R b substituents in the biscarbazole of Chemical Formula 1 is 14.

In an exemplary embodiment of the present application, a1+a2 is 6, and the deuterium substitution rate of R1 and R2 may be 60% or more.

In still another exemplary embodiment, a1+a2 is 6, and the deuterium substitution rate of R1 and R2 may be 70% or more and 100% or less.

In yet another exemplary embodiment, a1+a2 is 6, and the deuterium substitution rate of R1 and R2 may be 80% or more and 100% or less.

In still yet another exemplary embodiment, a1+a2 is 6, and the deuterium substitution rate of R1 and R2 may be 90% or more and 100% or less.

In a further exemplary embodiment, a1+a2 is 6, and the deuterium substitution rate of R1 and R2 may be 100%.

In an exemplary embodiment of the present application, Chemical Formula 1 may be represented by any one of the following Chemical Formulae 1-1 to 1-4.

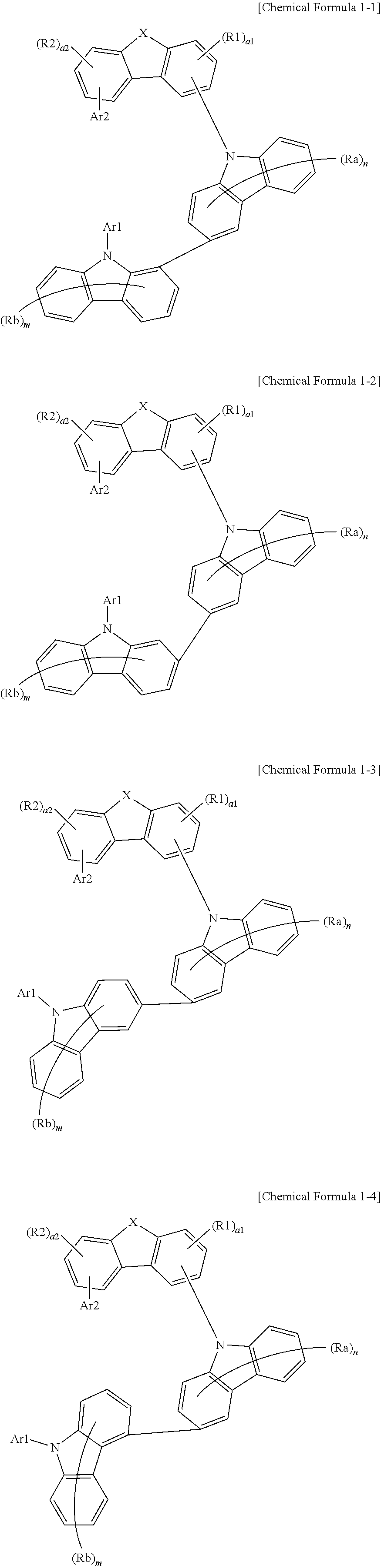

In Chemical Formulae 1-1 to 1-4,
the definitions of X, Ar1, Ar2, R1, R2, Ra, Rb, a1, a2, m, and n are the same as the definitions in Chemical Formula 1.

In an exemplary embodiment of the present application, Chemical Formula 1 may be represented by any one of the following Chemical Formulae 1-5 to 1-8.

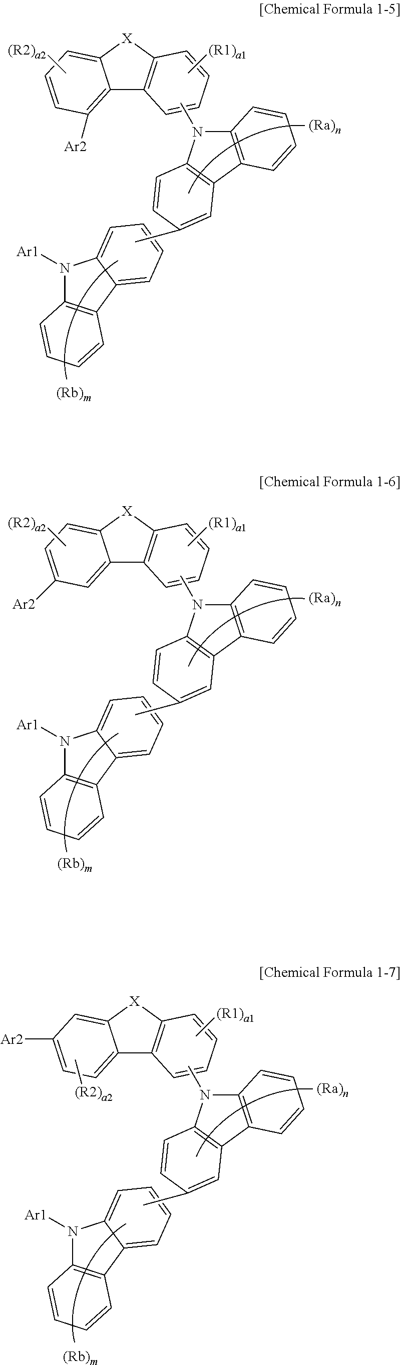

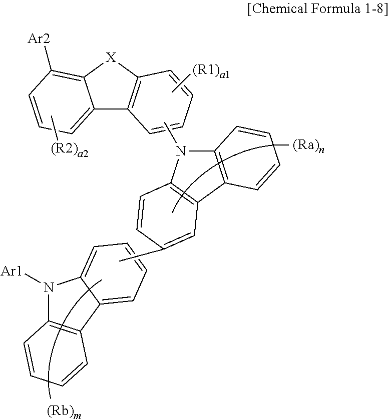

In Chemical Formulae 1-5 to 1-8,
the definitions of X, Ar1, Ar2, R1, R2, Ra, Rb, a1, a2, m, and n are the same as the definitions in Chemical Formula 1.

In an exemplary embodiment of the present application, Ar1 may be a substituted or unsubstituted C6 to C40 aryl group.

In another exemplary embodiment, A r may be a C6 to C20 aryl group.

In still another exemplary embodiment, An may be a phenyl group; a biphenyl group; a terphenyl group; a naphthyl group; a phenanthrene group; or a triphenylene group.

In yet another exemplary embodiment, A r may be a phenyl group; or a biphenyl group.

In an exemplary embodiment of the present application, Ar2 may be a C6 to C40 aryl group substituted with at least one deuterium.

In another exemplary embodiment, Ar2 may be a C6 to C20 aryl group substituted with at least one deuterium.

In still another exemplary embodiment, Ar2 may be a phenyl group substituted with at least one deuterium; a biphenyl group substituted with at least one deuterium; a terphenyl group substituted with at least one deuterium; a naphthyl group substituted with at least one deuterium; a phenanthrene group substituted with at least one deuterium; or a triphenylene group substituted with at least one deuterium.

In yet another exemplary embodiment, Ar2 may be any one selected from the following structural formulae.

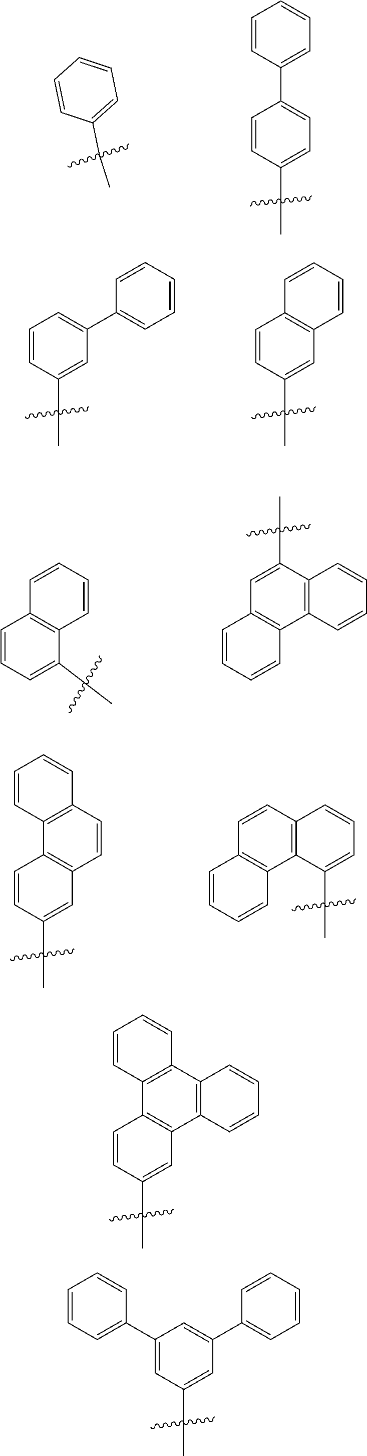

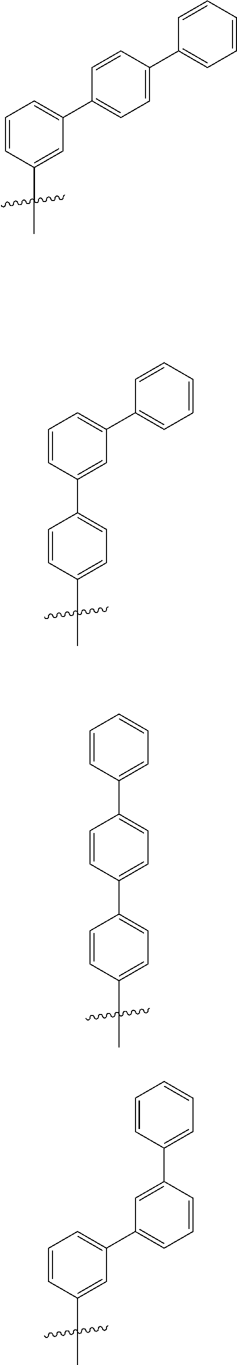 [SMILES: *c1cccc(-c2ccc(-c3ccccc3)cc2)c1 ; *c1ccc(-c2cccc(-c3ccccc3)c2)cc1 ; *c1ccc(-c2ccc(-c3ccccc3)cc2)cc1 ; *c1cccc(-c2cccc(-c3ccccc3)c2)c1]

In the structural formulae, a substitutable position may be substituted with at least one deuterium.

In still yet another exemplary embodiment, Ar2 may be a phenyl group substituted with at least one deuterium; a biphenyl group substituted with at least one deuterium; or a terphenyl group substituted with at least one deuterium.

In an exemplary embodiment of the present application, R1, R2, Ra, and Rb are the same as or different from each other, and may be each independently hydrogen; deuterium; a halogen; a cyano group; a substituted or unsubstituted C1 to C40 alkyl group; a substituted or unsubstituted C6 to C40 aryl group; or a substituted or unsubstituted C2 to C40 heteroaryl group.

In another exemplary embodiment, R1, R2, Ra, and Rb are the same as or different from each other, and may be each independently hydrogen; deuterium; a halogen; a cyano group; a substituted or unsubstituted C1 to C20 alkyl group; a substituted or unsubstituted C6 to C20 aryl group; or a substituted or unsubstituted C2 to C20 heteroaryl group.

In still another exemplary embodiment, R1, R2, Ra, and Rc are the same as or different from each other, and may be each independently hydrogen; deuterium; or a substituted or unsubstituted C1 to C20 alkyl group.

In yet another exemplary embodiment, R1, R2, Ra, and Rb are the same as or different from each other, and may be each independently hydrogen; deuterium; a substituted or unsubstituted methyl group; a substituted or unsubstituted ethyl group; or a substituted or unsubstituted propyl group.

In still yet another exemplary embodiment, R1, R2, Ra, and Rb are the same as or different from each other, and may be each independently hydrogen; or deuterium.

In an exemplary embodiment of the present application, a1 and a2 are each an integer from 1 to 3, and when a1 and a2 are each 2 or higher, substituents in the parenthesis may be the same as or different from each other.

In another exemplary embodiment, a1 and a2 are each an integer from 2 and 3, and substituents in the parenthesis may be the same as or different from each other.

In still another exemplary embodiment, a1 and a2 are each 3, and substituents in the parenthesis may be the same as or different from each other.

In an exemplary embodiment of the present application, m and n are each an integer from 1 to 7, and when m is 2 or higher, substituents in the parenthesis may be the same as or different from each other.

In another exemplary embodiment, m and n are each an integer from 2 to 7, and substituents in the parenthesis may be the same as or different from each other.

In still another exemplary embodiment, m and n are each an integer from 3 to 7, and substituents in the parenthesis may be the same as or different from each other.

In yet another exemplary embodiment, m and n are each an integer from 4 to 7, and substituents in the parenthesis may be the same as or different from each other.

In still yet another exemplary embodiment, m and n are each an integer from 5 to 7, and substituents in the parenthesis may be the same as or different from each other.

In a further exemplary embodiment, m and n are each an integer from 6 to 7, and substituents in the parenthesis may be the same as or different from each other.

In another further exemplary embodiment, m and n are each 7, and substituents in the parenthesis may be the same as or different from each other.
n is an integer from 0 to 7, and when n is 2 or higher, substituents in the parenthesis are the same as or different from each other, and
in an exemplary embodiment of the present application, Chemical Formula 1 may be represented by any one of the following Chemical Formulae 1-9 and 1-10.

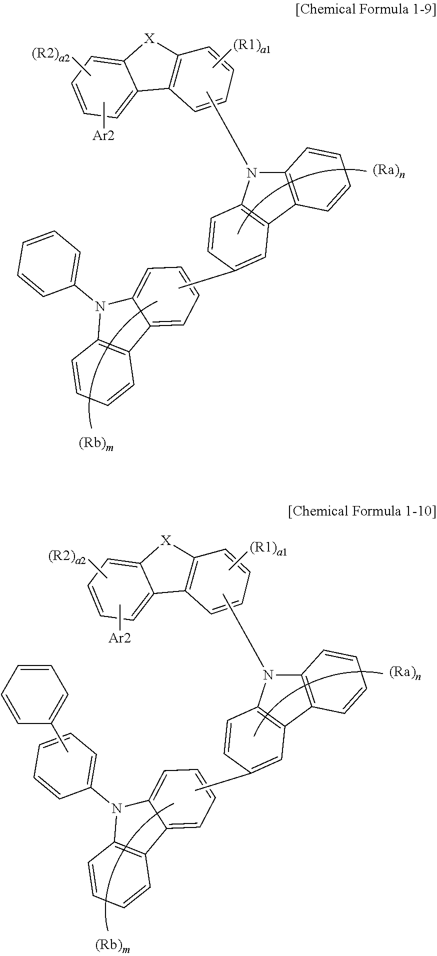

In Chemical Formulae 1-9 and 1-10,
the definitions of X, Ar2, R1, R2, Ra, Rb, a1, a2, m, and n are the same as the definitions in Chemical Formula 1.

In an exemplary embodiment of the present application, provided is a compound in which Chemical Formula 1 is represented by any one of the following compounds. However, in Chemical Formula 1, the positions and number of deuterium substitutions are not limited to the following compounds.

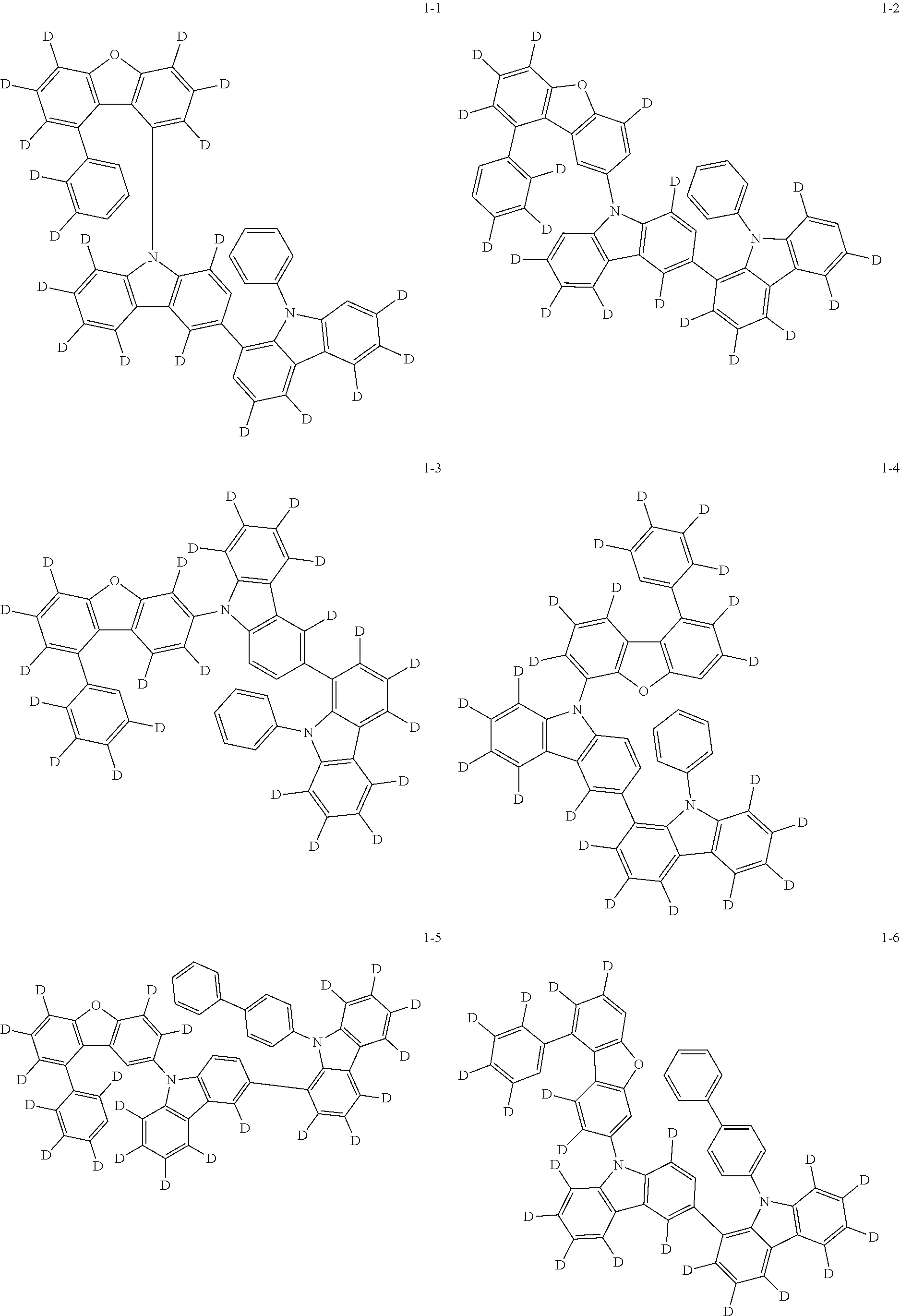

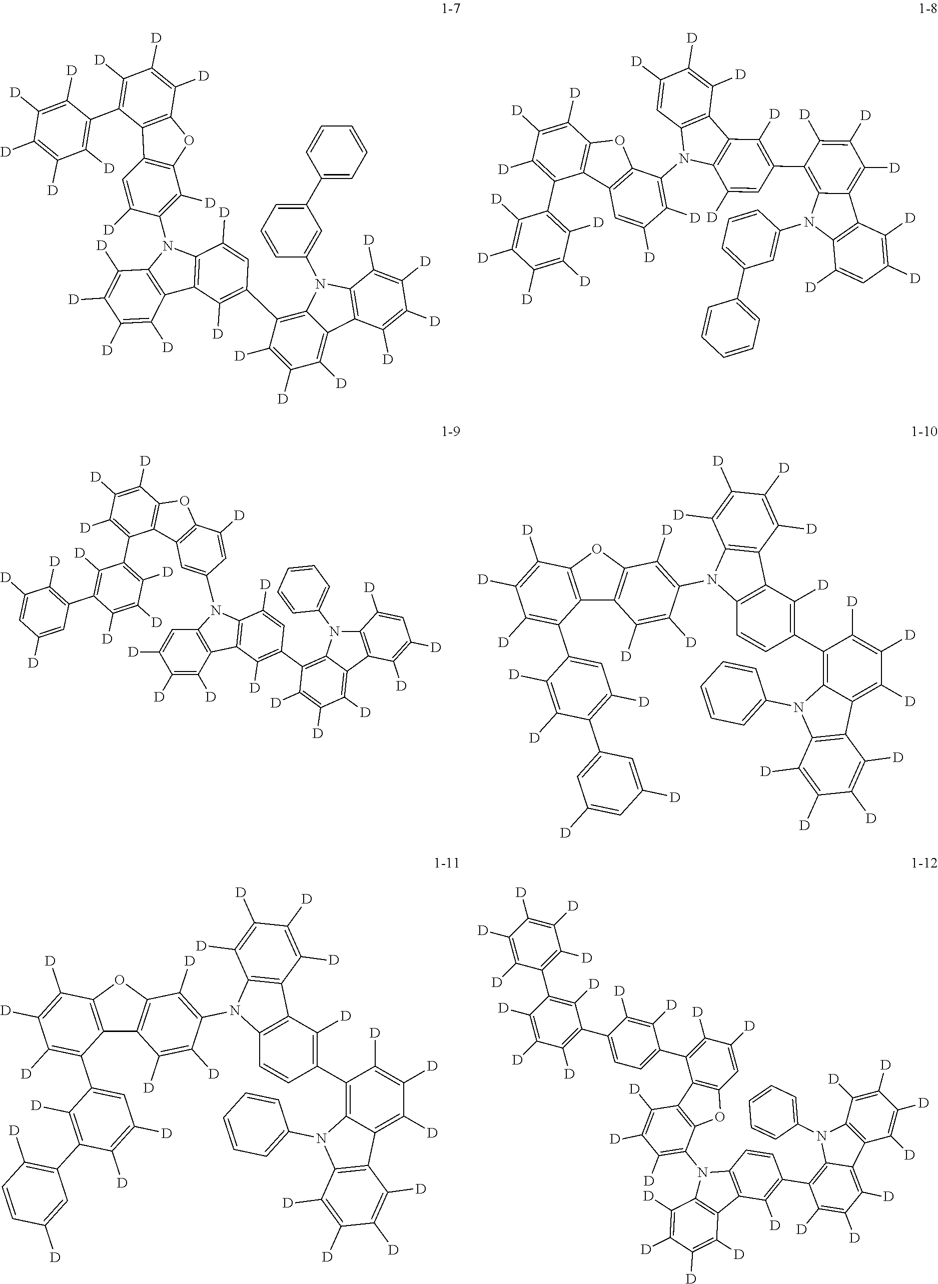

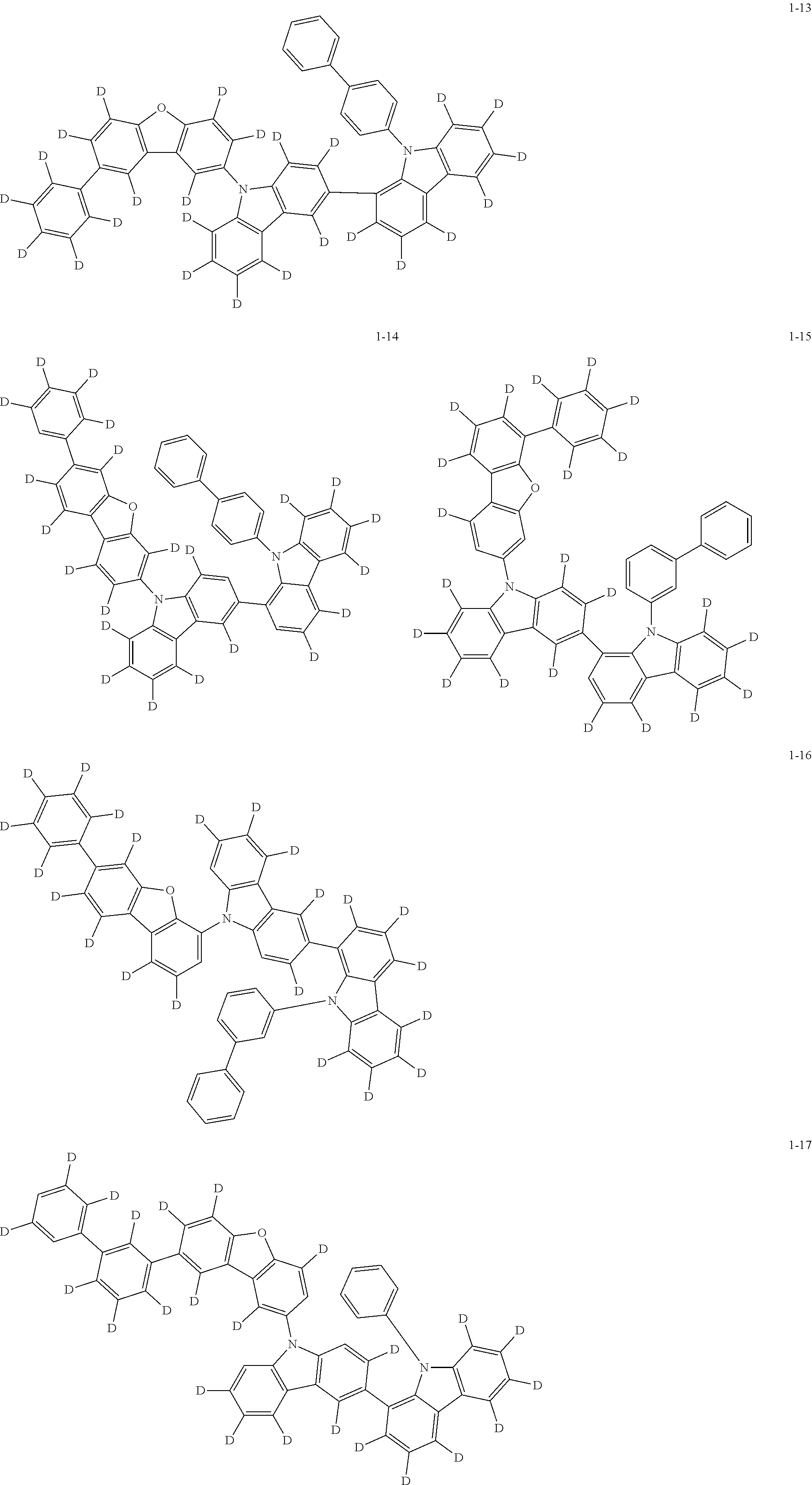

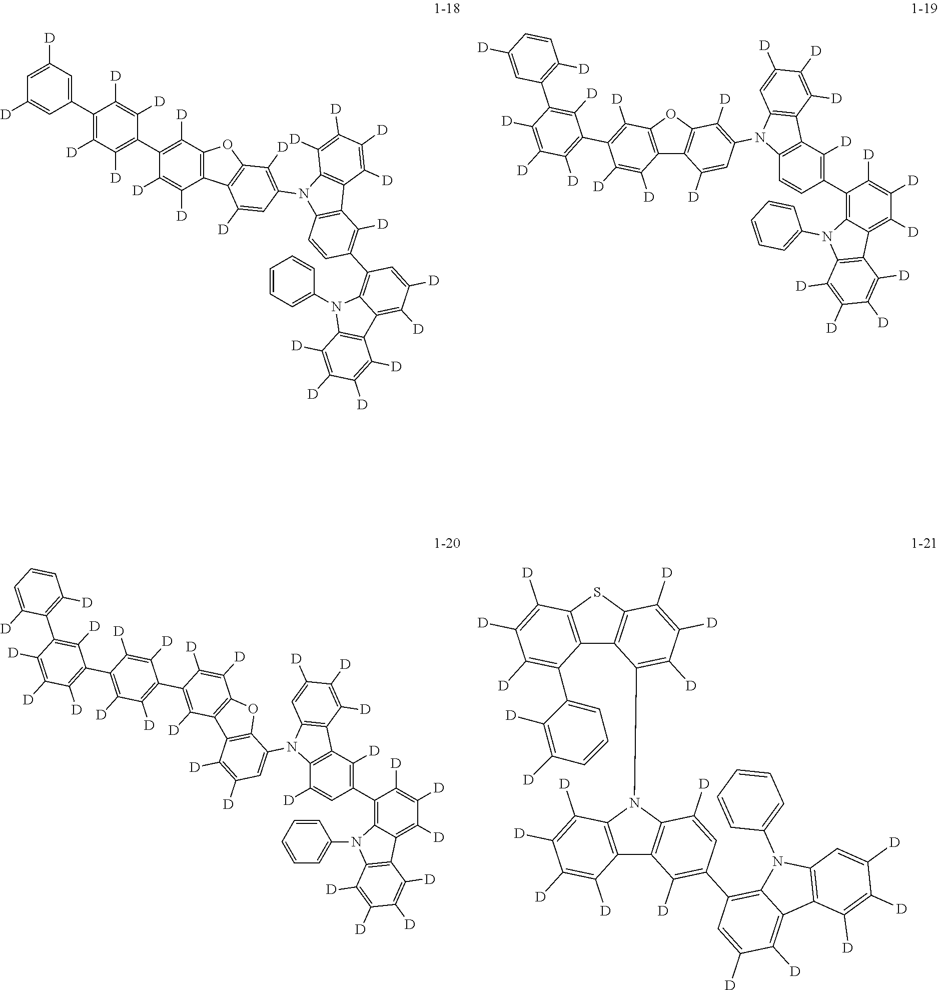

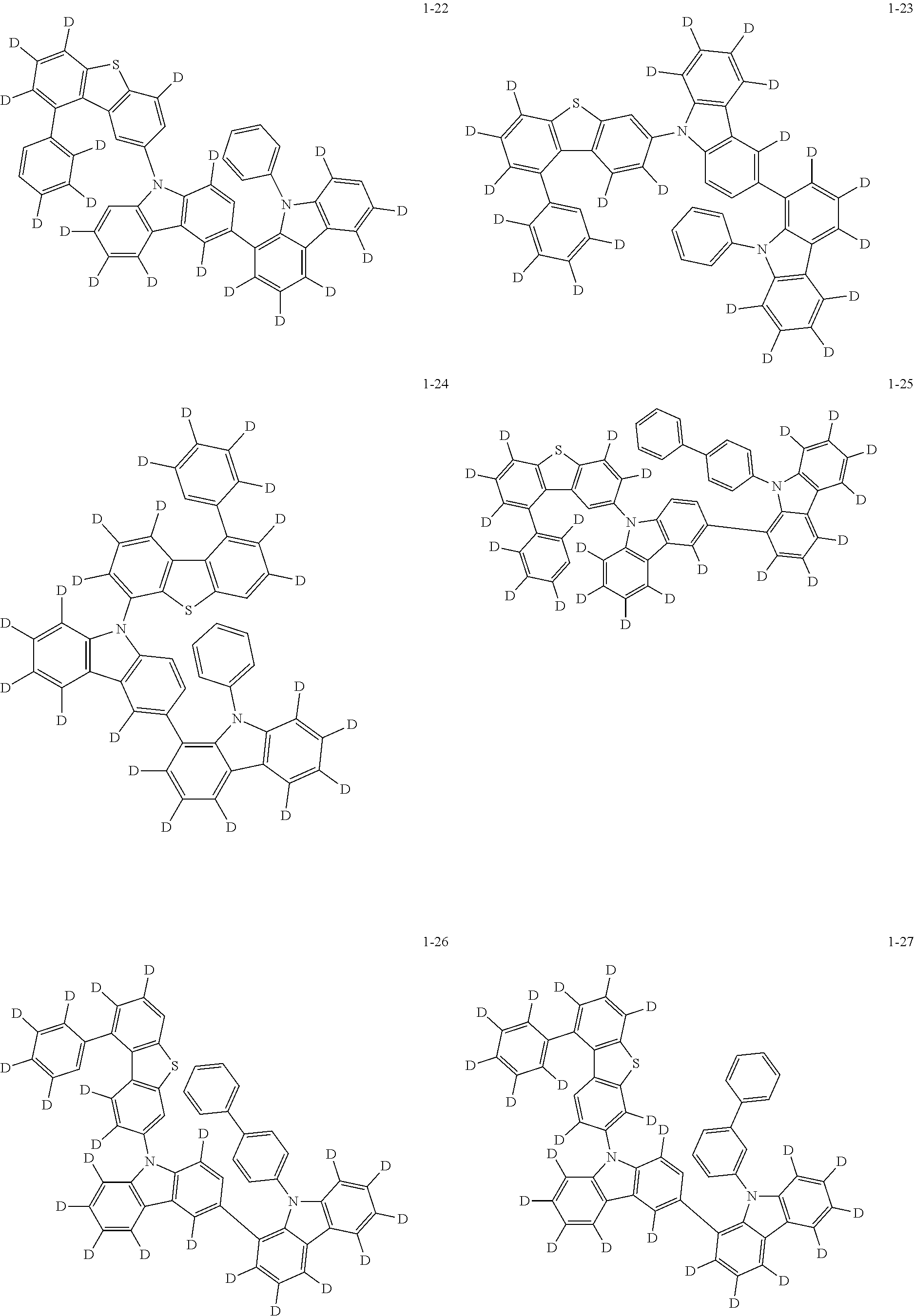

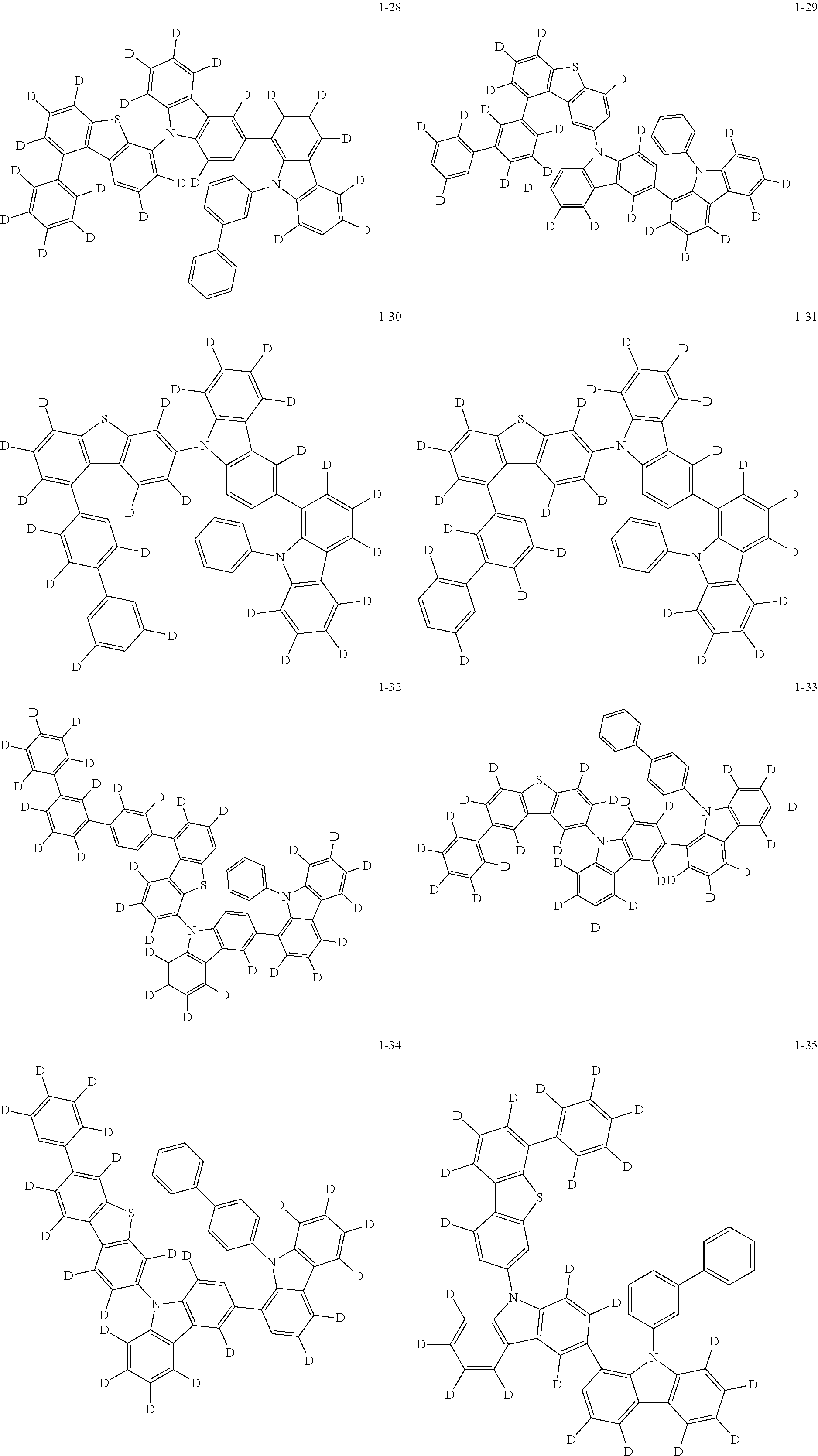

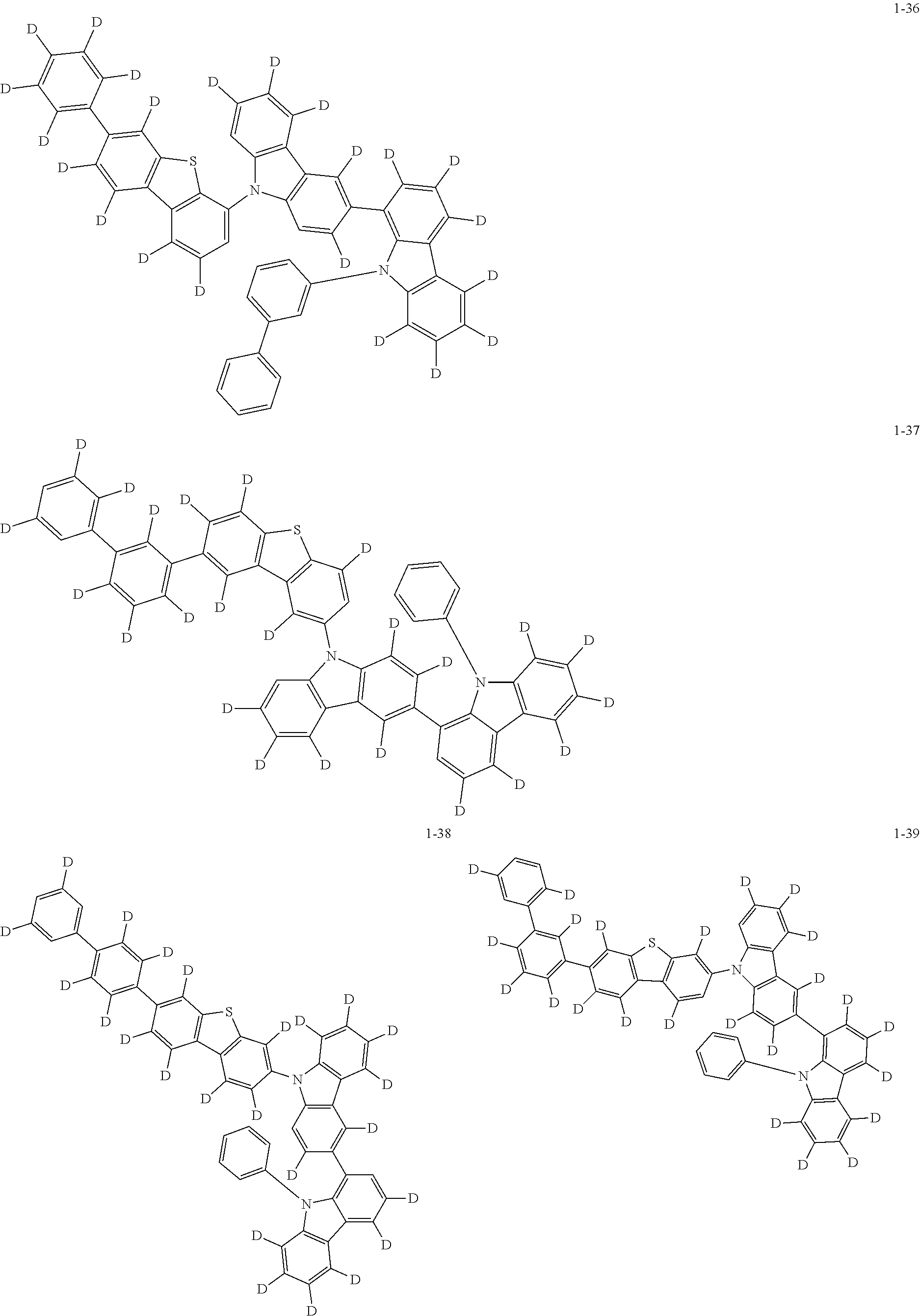

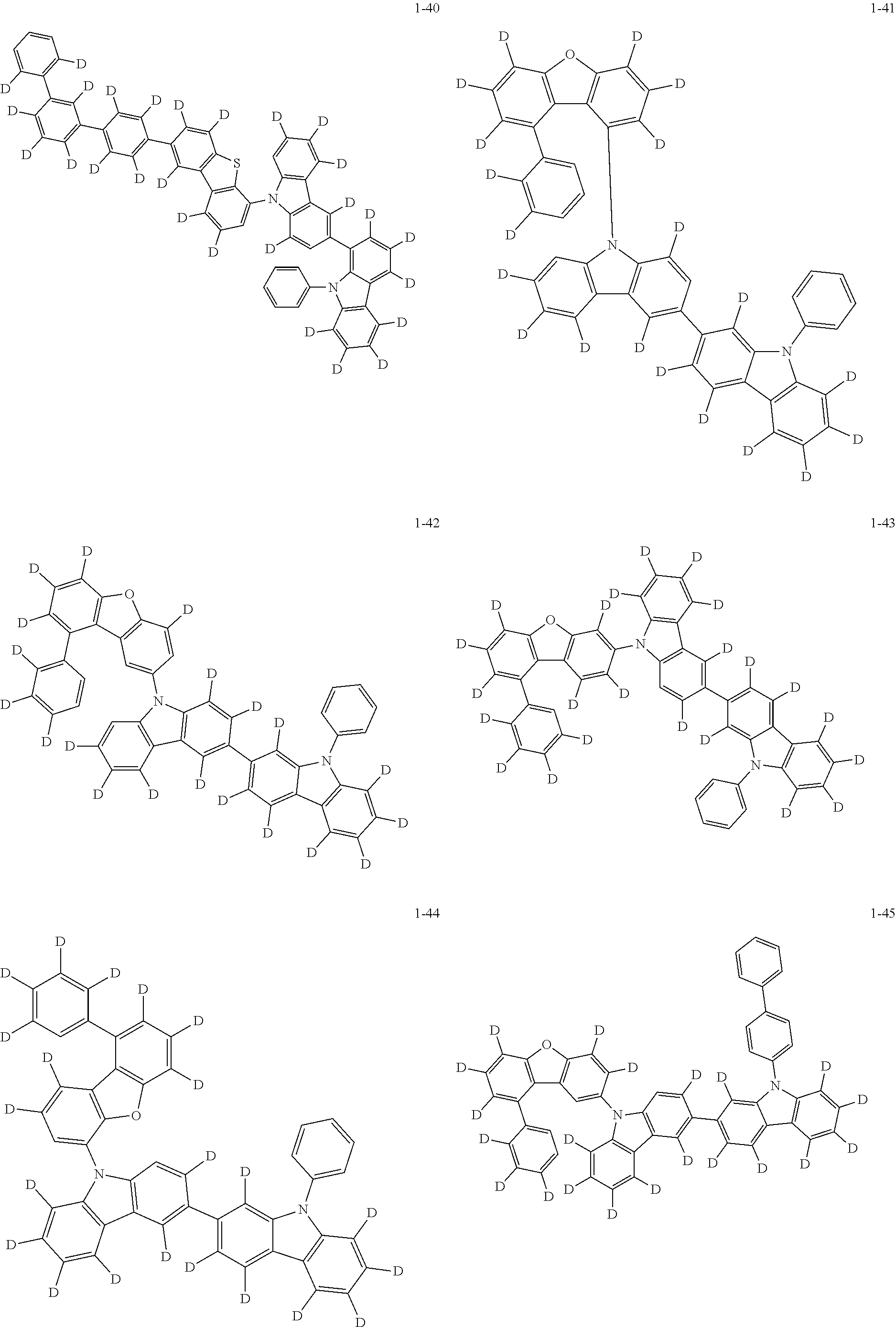

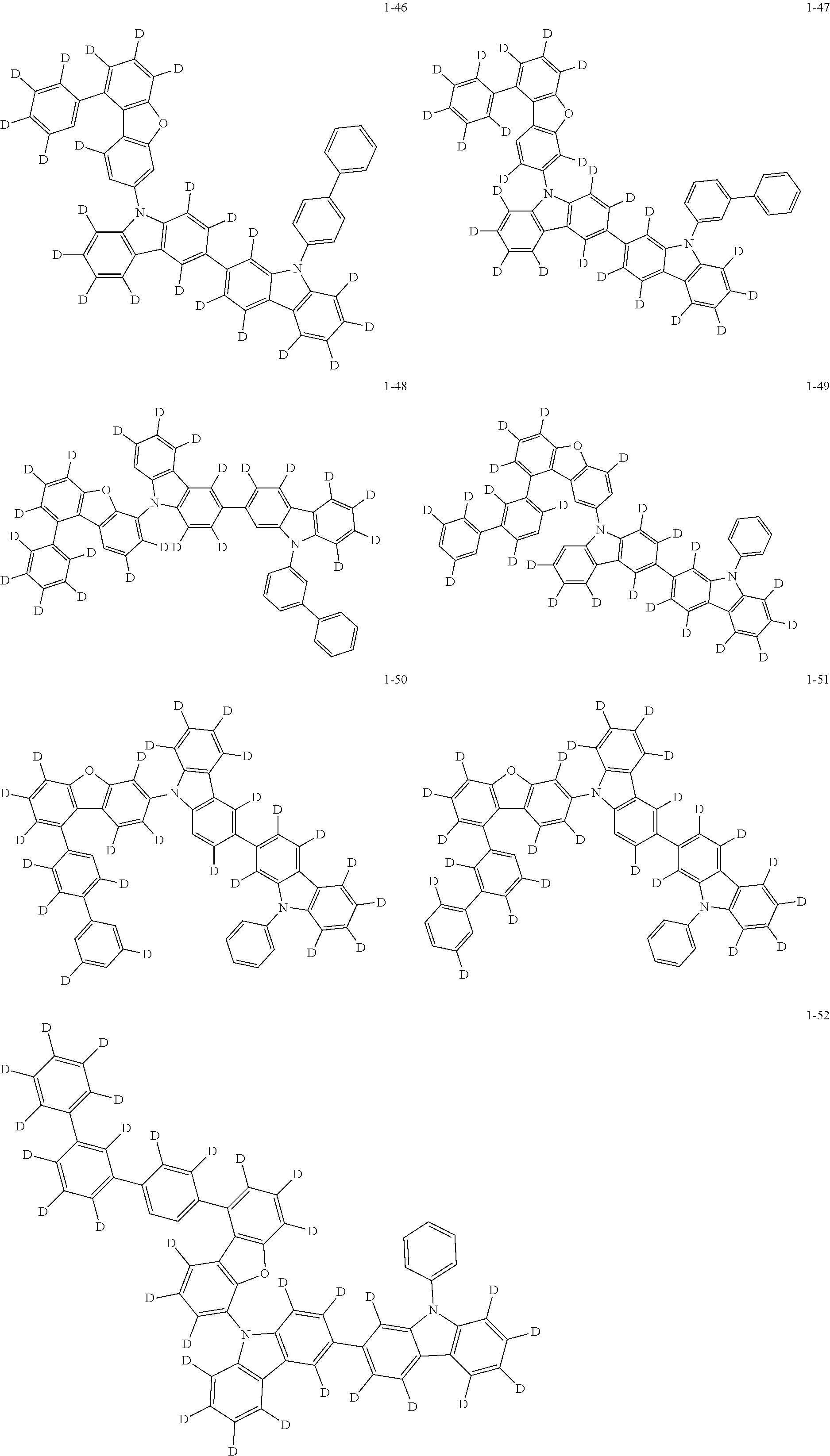

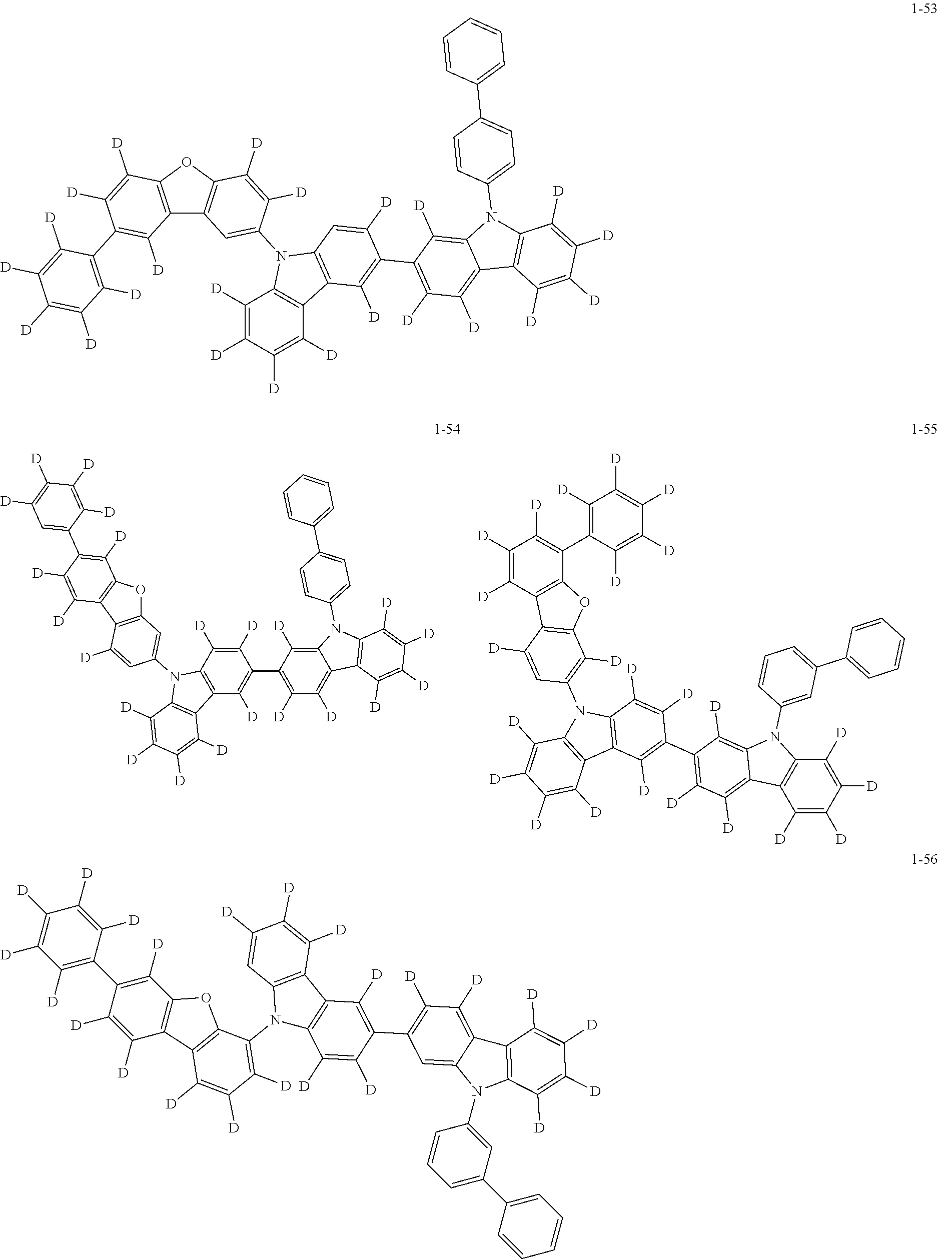

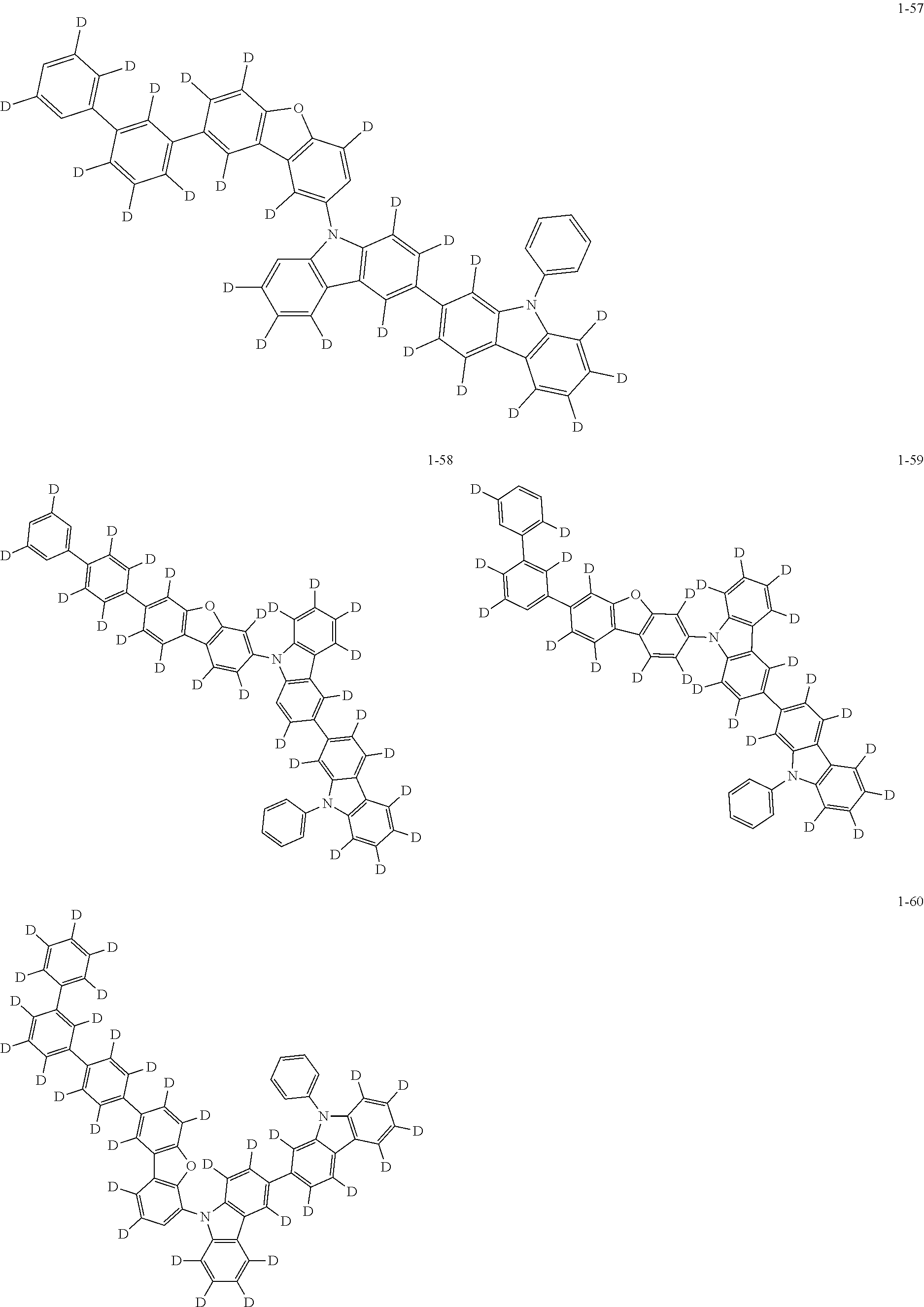

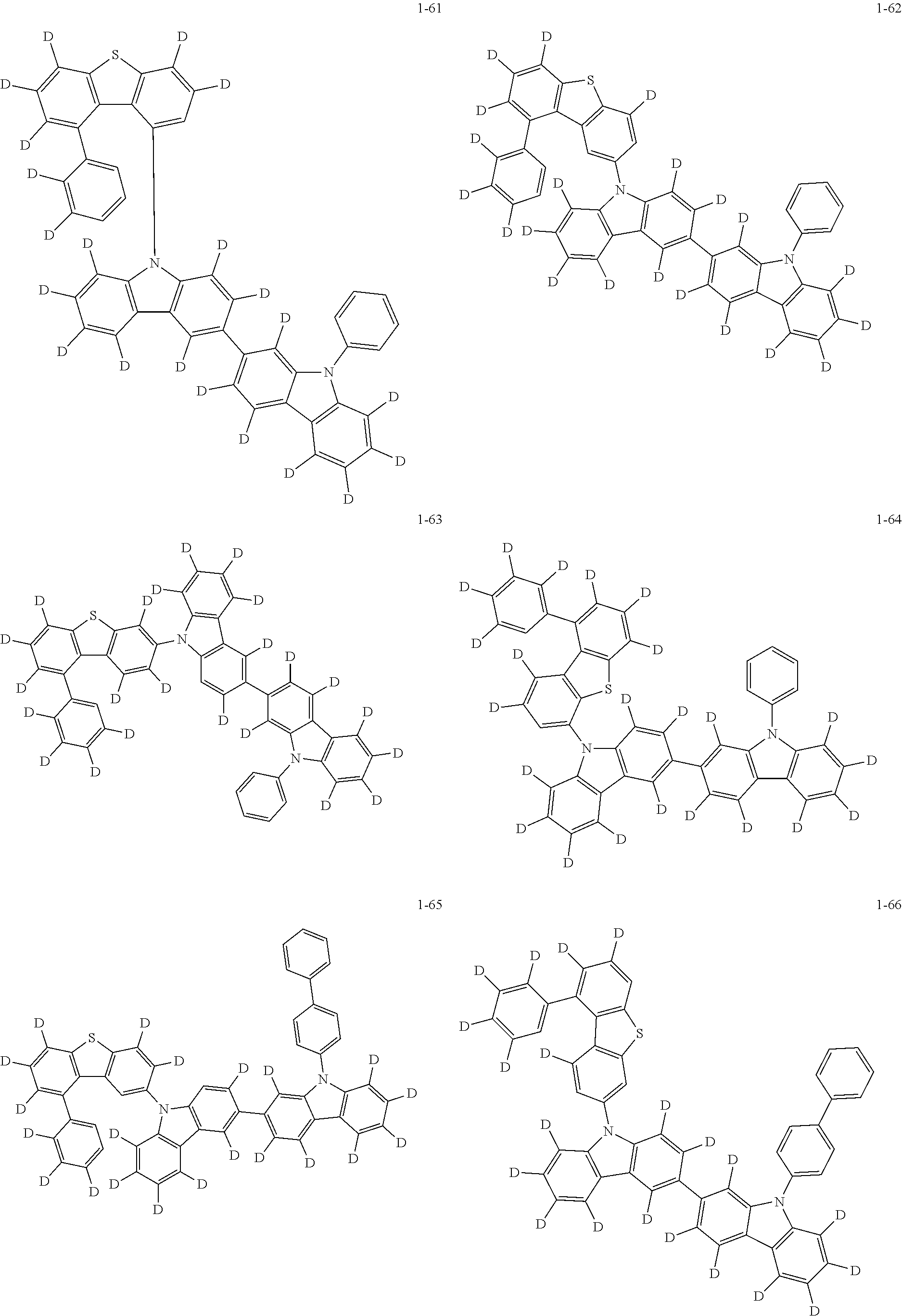

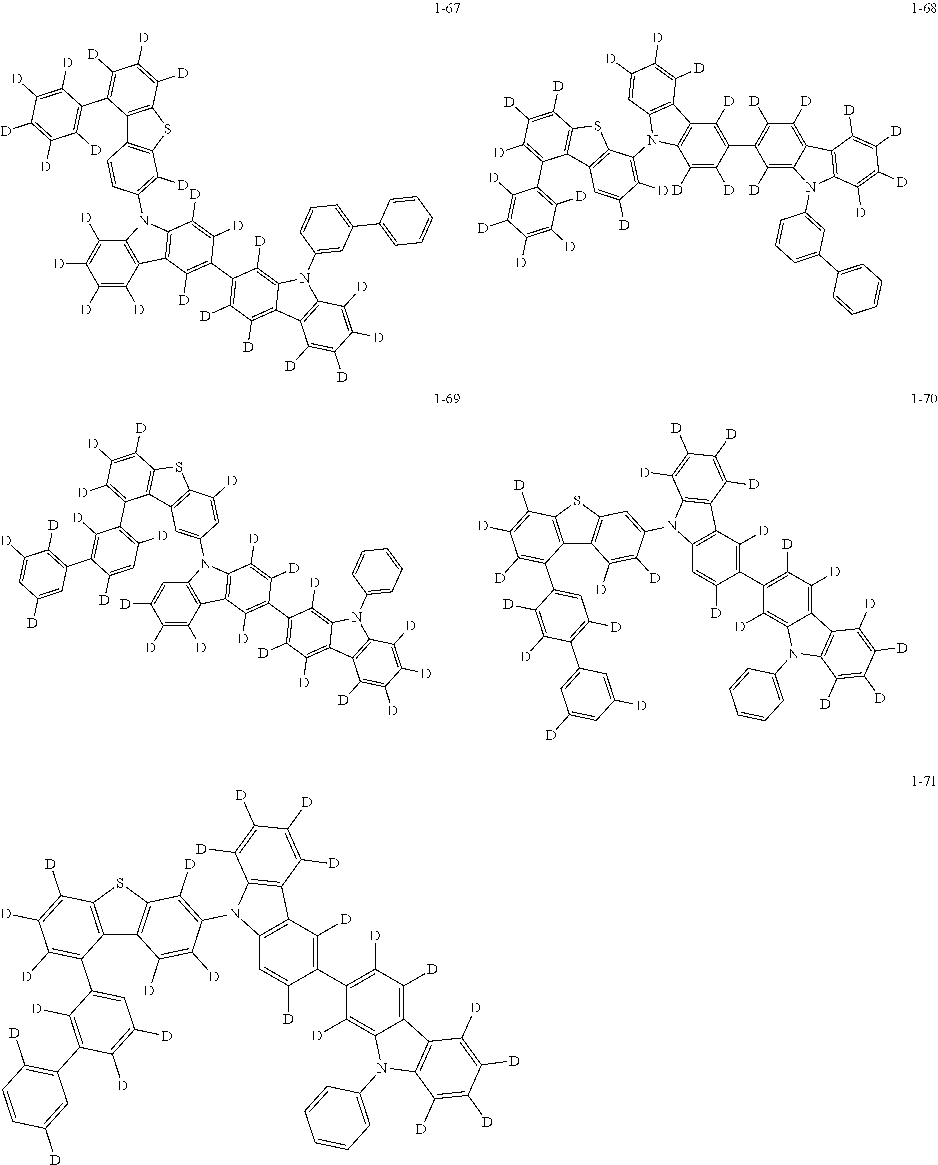

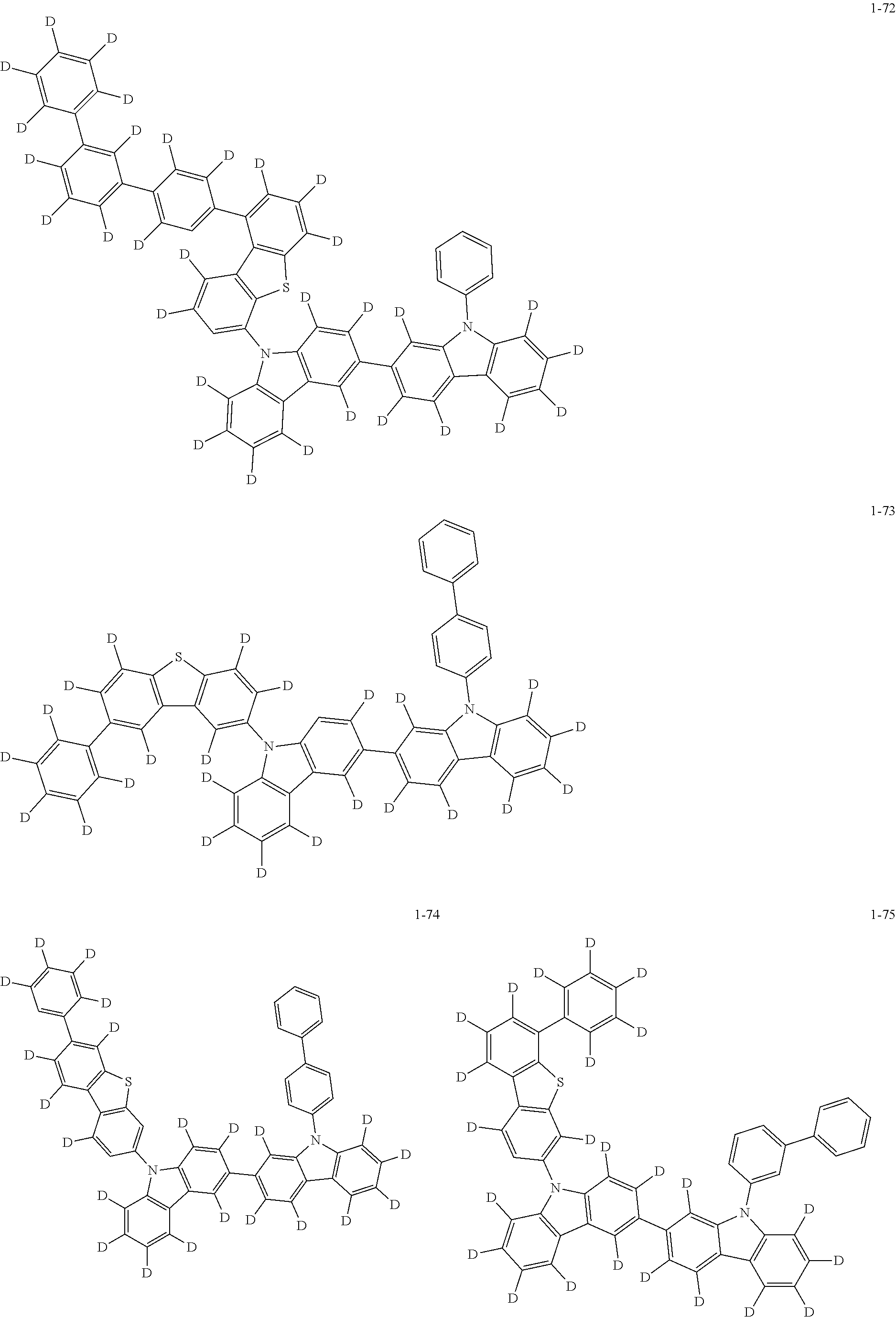

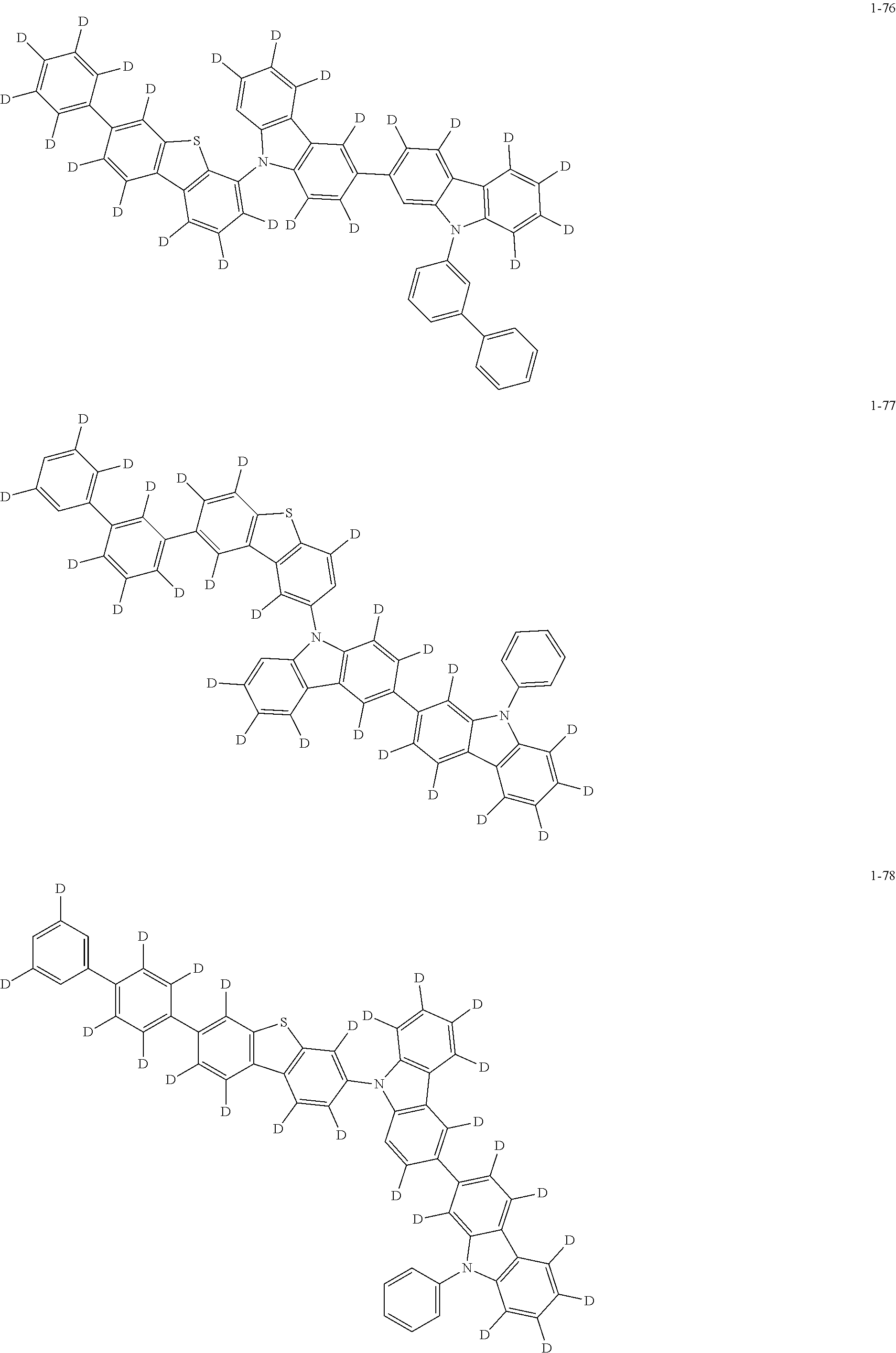

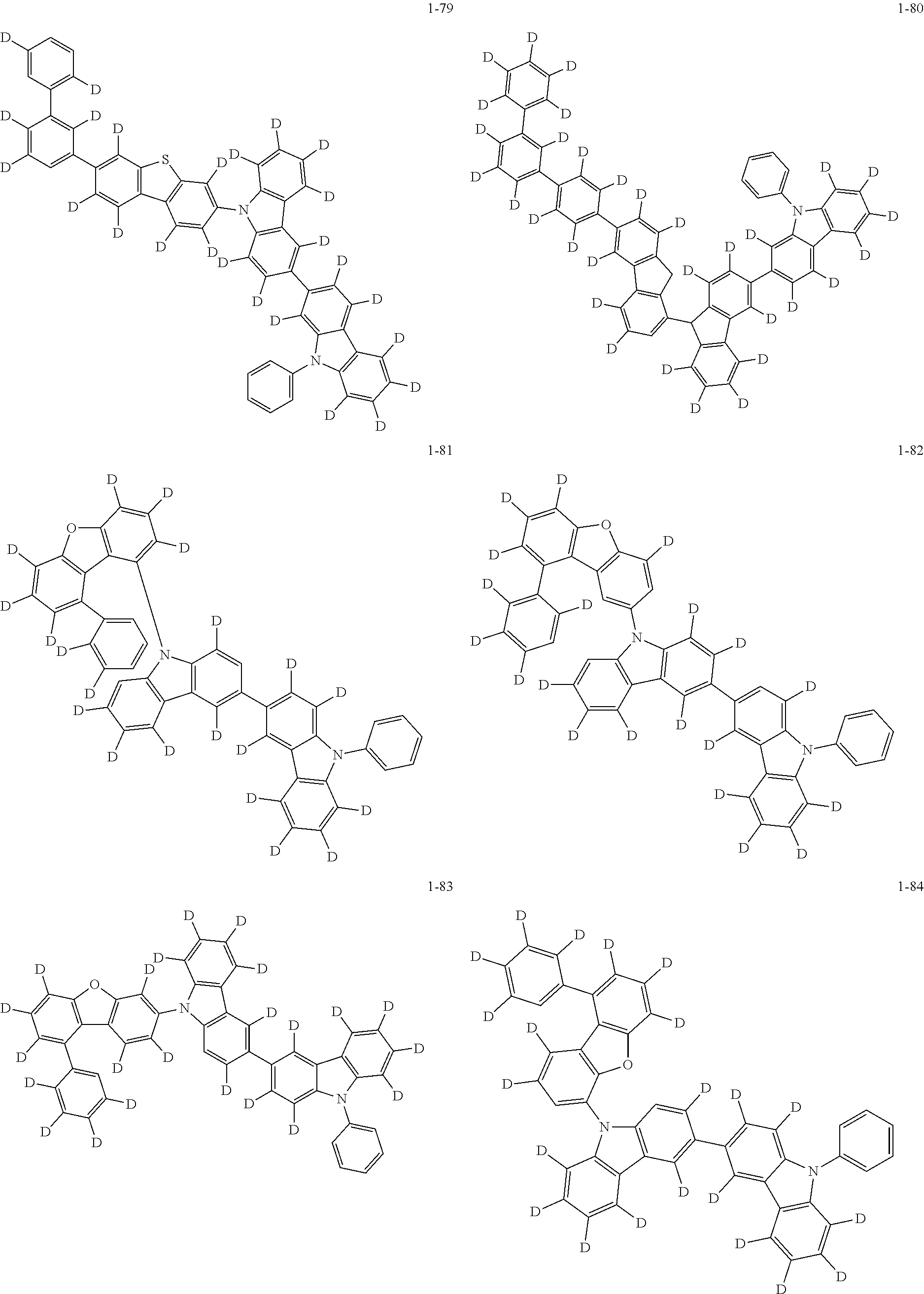

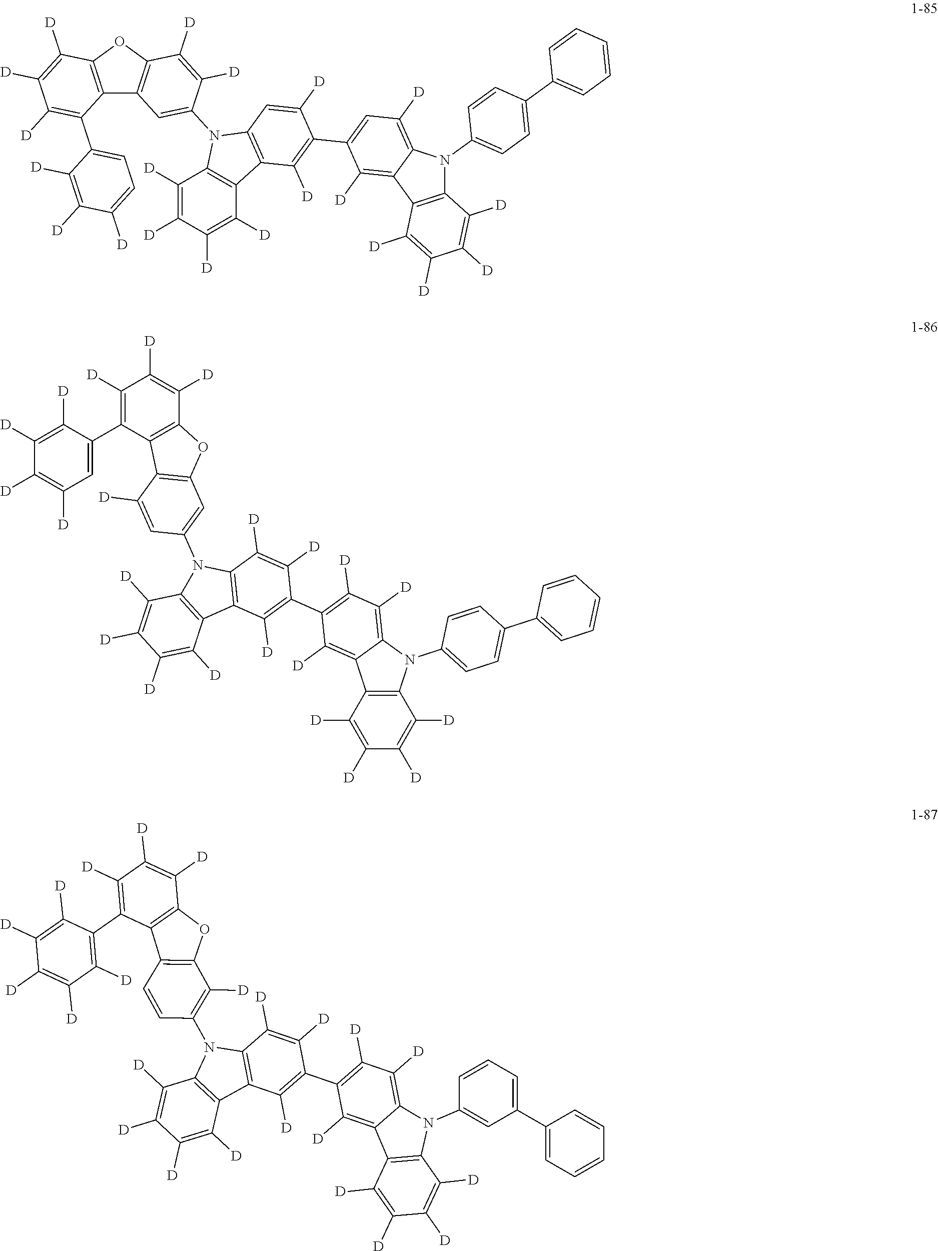

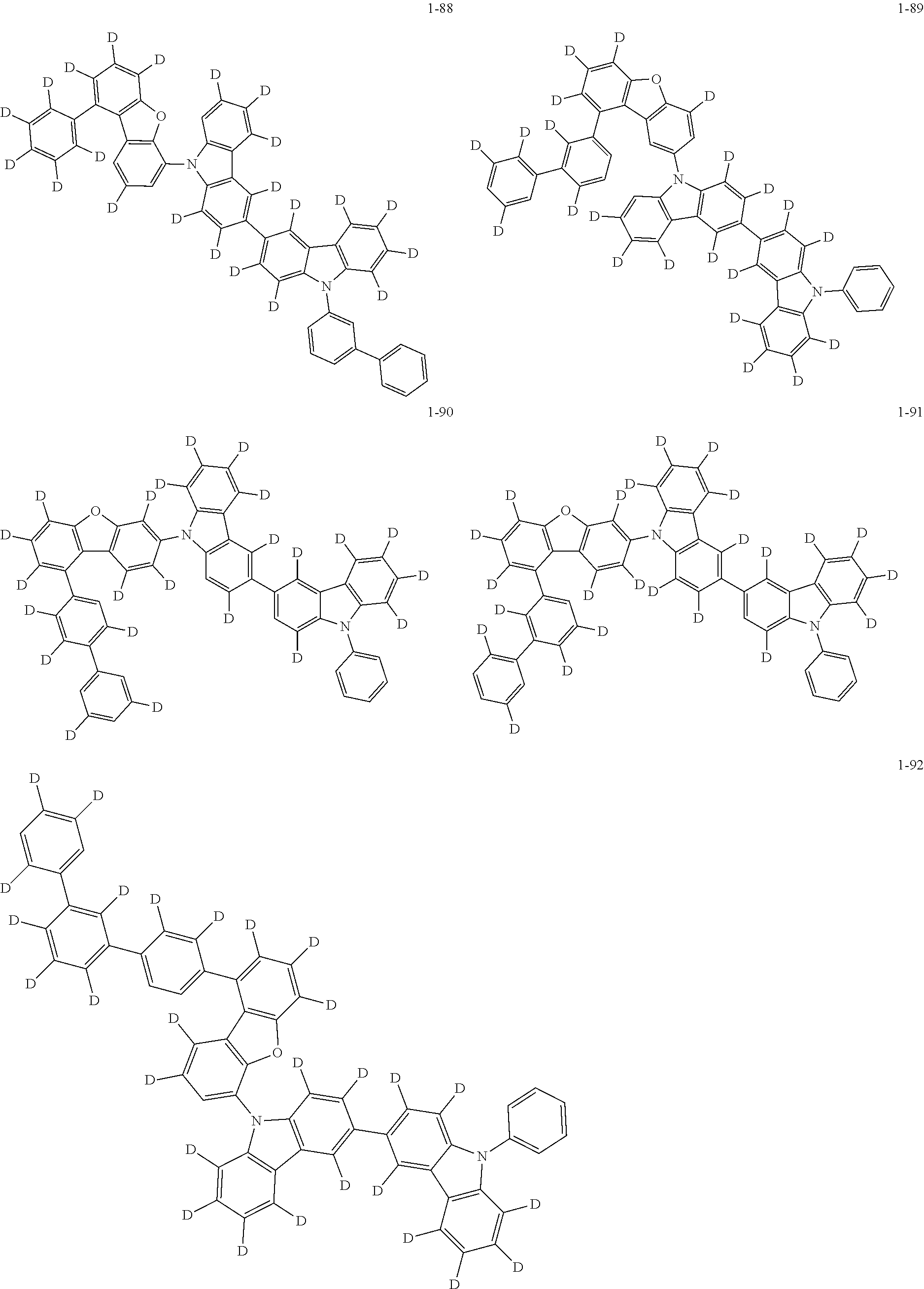

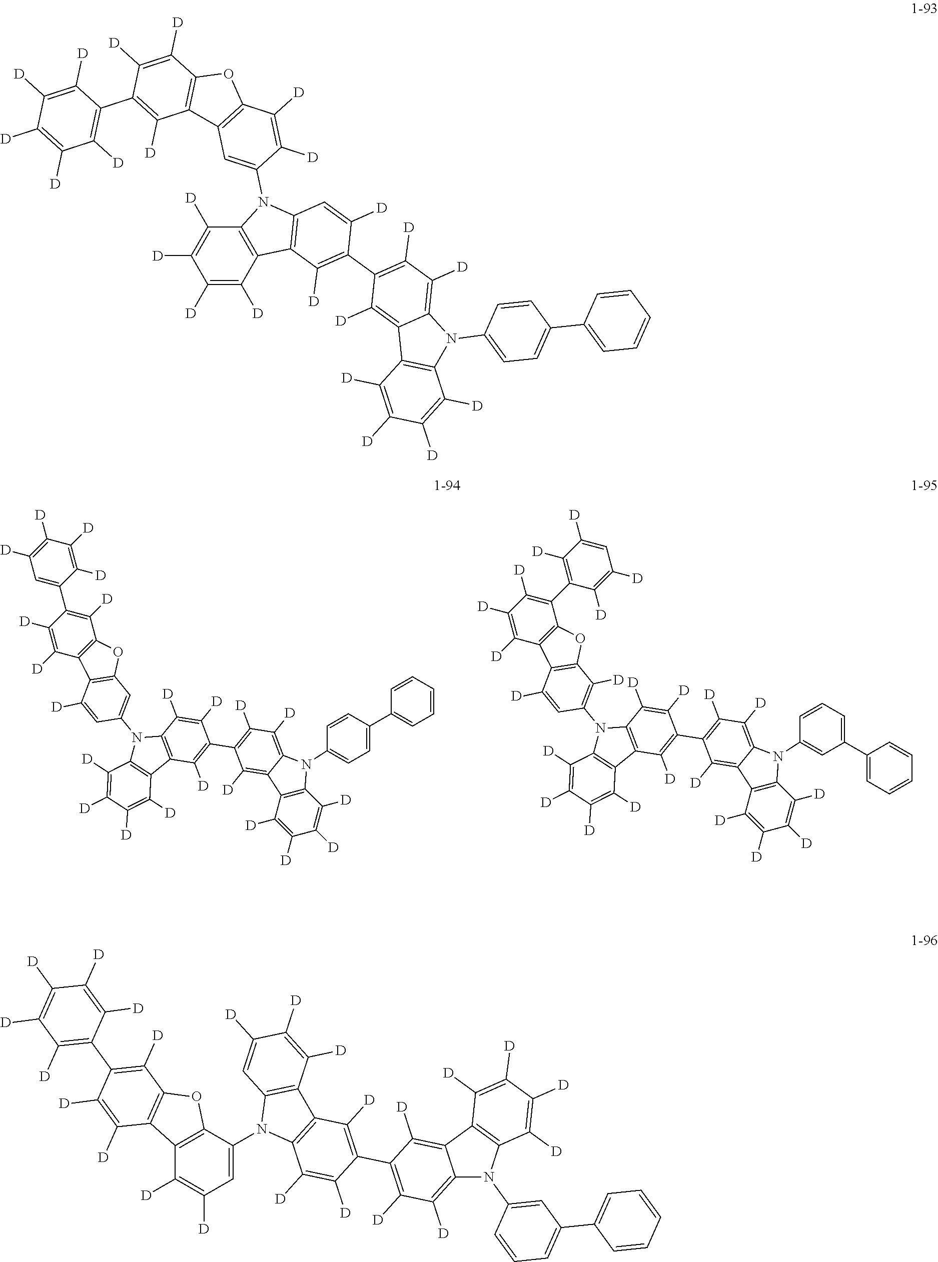

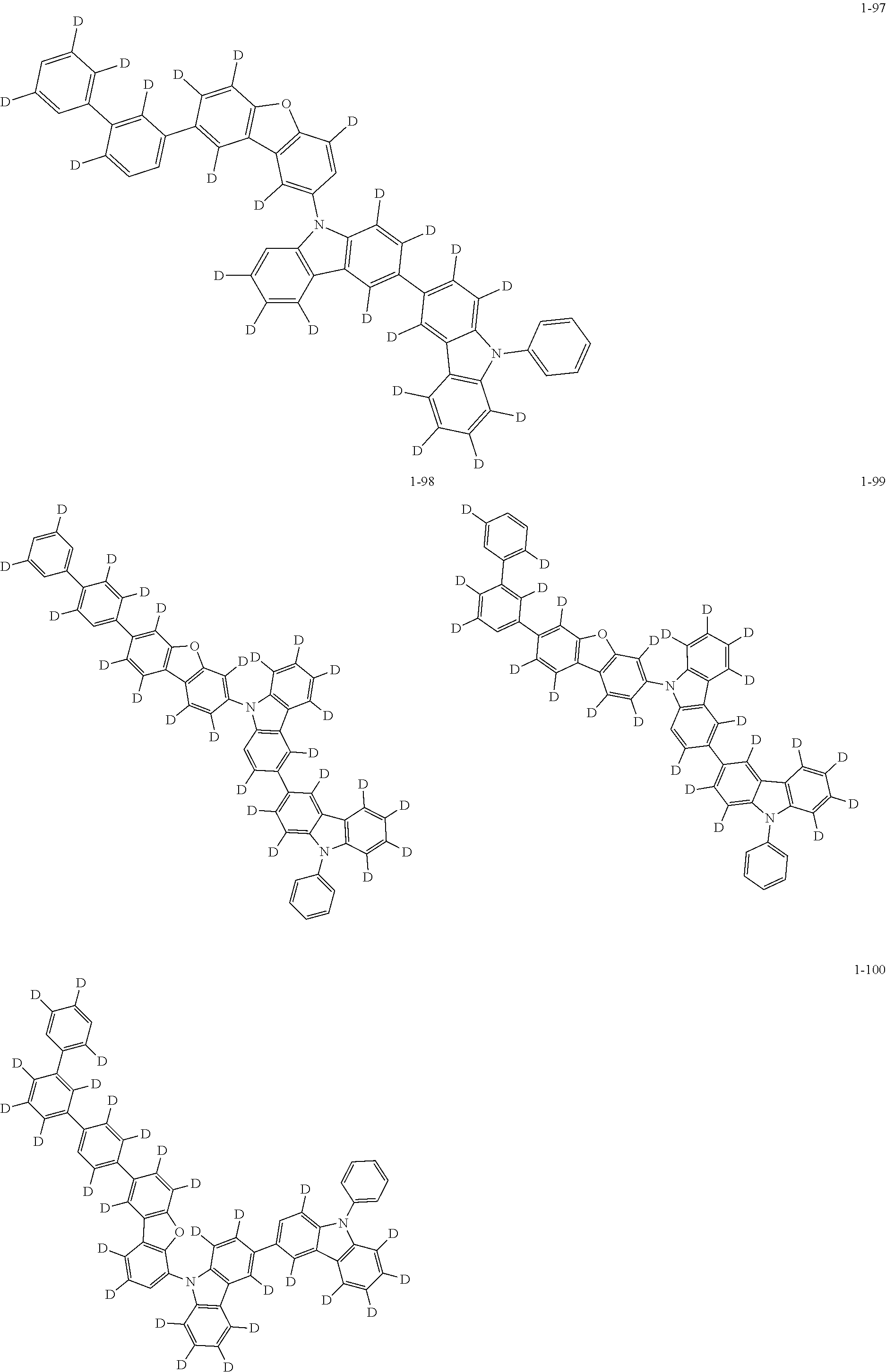

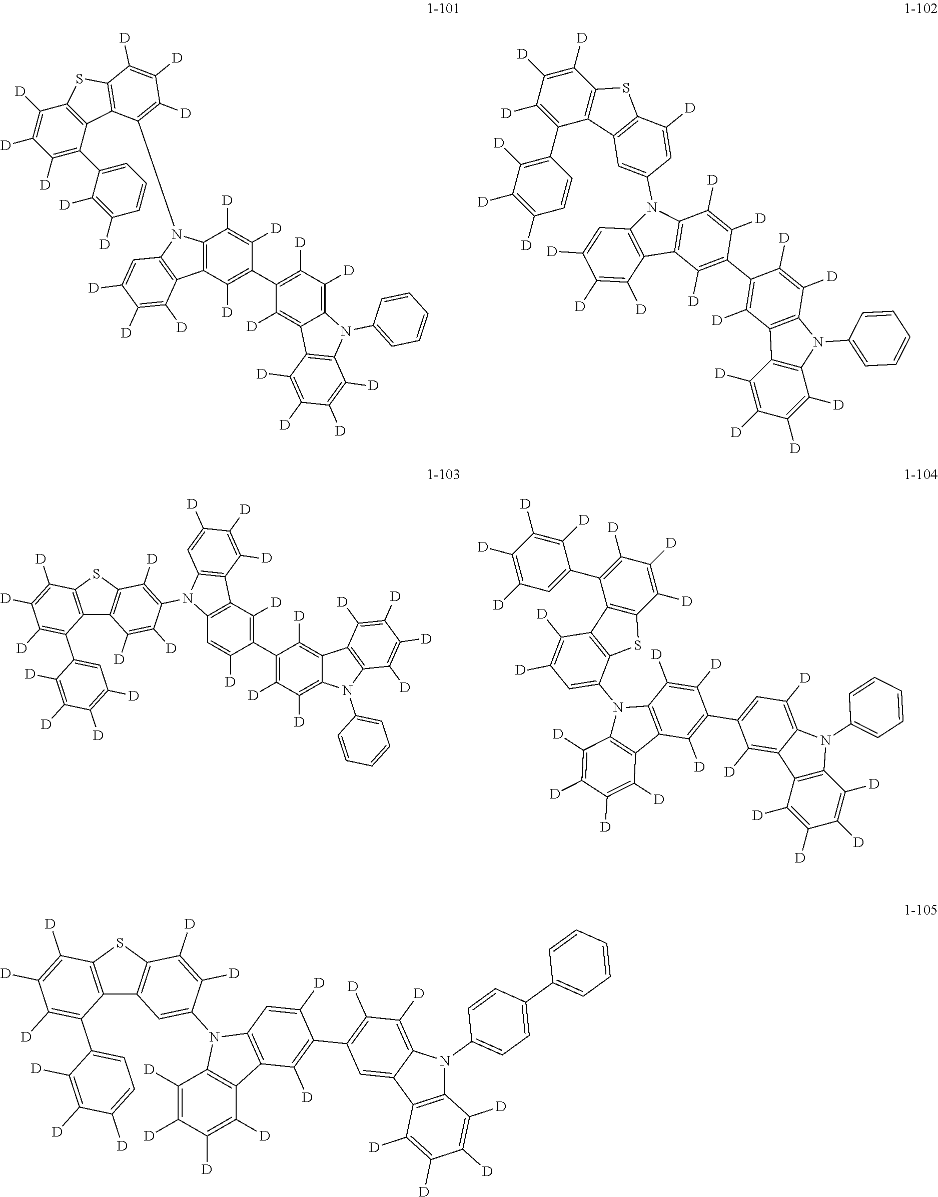

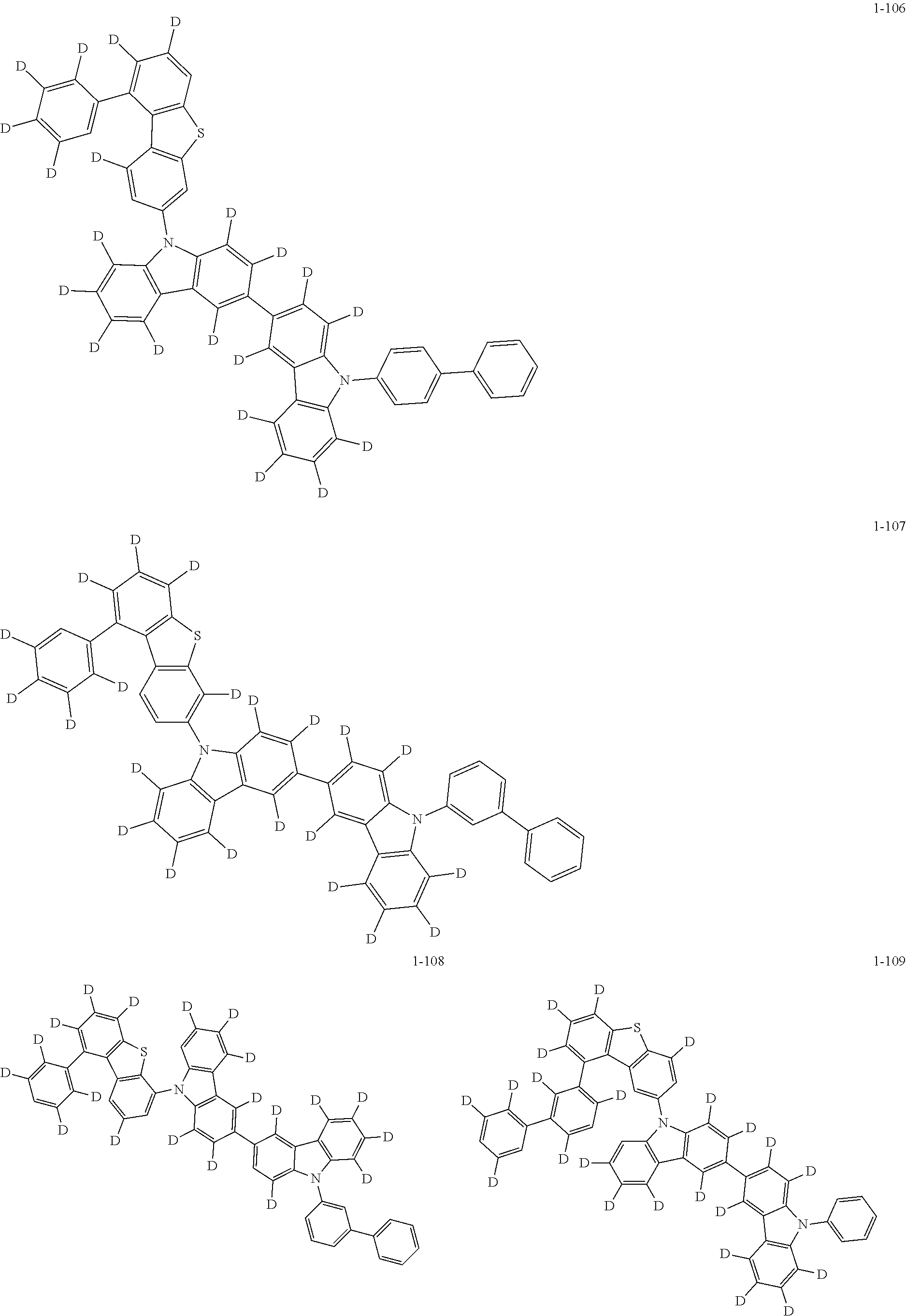

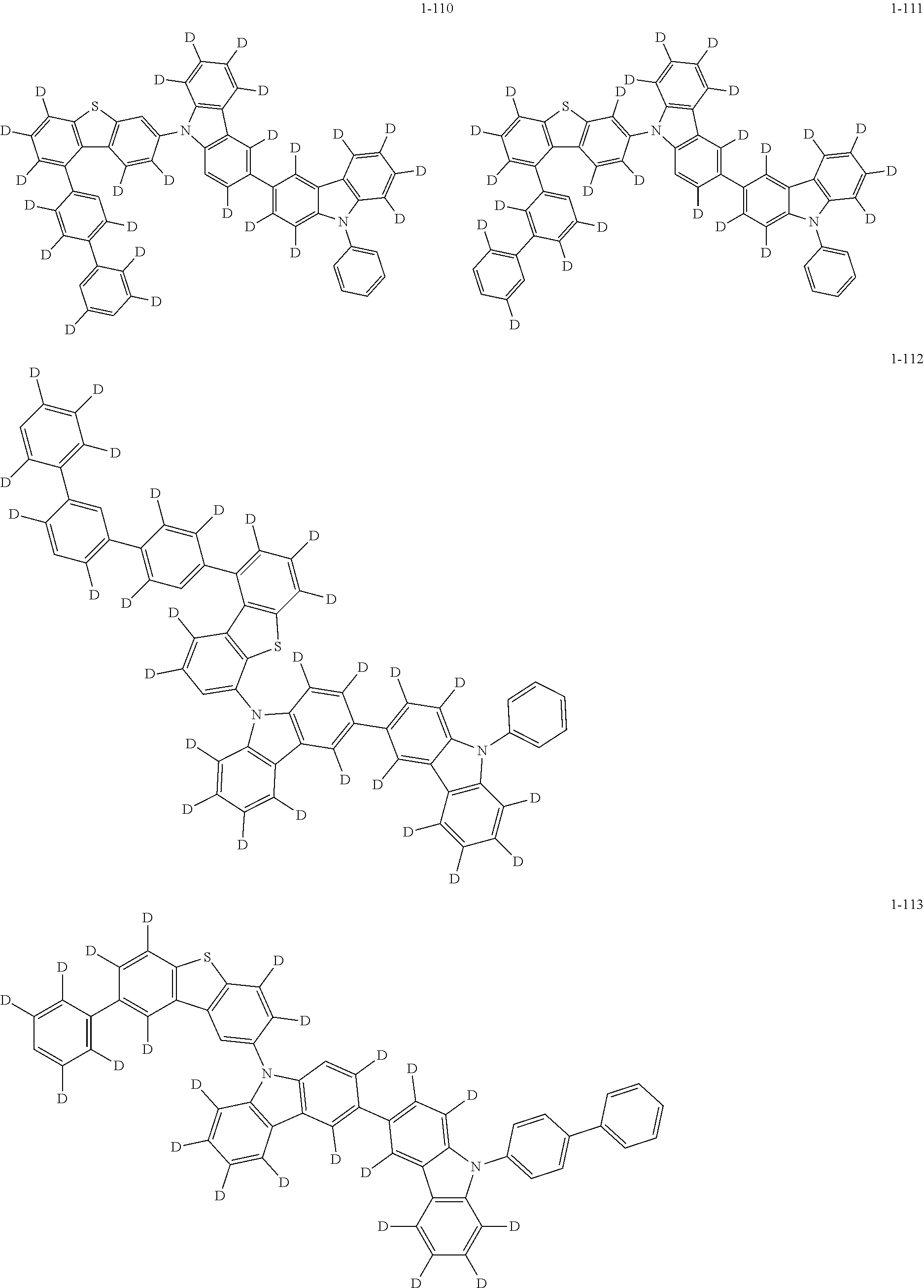

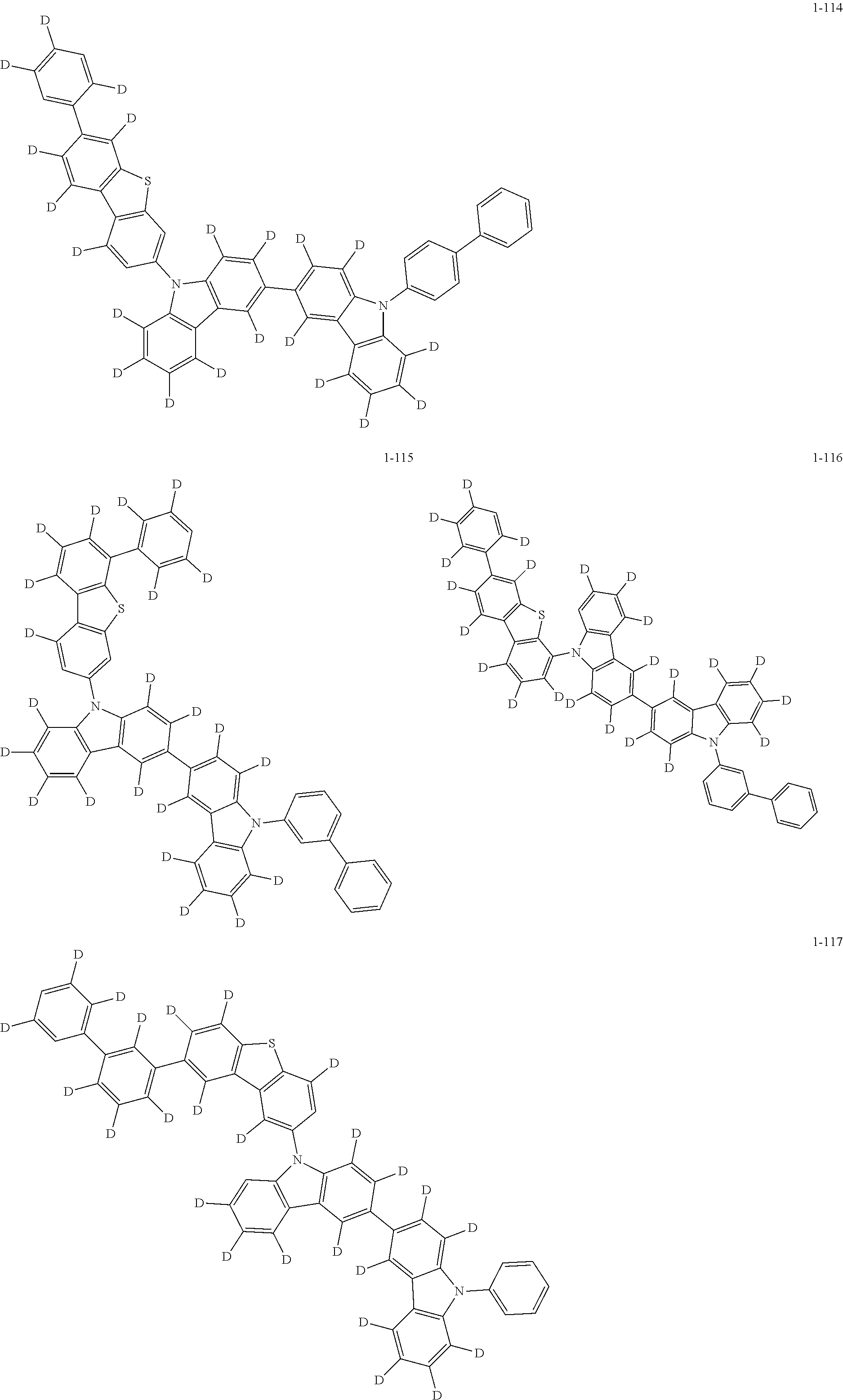

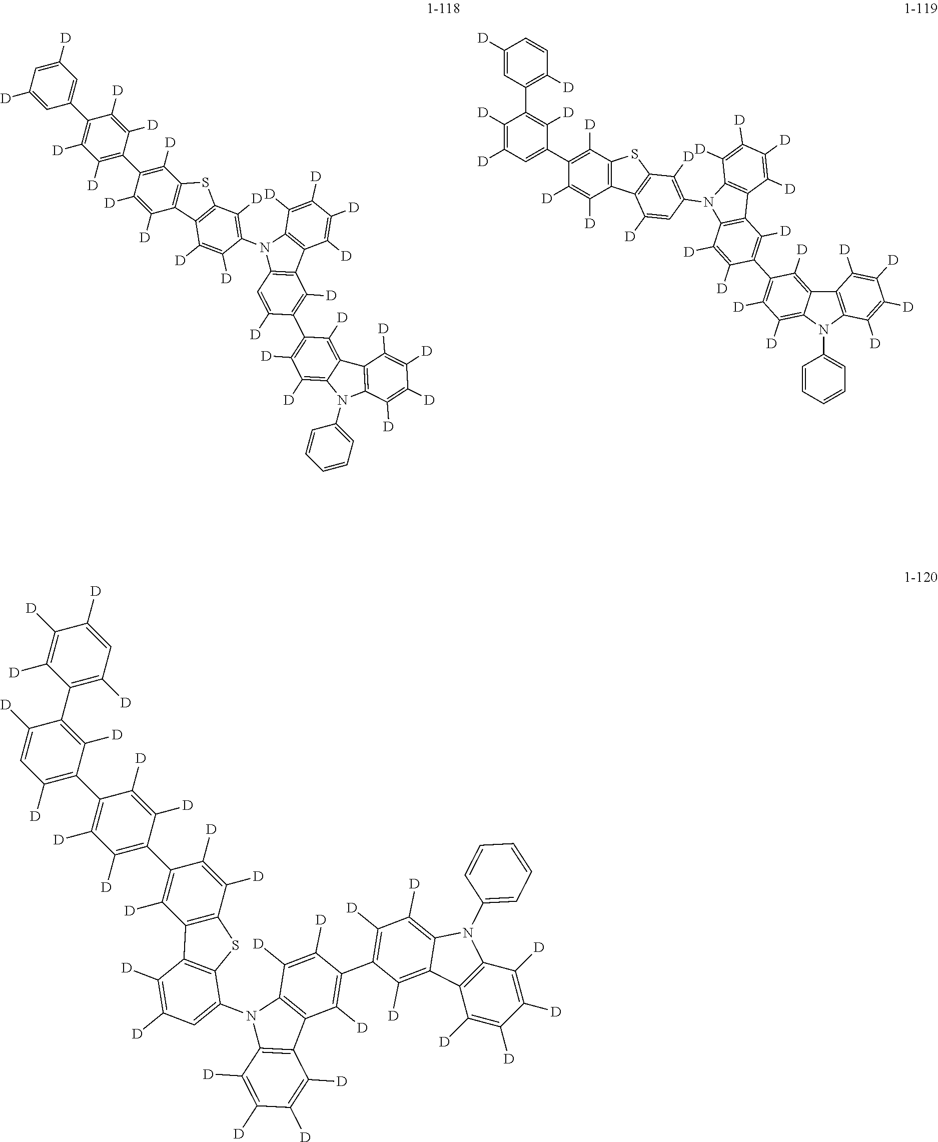

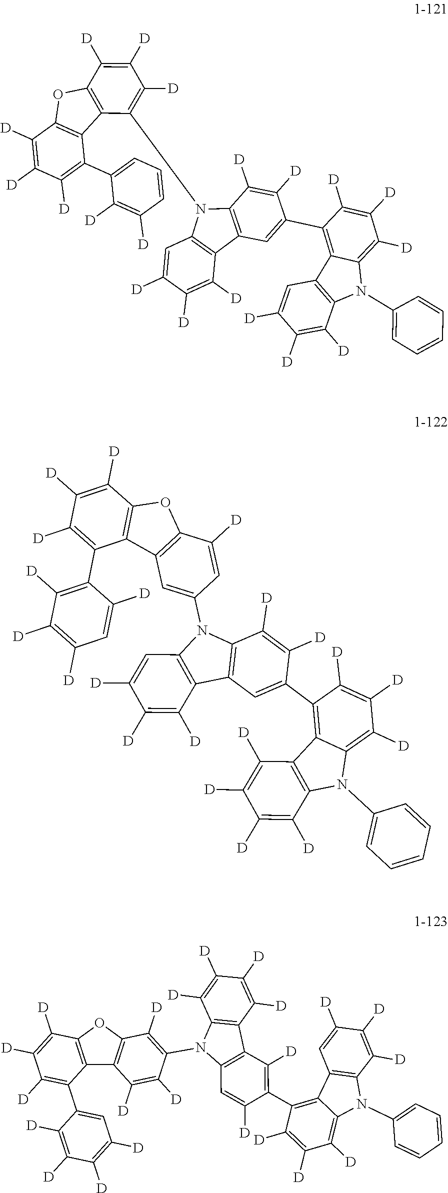

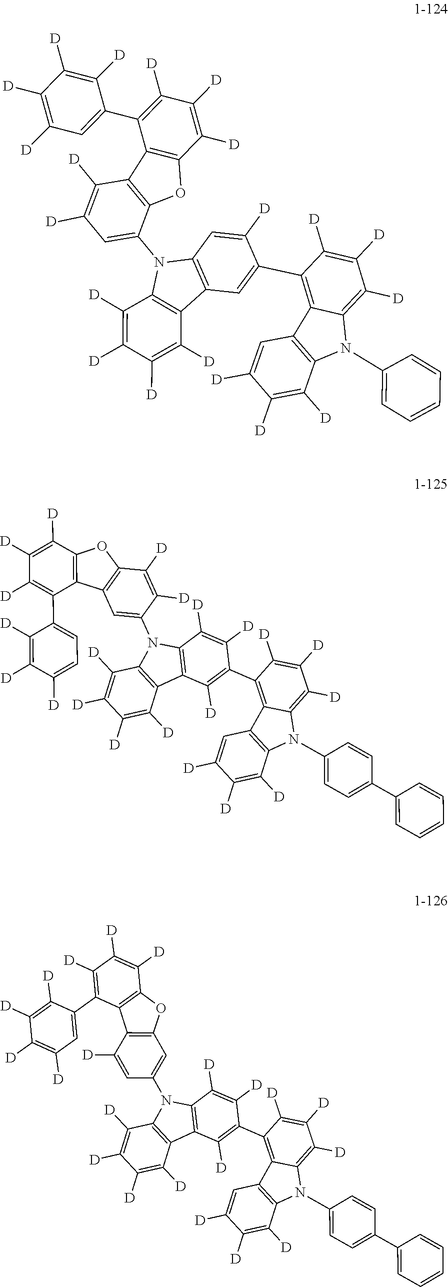

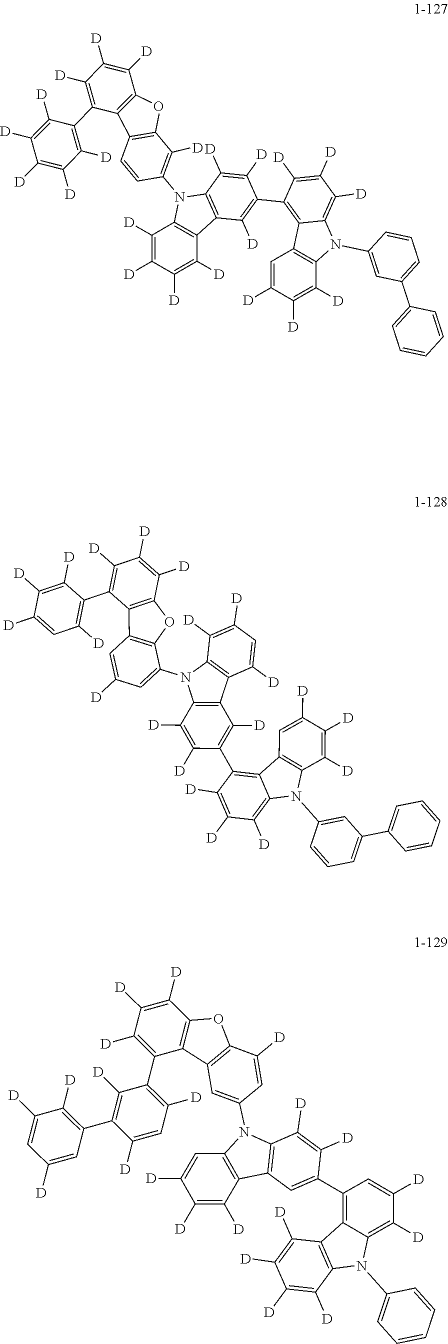

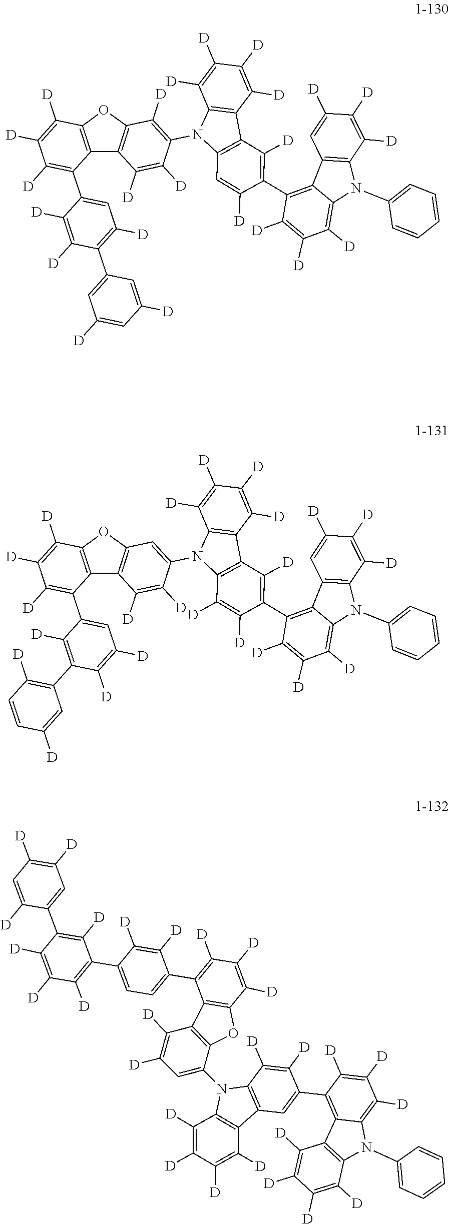

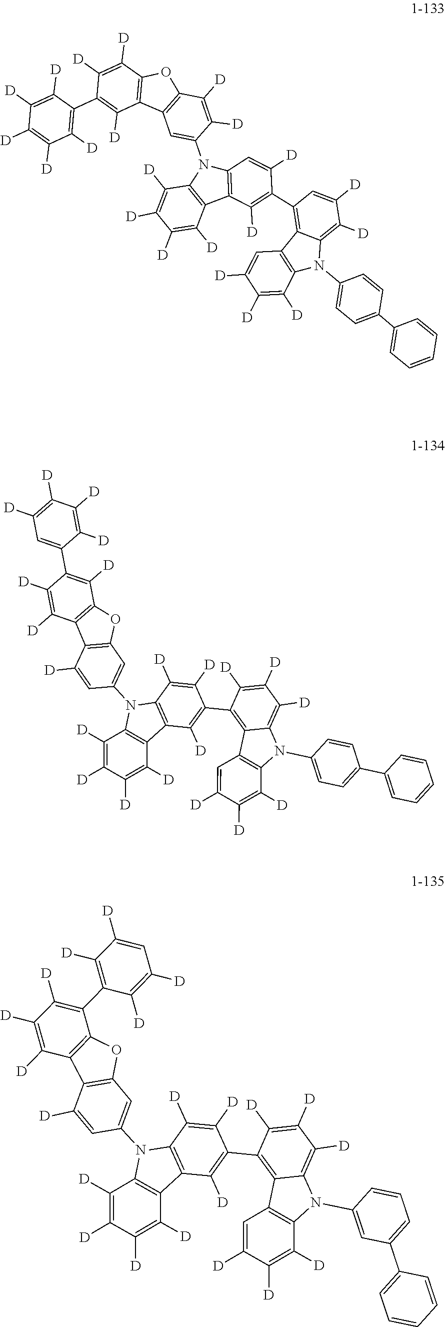

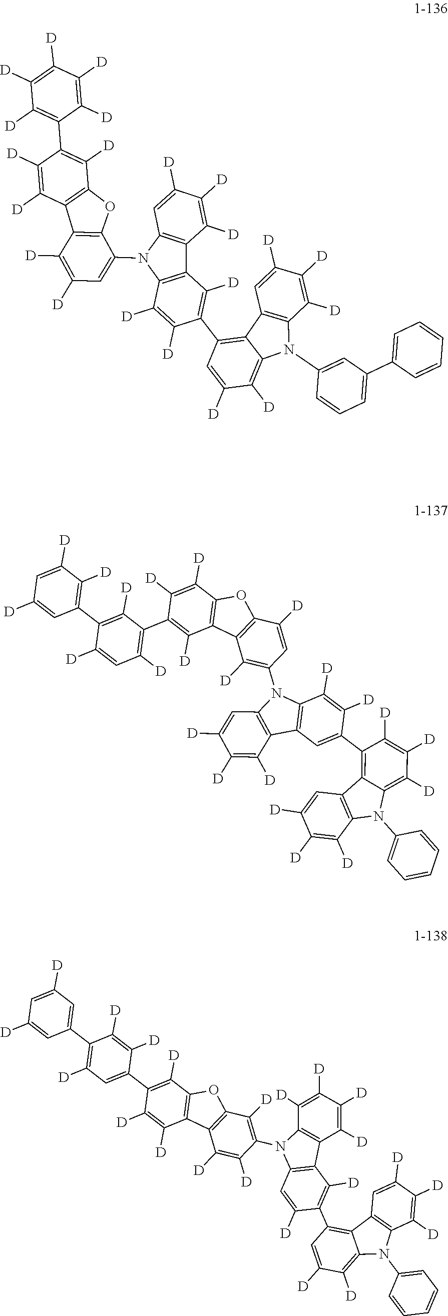

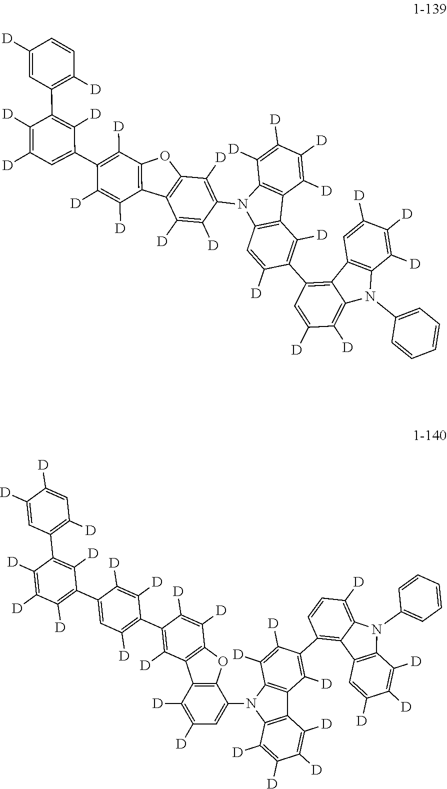 [SMILES: [2H]c1ccc([2H])c(-c2c([2H])c([2H])cc(-c3c([2H])c([2H])c4c(oc5c([2H])c(-n6c7cc([2H])c(-c8cc([2H])c([2H])c9c8c8cc([2H])c([2H])c([2H])c8n9-c8ccccc8)c([2H])c7c7c([2H])c([2H])c([2H])c([2H])c76)c([2H])c([2H])c54)c3[2H])c2[2H])c1 ; [2H]c1cc([2H])c(-c2c([2H])c([2H])c([2H])c(-c3cc([2H])c(-c4c([2H])c([2H])c5oc6c(-n7c8c([2H])c([2H])c([2H])c([2H])c8c8c([2H])c(-c9ccc([2H])c%10c9c9cc([2H])c([2H])c([2H])c9n%10-c9ccccc9)c([2H])c([2H])c87)cc([2H])c([2H])c6c5c4[2H])c([2H])c3[2H])c2[2H])cc1[2H]]

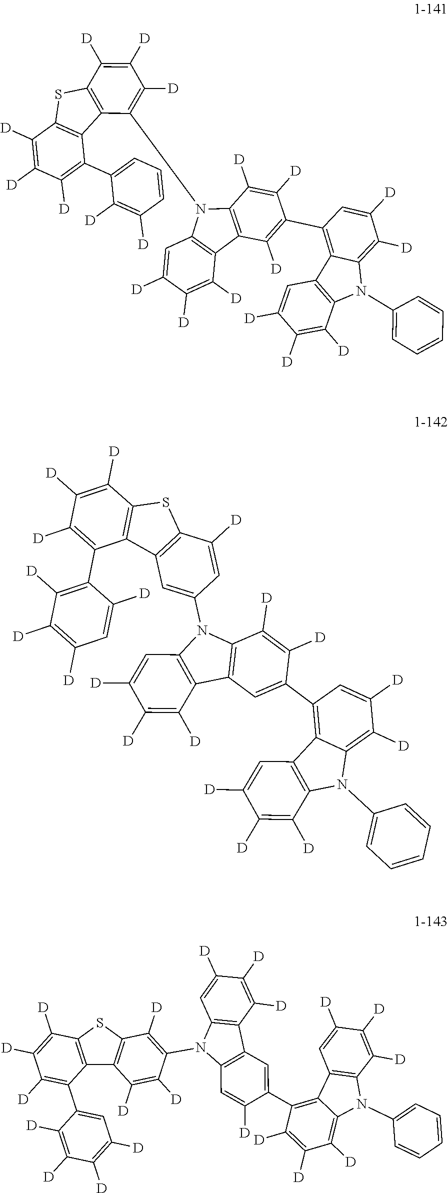

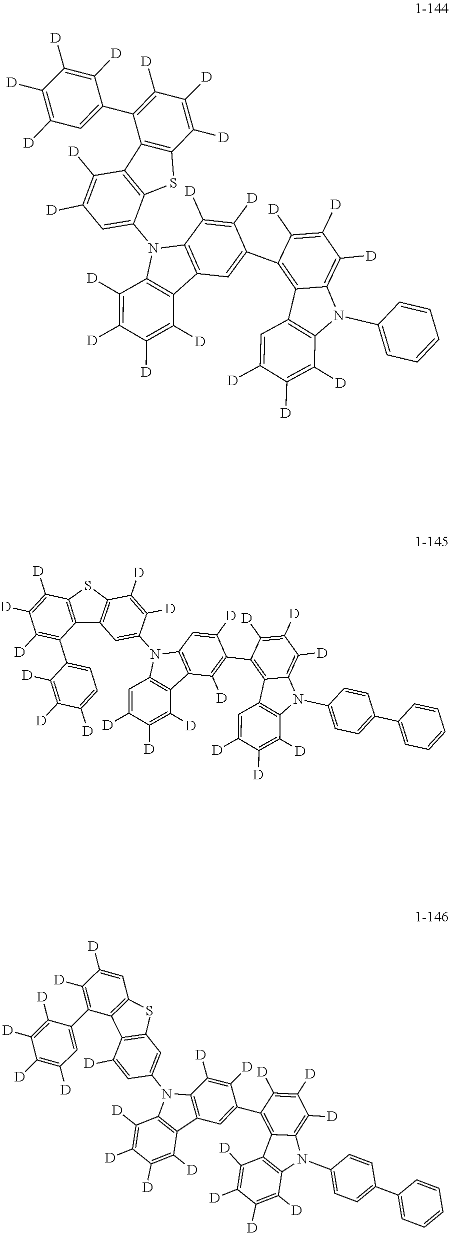

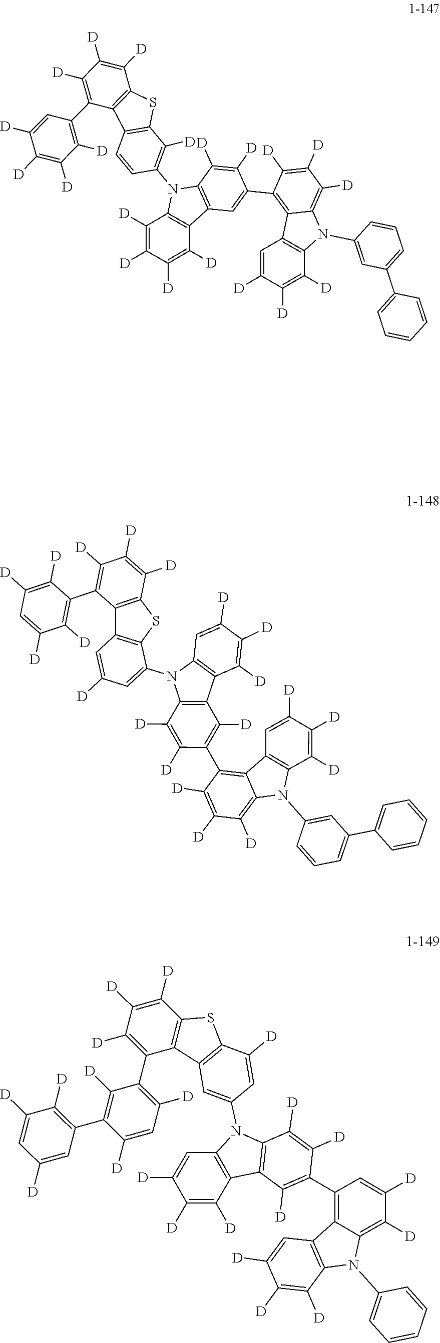

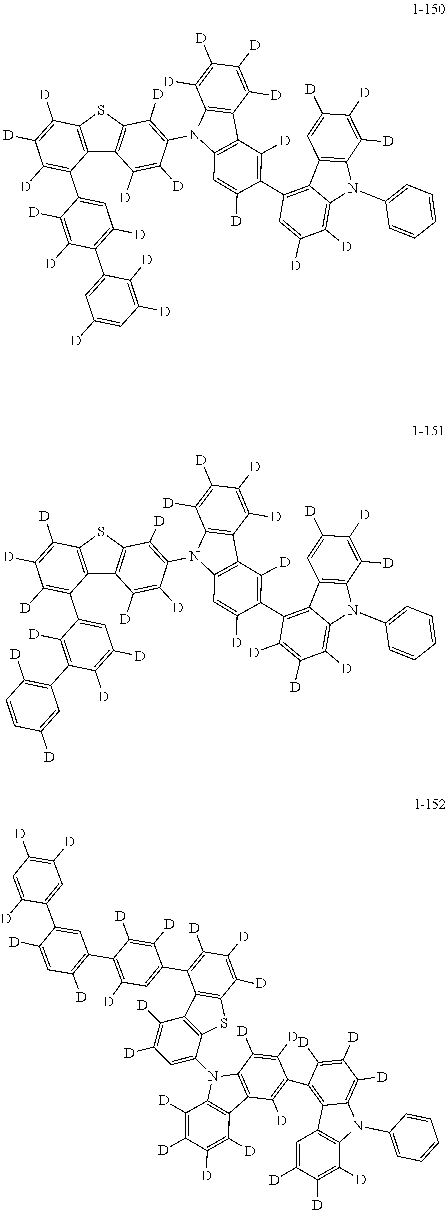

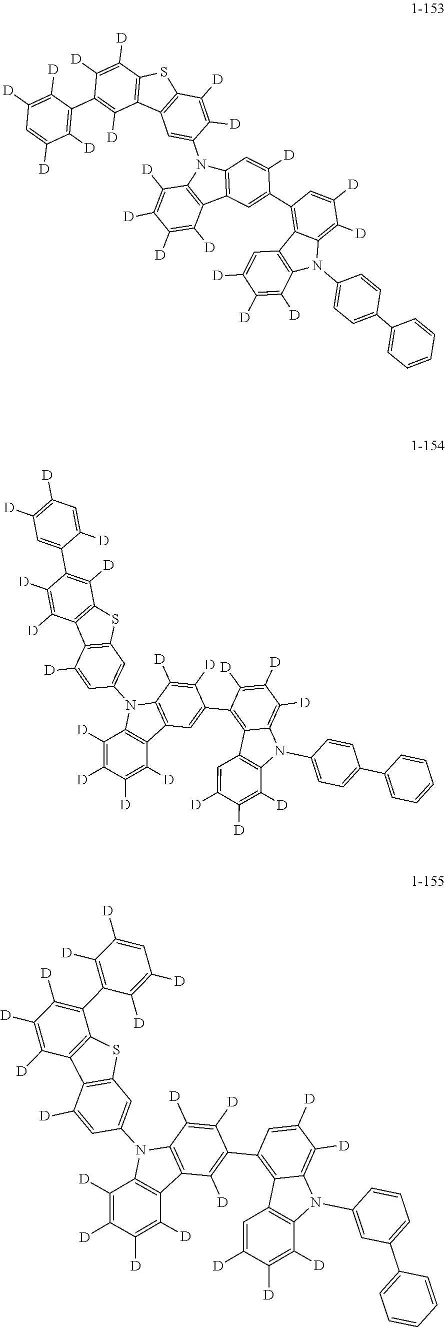

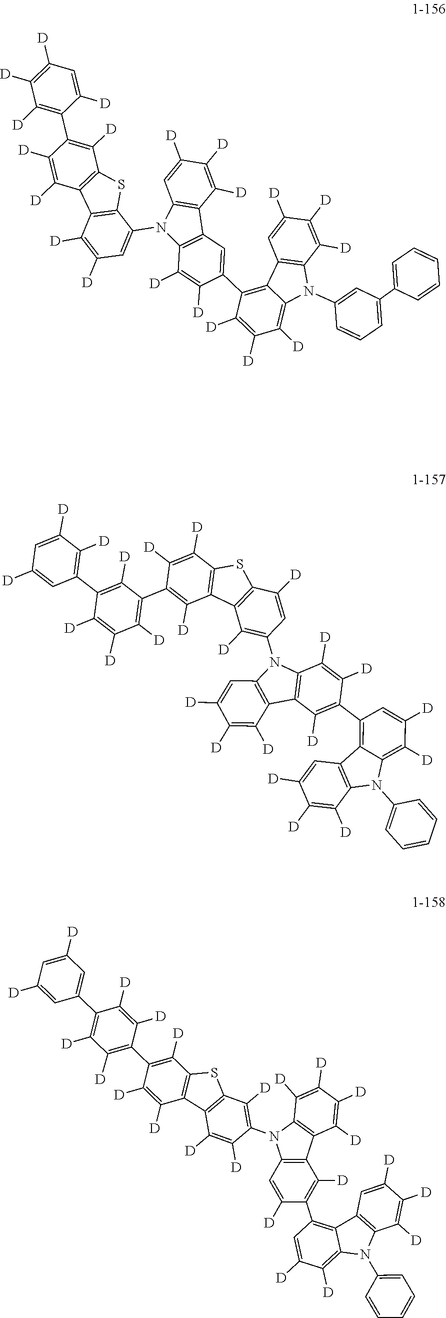

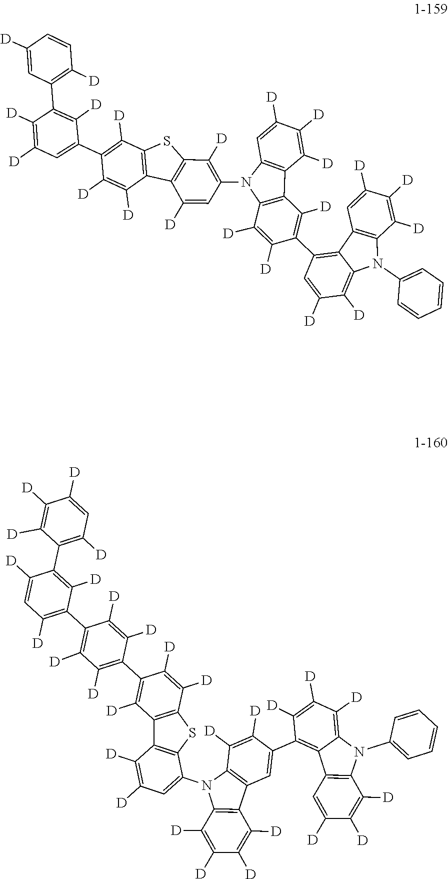 [SMILES: [2H]c1ccc([2H])c(-c2c([2H])c([2H])cc(-c3c([2H])c([2H])c4c(sc5c([2H])c(-n6c7cc([2H])c([2H])c([2H])c7c7c([2H])c(-c8cc([2H])c([2H])c9c8c8cc([2H])c([2H])c([2H])c8n9-c8ccccc8)c([2H])c([2H])c76)cc([2H])c54)c3[2H])c2[2H])c1 ; [2H]c1cc([2H])c(-c2c([2H])cc([2H])c(-c3c([2H])c([2H])c(-c4c([2H])c([2H])c5sc6c(-n7c8c([2H])c([2H])c(-c9c([2H])c([2H])c([2H])c%10c9c9cc([2H])c([2H])c([2H])c9n%10-c9ccccc9)cc8c8c([2H])c([2H])c([2H])c([2H])c87)cc([2H])c([2H])c6c5c4[2H])c([2H])c3[2H])c2[2H])c([2H])c1[2H]]

deuterium of the compound, a specific position is excluded and hydrogen and deuterium may be present in a mixed state during the process of deuterium substitution and synthesis process.

Further, various substituents may be introduced into the structure of Chemical Formula 1 to synthesize a compound having inherent characteristics of a substituent introduced. For example, a substituent usually used for a hole injection material, a hole transport material, a light emitting material, an electron transport material and an electron injection material, which are used when manufacturing an organic light emitting device, may be introduced into the core structure to synthesize a material which satisfies conditions required for each organic material layer.

In addition, by introducing various substituents into the structure of Chemical Formula 1 or changing the binding position, the bandgap may be finely adjusted, and meanwhile, the characteristics at the interface between the organic material layers may be improved.

In addition, the compound of Chemical Formula 1 has excellent thermal stability, and such thermal stability provides driving stability to the organic light emitting device and improves service life characteristics.

In an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In another exemplary embodiment, provided is an organic light emitting device including: a first electrode; a second electrode provided to face the first electrode; and an organic material layer having one or more layers provided between the first electrode and the second electrode, in which one or more layers of the organic material layer include one type of the heterocyclic compound represented by Chemical Formula 1.

In still another exemplary embodiment, provided is an organic light emitting device including: a first electrode; a second electrode provided to face the first electrode; and an organic material layer having one or more layers provided between the first electrode and the second electrode, in which one or more layers of the organic material layer include two or more types of the heterocyclic compound represented by Chemical Formula 1.

In yet another exemplary embodiment, the heterocyclic compound represented by Chemical Formula 1 can be used as a light emitting material for a light emitting layer of the organic light emitting device.

In still yet another exemplary embodiment, the heterocyclic compound represented by Chemical Formula 1 can be used as a host material for a light emitting layer of the organic light emitting device.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device.

In an exemplary embodiment of the present application, the organic light emitting device may be a green organic light emitting device, and the heterocyclic compound represented by Chemical Formula 1 may be used as a material for the green organic light emitting device.

In an exemplary embodiment of the present application, the organic light emitting device may be a red organic light emitting device, and the heterocyclic compound represented by Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for a light emitting layer of the blue organic light emitting device.

In an exemplary embodiment of the present application, the organic light emitting device may be a green organic light emitting device, and the heterocyclic compound represented by Chemical Formula 1 may be used as a material for a light emitting layer of the green organic light emitting device.

In an exemplary embodiment of the present application, the organic light emitting device may be a red organic light emitting device, and the heterocyclic compound represented by Chemical Formula 1 may be used as a material for a light emitting layer of the red organic light emitting device.

In an exemplary embodiment of the present application, the specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃, which is a green phosphorescent dopant, may be used.

In an exemplary embodiment of the present application, as the iridium-based dopant, (piq)₂(Ir)(acac), which is a red phosphorescent dopant, may be used.

In an exemplary embodiment of the present application, provided is an organic light emitting device, in which the organic material layer of the organic light emitting device includes a light emitting layer, and the light emitting layer includes the heterocyclic compound.

In an exemplary embodiment of the present application, provided is an organic light emitting device, in which the organic material layer of the organic light emitting device includes a light emitting layer, and the light emitting layer includes a host material, and the host material includes the heterocyclic compound.

In the organic light emitting device of the present invention, the organic material layer includes an electron injection layer or an electron transport layer, and the electron injection layer or electron transport layer may include the heterocyclic compound.

In another organic light emitting device, the organic material layer includes a hole blocking layer, and the hole blocking layer may include the heterocyclic compound.

In still another organic light emitting device, the organic material layer includes an electron blocking layer, and the electron blocking layer may include the heterocyclic compound.

In yet another organic light emitting device, the organic material layer includes a hole transport layer, a light emitting layer or an electron blocking layer, and the hole transport layer, the light emitting layer or the electron blocking layer may include the heterocyclic compound.

In still yet another organic light emitting device, the organic material layer includes a hole transport layer or a hole transport auxiliary layer, and the hole transport layer or the hole transport auxiliary layer may include the heterocyclic compound.

In the organic light emitting device of the present application, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PE DOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

A s an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

The organic light emitting device of the present invention may further include one or two or more layers selected from the group consisting of a light emitting layer, a hole injection layer, a hole transport layer, a hole transport auxiliary layer, an electron blocking layer, a hole blocking layer, an electron transport layer and an electron injection layer.

The organic light emitting device of the present invention may further include one or two or more layers selected from the group consisting of a light emitting layer, a hole injection layer, a hole transport layer, an electron blocking layer, a hole blocking layer, an electron transport layer and an electron injection layer.

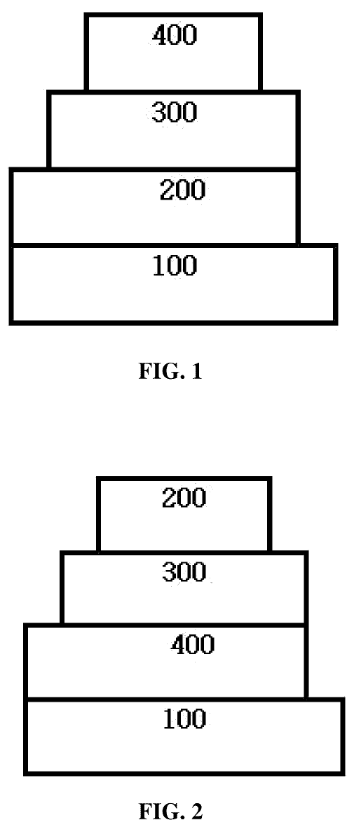

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented.

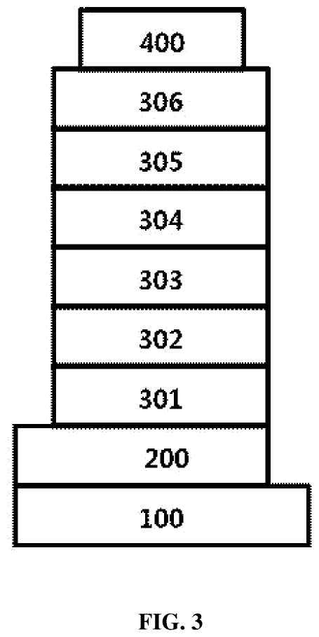

FIG. 3 exemplifies a case where an organic material layer is a multilayer.

An organic light emitting device according to FIG. 3 includes a hole injection layer 301, a hole transport layer 302, a light emitting layer 303, a hole blocking layer 304, an electron transport layer 305, and an electron injection layer 306.

However, the scope of the present application is not limited by the stacking structure as described above, and if necessary, the other layers except for the light emitting layer may be omitted, and another necessary functional layer may be further added.

In an exemplary embodiment of the present application, provided is an organic light emitting device in which the organic material layer of the organic light emitting device including the heterocyclic compound represented by Chemical Formula 1 further includes a heterocyclic compound represented by the following Chemical Formula 2.

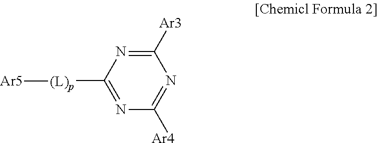

In Chemical Formula 2,
L is a direct bond; a substituted or unsubstituted C6 to C60 arylene group; or a substituted or unsubstituted C2 to C60 heteroarylene group,
p is an integer from 0 to 4, and when p is 2 or higher, substituents in the parenthesis are the same as or different from each other, and
Ar3 to Ar5 are the same as or different from each other, and are each independently a substituted or unsubstituted C6 to C60 aryl group; or a substituted or unsubstituted C2 to C60 heteroaryl group.

In an exemplary embodiment of the present application, L may be a direct bond; a substituted or unsubstituted C6 to C20 arylene group; or a substituted or unsubstituted C2 to C20 heteroarylene group.

In another exemplary embodiment, L may be a direct bond; a substituted or unsubstituted C6 to C20 arylene group; or a substituted or unsubstituted C2 to C20 heteroarylene group.

In still another exemplary embodiment, L may be a direct bond; a substituted or unsubstituted phenylene group; a substituted or unsubstituted biphenylene group; a substituted or unsubstituted terphenylene group; a substituted or unsubstituted dibenzofuranylene group; a substituted or unsubstituted dibenzothiophenylene group; or a substituted or unsubstituted divalent carbazole group.

In yet another exemplary embodiment, L may be a direct bond; a phenylene group; a biphenylene group; a terphenylene group; a dibenzofuranylene group; a dibenzothiophenylene group; or a divalent carbazole group.

In still yet another exemplary embodiment, L may be a direct bond; a phenylene group; a biphenylene group; a dibenzofuranylene group; a dibenzothiophenylene group; or a divalent carbazole group.

In an exemplary embodiment of the present application, Ar3 to Ar5 are the same as or different from each other, and may be each independently a substituted or unsubstituted C6 to C40 aryl group; or a substituted or unsubstituted C2 to C40 heteroaryl group. In another exemplary embodiment, Ar3 to Ar5 are the same as or different from each other, and may be each independently a substituted or unsubstituted C6 to C20 aryl group; or a substituted or unsubstituted C2 to C20 heteroaryl group.

In still another exemplary embodiment, Ar3 to Ar5 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrene group; a substituted or unsubstituted triphenylene group; a substituted or unsubstituted dibenzofuran group; a substituted or unsubstituted dibenzothiophene group; a substituted or unsubstituted indolocarbazole group; a substituted or unsubstituted benzothienocarbazole group; a substituted or unsubstituted carbazole group; a substituted or unsubstituted benzofurocarbazole group; or a substituted or unsubstituted indenocarbazole group.

In yet another exemplary embodiment, Ar3 and Ar4 are the same as or different from each other, and may be each independently a substituted or unsubstituted phenyl group; a substituted or unsubstituted biphenyl group; a substituted or unsubstituted dibenzofuran group; or a substituted or unsubstituted dibenzothiophene group.

In still yet another exemplary embodiment, Ar3 and Ar4 are the same as or different from each other, and may be each independently a phenyl group; a biphenyl group; a dibenzofuran group; or a dibenzothiophene group.

In a further exemplary embodiment, Ar5 may be a phenyl group; a biphenyl group; a terphenyl group; a naphthyl group; a phenanthrene group; a triphenylene group; a dibenzofuran group; a dibenzothiophene group; an indolocarbazole group unsubstituted or substituted with an aryl group; a benzothienocarbazole group; a carbazole group unsubstituted or substituted with an aryl group; a benzofurocarbazole group; or an indenocarbazole group unsubstituted or substituted with an alkyl group.

In another further exemplary embodiment, Ar5 may be a phenyl group; a biphenyl group; a terphenyl group; a triphenylene group; a dibenzofuran group; a dibenzothiophene group; an indolocarbazole group unsubstituted or substituted with an aryl group; a benzothienocarbazole group; a carbazole group unsubstituted or substituted with an aryl group; a benzofurocarbazole group; or an indenocarbazole group unsubstituted or substituted with an alkyl group.

In an exemplary embodiment of the present application, p is an integer from 0 to 3, and when p is 2 or higher, substituents in the parenthesis may be the same as or different from each other.

In another exemplary embodiment, p is an integer from 0 to 2, and when p is 2 or higher, substituents in the parenthesis may be the same as or different from each other.

In still another exemplary embodiment, p is 0 or 1.

In yet another exemplary embodiment, p is 1.

In still yet another exemplary embodiment, p is 0.

In an exemplary embodiment of the present application, when both Ar3 and Ar4 are a substituted or unsubstituted C6 to C60 aryl group, L may be a substituted or unsubstituted C2 to C60 heteroarylene group, or Ar5 may be a substituted or unsubstituted C2 to C60 heteroaryl group.

In another exemplary embodiment, when both Ar3 and Ar4 are a substituted or unsubstituted C6 to C40 aryl group, L may be a substituted or unsubstituted C2 to C40 heteroarylene group, or Ar5 may be a substituted or unsubstituted C2 to C40 heteroaryl group.

In still another exemplary embodiment, when both Ar3 and Ar4 are a substituted or unsubstituted phenyl group; or a substituted or unsubstituted biphenyl group, L may be a substituted or unsubstituted dibenzofuranylene group; a substituted or unsubstituted dibenzothiophenylene group; or a substituted or unsubstituted divalent carbazole group, or Ar5 may be a substituted or unsubstituted dibenzofuran group; a substituted or unsubstituted dibenzothiophene group; a substituted or unsubstituted indolocarbazole group; a substituted or unsubstituted benzothienocarbazole group; a substituted or unsubstituted carbazole group; a substituted or unsubstituted benzofurocarbazole group; or a substituted or unsubstituted indenocarbazole group.

In yet another exemplary embodiment, when both Ar3 and Ar4 are a substituted or unsubstituted phenyl group; or a substituted or unsubstituted biphenyl group, L may be a dibenzofuranylene group; a dibenzothiopheneylene group; or a divalent carbazole group, or Ar5 may be a dibenzofuran group; a dibenzothiophene group; an indolocarbazole group unsubstituted or substituted with an aryl group; a benzothienocarbazole group; a carbazole group unsubstituted or substituted with an aryl group; a benzofurocarbazole group; or an indenocarbazole group unsubstituted or substituted with an alkyl group.

In an exemplary embodiment of the present application, the deuterium content of the heterocyclic compound of Chemical Formula 2 may be 0% to 100%.

In an exemplary embodiment of the present application, the deuterium content of the heterocyclic compound of Chemical Formula 2 may be 0%.

In an exemplary embodiment of the present application, the deuterium content of the heterocyclic compound of Chemical Formula 2 may be 10% to 100%.

In an exemplary embodiment of the present application, the deuterium content of the heterocyclic compound of Chemical Formula 2 may be 30% to 100%.

In an exemplary embodiment of the present application, the deuterium content of the heterocyclic compound of Chemical Formula 2 may be 70% to 100%.

In an exemplary embodiment of the present application, the deuterium content of the heterocyclic compound of Chemical Formula 2 may be 90% to 100%.

In an exemplary embodiment of the present application, provided is a compound in which Chemical Formula 2 is represented by any one of the following compounds.

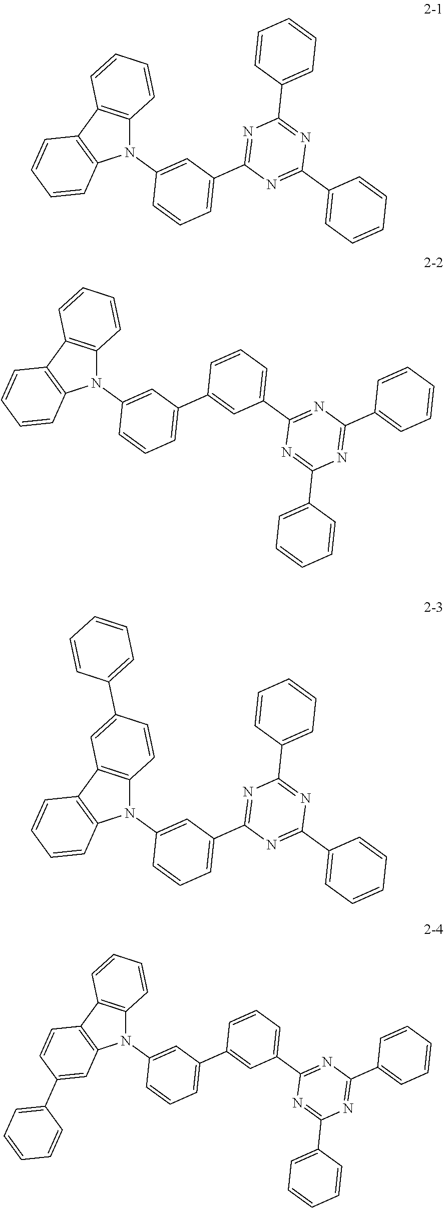 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cccc(-c4cccc(-n5c6ccccc6c6ccccc65)c4)c3)n2)cc1 ; c1ccc(-c2ccc3c(c2)c2ccccc2n3-c2cccc(-c3nc(-c4ccccc4)nc(-c4ccccc4)n3)c2)cc1 ; c1ccc(-c2ccc3c4ccccc4n(-c4cccc(-c5cccc(-c6nc(-c7ccccc7)nc(-c7ccccc7)n6)c5)c4)c3c2)cc1]

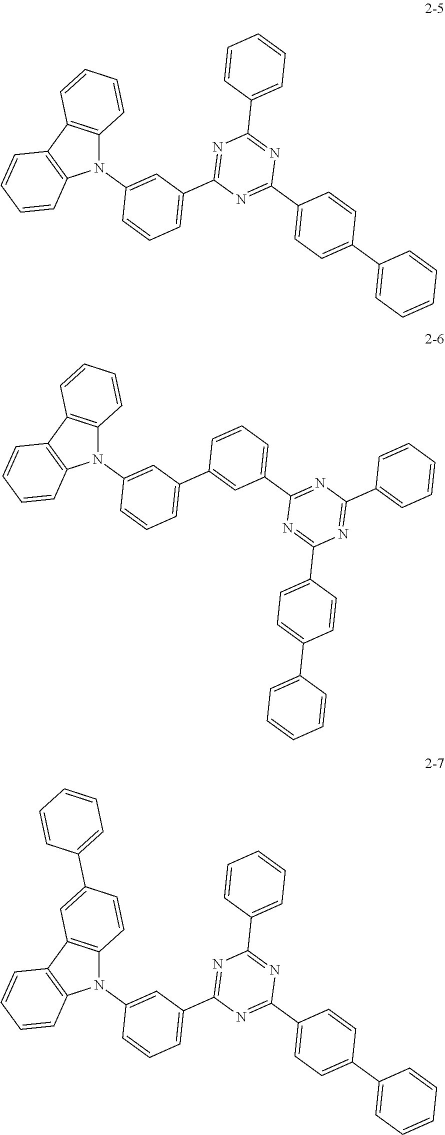 [SMILES: c1ccc(-c2ccc(-c3nc(-c4ccccc4)nc(-c4cccc(-n5c6ccccc6c6ccccc65)c4)n3)cc2)cc1 ; c1ccc(-c2ccc(-c3nc(-c4ccccc4)nc(-c4cccc(-c5cccc(-n6c7ccccc7c7ccccc76)c5)c4)n3)cc2)cc1 ; c1ccc(-c2ccc(-c3nc(-c4ccccc4)nc(-c4cccc(-n5c6ccccc6c6cc(-c7ccccc7)ccc65)c4)n3)cc2)cc1]

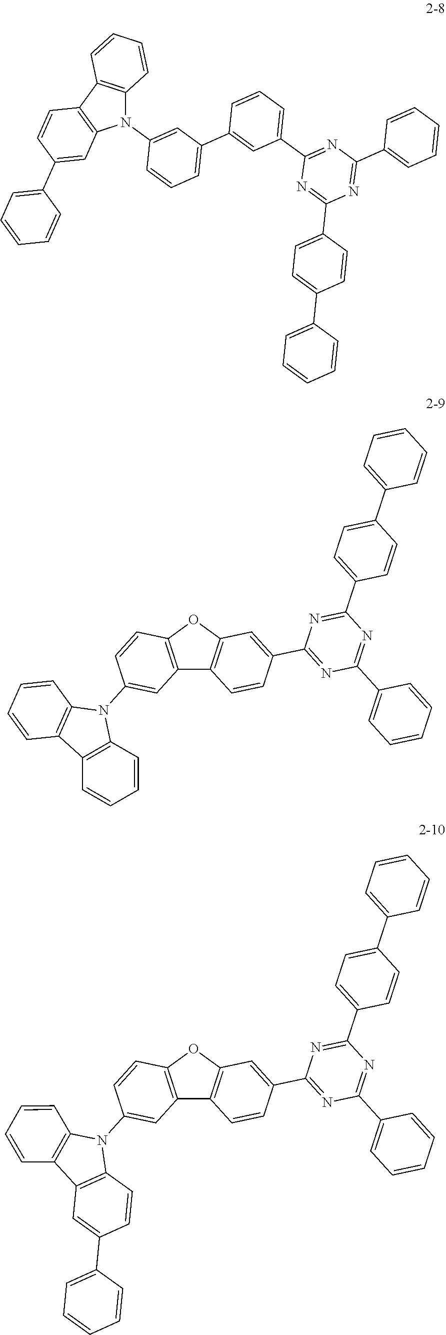 [SMILES: c1ccc(-c2ccc(-c3nc(-c4ccccc4)nc(-c4cccc(-c5cccc(-n6c7ccccc7c7ccc(-c8ccccc8)cc76)c5)c4)n3)cc2)cc1 ; c1ccc(-c2ccc(-c3nc(-c4ccccc4)nc(-c4ccc5c(c4)oc4ccc(-n6c7ccccc7c7ccccc76)cc45)n3)cc2)cc1 ; c1ccc(-c2ccc(-c3nc(-c4ccccc4)nc(-c4ccc5c(c4)oc4ccc(-n6c7ccccc7c7cc(-c8ccccc8)ccc76)cc45)n3)cc2)cc1]

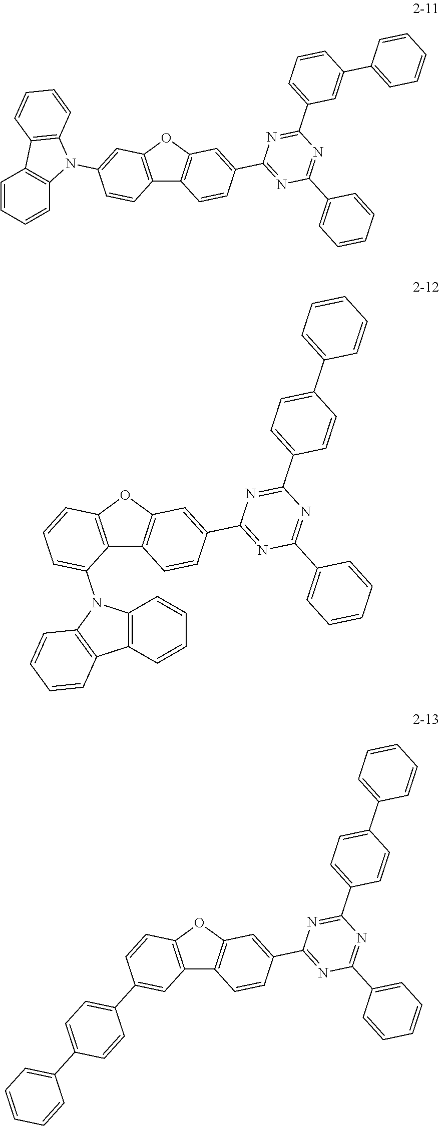 [SMILES: c1ccc(-c2cccc(-c3nc(-c4ccccc4)nc(-c4ccc5c(c4)oc4cc(-n6c7ccccc7c7ccccc76)ccc45)n3)c2)cc1 ; c1ccc(-c2ccc(-c3nc(-c4ccccc4)nc(-c4ccc5c(c4)oc4cccc(-n6c7ccccc7c7ccccc76)c45)n3)cc2)cc1 ; c1ccc(-c2ccc(-c3ccc4oc5cc(-c6nc(-c7ccccc7)nc(-c7ccc(-c8ccccc8)cc7)n6)ccc5c4c3)cc2)cc1]

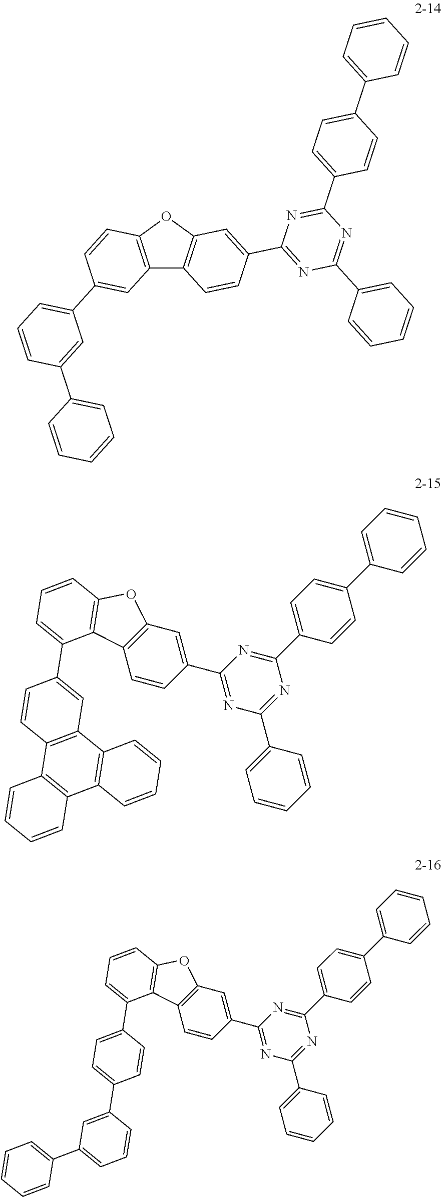 [SMILES: c1ccc(-c2ccc(-c3nc(-c4ccccc4)nc(-c4ccc5c(c4)oc4ccc(-c6cccc(-c7ccccc7)c6)cc45)n3)cc2)cc1 ; c1ccc(-c2ccc(-c3nc(-c4ccccc4)nc(-c4ccc5c(c4)oc4cccc(-c6ccc7c8ccccc8c8ccccc8c7c6)c45)n3)cc2)cc1 ; c1ccc(-c2ccc(-c3nc(-c4ccccc4)nc(-c4ccc5c(c4)oc4cccc(-c6ccc(-c7cccc(-c8ccccc8)c7)cc6)c45)n3)cc2)cc1]

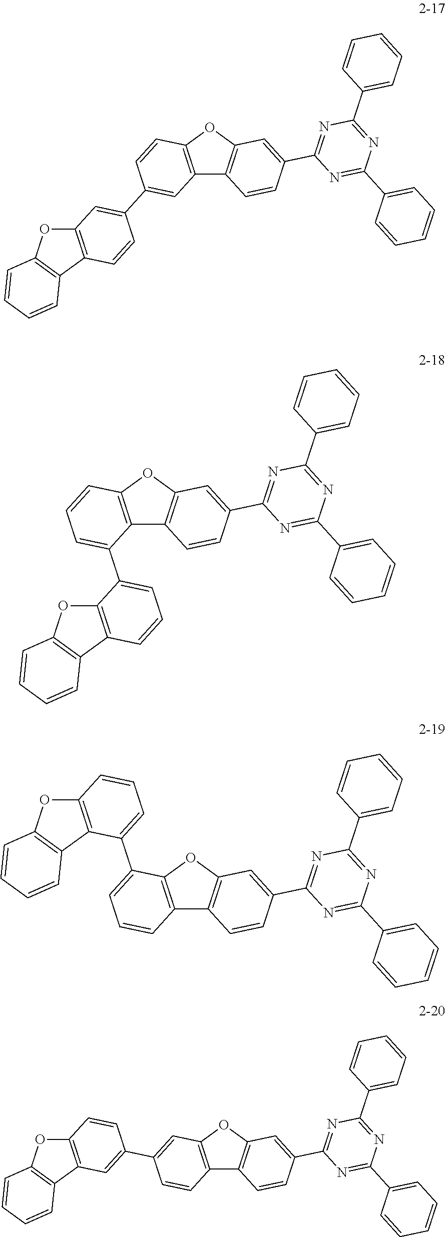 [SMILES: c1ccc(-c2nc(-c3ccccc3)nc(-c3ccc4c(c3)oc3ccc(-c5ccc6c(c5)oc5ccccc56)cc34)n2)cc1 ; c1ccc(-c2nc(-c3ccccc3)nc(-c3ccc4c(c3)oc3cccc(-c5cccc6c5oc5ccccc56)c34)n2)cc1 ; c1ccc(-c2nc(-c3ccccc3)nc(-c3ccc4c(c3)oc3c(-c5cccc6oc7ccccc7c56)cccc34)n2)cc1 ; c1ccc(-c2nc(-c3ccccc3)nc(-c3ccc4c(c3)oc3cc(-c5ccc6oc7ccccc7c6c5)ccc34)n2)cc1]

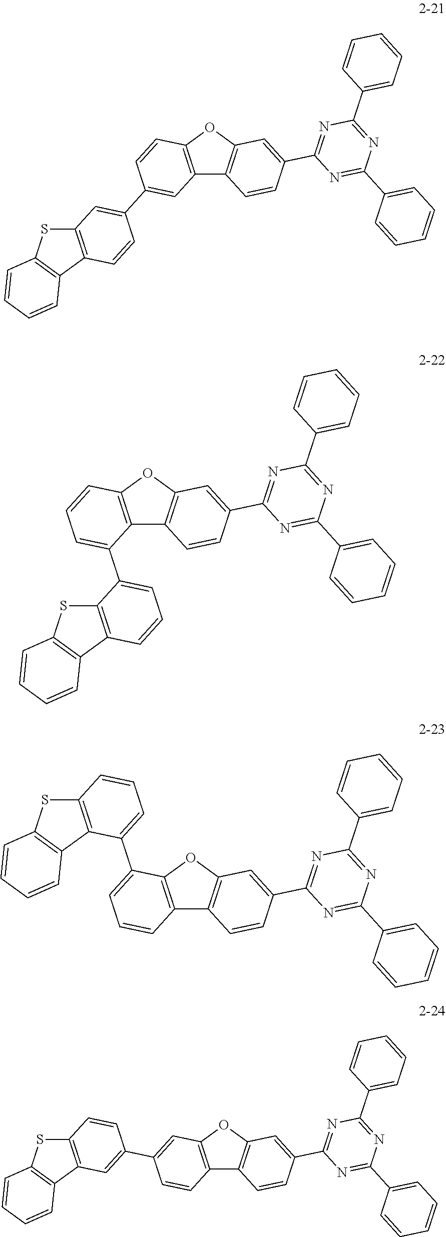 [SMILES: c1ccc(-c2nc(-c3ccccc3)nc(-c3ccc4c(c3)oc3ccc(-c5ccc6c(c5)sc5ccccc56)cc34)n2)cc1 ; c1ccc(-c2nc(-c3ccccc3)nc(-c3ccc4c(c3)oc3cccc(-c5cccc6c5sc5ccccc56)c34)n2)cc1 ; c1ccc(-c2nc(-c3ccccc3)nc(-c3ccc4c(c3)oc3c(-c5cccc6sc7ccccc7c56)cccc34)n2)cc1 ; c1ccc(-c2nc(-c3ccccc3)nc(-c3ccc4c(c3)oc3cc(-c5ccc6sc7ccccc7c6c5)ccc34)n2)cc1]

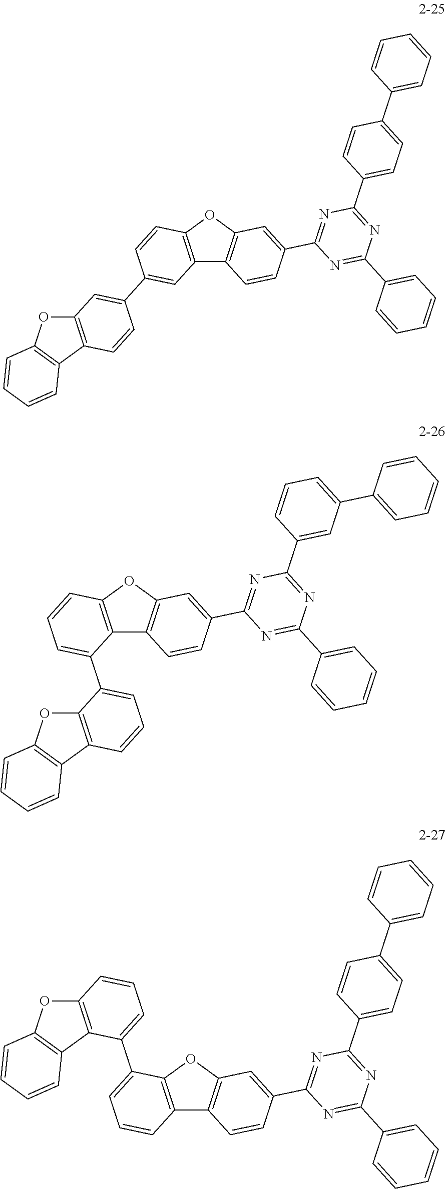 [SMILES: c1ccc(-c2ccc(-c3nc(-c4ccccc4)nc(-c4ccc5c(c4)oc4ccc(-c6ccc7c(c6)oc6ccccc67)cc45)n3)cc2)cc1 ; c1ccc(-c2cccc(-c3nc(-c4ccccc4)nc(-c4ccc5c(c4)oc4cccc(-c6cccc7c6oc6ccccc67)c45)n3)c2)cc1 ; c1ccc(-c2ccc(-c3nc(-c4ccccc4)nc(-c4ccc5c(c4)oc4c(-c6cccc7oc8ccccc8c67)cccc45)n3)cc2)cc1]

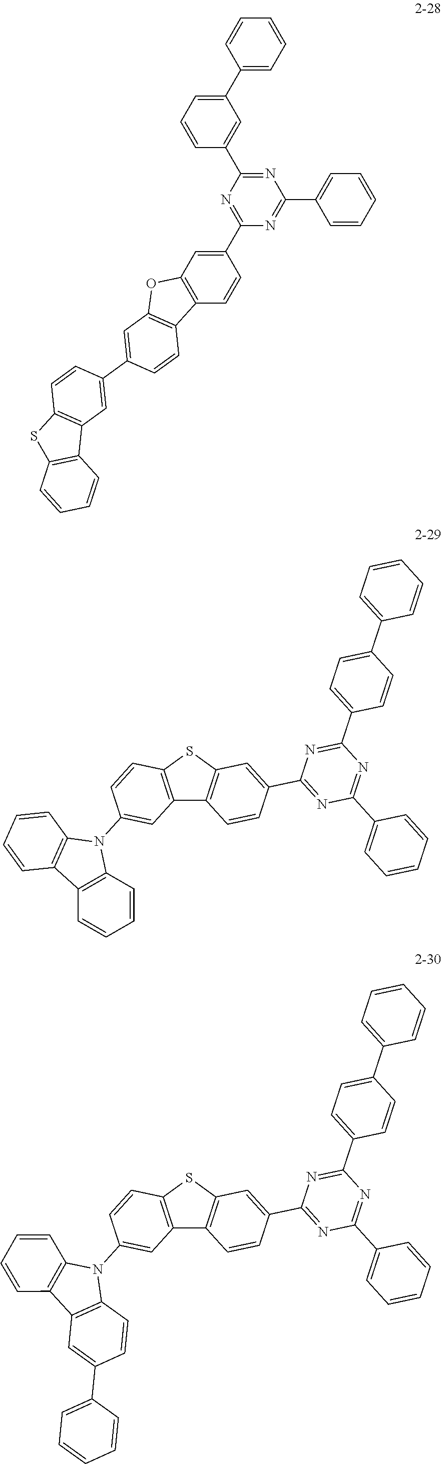 [SMILES: c1ccc(-c2cccc(-c3nc(-c4ccccc4)nc(-c4ccc5c(c4)oc4cc(-c6ccc7sc8ccccc8c7c6)ccc45)n3)c2)cc1 ; c1ccc(-c2ccc(-c3nc(-c4ccccc4)nc(-c4ccc5c(c4)sc4ccc(-n6c7ccccc7c7ccccc76)cc45)n3)cc2)cc1 ; c1ccc(-c2ccc(-c3nc(-c4ccccc4)nc(-c4ccc5c(c4)sc4ccc(-n6c7ccccc7c7cc(-c8ccccc8)ccc76)cc45)n3)cc2)cc1]

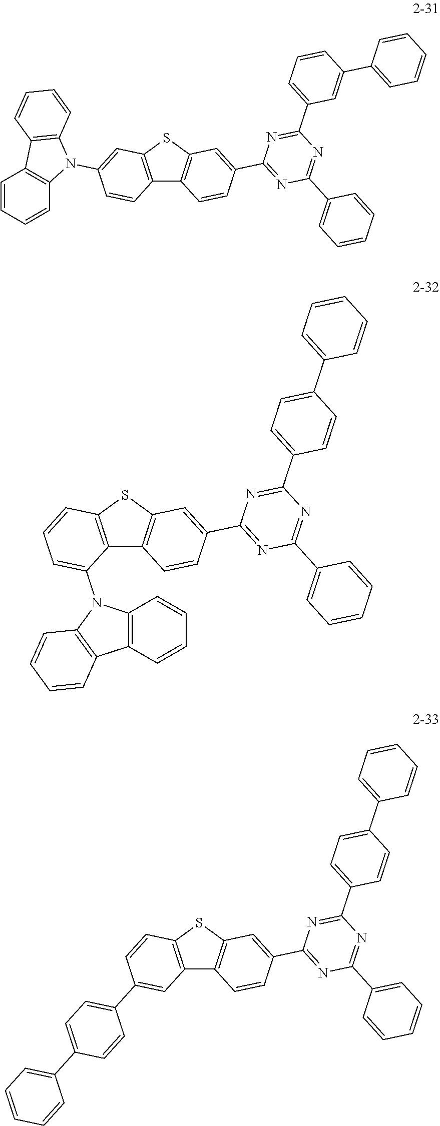 [SMILES: c1ccc(-c2cccc(-c3nc(-c4ccccc4)nc(-c4ccc5c(c4)sc4cc(-n6c7ccccc7c7ccccc76)ccc45)n3)c2)cc1 ; c1ccc(-c2ccc(-c3nc(-c4ccccc4)nc(-c4ccc5c(c4)sc4cccc(-n6c7ccccc7c7ccccc76)c45)n3)cc2)cc1 ; c1ccc(-c2ccc(-c3ccc4sc5cc(-c6nc(-c7ccccc7)nc(-c7ccc(-c8ccccc8)cc7)n6)ccc5c4c3)cc2)cc1]

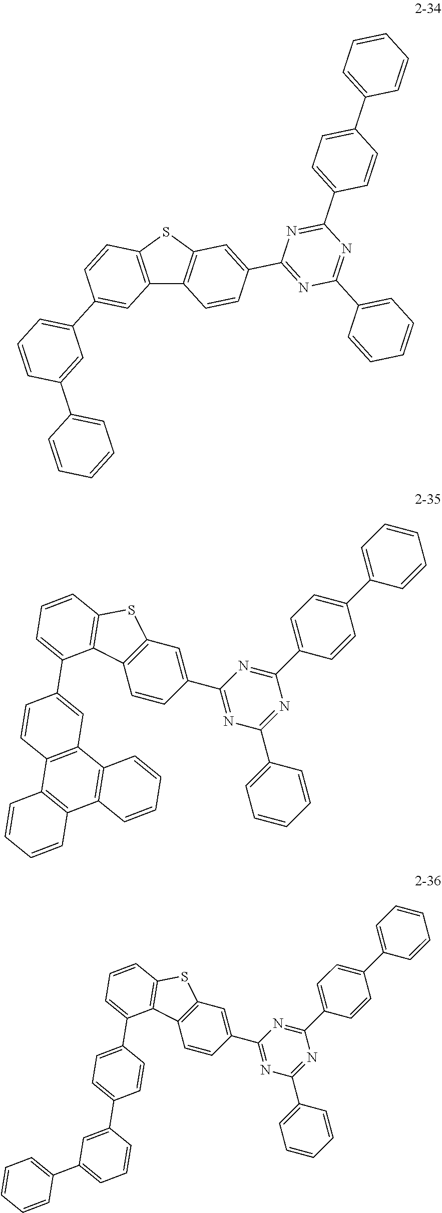 [SMILES: c1ccc(-c2ccc(-c3nc(-c4ccccc4)nc(-c4ccc5c(c4)sc4ccc(-c6cccc(-c7ccccc7)c6)cc45)n3)cc2)cc1 ; c1ccc(-c2ccc(-c3nc(-c4ccccc4)nc(-c4ccc5c(c4)sc4cccc(-c6ccc7c8ccccc8c8ccccc8c7c6)c45)n3)cc2)cc1 ; c1ccc(-c2ccc(-c3nc(-c4ccccc4)nc(-c4ccc5c(c4)sc4cccc(-c6ccc(-c7cccc(-c8ccccc8)c7)cc6)c45)n3)cc2)cc1]

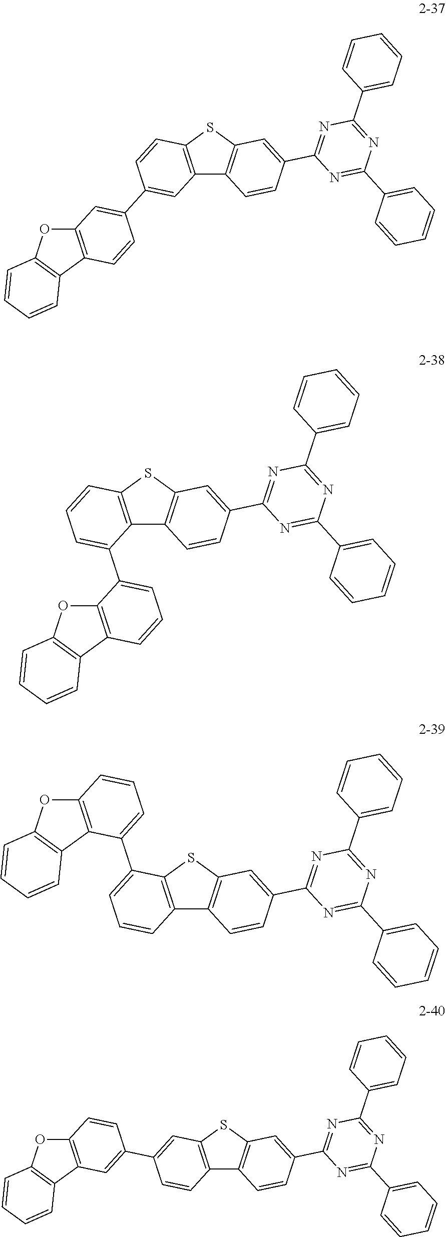 [SMILES: c1ccc(-c2nc(-c3ccccc3)nc(-c3ccc4c(c3)sc3ccc(-c5ccc6c(c5)oc5ccccc56)cc34)n2)cc1 ; c1ccc(-c2nc(-c3ccccc3)nc(-c3ccc4c(c3)sc3cccc(-c5cccc6c5oc5ccccc56)c34)n2)cc1 ; c1ccc(-c2nc(-c3ccccc3)nc(-c3ccc4c(c3)sc3c(-c5cccc6oc7ccccc7c56)cccc34)n2)cc1 ; c1ccc(-c2nc(-c3ccccc3)nc(-c3ccc4c(c3)sc3cc(-c5ccc6oc7ccccc7c6c5)ccc34)n2)cc1]

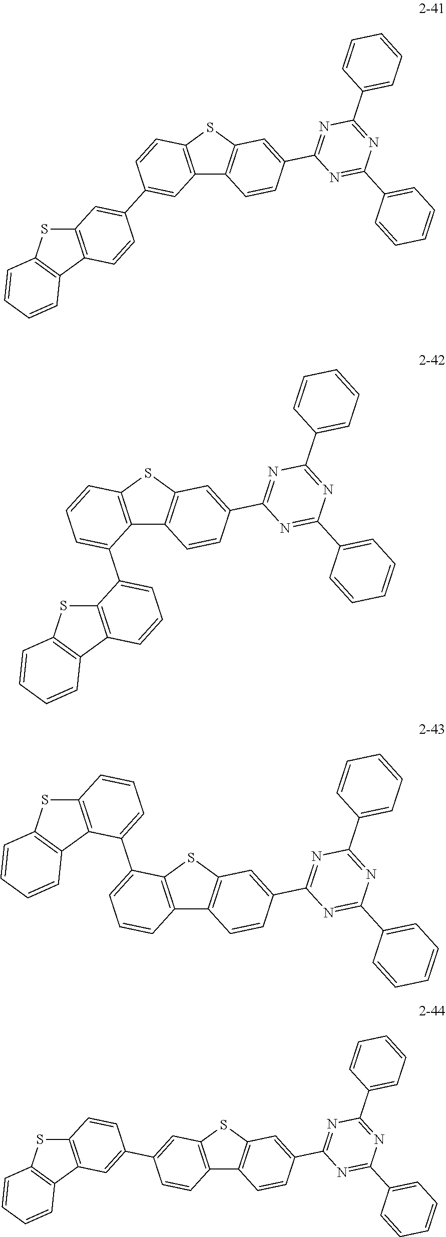 [SMILES: c1ccc(-c2nc(-c3ccccc3)nc(-c3ccc4c(c3)sc3ccc(-c5ccc6c(c5)sc5ccccc56)cc34)n2)cc1 ; c1ccc(-c2nc(-c3ccccc3)nc(-c3ccc4c(c3)sc3cccc(-c5cccc6c5sc5ccccc56)c34)n2)cc1 ; c1ccc(-c2nc(-c3ccccc3)nc(-c3ccc4c(c3)sc3c(-c5cccc6sc7ccccc7c56)cccc34)n2)cc1 ; c1ccc(-c2nc(-c3ccccc3)nc(-c3ccc4c(c3)sc3cc(-c5ccc6sc7ccccc7c6c5)ccc34)n2)cc1]

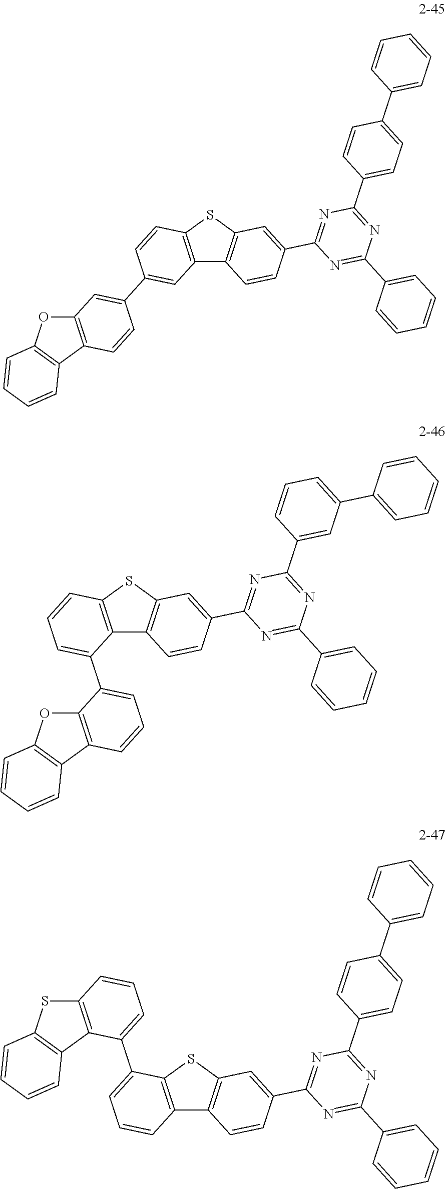 [SMILES: c1ccc(-c2ccc(-c3nc(-c4ccccc4)nc(-c4ccc5c(c4)sc4ccc(-c6ccc7c(c6)oc6ccccc67)cc45)n3)cc2)cc1 ; c1ccc(-c2cccc(-c3nc(-c4ccccc4)nc(-c4ccc5c(c4)sc4cccc(-c6cccc7c6oc6ccccc67)c45)n3)c2)cc1 ; c1ccc(-c2ccc(-c3nc(-c4ccccc4)nc(-c4ccc5c(c4)sc4c(-c6cccc7sc8ccccc8c67)cccc45)n3)cc2)cc1]

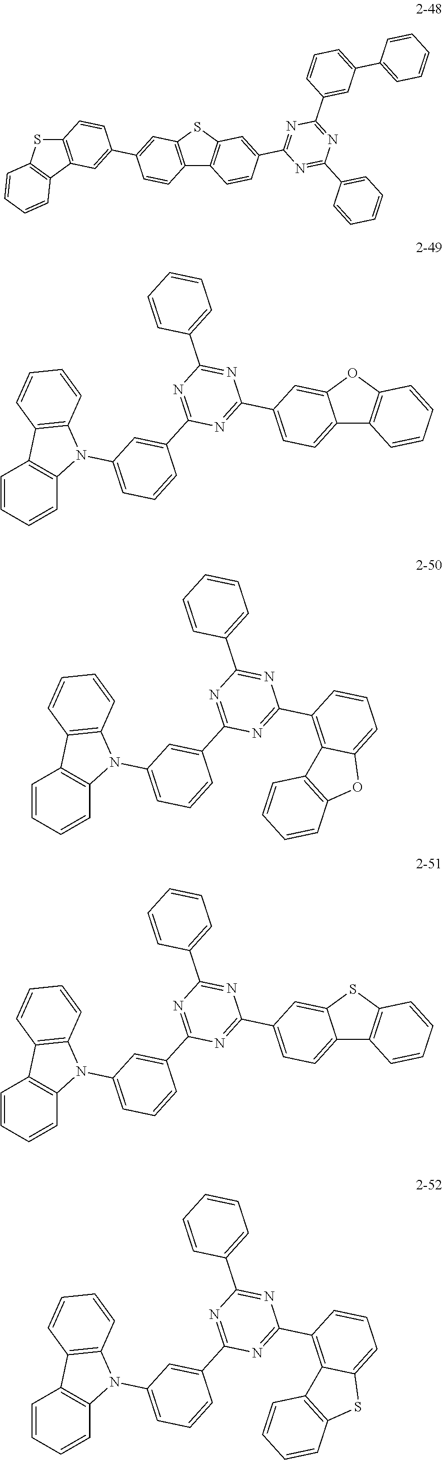 [SMILES: c1ccc(-c2cccc(-c3nc(-c4ccccc4)nc(-c4ccc5c(c4)sc4cc(-c6ccc7sc8ccccc8c7c6)ccc45)n3)c2)cc1 ; c1ccc(-c2nc(-c3cccc(-n4c5ccccc5c5ccccc54)c3)nc(-c3ccc4c(c3)oc3ccccc34)n2)cc1 ; c1ccc(-c2nc(-c3cccc(-n4c5ccccc5c5ccccc54)c3)nc(-c3cccc4oc5ccccc5c34)n2)cc1 ; c1ccc(-c2nc(-c3cccc(-n4c5ccccc5c5ccccc54)c3)nc(-c3ccc4c(c3)sc3ccccc34)n2)cc1 ; c1ccc(-c2nc(-c3cccc(-n4c5ccccc5c5ccccc54)c3)nc(-c3cccc4sc5ccccc5c34)n2)cc1]

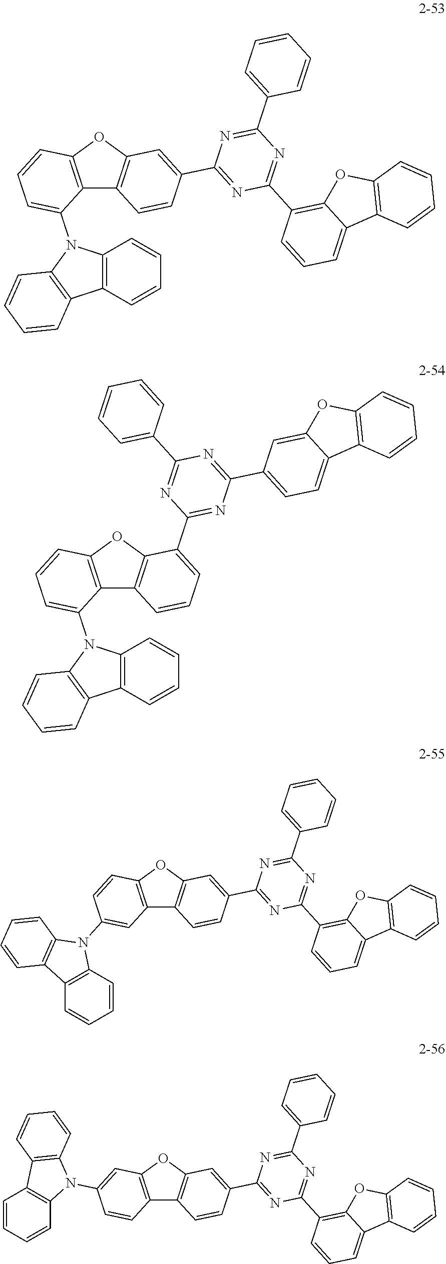 [SMILES: c1ccc(-c2nc(-c3ccc4c(c3)oc3cccc(-n5c6ccccc6c6ccccc65)c34)nc(-c3cccc4c3oc3ccccc34)n2)cc1 ; c1ccc(-c2nc(-c3ccc4c(c3)oc3ccccc34)nc(-c3cccc4c3oc3cccc(-n5c6ccccc6c6ccccc65)c34)n2)cc1 ; c1ccc(-c2nc(-c3ccc4c(c3)oc3ccc(-n5c6ccccc6c6ccccc65)cc34)nc(-c3cccc4c3oc3ccccc34)n2)cc1 ; c1ccc(-c2nc(-c3ccc4c(c3)oc3cc(-n5c6ccccc6c6ccccc65)ccc34)nc(-c3cccc4c3oc3ccccc34)n2)cc1]

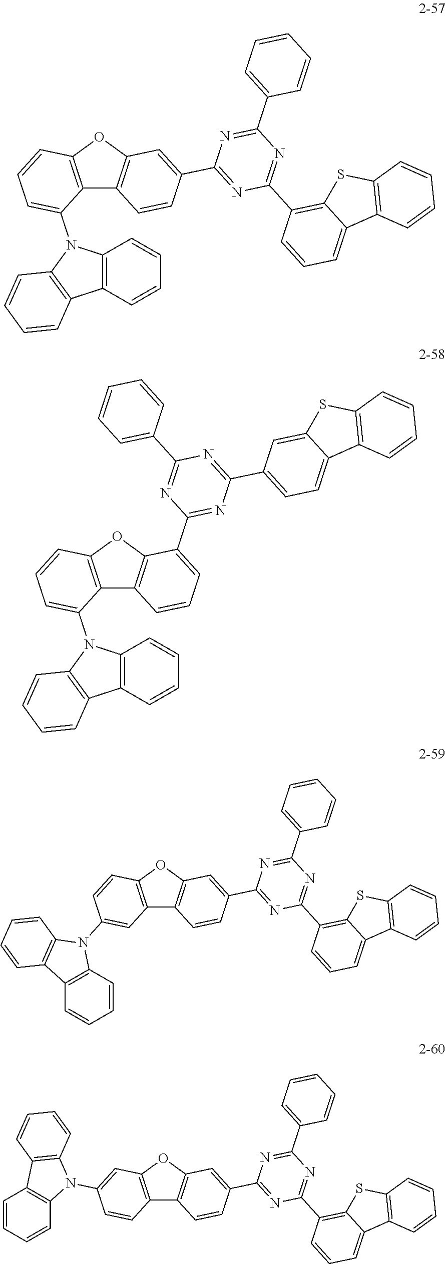 [SMILES: c1ccc(-c2nc(-c3ccc4c(c3)oc3cccc(-n5c6ccccc6c6ccccc65)c34)nc(-c3cccc4c3sc3ccccc34)n2)cc1 ; c1ccc(-c2nc(-c3ccc4c(c3)sc3ccccc34)nc(-c3cccc4c3oc3cccc(-n5c6ccccc6c6ccccc65)c34)n2)cc1 ; c1ccc(-c2nc(-c3ccc4c(c3)oc3ccc(-n5c6ccccc6c6ccccc65)cc34)nc(-c3cccc4c3sc3ccccc34)n2)cc1 ; c1ccc(-c2nc(-c3ccc4c(c3)oc3cc(-n5c6ccccc6c6ccccc65)ccc34)nc(-c3cccc4c3sc3ccccc34)n2)cc1]

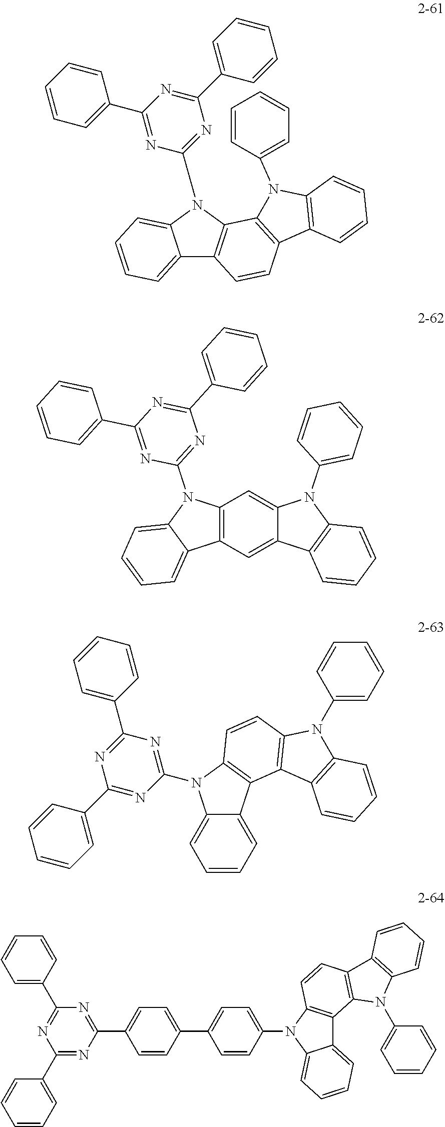 [SMILES: c1ccc(-c2nc(-c3ccccc3)nc(-n3c4ccccc4c4ccc5c6ccccc6n(-c6ccccc6)c5c43)n2)cc1 ; c1ccc(-c2nc(-c3ccccc3)nc(-n3c4ccccc4c4cc5c6ccccc6n(-c6ccccc6)c5cc43)n2)cc1 ; c1ccc(-c2nc(-c3ccccc3)nc(-n3c4ccccc4c4c5c6ccccc6n(-c6ccccc6)c5ccc43)n2)cc1 ; c1ccc(-c2nc(-c3ccccc3)nc(-c3ccc(-c4ccc(-n5c6ccccc6c6c5ccc5c7ccccc7n(-c7ccccc7)c56)cc4)cc3)n2)cc1]

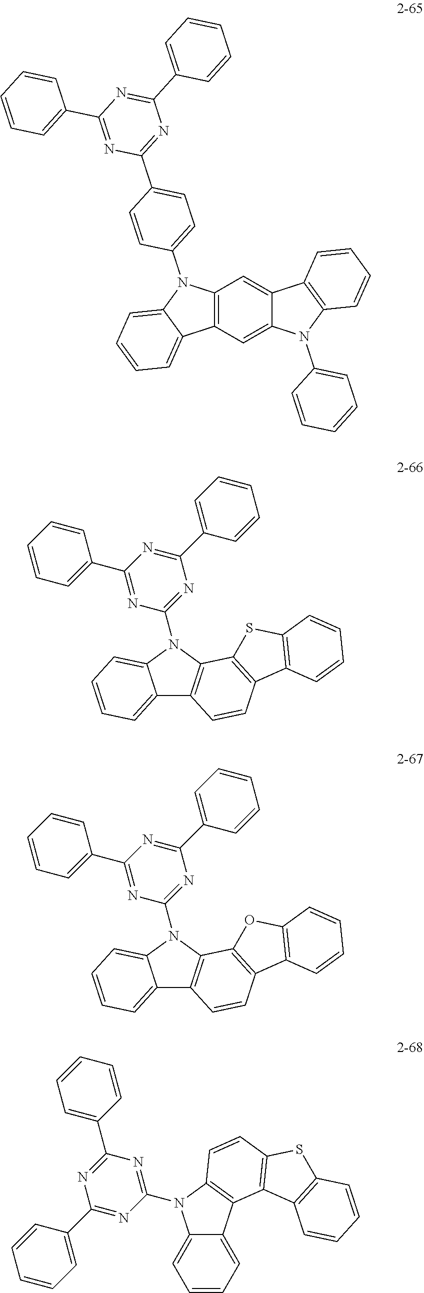 [SMILES: c1ccc(-c2nc(-c3ccccc3)nc(-c3ccc(-n4c5ccccc5c5cc6c(cc54)c4ccccc4n6-c4ccccc4)cc3)n2)cc1 ; c1ccc(-c2nc(-c3ccccc3)nc(-n3c4ccccc4c4ccc5c6ccccc6sc5c43)n2)cc1 ; c1ccc(-c2nc(-c3ccccc3)nc(-n3c4ccccc4c4ccc5c6ccccc6oc5c43)n2)cc1 ; c1ccc(-c2nc(-c3ccccc3)nc(-n3c4ccccc4c4c5c(ccc43)sc3ccccc35)n2)cc1]

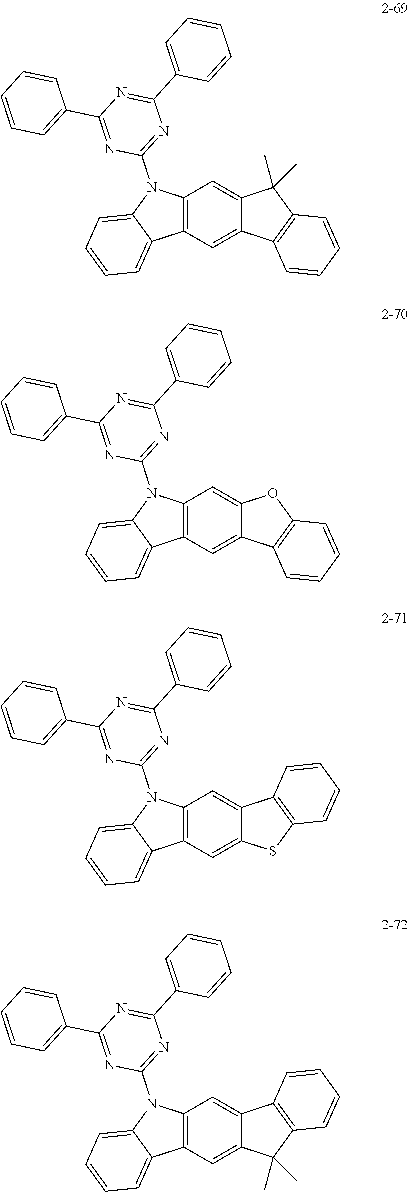 [SMILES: CC1(C)c2ccccc2-c2cc3c4ccccc4n(-c4nc(-c5ccccc5)nc(-c5ccccc5)n4)c3cc21 ; c1ccc(-c2nc(-c3ccccc3)nc(-n3c4ccccc4c4cc5c(cc43)oc3ccccc35)n2)cc1 ; c1ccc(-c2nc(-c3ccccc3)nc(-n3c4ccccc4c4cc5sc6ccccc6c5cc43)n2)cc1 ; CC1(C)c2ccccc2-c2cc3c(cc21)c1ccccc1n3-c1nc(-c2ccccc2)nc(-c2ccccc2)n1]

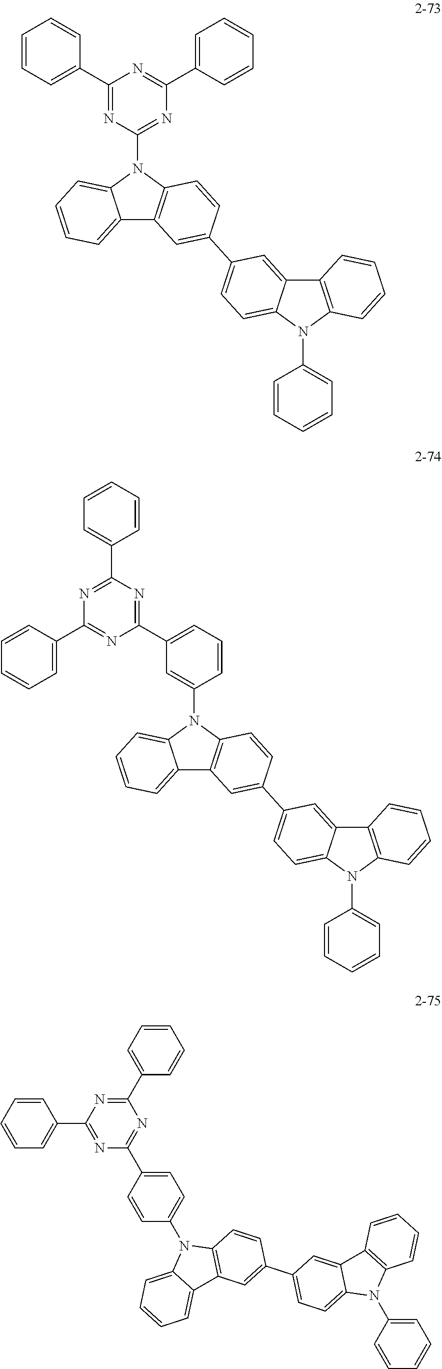 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

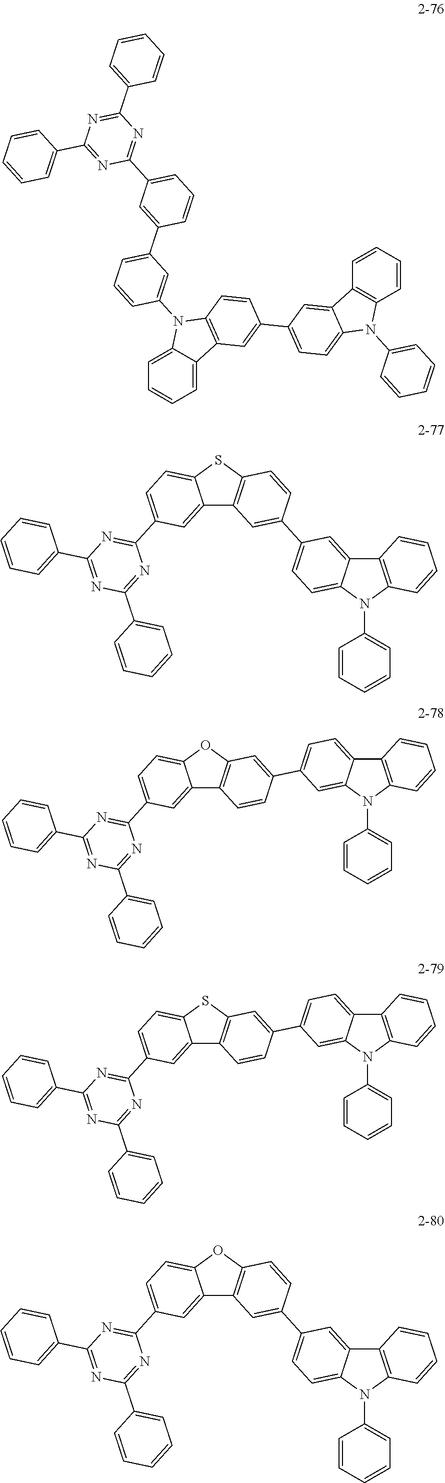 [SMILES: c1ccc(-c2nc(-c3ccccc3)nc(-c3cccc(-c4cccc(-n5c6ccccc6c6cc(-c7ccc8c(c7)c7ccccc7n8-c7ccccc7)ccc65)c4)c3)n2)cc1 ; c1ccc(-c2nc(-c3ccccc3)nc(-c3ccc4sc5ccc(-c6ccc7c(c6)c6ccccc6n7-c6ccccc6)cc5c4c3)n2)cc1 ; c1ccc(-c2nc(-c3ccccc3)nc(-c3ccc4oc5cc(-c6ccc7c8ccccc8n(-c8ccccc8)c7c6)ccc5c4c3)n2)cc1 ; c1ccc(-c2nc(-c3ccccc3)nc(-c3ccc4sc5cc(-c6ccc7c8ccccc8n(-c8ccccc8)c7c6)ccc5c4c3)n2)cc1 ; c1ccc(-c2nc(-c3ccccc3)nc(-c3ccc4oc5ccc(-c6ccc7c(c6)c6ccccc6n7-c6ccccc6)cc5c4c3)n2)cc1]

A content on the organic light emitting device including the heterocyclic compound represented by Chemical Formula 1 described above may be applied to the organic light emitting device further including the heterocyclic compound represented by Chemical Formula 2.

In another exemplary embodiment, the heterocyclic compound represented by Chemical Formula 2 can be used as a light emitting material for a light emitting layer of the organic light emitting device.

In still another exemplary embodiment, the heterocyclic compound represented by Chemical Formula 2 may be used as a light emitting material for a light emitting layer of an organic light emitting device, and may be used as a p-type host material.

In the organic light emitting device of the present invention, the organic material layer may include the heterocyclic compound represented by Chemical Formula 1 and the heterocyclic compound represented by Chemical Formula 2. The organic material layer may be formed by pre-mixing the heterocyclic compound represented by Chemical Formula 1 and the heterocyclic compound represented by Chemical Formula 2 and using a thermal vacuum deposition method.

In another organic light emitting device, the organic material layer includes a light emitting layer, the light emitting layer includes a host material, and the host material may include the heterocyclic compound represented by Chemical Formula 1 and the heterocyclic compound represented by Chemical Formula 2.

In still another organic light emitting device, the organic material layer includes a light emitting layer, includes the heterocyclic compound represented by Chemical Formula 1 as an n-type host material for the light emitting layer, and may include the heterocyclic compound represented by Chemical Formula 2 as a p-type host material.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by supplying the heterocyclic compound represented by Chemical Formula 1 and the heterocyclic compound represented by Chemical Formula 2 as each individual supply source, and then using a thermal vacuum deposition method.

In an exemplary embodiment of the present application, provided is a method for manufacturing an organic light emitting device, in which the forming of the organic material layer forms the organic material layer by pre-mixing the heterocyclic compound represented by Chemical Formula 1 and the heterocyclic compound represented by Chemical Formula 2, and using a thermal vacuum deposition method.

The organic light emitting device according to an exemplary embodiment of the present application may be manufactured by typical manufacturing methods and materials of the organic light emitting device, except that the above-described heterocyclic compound is used to form an organic material layer.

The organic light emitting device of the present invention may further include one or two or more layers selected from the group consisting of a light emitting layer, a hole injection layer, a hole transport layer, an electron blocking layer, an electron injection layer, an electron transport layer, a hole auxiliary layer, and a hole blocking layer.

In an exemplary embodiment of the present application, provided is a composition for an organic material layer of an organic light emitting device, which includes the heterocyclic compound represented by Chemical Formula 1 and the heterocyclic compound represented by Chemical Formula 2.

The weight ratio of the heterocyclic compound represented by Chemical Formula 1:the heterocyclic compound represented by Chemical Formula 2 in the composition may be 1:10 to 10:1, 1:8 to 8:1, 1:5 to 5:1, and 1:2 to 2:1, but is not limited thereto.

The composition may be used when an organic material layer of an organic light emitting device is formed, and particularly, may be more preferably used as a host material for the light emitting layer.

The composition is in a form in which two or more compounds are simply mixed, materials in a powder state may also be mixed before an organic material layer of an organic light emitting device is formed, and it is possible to mix compounds in a liquid state at a temperature which is equal to or more than a suitable temperature. The composition is in a solid state at a temperature which is equal to or less than the melting point of each material, and may be maintained as a liquid phase when the temperature is adjusted.

The composition may additionally include materials publicly known in the art such as solvents and additives.

### Mode for Invention

Hereinafter, the present specification will be described in more detail through Examples, but these Examples are provided only for exemplifying the present application, and are not intended to limit the scope of the present application.

### SYNTHESIS EXAMPLES

### <Preparation Example 1> Preparation of Compound 1-3

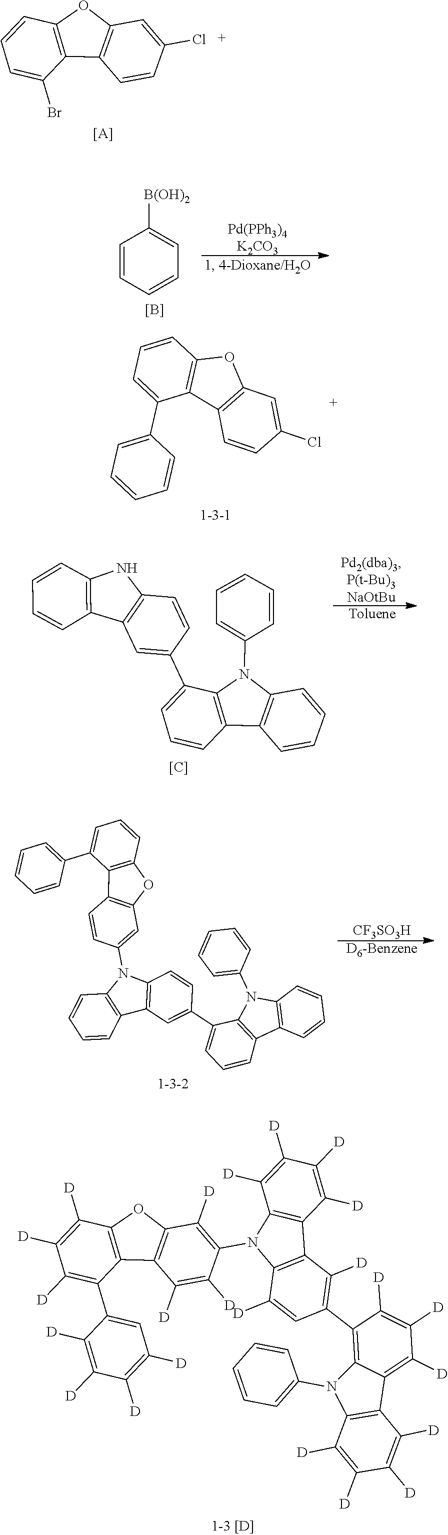

### 1) Preparation of Compound 1-3-1

After 10 g (35.5 mM) of 1-bromo-7-chlorodibenzo[b,d]furan, 4.3 g (35.5 mM) of phenylboronic acids, 2.05 g (1.78 mmol) of Pd(PPh₃)₄, and 12.2 g (88.7 mM) of K₂CO₃ were dissolved in 100 mL/20 mL of 1,4 dioxane/H₂O, the resulting solution was refluxed at 110° C. for 6 hours. After the reaction was completed, distilled water and dichloromethane (DCM) were added thereto at room temperature, extraction was performed, the organic layer was dried over MgSo₄, and then the solvent was removed by a rotary evaporator. The reaction product was purified with column chromatography (DCM:hexane=1:2) to obtain 8.4 g (yield 85%) of Target Compound 1-3-1.

### 2) Preparation of Compound 1-3-2

After 8.4 g (30.2 mM) of Compound 1-3-1, 12.3 g (30.2 mM) of 9-phenyl-9H,9′H-1,3′-bicarbazole, 2.77 g (3.02 mM) of Pd₂(dba)₃, 0.61 g (3.02 mM) of P(t-Bu)₃, and 5.80 g (60.4 mM) of NaOtBu were dissolved in 85 mL of toluene, the resulting solution was refluxed at 110° C. for 6 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:3) and recrystallized with methanol to obtain 11.6 g (59%) of Target Compound 1-3-2.

### 3) Preparation of Compound 1-3[D]

A mixture of 11.6 g (17.82 mM) of Compound 1-3-2, 13.5 g (445.5 mM) of triflic acid, and 100 mL of D₆-benzene was refluxed at 50° C. for 2 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:5) to obtain 11.3 g (88%) of Target Compound 1-3[D].

The results of test experiments on the preparation reaction conditions of Compound 1-3[D] under the conditions of Reference Examples 1 to 9 in the following Tables 1 and 2 are shown below.

TABLE 1

| | | | | Temperature/ | | |
|---|---|---|---|---|---|---|
| Reference | Compound | Solvent | Catalyst | time | | |
| Example | (g, equiv.) | (mL) | (mol %) | (° C., d) | Condition | Yield |
| 1 | 9-phenyl-9′- | D20 | Pt/C | 150° C., 4 d | Round | NO |
| | (9-phenyl- | (100 mL) | (10 mol %) | | flask | reaction |
| | dibenzo[b,d]furan- | | | | Under Ar | |
| | 3-yl)-9H,9′H-1,3′- | | | | bag | |
| | bicarbazole | | | | | |
| | (1 g, 1 eq) | | | | | |
| 2 | 9-phenyl-9′- | | Pt/C, Pd/C | 150° C., 4 d | Round | NO |
| | (9-phenyl- | D20, | (10 mol %) | | flask | reaction |
| | dibenzo[b,d]furan- | i-PrOH, | | | Under Ar | |
| | 3-yl)-9H,9′H-1,3′- | cyclohexane | | | bag | |
| | bicarbazole | (100 mL, | | | | |
| | (1 g, 1 eq) | 50 mL, | | | | |
| | | 50 mL) | | | | |
| 3 | 9-phenyl-9′- | D20, | Pt/C, Pd/C | 200° C., 2 d | sealed tube | NO |
| | (9-phenyl- | i-PrOH, | (10 mol %) | | | reaction |
| | dibenzo[b,d]furan- | cyclohexane | | | | |
| | 3-yl)-9H,9′H-1,3′- | (100 mL, | | | | |
| | bicarbazole | 50 mL, | | | | |
| | (1 g, 1 eq) | 50 mL) | | | | |
| 4 | 9-phenyl-9′- | | | 150° C., 4 d | sealed tube | NO |
| | (9-phenyl- | D20, | Pt/C, Pd/C | | | reaction |
| | dibenzo[b,d]furan- | i-PrOH, | (10 mol %) | | | |
| | 3-yl)-9H,9′H-1,3′- | cyclohexane | | | | |
| | bicarbazole | (100 ml, | | | | |
| | (1 g, 1 eq) | 50 mL, | | | | |
| | | 50 mL) | | | | |

As a result of carrying out a test experiment on the preparation reaction conditions of Compound 1-3[D] under the conditions of Reference Examples 1 to 4 in Table 1 above, it could be confirmed that Compound 1-3 was not synthesized.

TABLE 2

| Reference | Compound | Solvent | Acid | | | Substitution |
|---|---|---|---|---|---|---|
| Example | (g, equiv.) | (mL) | (g, equiv.) | Temperature | Yield | rate |
| 5 | 12H- | Benzene-D6 | CF₃SO₃H | RT | 96% | 43% |
| | benzo[4,5]thieno[2,3- | (10 mL) | (5.77 g, | | | |
| | a]carbazole | | 25 eq) | | | |
| | (1 g, 1 eq) | | | | | |
| 6 | 12H- | Benzene-D6 | CF₃SO₃H | 30° C. | 93% | 61% |
| | benzo[4,5]thieno[2,3- | (10 mL) | (5.77 g, | | | |
| | a]carbazole | | 25 eq) | | | |
| | (1 g, 1 eq) | | | | | |
| 7 | 12H- | Benzene-D6 | CF₃SO₃H | 50° C. | 87% | 88% |
| | benzo[4,5]thieno[2,3- | (10 mL) | (5.77 g, | | | |
| | a]carbazole | | 25 eq) | | | |
| | (1 g, 1 eq) | | | | | |
| 8 | 12H- | DMSO-D6 | CF₃SO₃H | 30° C. | 80% | 52% |
| | benzo[4,5]thieno[2,3- | (10 mL) | (5.77 g, | | | |
| | a]carbazole | | 25 eq) | | | |
| | (1 g, 1 eq) | | | | | |
| 9 | 12H- | DMF-D6 | CF₃SO₃H | 50° C. | 61% | 63% |
| | benzo[4,5]thieno[2,3- | (10 mL) | (5.77 g, | | | |
| | a]carbazole | | 25 eq) | | | |
| | (1 g, 1 eq) | | | | | |

Referring to Table 2, it can be confirmed that in the reaction of substituting hydrogen in an organic compound with deuterium, there is a tendency that the higher the reaction temperature, the higher the substitution rate from hydrogen to deuterium and the lower the yield.

Here, the substitution rate was calculated by [(the number of deuteriums substituted after the chemical reaction)/(the number of hydrogens in the compound before the chemical reaction)]*100. (Based on the number of hydrogen atoms in the remaining portion except for an Ar1 group in Chemical Formula 1 of the present application)

Through a test experiment on the reaction conditions for preparing Compound 1-3[D], it was confirmed that the compound can be obtained with a high deuterium substitution rate and an optimal yield under the reaction conditions of Reference Example 7.

In addition, as a result of N M R analysis, it was confirmed that the portion corresponding to Ar1 in Chemical Formula 1 of the present application was not selectively substituted with deuterium.

That is, the heterocyclic compound of the present invention is prepared under the specific reaction conditions, and thus, has an advantage in that substituent portions including N, O, and S atoms, which have a higher electronegativity than carbon, can be selectively substituted with deuterium at a relatively high substitution rate.

Target Compound D was synthesized in the same manner as in the preparation of Preparation Example 1, except that Intermediate A, Intermediate B, and Intermediate C in the following Table 3 were used instead of 1-bromo-7-chlorodibenzo[b,d]furan, phenylboronic acid, and 9-phenyl-9H,9′H-1,3′-bicarbazole, respectively, in Preparation Example 1.

TABLE 3

| | Intermediate | Intermediate | | | |
|---|---|---|---|---|---|
| Compound No. | A | B | Intermediate C | Target Compound D | Yield |
| 1-3 | 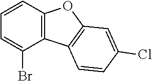 | 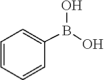 | 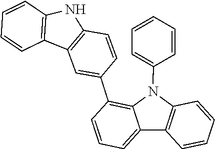 [SMILES: c1ccc(-n2c3ccccc3c3cccc(-c4ccc5[nH]c6ccccc6c5c4)c32)cc1] | 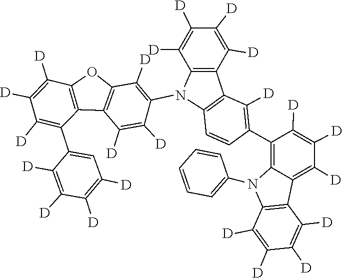 [SMILES: [2H]c1cc(-c2c([2H])c([2H])c([2H])c3oc4c([2H])c(-n5c6ccc(-c7c([2H])c([2H])c([2H])c8c9c([2H])c([2H])c([2H])c([2H])c9n(-c9ccccc9)c78)c([2H])c6c6c([2H])c([2H])c([2H])c([2H])c65)c([2H])c([2H])c4c23)c([2H])c([2H])c1[2H]] | 88% |
| 1-4 | 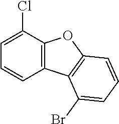 | 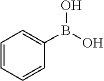 | 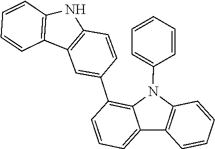 [SMILES: c1ccc(-n2c3ccccc3c3cccc(-c4ccc5[nH]c6ccccc6c5c4)c32)cc1] | 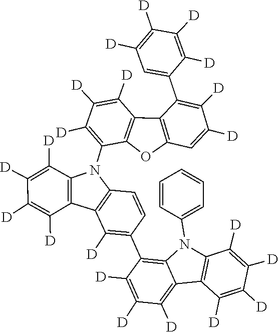 [SMILES: [2H]c1cc(-c2c([2H])c([2H])cc3oc4c(-n5c6ccc(-c7c([2H])c([2H])c([2H])c8c9c([2H])c([2H])c([2H])c([2H])c9n(-c9ccccc9)c78)c([2H])c6c6c([2H])c([2H])c([2H])c([2H])c65)c([2H])c([2H])c([2H])c4c23)c([2H])c([2H])c1[2H]] | 56% |
| 1-7 | 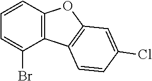 | 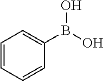 | 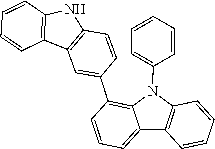 [SMILES: c1ccc(-n2c3ccccc3c3cccc(-c4ccc5[nH]c6ccccc6c5c4)c32)cc1] | 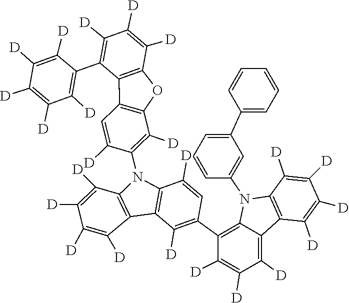 [SMILES: [2H]c1cc2c(oc3c([2H])c([2H])c([2H])c(-c4c([2H])c([2H])c([2H])c([2H])c4[2H])c32)c([2H])c1-n1c2c([2H])cc(-c3c([2H])c([2H])c([2H])c4c5c([2H])c([2H])c([2H])c([2H])c5n(-c5cccc(-c6ccccc6)c5)c34)c([2H])c2c2c([2H])c([2H])c([2H])c([2H])c21] | 43% |
| 1-10 | 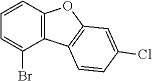 | 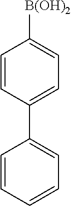 | 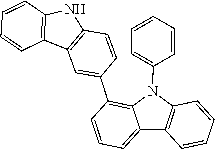 [SMILES: c1ccc(-n2c3ccccc3c3cccc(-c4ccc5[nH]c6ccccc6c5c4)c32)cc1] | 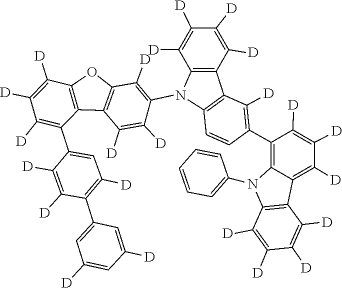 [SMILES: [2H]c1cc([2H])cc(-c2c([2H])cc(-c3c([2H])c([2H])c([2H])c4oc5c([2H])c(-n6c7ccc(-c8c([2H])c([2H])c([2H])c9c%10c([2H])c([2H])c([2H])c([2H])c%10n(-c%10ccccc%10)c89)c([2H])c7c7c([2H])c([2H])c([2H])c([2H])c76)c([2H])c([2H])c5c34)c([2H])c2[2H])c1] | 89% |
| 1-14 | 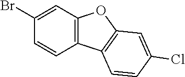 | 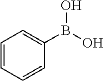 | 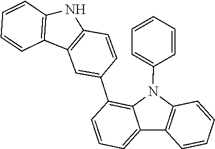 [SMILES: c1ccc(-n2c3ccccc3c3cccc(-c4ccc5[nH]c6ccccc6c5c4)c32)cc1] | 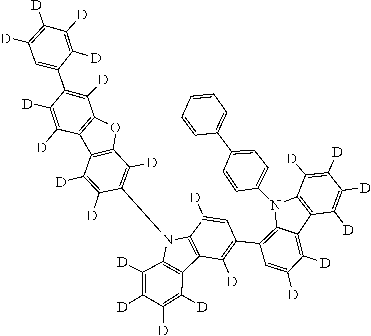 [SMILES: [2H]Cc1c(/C([2H])=C(\C=C)Cc2cc([2H])c([2H])c3c4c([2H])c([2H])c([2H])c([2H])c4n(-c4ccc(-c5ccccc5)cc4)c23)c2c([2H])c([2H])c([2H])c([2H])c2n1-c1c([2H])c([2H])c2c(oc3c([2H])c(-c4cc([2H])c([2H])c([2H])c4[2H])c([2H])c([2H])c32)c1[2H]] | 71% |
| 1-17 | 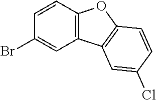 | 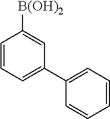 | 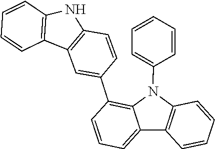 [SMILES: c1ccc(-n2c3ccccc3c3cccc(-c4ccc5[nH]c6ccccc6c5c4)c32)cc1] | 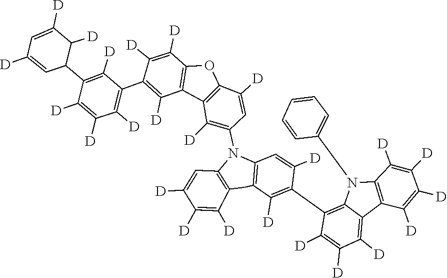 [SMILES: [2H]C1=CC(c2c([2H])c([2H])c([2H])c(-c3c([2H])c([2H])c4oc5c([2H])cc(N6c7cc([2H])c([2H])c([2H])c7Cc7c6cc([2H])c(-c6c([2H])c([2H])c([2H])c8c9c([2H])c([2H])c([2H])c([2H])c9n(-c9ccccc9)c68)c7[2H])c([2H])c5c4c3[2H])c2[2H])C([2H])C([2H])=C1] | 62% |
| 1-23 | 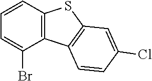 | 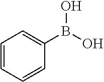 | 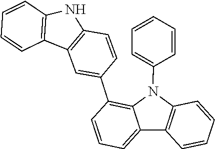 [SMILES: c1ccc(-n2c3ccccc3c3cccc(-c4ccc5[nH]c6ccccc6c5c4)c32)cc1] | 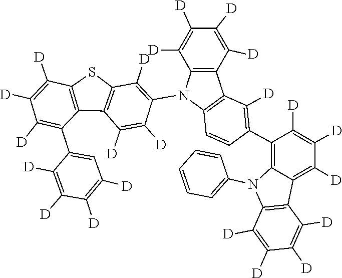 [SMILES: [2H]c1cc(-c2c([2H])c([2H])c([2H])c3sc4c([2H])c(-n5c6ccc(-c7c([2H])c([2H])c([2H])c8c9c([2H])c([2H])c([2H])c([2H])c9n(-c9ccccc9)c78)c([2H])c6c6c([2H])c([2H])c([2H])c([2H])c65)c([2H])c([2H])c4c23)c([2H])c([2H])c1[2H]] | 66% |
| 1-26 | 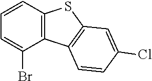 | 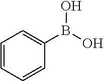 | 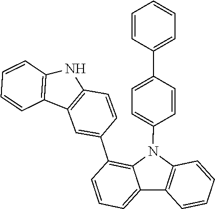 [SMILES: c1ccc(-c2ccc(-n3c4ccccc4c4cccc(-c5ccc6[nH]c7ccccc7c6c5)c43)cc2)cc1] | 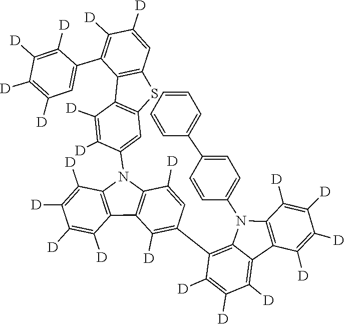 [SMILES: [2H]c1cc(-c2c([2H])c([2H])cc3sc4cc(-n5c6c([2H])cc(-c7c([2H])c([2H])c([2H])c8c9c([2H])c([2H])c([2H])c([2H])c9n(-c9ccc(-c%10ccccc%10)cc9)c78)c([2H])c6c6c([2H])c([2H])c([2H])c([2H])c65)c([2H])c([2H])c4c23)c([2H])c([2H])c1[2H]] | 59% |
| 1-35 | 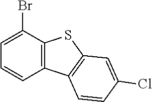 | 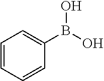 | 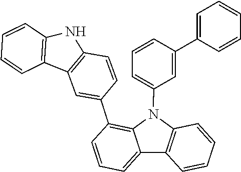 [SMILES: c1ccc(-c2cccc(-n3c4ccccc4c4cccc(-c5ccc6[nH]c7ccccc7c6c5)c43)c2)cc1] | 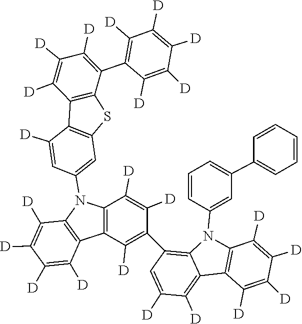 [SMILES: [2H]c1cc(-c2c([2H])c([2H])c3c(c2[2H])c2c([2H])c([2H])c([2H])c([2H])c2n3-c2cc([2H])c3c(c2)sc2c(-c4c([2H])c([2H])c([2H])c([2H])c4[2H])c([2H])c([2H])c([2H])c23)c2c(c1[2H])c1c([2H])c([2H])c([2H])c([2H])c1n2-c1cccc(-c2ccccc2)c1] | 65% |
| 1-38 | 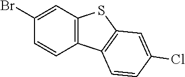 | 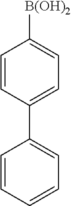 | 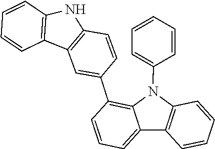 [SMILES: c1ccc(-n2c3ccccc3c3cccc(-c4ccc5[nH]c6ccccc6c5c4)c32)cc1] | 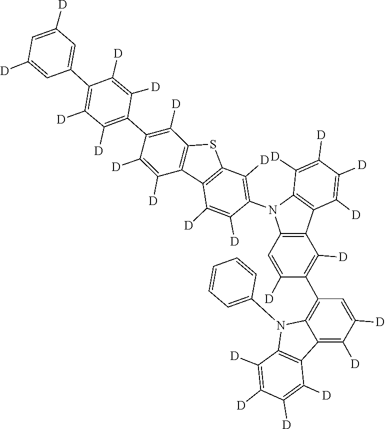 [SMILES: [2H]c1cc([2H])cc(-c2c([2H])c([2H])c(-c3c([2H])c([2H])c4c(sc5c([2H])c(-n6c7cc([2H])c(-c8cc([2H])c([2H])c9c%10c([2H])c([2H])c([2H])c([2H])c%10n(-c%10ccccc%10)c89)c([2H])c7c7c([2H])c([2H])c([2H])c([2H])c76)c([2H])c([2H])c54)c3[2H])c([2H])c2[2H])c1] | 53% |
| 1-43 | 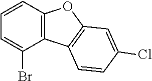 | 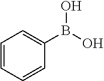 | 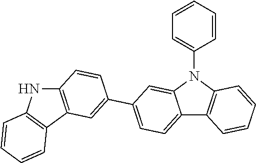 [SMILES: c1ccc(-n2c3ccccc3c3ccc(-c4ccc5[nH]c6ccccc6c5c4)cc32)cc1] | 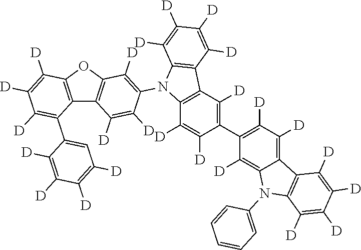 [SMILES: [2H]c1cc(-c2c([2H])c([2H])c([2H])c3oc4c([2H])c(-n5c6c([2H])c([2H])c([2H])c([2H])c6c6c([2H])c(-c7c([2H])c([2H])c8c9c([2H])c([2H])c([2H])c([2H])c9n(-c9ccccc9)c8c7[2H])c([2H])c([2H])c65)c([2H])c([2H])c4c23)c([2H])c([2H])c1[2H]] | 87% |
| 1-44 | 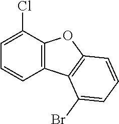 | 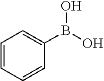 | 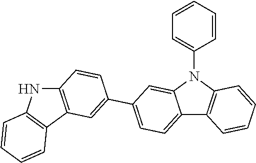 [SMILES: c1ccc(-n2c3ccccc3c3ccc(-c4ccc5[nH]c6ccccc6c5c4)cc32)cc1] | 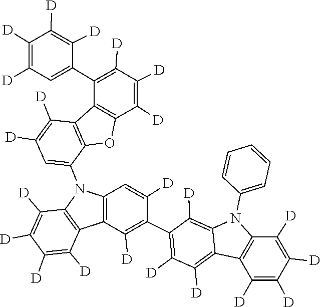 [SMILES: [2H]c1cc(-c2c([2H])c([2H])c([2H])c3oc4c(-n5c6cc([2H])c(-c7c([2H])c([2H])c8c9c([2H])c([2H])c([2H])c([2H])c9n(-c9ccccc9)c8c7[2H])c([2H])c6c6c([2H])c([2H])c([2H])c([2H])c65)cc([2H])c([2H])c4c23)c([2H])c([2H])c1[2H]] | 89% |
| 1-46 | 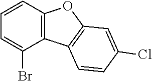 | 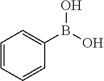 | 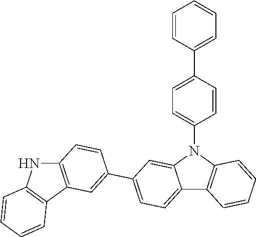 [SMILES: c1ccc(-c2ccc(-n3c4ccccc4c4ccc(-c5ccc6[nH]c7ccccc7c6c5)cc43)cc2)cc1] | 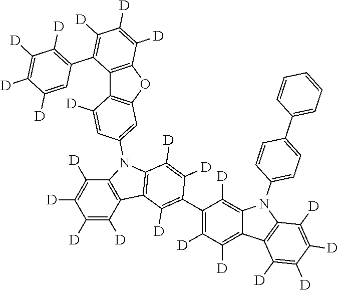 [SMILES: [2H]c1cc(-c2c([2H])c([2H])c([2H])c3oc4cc(-n5c6c([2H])c([2H])c([2H])c([2H])c6c6c([2H])c(-c7c([2H])c([2H])c8c9c([2H])c([2H])c([2H])c([2H])c9n(-c9ccc(-c%10ccccc%10)cc9)c8c7[2H])c([2H])c([2H])c65)cc([2H])c4c23)c([2H])c([2H])c1[2H]] | 93% |
| 1-51 | 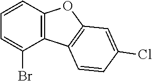 | 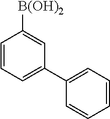 | 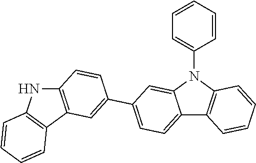 [SMILES: c1ccc(-n2c3ccccc3c3ccc(-c4ccc5[nH]c6ccccc6c5c4)cc32)cc1] | 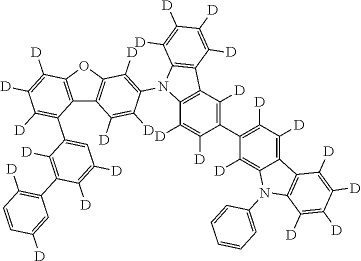 [SMILES: [2H]c1ccc([2H])c(-c2c([2H])c([2H])cc(-c3c([2H])c([2H])c([2H])c4oc5c([2H])c(-n6c7c([2H])c([2H])c([2H])c([2H])c7c7c([2H])c(-c8c([2H])c([2H])c9c%10c([2H])c([2H])c([2H])c([2H])c%10n(-c%10ccccc%10)c9c8[2H])c([2H])c([2H])c76)c([2H])c([2H])c5c34)c2[2H])c1] | 91% |
| 1-54 | 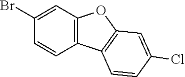 | 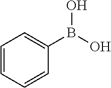 | 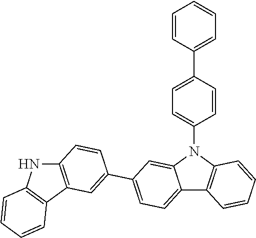 [SMILES: c1ccc(-c2ccc(-n3c4ccccc4c4ccc(-c5ccc6[nH]c7ccccc7c6c5)cc43)cc2)cc1] | 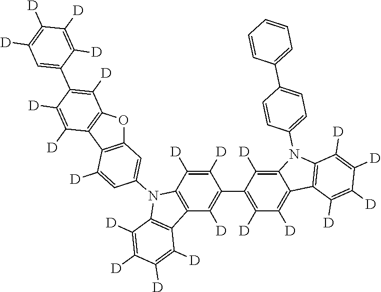 [SMILES: [2H]c1cc(-c2c([2H])c([2H])c3c(oc4cc(-n5c6c([2H])c([2H])c([2H])c([2H])c6c6c([2H])c(-c7c([2H])c([2H])c8c9c([2H])c([2H])c([2H])c([2H])c9n(-c9ccc(-c%10ccccc%10)cc9)c8c7[2H])c([2H])c([2H])c65)cc([2H])c43)c2[2H])c([2H])c([2H])c1[2H]] | 88% |
| 1-56 | 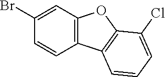 | 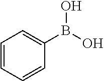 | 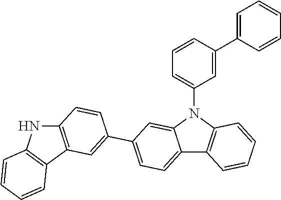 [SMILES: c1ccc(-c2cccc(-n3c4ccccc4c4ccc(-c5ccc6[nH]c7ccccc7c6c5)cc43)c2)cc1] | 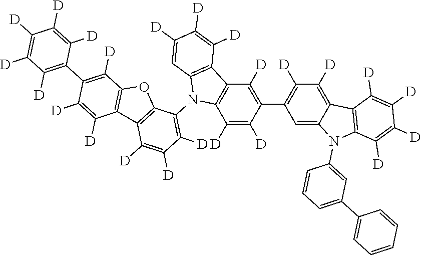 [SMILES: [2H]c1cc2c(c([2H])c1[2H])c1c([2H])c(-c3cc4c(c([2H])c3[2H])c3c([2H])c([2H])c([2H])c([2H])c3n4-c3cccc(-c4ccccc4)c3)c([2H])c([2H])c1n2-c1c([2H])c([2H])c([2H])c2c1oc1c([2H])c(-c3c([2H])c([2H])c([2H])c([2H])c3[2H])c([2H])c([2H])c12] | 71% |
| 1-62 | 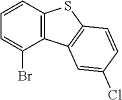 | 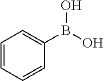 | 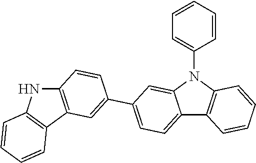 [SMILES: c1ccc(-n2c3ccccc3c3ccc(-c4ccc5[nH]c6ccccc6c5c4)cc32)cc1] | 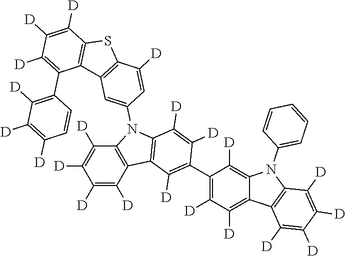 [SMILES: [2H]c1ccc(-c2c([2H])c([2H])c([2H])c3sc4c([2H])cc(-n5c6c([2H])c([2H])c([2H])c([2H])c6c6c([2H])c(-c7c([2H])c([2H])c8c9c([2H])c([2H])c([2H])c([2H])c9n(-c9ccccc9)c8c7[2H])c([2H])c([2H])c65)cc4c23)c([2H])c1[2H]] | 83% |
| 1-68 | 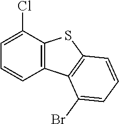 | 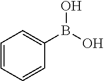 | 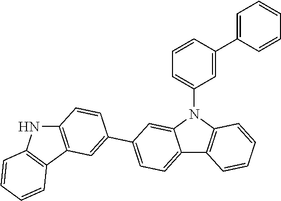 [SMILES: c1ccc(-c2cccc(-n3c4ccccc4c4ccc(-c5ccc6[nH]c7ccccc7c6c5)cc43)c2)cc1] | 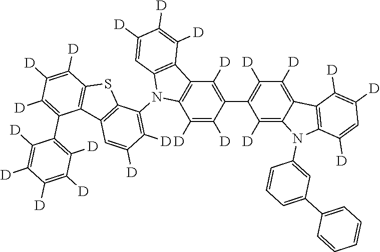 [SMILES: [2H]c1cc2c(sc3c([2H])c([2H])c([2H])c(-c4c([2H])c([2H])c([2H])c([2H])c4[2H])c32)c(-n2c3cc([2H])c([2H])c([2H])c3c3c([2H])c(-c4c([2H])c([2H])c5c6c([2H])c([2H])cc([2H])c6n(-c6cccc(-c7ccccc7)c6)c5c4[2H])c([2H])c([2H])c32)c1[2H]] | 90% |
| 1-70 | 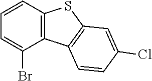 | 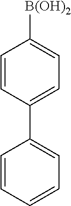 | 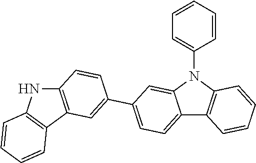 [SMILES: c1ccc(-n2c3ccccc3c3ccc(-c4ccc5[nH]c6ccccc6c5c4)cc32)cc1] | 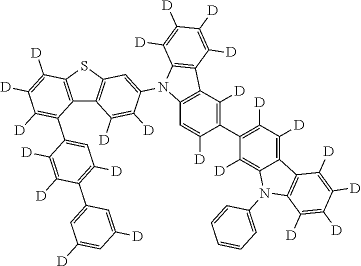 [SMILES: [2H]c1cc([2H])cc(-c2c([2H])cc(-c3c([2H])c([2H])c([2H])c4sc5cc(-n6c7cc([2H])c(-c8c([2H])c([2H])c9c%10c([2H])c([2H])c([2H])c([2H])c%10n(-c%10ccccc%10)c9c8[2H])c([2H])c7c7c([2H])c([2H])c([2H])c([2H])c76)c([2H])c([2H])c5c34)c([2H])c2[2H])c1] | 83% |
| 1-72 | 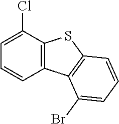 | 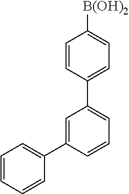 | 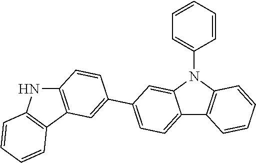 [SMILES: c1ccc(-n2c3ccccc3c3ccc(-c4ccc5[nH]c6ccccc6c5c4)cc32)cc1] | 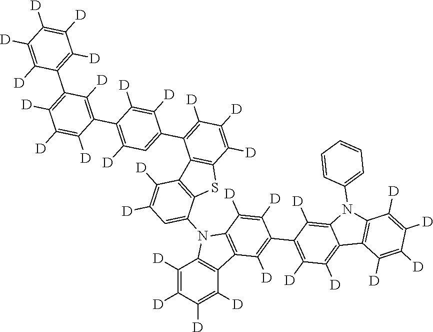 [SMILES: [2H]c1cc(-c2c([2H])c([2H])c([2H])c3sc4c(-n5c6c([2H])c([2H])c([2H])c([2H])c6c6c([2H])c(-c7c([2H])c([2H])c8c9c([2H])c([2H])c([2H])c([2H])c9n(-c9ccccc9)c8c7[2H])c([2H])c([2H])c65)cc([2H])c([2H])c4c23)c([2H])c([2H])c1-c1c([2H])c([2H])c([2H])c(-c2c([2H])c([2H])c([2H])c([2H])c2[2H])c1[2H]] | 77% |
| 1-75 | 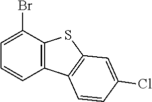 | 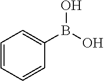 | 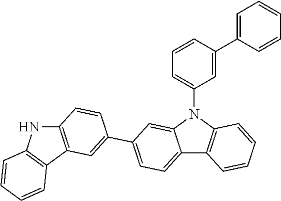 [SMILES: c1ccc(-c2cccc(-n3c4ccccc4c4ccc(-c5ccc6[nH]c7ccccc7c6c5)cc43)c2)cc1] | 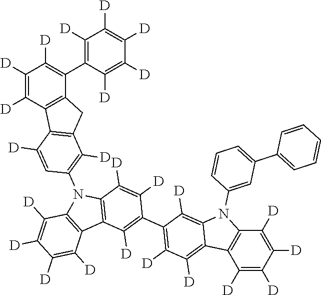 [SMILES: [2H]c1cc(-n2c3c([2H])c([2H])c([2H])c([2H])c3c3c([2H])c(-c4c([2H])c([2H])c5c6c([2H])c([2H])c([2H])c([2H])c6n(-c6cccc(-c7ccccc7)c6)c5c4[2H])c([2H])c([2H])c32)c([2H])c2c1-c1c([2H])c([2H])c([2H])c(-c3c([2H])c([2H])c([2H])c([2H])c3[2H])c1C2] | 89% |
| 1-78 | 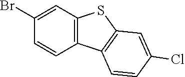 | 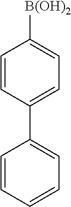 | 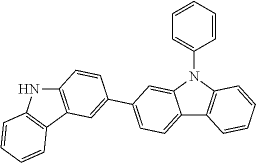 [SMILES: c1ccc(-n2c3ccccc3c3ccc(-c4ccc5[nH]c6ccccc6c5c4)cc32)cc1] | 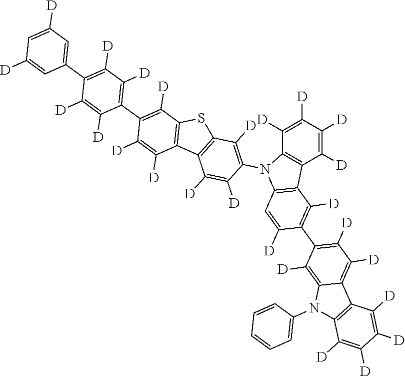 [SMILES: [2H]c1cc([2H])cc(-c2c([2H])c([2H])c(-c3c([2H])c([2H])c4c(sc5c([2H])c(-n6c7cc([2H])c(-c8c([2H])c([2H])c9c%10c([2H])c([2H])c([2H])c([2H])c%10n(-c%10ccccc%10)c9c8[2H])c([2H])c7c7c([2H])c([2H])c([2H])c([2H])c76)c([2H])c([2H])c54)c3[2H])c([2H])c2[2H])c1] | 71% |
| 1-83 | 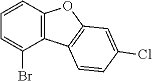 | 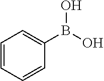 | 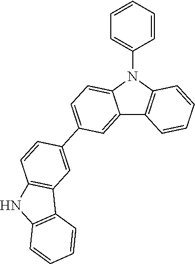 [SMILES: c1ccc(-n2c3ccccc3c3cc(-c4ccc5[nH]c6ccccc6c5c4)ccc32)cc1] | 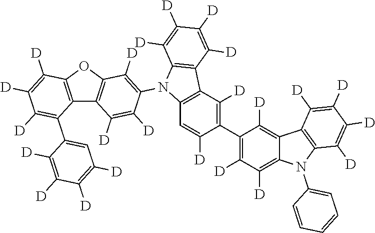 [SMILES: [2H]c1cc(-c2c([2H])c([2H])c([2H])c3oc4c([2H])c(-n5c6cc([2H])c(-c7c([2H])c([2H])c8c(c7[2H])c7c([2H])c([2H])c([2H])c([2H])c7n8-c7ccccc7)c([2H])c6c6c([2H])c([2H])c([2H])c([2H])c65)c([2H])c([2H])c4c23)c([2H])c([2H])c1[2H]] | 63% |
| 1-85 | 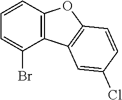 | 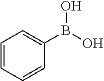 | 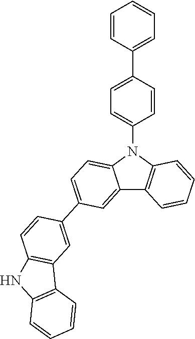 [SMILES: c1ccc(-c2ccc(-n3c4ccccc4c4cc(-c5ccc6[nH]c7ccccc7c6c5)ccc43)cc2)cc1] | 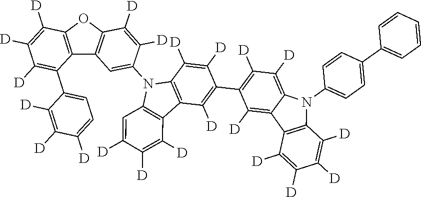 [SMILES: [2H]c1ccc(-c2c([2H])c([2H])c([2H])c3oc4c([2H])c([2H])c(-n5c6cc([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)cc4c23)c([2H])c1[2H]] | 98% |
| 1-86 | 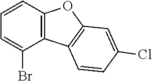 | 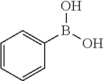 | 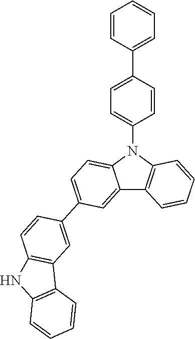 [SMILES: c1ccc(-c2ccc(-n3c4ccccc4c4cc(-c5ccc6[nH]c7ccccc7c6c5)ccc43)cc2)cc1] | 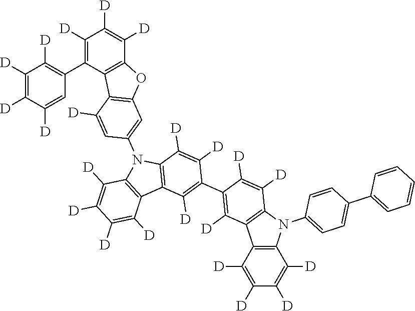 [SMILES: [2H]c1cc(-c2c([2H])c([2H])c([2H])c3oc4cc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)cc([2H])c4c23)c([2H])c([2H])c1[2H]] | 72% |
| 1-87 | 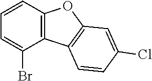 | 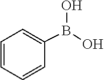 | 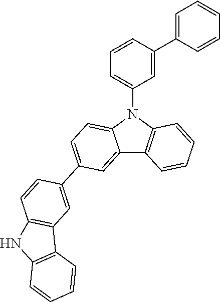 [SMILES: c1ccc(-c2cccc(-n3c4ccccc4c4cc(-c5ccc6[nH]c7ccccc7c6c5)ccc43)c2)cc1] | 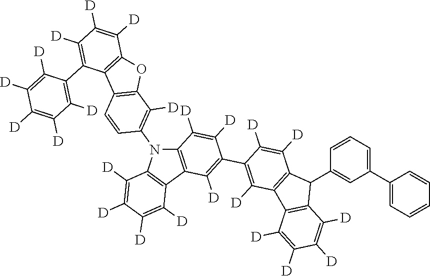 [SMILES: [2H]c1c([2H])c([2H])c(-c2c([2H])c([2H])c([2H])c3oc4c([2H])c(-n5c6c([2H])c([2H])c([2H])c([2H])c6c6c([2H])c(-c7c([2H])c([2H])c8c(c7[2H])-c7c([2H])c([2H])c([2H])c([2H])c7C8c7cccc(-c8ccccc8)c7)c([2H])c([2H])c65)ccc4c23)c([2H])c1[2H]] | 83% |
| 1-90 | 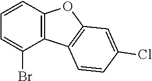 | 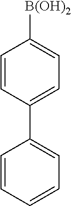 | 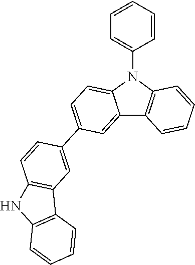 [SMILES: c1ccc(-n2c3ccccc3c3cc(-c4ccc5[nH]c6ccccc6c5c4)ccc32)cc1] | 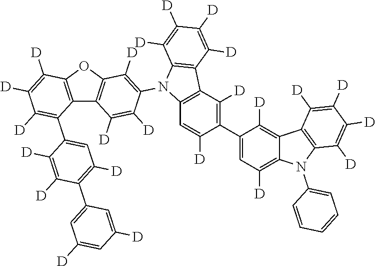 [SMILES: [2H]c1cc([2H])cc(-c2c([2H])cc(-c3c([2H])c([2H])c([2H])c4oc5c([2H])c(-n6c7cc([2H])c(-c8cc([2H])c9c(c8[2H])c8c([2H])c([2H])c([2H])c([2H])c8n9-c8ccccc8)c([2H])c7c7c([2H])c([2H])c([2H])c([2H])c76)c([2H])c([2H])c5c34)c([2H])c2[2H])c1] | 64% |
| 1-93 | 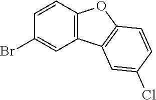 | 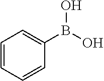 | 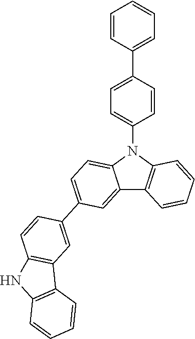 [SMILES: c1ccc(-c2ccc(-n3c4ccccc4c4cc(-c5ccc6[nH]c7ccccc7c6c5)ccc43)cc2)cc1] | 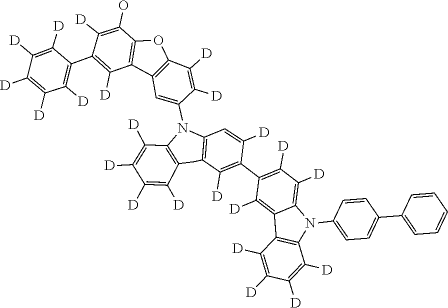 | 76% |
| 1-94 | 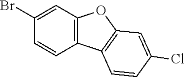 | 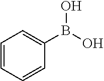 | 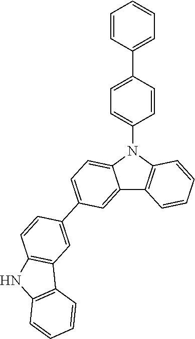 [SMILES: c1ccc(-c2ccc(-n3c4ccccc4c4cc(-c5ccc6[nH]c7ccccc7c6c5)ccc43)cc2)cc1] | 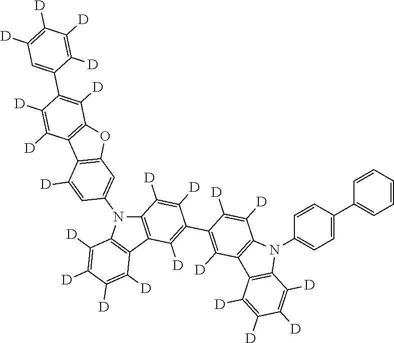 [SMILES: [2H]c1cc(-c2c([2H])c([2H])c3c(oc4cc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)cc([2H])c43)c2[2H])c([2H])c([2H])c1[2H]] | 59% |
| 1-96 | 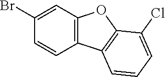 | 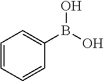 | 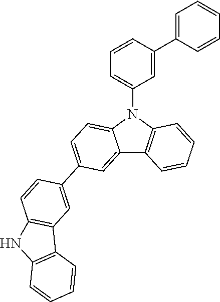 [SMILES: c1ccc(-c2cccc(-n3c4ccccc4c4cc(-c5ccc6[nH]c7ccccc7c6c5)ccc43)c2)cc1] | 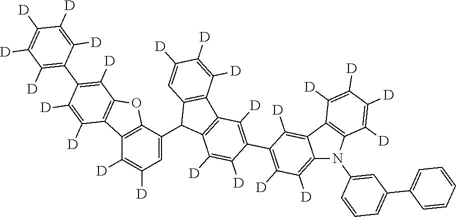 [SMILES: [2H]c1cc2c(c([2H])c1[2H])-c1c([2H])c(-c3c([2H])c([2H])c4c(c3[2H])c3c([2H])c([2H])c([2H])c([2H])c3n4-c3cccc(-c4ccccc4)c3)c([2H])c([2H])c1C2c1cc([2H])c([2H])c2c1oc1c([2H])c(-c3c([2H])c([2H])c([2H])c([2H])c3[2H])c([2H])c([2H])c12] | 66% |
| 1-97 | 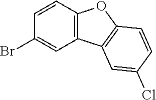 | 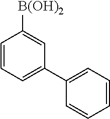 | 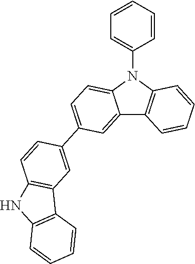 [SMILES: c1ccc(-n2c3ccccc3c3cc(-c4ccc5[nH]c6ccccc6c5c4)ccc32)cc1] | 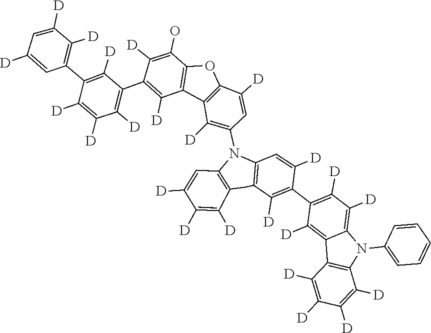 | 69% |
| 1-103 | 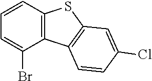 | 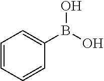 | 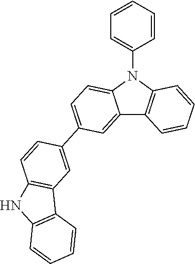 [SMILES: c1ccc(-n2c3ccccc3c3cc(-c4ccc5[nH]c6ccccc6c5c4)ccc32)cc1] | 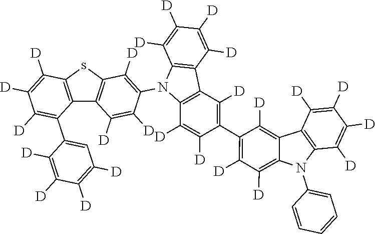 | 92% |
| 1-104 | 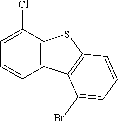 | 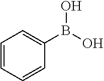 | 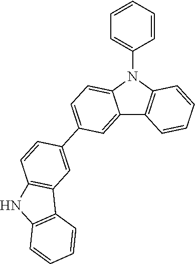 [SMILES: c1ccc(-n2c3ccccc3c3cc(-c4ccc5[nH]c6ccccc6c5c4)ccc32)cc1] | 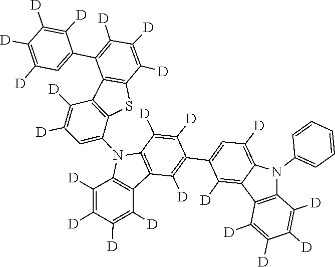 [SMILES: [2H]c1cc(-c2c([2H])c([2H])c([2H])c3sc4c(-n5c6c([2H])c([2H])c([2H])c([2H])c6c6c([2H])c(-c7cc([2H])c8c(c7[2H])c7c([2H])c([2H])c([2H])c([2H])c7n8-c7ccccc7)c([2H])c([2H])c65)cc([2H])c([2H])c4c23)c([2H])c([2H])c1[2H]] | 94% |
| 1-106 | 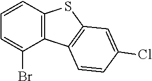 | 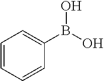 | 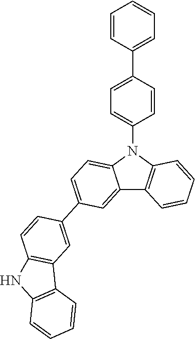 [SMILES: c1ccc(-c2ccc(-n3c4ccccc4c4cc(-c5ccc6[nH]c7ccccc7c6c5)ccc43)cc2)cc1] | 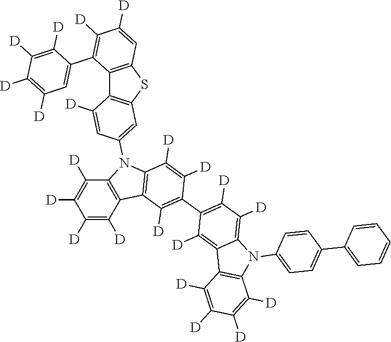 [SMILES: [2H]c1cc(-c2c([2H])c([2H])cc3sc4cc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)cc([2H])c4c23)c([2H])c([2H])c1[2H]] | 82% |
| 1-107 | 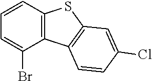 | 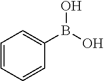 | 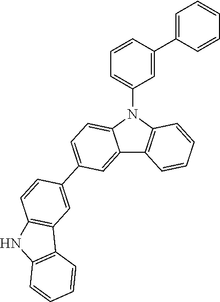 [SMILES: c1ccc(-c2cccc(-n3c4ccccc4c4cc(-c5ccc6[nH]c7ccccc7c6c5)ccc43)c2)cc1] | 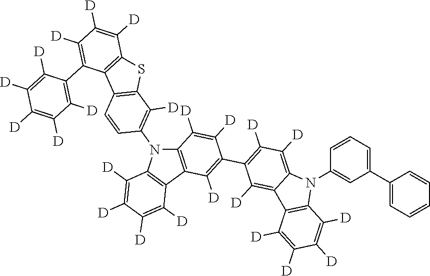 [SMILES: [2H]c1c([2H])c([2H])c(-c2c([2H])c([2H])c([2H])c3sc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7cccc(-c8ccccc8)c7)c([2H])c([2H])c65)ccc4c23)c([2H])c1[2H]] | 77% |
| 1-110 | 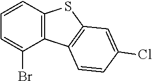 | 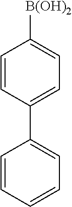 | 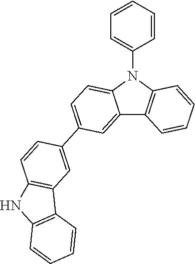 [SMILES: c1ccc(-n2c3ccccc3c3cc(-c4ccc5[nH]c6ccccc6c5c4)ccc32)cc1] | 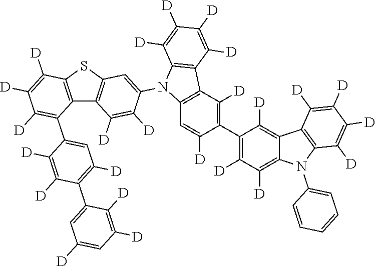 [SMILES: [2H]c1cc([2H])c([2H])c(-c2c([2H])cc(-c3c([2H])c([2H])c([2H])c4sc5cc(-n6c7cc([2H])c(-c8c([2H])c([2H])c9c(c8[2H])c8c([2H])c([2H])c([2H])c([2H])c8n9-c8ccccc8)c([2H])c7c7c([2H])c([2H])c([2H])c([2H])c76)c([2H])c([2H])c5c34)c([2H])c2[2H])c1] | 93% |
| 1-114 | 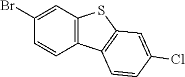 | 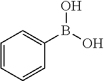 | 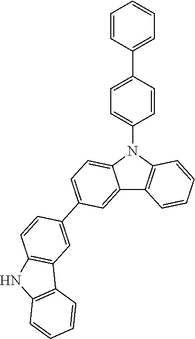 [SMILES: c1ccc(-c2ccc(-n3c4ccccc4c4cc(-c5ccc6[nH]c7ccccc7c6c5)ccc43)cc2)cc1] | 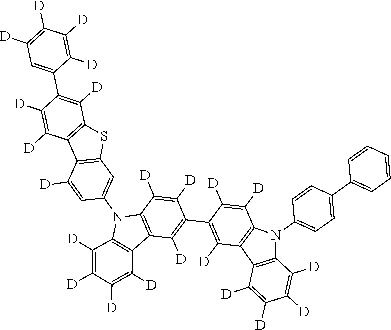 [SMILES: [2H]c1cc(-c2c([2H])c([2H])c3c(sc4cc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)cc([2H])c43)c2[2H])c([2H])c([2H])c1[2H]] | 96% |
| 1-118 | 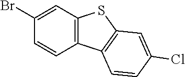 | 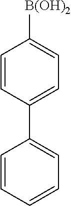 | 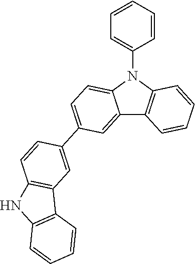 [SMILES: c1ccc(-n2c3ccccc3c3cc(-c4ccc5[nH]c6ccccc6c5c4)ccc32)cc1] | 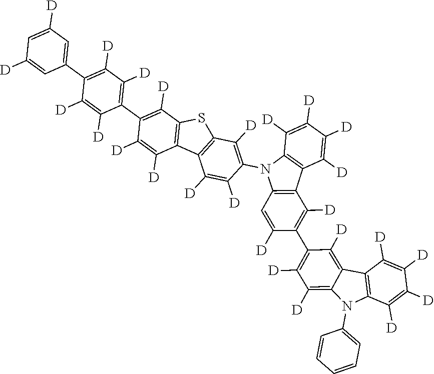 [SMILES: [2H]c1cc([2H])cc(-c2c([2H])c([2H])c(-c3c([2H])c([2H])c4c(sc5c([2H])c(-n6c7cc([2H])c(-c8c([2H])c([2H])c9c(c8[2H])c8c([2H])c([2H])c([2H])c([2H])c8n9-c8ccccc8)c([2H])c7c7c([2H])c([2H])c([2H])c([2H])c76)c([2H])c([2H])c54)c3[2H])c([2H])c2[2H])c1] | 84% |
| 1-122 | 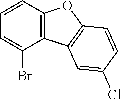 | 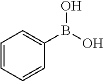 | 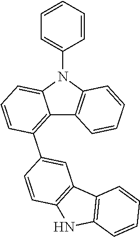 [SMILES: c1ccc(-n2c3ccccc3c3c(-c4ccc5[nH]c6ccccc6c5c4)cccc32)cc1] | 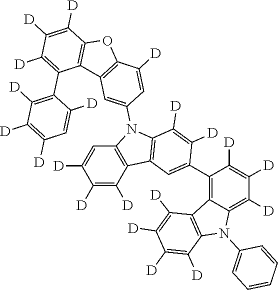 [SMILES: [2H]c1cc([2H])c(-c2c([2H])c([2H])c([2H])c3oc4c([2H])cc(-n5c6cc([2H])c([2H])c([2H])c6c6cc(-c7c([2H])c([2H])c([2H])c8c7c7c([2H])c([2H])c([2H])c([2H])c7n8-c7ccccc7)c([2H])c([2H])c65)cc4c23)c([2H])c1[2H]] | 56% |
| 1-128 | 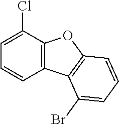 | 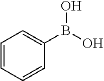 | 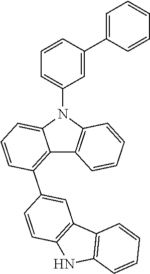 [SMILES: c1ccc(-c2cccc(-n3c4ccccc4c4c(-c5ccc6[nH]c7ccccc7c6c5)cccc43)c2)cc1] | 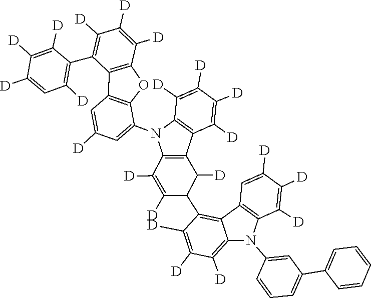 [SMILES: [2H]Cc1c([2H])c([2H])c2c(c1C1C([2H])=C([2H])c3c(c4c([2H])c([2H])c([2H])c([2H])c4n3-c3cc([2H])cc4c3oc3c([2H])c([2H])c([2H])c(-c5c([2H])cc([2H])c([2H])c5[2H])c34)C1[2H])c1cc([2H])c([2H])c([2H])c1n2-c1cccc(-c2ccccc2)c1] | 45% |
| 1-129 | 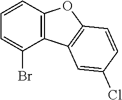 | 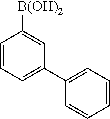 | 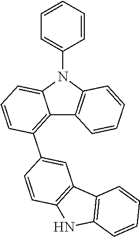 [SMILES: c1ccc(-n2c3ccccc3c3c(-c4ccc5[nH]c6ccccc6c5c4)cccc32)cc1] | 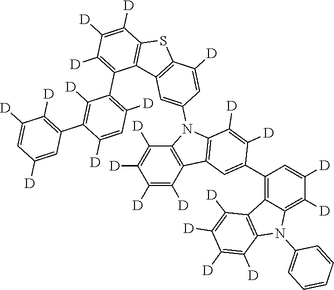 [SMILES: [2H]c1cc([2H])c([2H])c(-c2c([2H])cc([2H])c(-c3c([2H])c([2H])c([2H])c4sc5c([2H])cc(-n6c7c([2H])c([2H])c(-c8cc([2H])c([2H])c9c8c8c([2H])c([2H])c([2H])c([2H])c8n9-c8ccccc8)cc7c7c([2H])c([2H])c([2H])c([2H])c76)cc5c34)c2[2H])c1] | 63% |
| 1-134 | 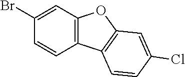 | 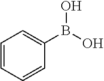 | 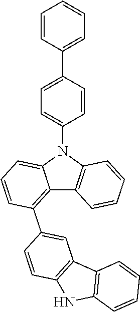 [SMILES: c1ccc(-c2ccc(-n3c4ccccc4c4c(-c5ccc6[nH]c7ccccc7c6c5)cccc43)cc2)cc1] | 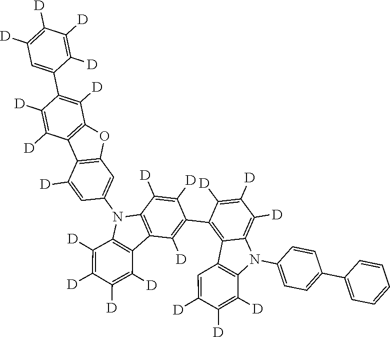 [SMILES: [2H]c1cc(-c2c([2H])c([2H])c3c(oc4cc(-n5c6c([2H])c([2H])c([2H])c([2H])c6c6c([2H])c(-c7c([2H])c([2H])c([2H])c8c7c7cc([2H])c([2H])c([2H])c7n8-c7ccc(-c8ccccc8)cc7)c([2H])c([2H])c65)cc([2H])c43)c2[2H])c([2H])c([2H])c1[2H]] | 66% |
| 1-136 | 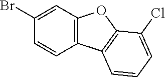 | 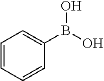 | 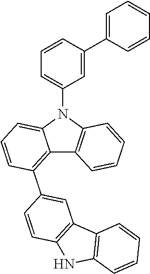 [SMILES: c1ccc(-c2cccc(-n3c4ccccc4c4c(-c5ccc6[nH]c7ccccc7c6c5)cccc43)c2)cc1] | 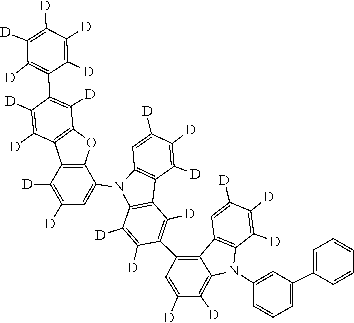 [SMILES: [2H]c1cc2c(c([2H])c1[2H])c1c([2H])c(-c3cc([2H])c([2H])c4c3c3cc([2H])c([2H])c([2H])c3n4-c3cccc(-c4ccccc4)c3)c([2H])c([2H])c1n2-c1cc([2H])c([2H])c2c1oc1c([2H])c(-c3c([2H])c([2H])c([2H])c([2H])c3[2H])c([2H])c([2H])c12] | 76% |
| 1-143 | 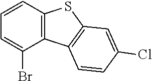 | 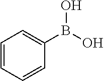 | 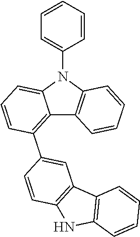 [SMILES: c1ccc(-n2c3ccccc3c3c(-c4ccc5[nH]c6ccccc6c5c4)cccc32)cc1] | 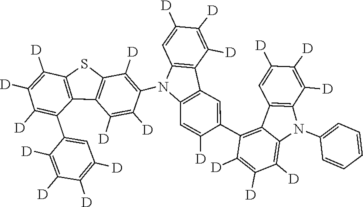 [SMILES: [2H]c1cc2c(cc1-c1c([2H])c([2H])c([2H])c3c1c1cc([2H])c([2H])c([2H])c1n3-c1ccccc1)c1c([2H])c([2H])c([2H])cc1n2-c1c([2H])c([2H])c2c(sc3c([2H])c([2H])c([2H])c(-c4cc([2H])c([2H])c([2H])c4[2H])c32)c1[2H]] | 81% |
| 1-146 | 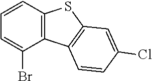 | 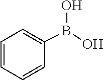 | 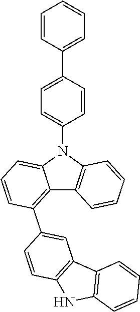 [SMILES: c1ccc(-c2ccc(-n3c4ccccc4c4c(-c5ccc6[nH]c7ccccc7c6c5)cccc43)cc2)cc1] | 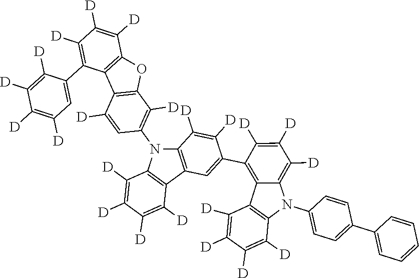 [SMILES: [2H]c1cc(-c2c([2H])c([2H])c([2H])c3oc4c([2H])c(-n5c6c([2H])c([2H])c(-c7c([2H])c([2H])c([2H])c8c7c7c([2H])c([2H])c([2H])c([2H])c7n8-c7ccc(-c8ccccc8)cc7)cc6c6c([2H])c([2H])c([2H])c([2H])c65)cc([2H])c4c23)c([2H])c([2H])c1[2H]] | 59% |
| 1-147 | 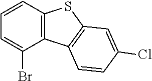 | 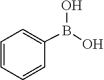 | 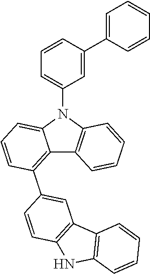 [SMILES: c1ccc(-c2cccc(-n3c4ccccc4c4c(-c5ccc6[nH]c7ccccc7c6c5)cccc43)c2)cc1] | 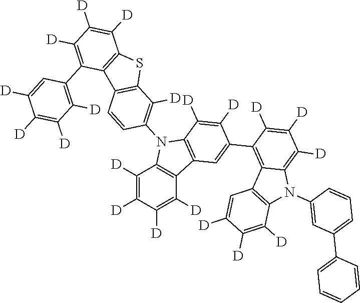 [SMILES: [2H]c1cc(-c2c([2H])c([2H])c([2H])c3sc4c([2H])c(-n5c6c([2H])c([2H])c(-c7c([2H])c([2H])c([2H])c8c7c7cc([2H])c([2H])c([2H])c7n8-c7cccc(-c8ccccc8)c7)cc6c6c([2H])c([2H])c([2H])c([2H])c65)ccc4c23)c([2H])c([2H])c1[2H]] | 67% |
| 1-150 | 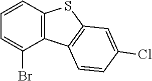 | 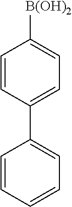 | 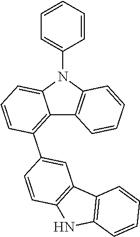 [SMILES: c1ccc(-n2c3ccccc3c3c(-c4ccc5[nH]c6ccccc6c5c4)cccc32)cc1] | 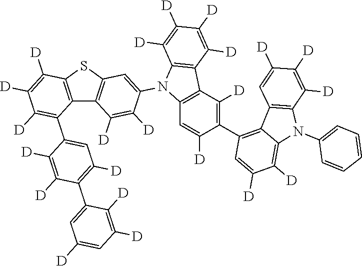 [SMILES: [2H]c1cc([2H])c([2H])c(-c2c([2H])cc(-c3c([2H])c([2H])c([2H])c4sc5cc(-n6c7cc([2H])c(-c8cc([2H])c([2H])c9c8c8cc([2H])c([2H])c([2H])c8n9-c8ccccc8)c([2H])c7c7c([2H])c([2H])c([2H])c([2H])c76)c([2H])c([2H])c5c34)c([2H])c2[2H])c1] | 53% |
| 1-155 | 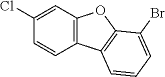 | 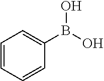 | 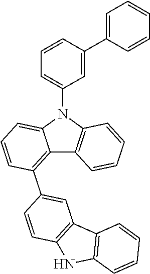 [SMILES: c1ccc(-c2cccc(-n3c4ccccc4c4c(-c5ccc6[nH]c7ccccc7c6c5)cccc43)c2)cc1] | 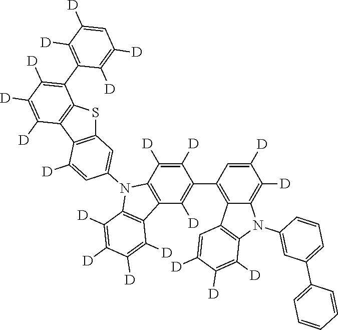 [SMILES: [2H]c1cc([2H])c([2H])c(-c2c([2H])c([2H])c([2H])c3c2sc2cc(-n4c5c([2H])c([2H])c([2H])c([2H])c5c5c([2H])c(-c6cc([2H])c([2H])c7c6c6cc([2H])c([2H])c([2H])c6n7-c6cccc(-c7ccccc7)c6)c([2H])c([2H])c54)cc([2H])c23)c1[2H]] | 93% |

### [Preparation Example 2] Preparation of Compound 2-9

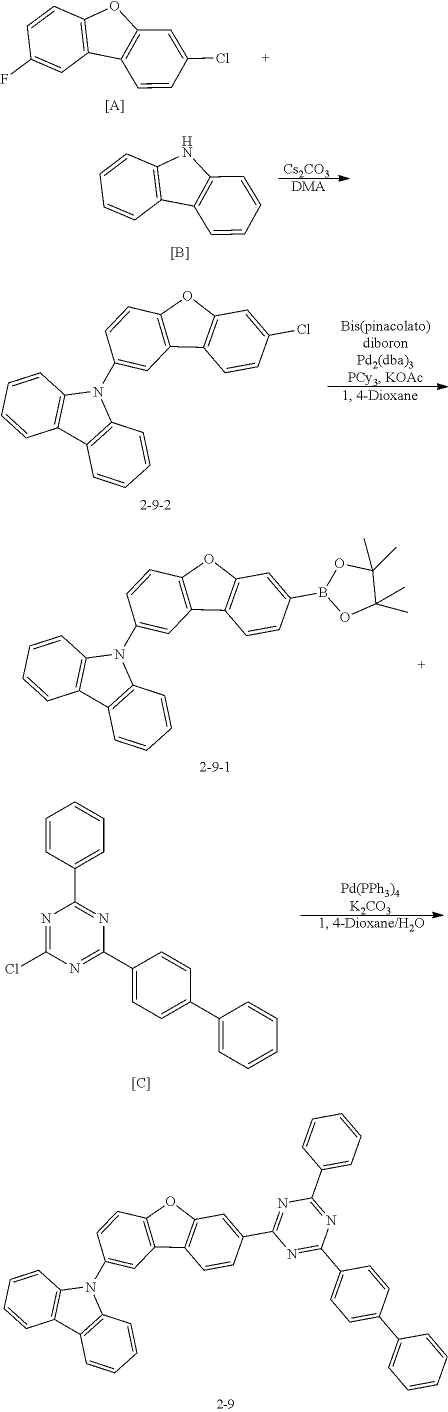

### 1) Preparation of Compound 2-9-2

A mixture of dimethylacetamide (60 ml) with 7-chloro-2-fluorodibenzo-[b,d]furan (6 g, 27.19 mmol), 9H-carbazole (5 g, 29.9 mmol) and Cs₂CO₃ (22 g, 101.7 mmol) was refluxed at 170° C. for 12 hours in a one-neck round-bottom flask. After the mixture was cooled, the mixture was filtered, the solvent of the filtrate was removed, and then column purification (DCM:hexane=1:3) was performed to obtain Compound 2-9-2. (9 g, 90%)

### 2) Preparation of Compound 2-9-1

A mixture of 1,4-dioxane (100 ml) with 9-(7-chlorodibenzo[b,d]furan-2-yl)-9H-carbazole (9 g, 24.4 mmol), bis(pinacolato)diboron (12.4 g, 48.9 mmol), Pcy3 (1.37 g, 4.89 mmol), potassium acetate (7.1 g, 73 mmol), and Pd₂(dba)₃ (2.2 g, 2.44 mmol) was refluxed at 140° C. for 3 hours in a one-neck round-bottom flask. After the mixture was cooled, the filtered filtrate was concentrated, and column purification (DCM:hexane=1:3) was performed to obtain Compound 2-9-1. (7.2 g, 64%)

### 3) Preparation of Compound 2-9

A mixture of 9-(7-(4,4,5,5-tetramethyl-1,3,2-dioxaborolan-2-yl)dibenzo[b,d]furan-2-yl)-9H-carbazole (7.2 g, 15.6 mmol), 2-([1,1′-biphenyl]-4-yl)-4-chloro-6-phenyl-1,3,5-triazine (5.90 g, 17.16 mmol), tetrakis(triphenyl phosphine)palladium(0) (0.90 g, 0.78 mmol), potassium carbonate (4.31 g, 31.2 mmol), and 1,4-dioxane/water (100 ml/25 ml) was refluxed at 120° C. for 4 hours in a one-neck round-bottom flask. The mixture was filtered at room temperature, and then washed with 1,4-dioxane, distilled water, and MeOH to obtain Compound 2-9. (7.50 g, 75%)

Synthesis was performed in the same manner as in Preparation Example 2, except that Intermediates A, B, and C in the following Table 4 were used instead of [A, [B],and [C], respectively, in Preparation Example 2.

TABLE 4

| Com- | | | | | |
|---|---|---|---|---|---|
| pound | | | | | |
| No. | Intermediate A | Intermediate B | Intermediate C | Target Compound | Yield |
| 2-9 | 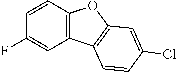 [SMILES: Fc1ccc2oc3cc(Cl)ccc3c2c1] | 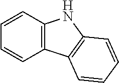 [SMILES: c1ccc2c(c1)[nH]c1ccccc12] | 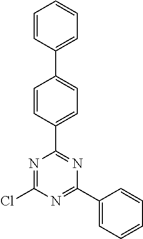 [SMILES: Clc1nc(-c2ccccc2)nc(-c2ccc(-c3ccccc3)cc2)n1] | 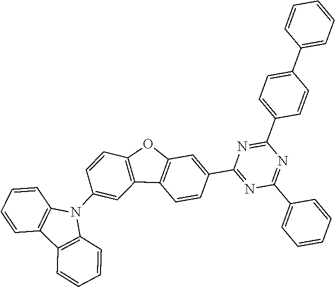 [SMILES: c1ccc(-c2ccc(-c3nc(-c4ccccc4)nc(-c4ccc5c(c4)oc4ccc(-n6c7ccccc7c7ccccc76)cc45)n3)cc2)cc1] | 75% |
| 2-10 | 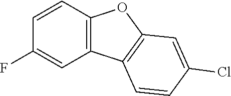 [SMILES: Fc1ccc2oc3cc(Cl)ccc3c2c1] | 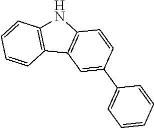 [SMILES: c1ccc(-c2ccc3[nH]c4ccccc4c3c2)cc1] | 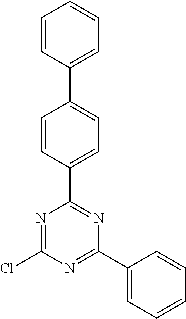 [SMILES: Clc1nc(-c2ccccc2)nc(-c2ccc(-c3ccccc3)cc2)n1] | 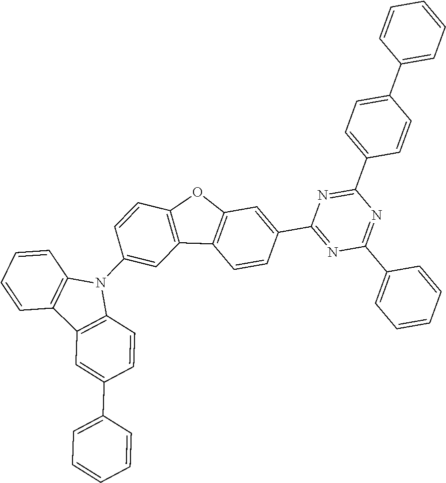 [SMILES: c1ccc(-c2ccc(-c3nc(-c4ccccc4)nc(-c4ccc5c(c4)oc4ccc(-n6c7ccccc7c7cc(-c8ccccc8)ccc76)cc45)n3)cc2)cc1] | 83% |
| 2-11 | 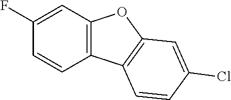 [SMILES: Fc1ccc2c(c1)oc1cc(Cl)ccc12] | 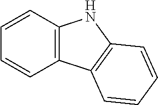 [SMILES: c1ccc2c(c1)[nH]c1ccccc12] | 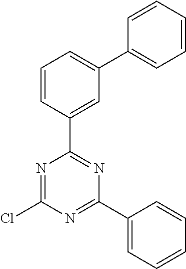 [SMILES: Clc1nc(-c2ccccc2)nc(-c2cccc(-c3ccccc3)c2)n1] | 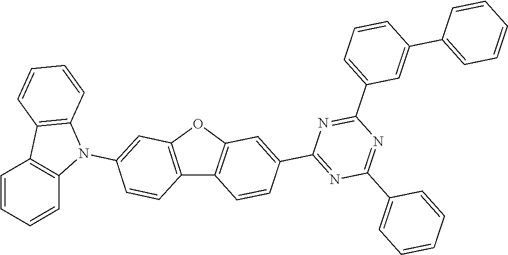 [SMILES: c1ccc(-c2cccc(-c3nc(-c4ccccc4)nc(-c4ccc5c(c4)oc4cc(-n6c7ccccc7c7ccccc76)ccc45)n3)c2)cc1] | 69% |
| 2-32 | 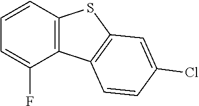 [SMILES: Fc1cccc2sc3cc(Cl)ccc3c12] | 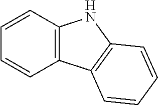 [SMILES: c1ccc2c(c1)[nH]c1ccccc12] | 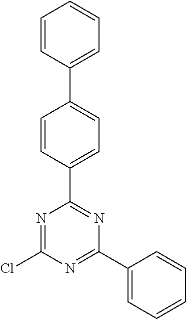 [SMILES: Clc1nc(-c2ccccc2)nc(-c2ccc(-c3ccccc3)cc2)n1] | 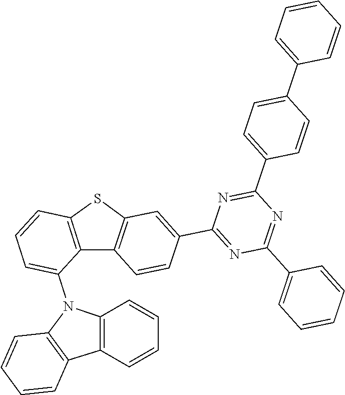 [SMILES: c1ccc(-c2ccc(-c3nc(-c4ccccc4)nc(-c4ccc5c(c4)sc4cccc(-n6c7ccccc7c7ccccc76)c45)n3)cc2)cc1] | 73% |
| 2-55 | 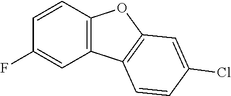 [SMILES: Fc1ccc2oc3cc(Cl)ccc3c2c1] | 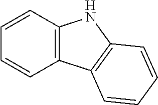 [SMILES: c1ccc2c(c1)[nH]c1ccccc12] | 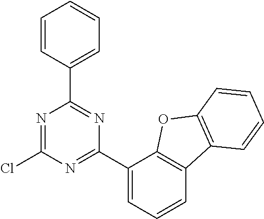 [SMILES: Clc1nc(-c2ccccc2)nc(-c2cccc3c2oc2ccccc23)n1] | 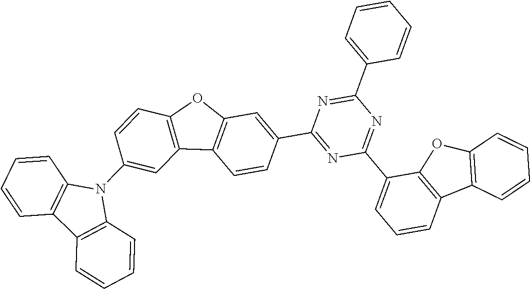 [SMILES: c1ccc(-c2nc(-c3ccc4c(c3)oc3ccc(-n5c6ccccc6c6ccccc65)cc34)nc(-c3cccc4c3oc3ccccc34)n2)cc1] | 68% |
| 2-56 | 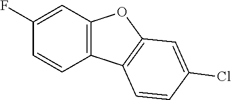 [SMILES: Fc1ccc2c(c1)oc1cc(Cl)ccc12] | 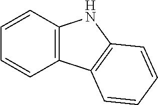 [SMILES: c1ccc2c(c1)[nH]c1ccccc12] | 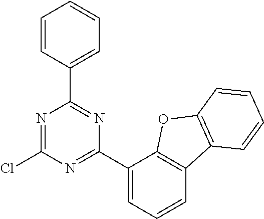 [SMILES: Clc1nc(-c2ccccc2)nc(-c2cccc3c2oc2ccccc23)n1] | 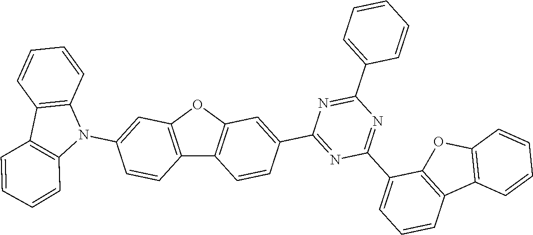 [SMILES: c1ccc(-c2nc(-c3ccc4c(c3)oc3cc(-n5c6ccccc6c6ccccc65)ccc34)nc(-c3cccc4c3oc3ccccc34)n2)cc1] | 83% |

### [Preparation Example 3] Preparation of Compound 2-13

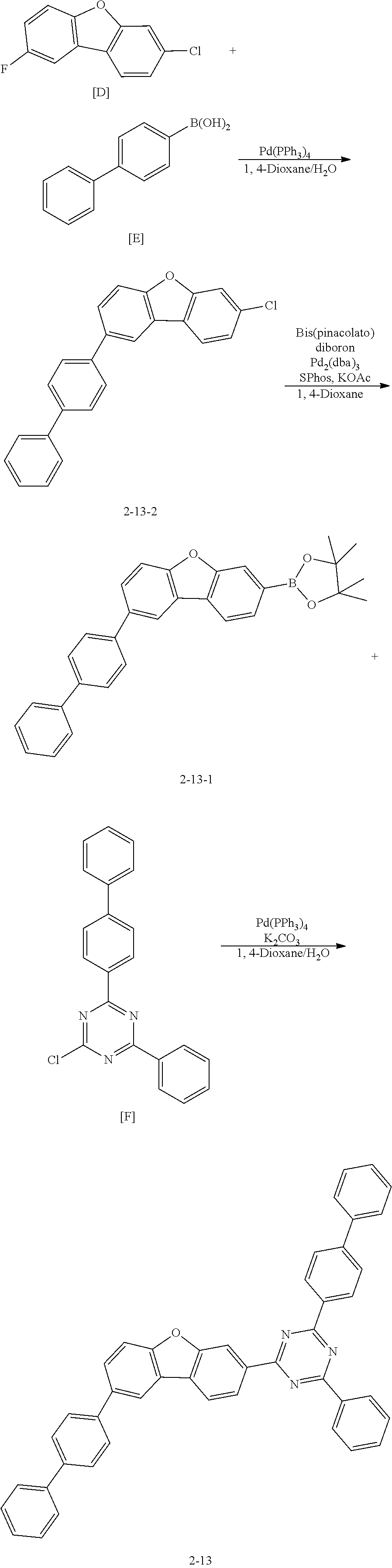

### 1) Preparation of Compound 2-13-2

A mixture of 7-chloro-2-fluorodibenzo[b,d]furan (6 g, 27.19 mmol), 1,1′-biphenyl]-4-ylboronic acid (5.92 g, 29.91 mmol), tetrakis(triphenylphosphine)palladium(0)(1.69 g, 1.46 mmol), potassium carbonate (7.52 g, 54.38 mmol), and 1,4-dioxane/water (60 ml/15 ml) was refluxed at 110° C. for 4 hours in a one-neck round-bottom flask. The mixture was filtered at 60° C., and then washed with 1,4-dioxane, distilled water, and MeOH to obtain Compound 2-13-2. (7.53 g, 78%)

### 2) Preparation of Compound 2-13-1

A mixture of 1,4-dioxane (70 ml) with 2-([1,1′-biphenyl]-4-yl)-7-chlorodibenzo[b,d]furan (7.53 g, 21.22 mmol), bis(pinacolato)diboron (10.78 g, 42.44 mmol), Pcy₃ (1.19 g, 4.24 mmol), potassium acetate (6.25 g, 63.66 mmol), and Pd₂(dba)₃ (1.94 g, 2.12 mmol) was refluxed at 110° C. in a one-neck round-bottom flask. After the mixture was cooled, the filtered filtrate was concentrated, and column purification (DCM:hexane=1:3) was performed to obtain Compound 2-13-1. (6.25 g, 66%)

### 3) Preparation of Compound 2-13

A mixture of 2-(8-([1,1′-biphenyl]-4-yl)dibenzo[b,d]furan-3-yl)-4,4,5,5-tetramethyl-1,3,2-dioxaborolane (6.25 g, 14.00 mmol), 2-([1,1′-biphenyl]-4-yl)-4-chloro-6-phenyl-1,3,5-triazine (5.29 g, 15.4 mmol), tetrakis(triphenylphosphine)palladium(0) (0.81 g, 0.7 mmol), potassium carbonate (3.87 g, 28.00 mmol), and 1,4-dioxane/water (60 ml/18 ml) was refluxed at 110° C. for 4 hours in a one-neck round-bottom flask. The mixture was filtered at 60° C., and then washed with 1,4-dioxane, distilled water, and MeOH to obtain Compound 2-13. (6.77 g, 77%)

Synthesis was performed in the same manner as in Preparation Example 3, except that Intermediates D, E, and F in the following Table 5 were used instead of [D],[E], and [F], respectively, in Preparation Example 3.

TABLE 5

| Com- | | | | | |
|---|---|---|---|---|---|
| pound | | | | | |
| No. | Intermediate D | Intermediate E | Intermediate F | Target Compound | Yield |
| 2-13 | 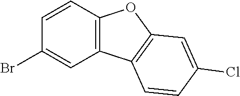 [SMILES: Clc1ccc2c(c1)oc1ccc(Br)cc12] | 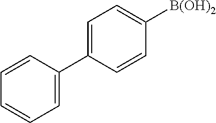 [SMILES: OB(O)c1ccc(-c2ccccc2)cc1] | 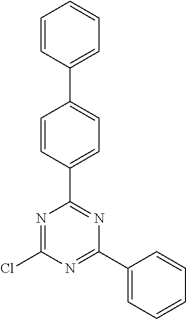 [SMILES: Clc1nc(-c2ccccc2)nc(-c2ccc(-c3ccccc3)cc2)n1] | 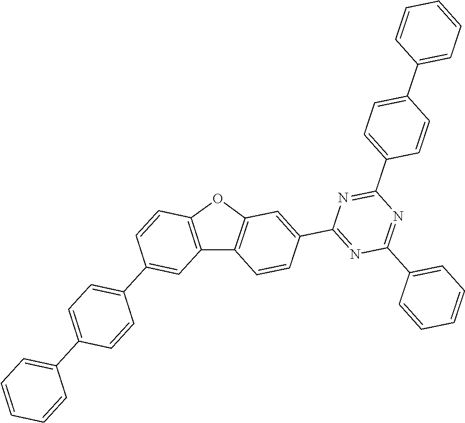 [SMILES: c1ccc(-c2ccc(-c3ccc4oc5cc(-c6nc(-c7ccccc7)nc(-c7ccc(-c8ccccc8)cc7)n6)ccc5c4c3)cc2)cc1] | 77 |
| 2-14 | 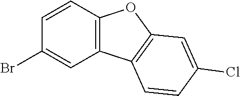 [SMILES: Clc1ccc2c(c1)oc1ccc(Br)cc12] | 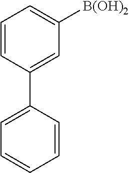 [SMILES: OB(O)c1cccc(-c2ccccc2)c1] | 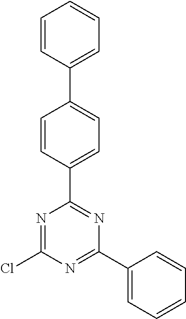 [SMILES: Clc1nc(-c2ccccc2)nc(-c2ccc(-c3ccccc3)cc2)n1] | 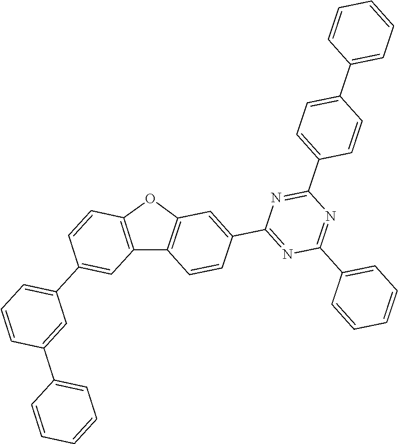 [SMILES: c1ccc(-c2ccc(-c3nc(-c4ccccc4)nc(-c4ccc5c(c4)oc4ccc(-c6cccc(-c7ccccc7)c6)cc45)n3)cc2)cc1] | 88% |
| 2-16 | 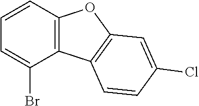 [SMILES: Clc1ccc2c(c1)oc1cccc(Br)c12] | 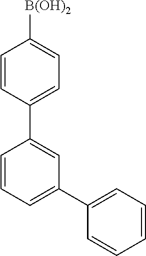 [SMILES: OB(O)c1ccc(-c2cccc(-c3ccccc3)c2)cc1] | 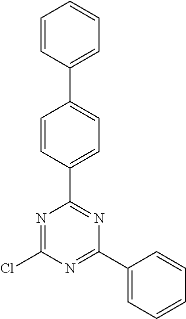 [SMILES: Clc1nc(-c2ccccc2)nc(-c2ccc(-c3ccccc3)cc2)n1] | 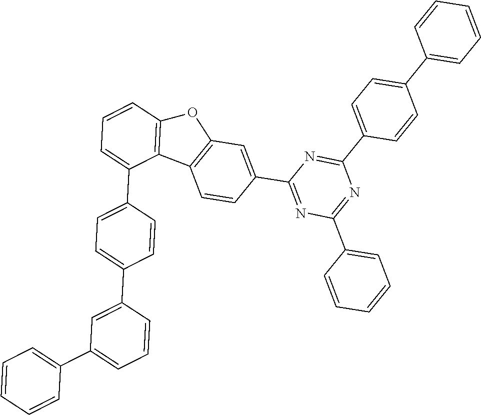 [SMILES: c1ccc(-c2ccc(-c3nc(-c4ccccc4)nc(-c4ccc5c(c4)oc4cccc(-c6ccc(-c7cccc(-c8ccccc8)c7)cc6)c45)n3)cc2)cc1] | 61% |
| 2-21 | 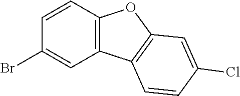 [SMILES: Clc1ccc2c(c1)oc1ccc(Br)cc12] | 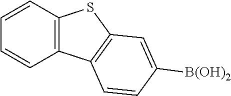 [SMILES: OB(O)c1ccc2c(c1)sc1ccccc12] | 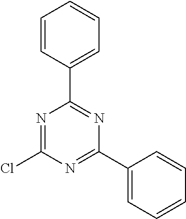 [SMILES: Clc1nc(-c2ccccc2)nc(-c2ccccc2)n1] | 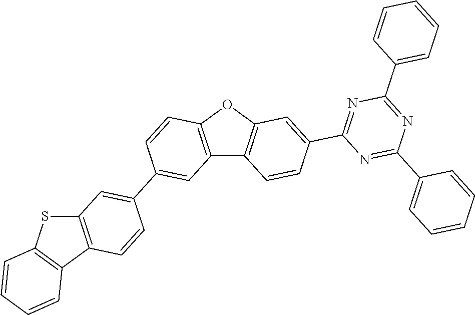 [SMILES: c1ccc(-c2nc(-c3ccccc3)nc(-c3ccc4c(c3)oc3ccc(-c5ccc6c(c5)sc5ccccc56)cc34)n2)cc1] | 69% |
| 2-78 | 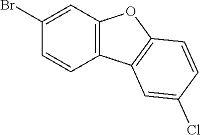 [SMILES: Clc1ccc2oc3cc(Br)ccc3c2c1] | 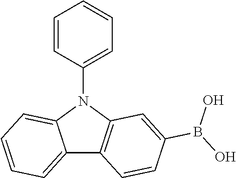 [SMILES: OB(O)c1ccc2c3ccccc3n(-c3ccccc3)c2c1] | 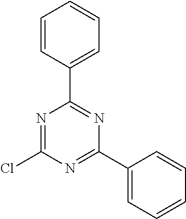 [SMILES: Clc1nc(-c2ccccc2)nc(-c2ccccc2)n1] | 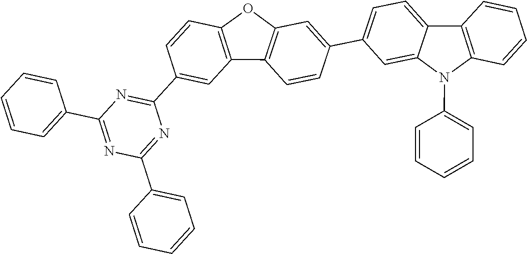 [SMILES: c1ccc(-c2nc(-c3ccccc3)nc(-c3ccc4oc5cc(-c6ccc7c8ccccc8n(-c8ccccc8)c7c6)ccc5c4c3)n2)cc1] | 72% |
| 2-79 | 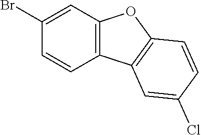 [SMILES: Clc1ccc2oc3cc(Br)ccc3c2c1] | 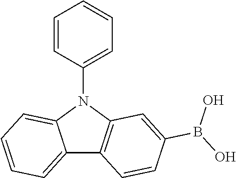 [SMILES: OB(O)c1ccc2c3ccccc3n(-c3ccccc3)c2c1] | 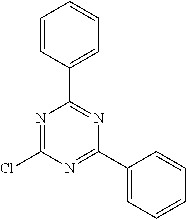 [SMILES: Clc1nc(-c2ccccc2)nc(-c2ccccc2)n1] | 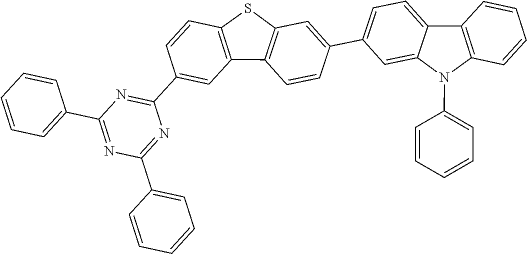 [SMILES: c1ccc(-c2nc(-c3ccccc3)nc(-c3ccc4sc5cc(-c6ccc7c8ccccc8n(-c8ccccc8)c7c6)ccc5c4c3)n2)cc1] | 68% |

### [Preparation Example 4] Preparation of Compound 2-62

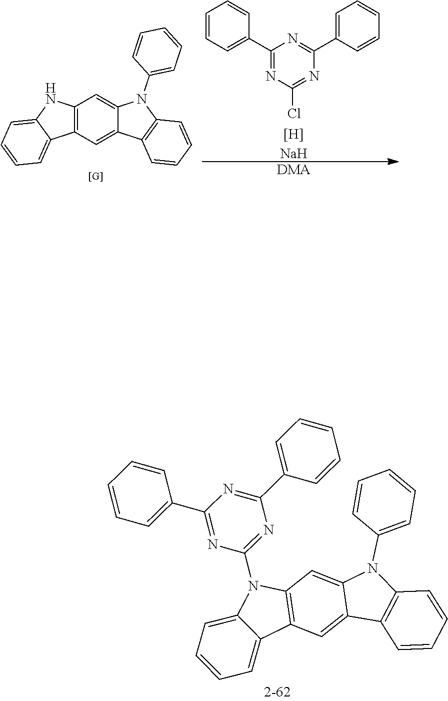

### 1) Preparation of Compound 2-62

5-phenyl-5,7-dihydroindolo[2,3-b]carbazole (10 g, 30.1 mmol) and dimethylacetamide (100 ml) were put into a one-neck round-bottom flask and stirred at room temperature for 10 minutes, NaH (1.44 g, 60.2 mmol) was slowly added thereto, and the resulting mixture was stirred for 1 hour. 2-Chloro-4,6-diphenyl-1,3,5-triazine (8.06 g, 30.1 mmol) was additionally added thereto, and the resulting mixture was stirred at room temperature for 12 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:3) and recrystallized with methanol to obtain 13 g (76%) of Target Compound 2-62.

Synthesis was performed in the same manner as in Preparation Example 4, except that Intermediates G and H in the following Table 6 were used instead of [G] and [H], respectively, in Preparation Example 4.

TABLE 6

| Com- | | | | |
|---|---|---|---|---|
| pound | | | | |
| No. | Intermediate G | Intermediate H | Target Compound | Yield |
| 2-62 | 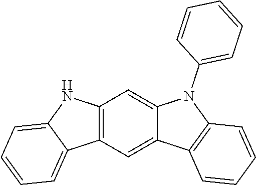 [SMILES: c1ccc(-n2c3ccccc3c3cc4c(cc32)[nH]c2ccccc24)cc1] | 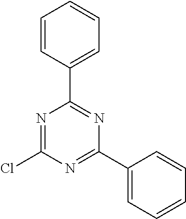 [SMILES: Clc1nc(-c2ccccc2)nc(-c2ccccc2)n1] | 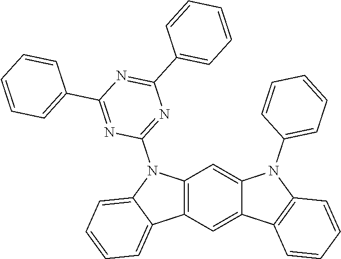 [SMILES: c1ccc(-c2nc(-c3ccccc3)nc(-n3c4ccccc4c4cc5c6ccccc6n(-c6ccccc6)c5cc43)n2)cc1] | 76% |
| 2-67 | 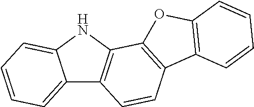 [SMILES: c1ccc2c(c1)[nH]c1c2ccc2c3ccccc3oc21] | 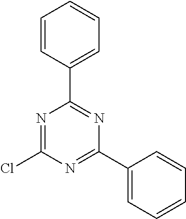 [SMILES: Clc1nc(-c2ccccc2)nc(-c2ccccc2)n1] | 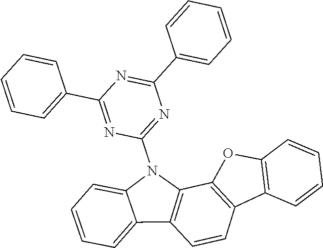 [SMILES: c1ccc(-c2nc(-c3ccccc3)nc(-n3c4ccccc4c4ccc5c6ccccc6oc5c43)n2)cc1] | 71% |
| 2-68 | 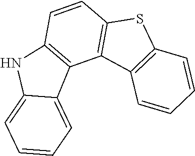 [SMILES: c1ccc2c(c1)[nH]c1ccc3sc4ccccc4c3c12] | 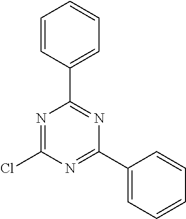 [SMILES: Clc1nc(-c2ccccc2)nc(-c2ccccc2)n1] | 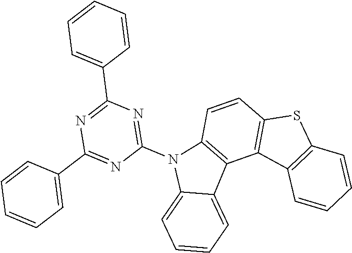 [SMILES: c1ccc(-c2nc(-c3ccccc3)nc(-n3c4ccccc4c4c5c(ccc43)sc3ccccc35)n2)cc1] | 69% |
| 2-71 | 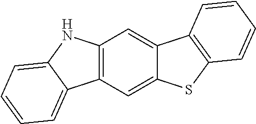 [SMILES: c1ccc2c(c1)[nH]c1cc3c(cc12)sc1ccccc13] | 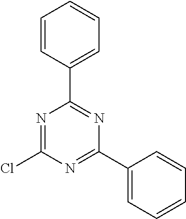 [SMILES: Clc1nc(-c2ccccc2)nc(-c2ccccc2)n1] | 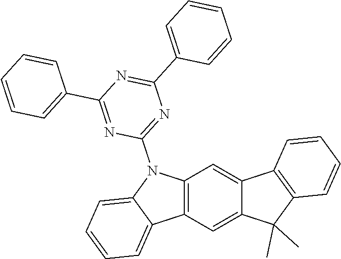 [SMILES: CC1(C)c2ccccc2-c2cc3c(cc21)c1ccccc1n3-c1nc(-c2ccccc2)nc(-c2ccccc2)n1] | 65% |
| 2-72 | 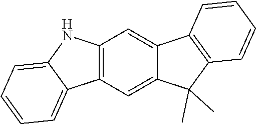 [SMILES: CC1(C)c2ccccc2-c2cc3[nH]c4ccccc4c3cc21] | 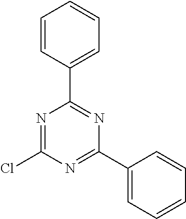 [SMILES: Clc1nc(-c2ccccc2)nc(-c2ccccc2)n1] | 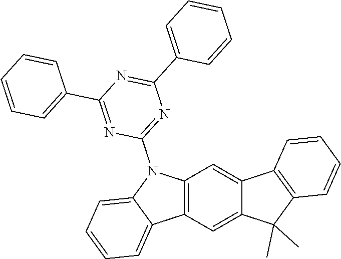 [SMILES: CC1(C)c2ccccc2-c2cc3c(cc21)c1ccccc1n3-c1nc(-c2ccccc2)nc(-c2ccccc2)n1] | 64% |
| 2-73 | 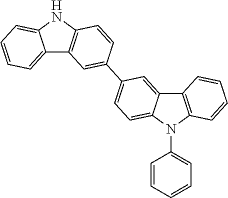 [SMILES: c1ccc(-n2c3ccccc3c3cc(-c4ccc5[nH]c6ccccc6c5c4)ccc32)cc1] | 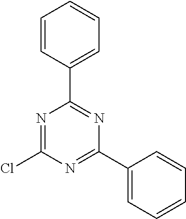 [SMILES: Clc1nc(-c2ccccc2)nc(-c2ccccc2)n1] | 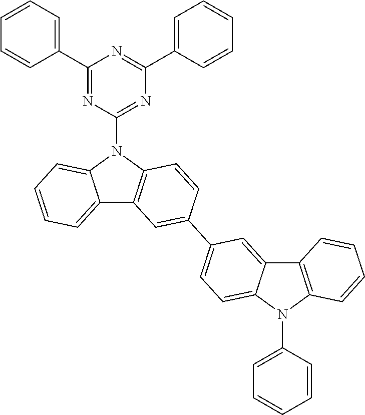 [SMILES: c1ccc(-c2nc(-c3ccccc3)nc(-n3c4ccccc4c4cc(-c5ccc6c(c5)c5ccccc5n6-c5ccccc5)ccc43)n2)cc1] | 74% |

### [Preparation Example 5] Preparation of Compound 2-64

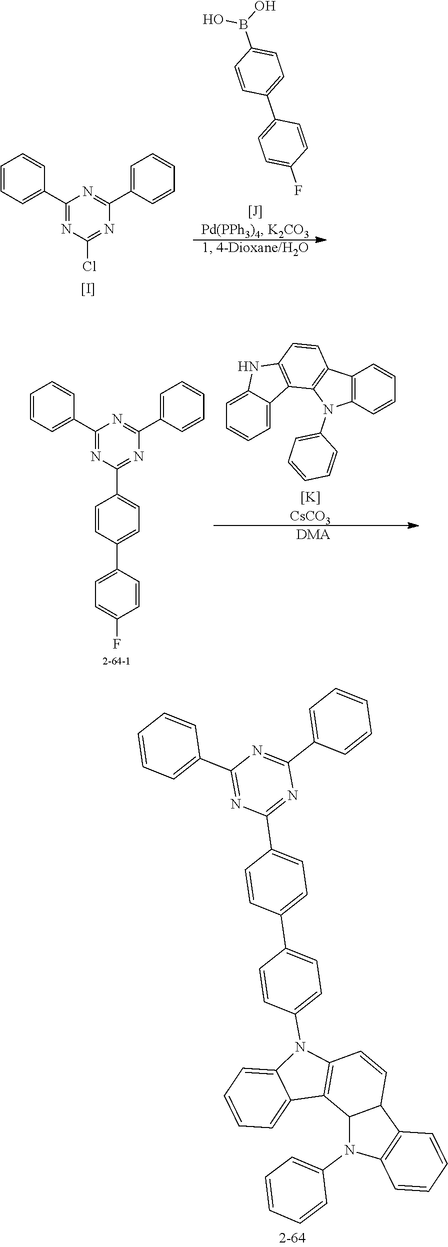

### 1) Preparation of Compound 2-64-1

After 10 g (37.4 mM) of 2-chloro-4,6-diphenyl-1,3,5-triazine, 8.08 g (37.4 mM) of (4′-fluoro-[1,1′-biphenyl]-4-yl)boronic acid, 2.15 g (1.87 mM) of Pd(PPh₃)₄, and 15.4 g (112 mM) of K₂CO₃ were dissolved in 100/20 mL of 1,4-dioxane/H₂O, the resulting solution was refluxed at 110° C. for 6 hours. After the reaction was completed, distilled water and dichloromethane (DCM) were added thereto at room temperature, extraction was performed, the organic layer was dried over MgSO₄, and then the solvent was removed by a rotary evaporator. The reaction product was purified with column chromatography (DCM:hexane=1:4) to obtain 10.7 g (71%) of Target Compound 2-64-1.

### 2) Preparation of Compound 2-64

A mixture of 2-(4′-fluoro-[1,1′-biphenyl]-4-yl)-4,6-diphenyl-1,3,5-triazine (10 g, 24.8 mmol), 12-phenyl-5,12-dihydroindolo[3,2-a]carbazole (8.2 g, 24.8 mmol), Cs₂CO₃ (16 g, 74.4 mmol), and dimethylacetamide (100 ml) was stirred at 170° C. for 12 hours in a one-neck round-bottom flask. After the mixture was cooled, the mixture was filtered, the solvent of the filtrate was removed, and then column purification (DCM:hexane=1:4) was performed to obtain 12.1 g (68%) of Target Compound 2-64.

Synthesis was performed in the same manner as in Preparation Example 5, except that Intermediates I, J, and K in the following Table 7 were used in Preparation Example 5.

TABLE 7

| Com- | | | | | |
|---|---|---|---|---|---|
| poun | | | | | |
| d No. | Intermediate I | Intermediate J | Intermediate K | Target Compound | Yield |
| 2-64 | 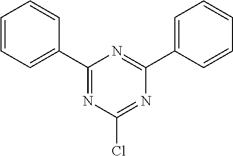 [SMILES: Clc1nc(-c2ccccc2)nc(-c2ccccc2)n1] | 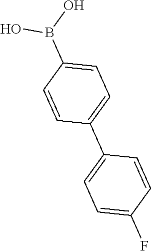 [SMILES: OB(O)c1ccc(-c2ccc(F)cc2)cc1] | 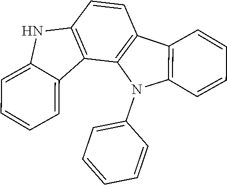 [SMILES: c1ccc(-n2c3ccccc3c3ccc4[nH]c5ccccc5c4c32)cc1] | 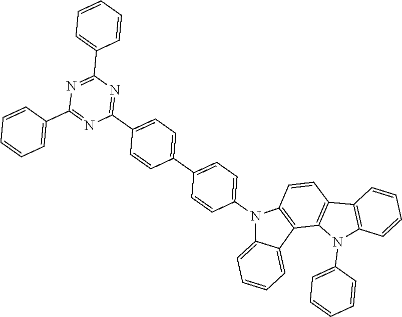 [SMILES: c1ccc(-c2nc(-c3ccccc3)nc(-c3ccc(-c4ccc(-n5c6ccccc6c6c5ccc5c7ccccc7n(-c7ccccc7)c56)cc4)cc3)n2)cc1] | 68% |
| 2-65 | 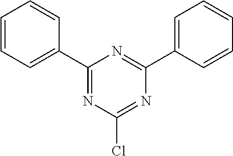 [SMILES: Clc1nc(-c2ccccc2)nc(-c2ccccc2)n1] | 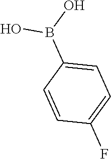 | 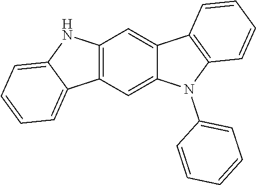 [SMILES: c1ccc(-n2c3ccccc3c3cc4[nH]c5ccccc5c4cc32)cc1] | 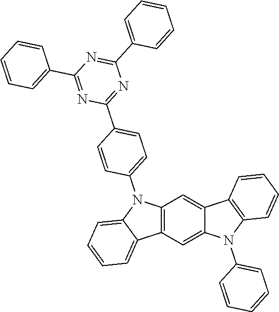 [SMILES: c1ccc(-c2nc(-c3ccccc3)nc(-c3ccc(-n4c5ccccc5c5cc6c(cc54)c4ccccc4n6-c4ccccc4)cc3)n2)cc1] | 71% |
| 2-75 | 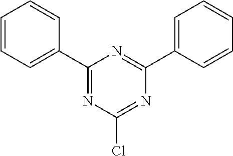 [SMILES: Clc1nc(-c2ccccc2)nc(-c2ccccc2)n1] | 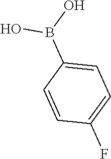 | 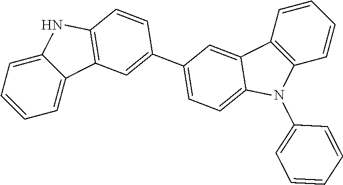 [SMILES: c1ccc(-n2c3ccccc3c3cc(-c4ccc5[nH]c6ccccc6c5c4)ccc32)cc1] | 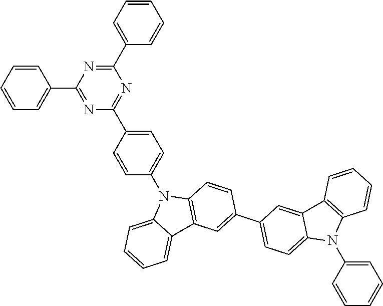 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1] | 73% |
| 2-76 | 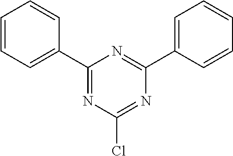 [SMILES: Clc1nc(-c2ccccc2)nc(-c2ccccc2)n1] | 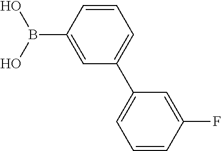 [SMILES: OB(O)c1cccc(-c2cccc(F)c2)c1] | 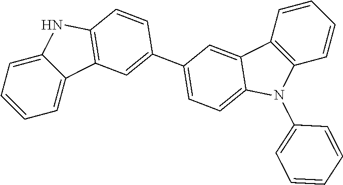 [SMILES: c1ccc(-n2c3ccccc3c3cc(-c4ccc5[nH]c6ccccc6c5c4)ccc32)cc1] | 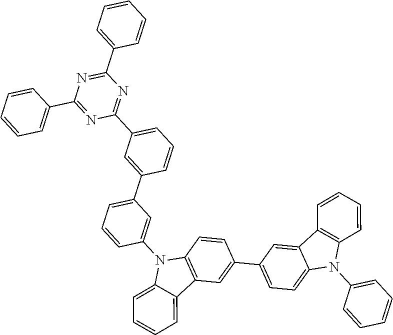 [SMILES: c1ccc(-c2nc(-c3ccccc3)nc(-c3cccc(-c4cccc(-n5c6ccccc6c6cc(-c7ccc8c(c7)c7ccccc7n8-c7ccccc7)ccc65)c4)c3)n2)cc1] | 70% |

The following Tables 8 to 11 are the FD-MS data and ¹H NMR data of the synthesized compounds, and it can be confirmed through the following data that the desired compound was synthesized.

TABLE 8

| Compound | FD-Mass | Compound | FD-Mass |
|---|---|---|---|
| 1-3 | m/z = 672.37 | 1-4 | m/z = 671.37 |
| | (C48H8D22N2O = 672.92) | | (C48H9D21N2O = 671.91) |
| 1-7 | m/z = 749.41 | 1-10 | m/z = 749.41 |
| | (C54H11D23N2O = 750.02) | | (C54H11D23N2O = 750.02) |
| 1-14 | m/z = 748.41 | 1-17 | m/z = 750.42 |
| | (C54H12D22N2O = 749.01) | | (C54H10D24N2O = 751.03) |
| 1-23 | m/z = 688.35 | 1-26 | m/z = 763.38 |
| | (C48H8D22N2S = 688.98) | | (C54H13D21N2S = 764.07) |
| 1-35 | m/z = 764.38 | 1-38 | m/z = 766.39 |
| | (C54H12D22N2S = 765.07) | | (C54H10D24N2S = 767.09) |
| 1-43 | m/z = 673.38 | 1-44 | m/z = 672.37 |
| | (C48H7D23N2O = 673.92) | | (C48H8D22N2O = 672.92) |
| 1-46 | m/z = 748.41 | 1-51 | m/z = 751.42 |
| | (C54H12D22N2O = 749.01) | | (C54H9D25N2O = 752.03) |
| 1-54 | m/z = 748.41 | 1-56 | m/z = 749.41 |
| | (C54H12D22N2O = 749.01) | | (C54H11D23N2O = 750.02) |
| 1-62 | m/z = 686.34 | 1-68 | m/z = 764.38 |
| | (C48H10D20N2S = 686.96) | | (C54H12D22N2S = 765.07) |
| 1-70 | m/z = 765.39 | 1-72 | m/z = 849.47 |
| | (C54H11D23N2S = 766.08) | | (C60H7D31N2S = 850.23) |
| 1-75 | m/z = 766.39 | 1-78 | m/z = 767.40 |
| | (C54H10D24N2S = 767.09) | | (C54H9D25N2S = 768.09) |
| 1-83 | m/z = 673.38 | 1-85 | m/z = 746.39 |
| | (C48H7D23N2O = 673.92) | | (C54H14D20N2O = 747.00) |
| 1-86 | m/z = 748.41 | 1-87 | m/z = 749.41 |
| | (C54H12D22N2O = 749.01) | | (C54H11D23N2O = 750.02) |
| 1-90 | m/z = 749.41 | 1-93 | m/z = 749.41 |
| | (C54H11D23N2O = 750.02) | | (C54H11D23N2O = 750.02) |
| 1-94 | m/z = 748.41 | 1-96 | m/z = 749.41 |
| | (C54H12D22N2O = 749.01) | | (C54H11D23N2O = 750.02) |
| 1-97 | m/z = 750.42 | 1-103 | m/z = 688.35 |
| | (C54H10D24N2O = 751.03) | | (C48H8D22N2S = 688.98) |
| 1-104 | m/z = 688.35 | 1-106 | m/z = 673.38 |
| | (C48H8D22N2S = 688.98) | | (C54H13D21N2S = 764.07) |
| 1-107 | m/z = 764.38 | 1-110 | m/z = 766.39 |
| | (C54H12D22N2S = 765.07) | | (C54H10D24N2S = 767.09) |
| 1-114 | m/z = 673.38 | 1-118 | m/z = 767.40 |
| | (C54H13D21N2S = 764.07) | | (C54H9D25N2S = 768.09) |
| 1-122 | m/z = 670.36 | 1-128 | m/z = 747.40 |
| | (C48H10D20N2O = 670.90) | | (C54H13D21N2O = 748.01) |
| 1-129 | m/z = 747.40 | 1-134 | m/z = 747.40 |
| | (C54H13D21N2O = 748.01) | | (C54H13D21N2O = 748.01) |
| 1-136 | m/z = 747.40 | 1-143 | m/z = 686.34 |
| | (C54H13D21N2O = 748.01) | | (C48H10D20N2S = 686.96) |
| 1-146 | m/z = 762.37 | 1-147 | m/z = 762.37 |
| | (C54H14D20N2S = 763.06) | | (C54H14D20N2S = 763.06) |
| 1-150 | m/z = 764.38 | 1-155 | m/z = 762.37 |
| | (C54H12D22N2S = 765.07) | | (C54H14D20N2S = 763.06) |

TABLE 9

| Compound | FD-Mass | Compound | FD-Mass |
|---|---|---|---|
| 2-9 | m/z = 640.23 | 2-10 | m/z = 716.26 |
| | (C45H28N4O = 640.75) | | (C51H32N4O = 716.84) |
| 2-11 | m/z = 640.23 | 2-13 | m/z = 627.23 |
| | (C45H28N4O = 640.75) | | (C45H29N3O = 627.75) |
| 2-14 | m/z = 627.23 | 2-16 | m/z = 703.26 |
| | (C45H29N3O = 627.75) | | (C51H33N3O = 703.85) |
| 2-21 | m/z = 581.16 | 2-32 | m/z = 656.20 |
| | (C39H23N3OS = 581.69) | | (C45H28N4S = 656.81) |
| 2-55 | m/z = 654.21 | 2-56 | m/z = 654.21 |
| | (C45H26N4O2 = 654.73) | | (C45H26N4O2 = 654.73) |
| 2-62 | m/z = 563.21 | 2-65 | m/z = 639.24 |
| | (C39H25N5 = 563.66) | | (C45H29N5 = 639.76) |
| 2-67 | m/z = 488.16 | 2-68 | m/z = 504.14 |
| | (C33H20N4O = 488.55) | | (C33H20N4S = 504.61) |
| 2-73 | m/z = 639.24 | 2-75 | m/z = 715.27 |
| | (C45H29N5 = 639.76) | | (C51H33N5 = 715.86) |

TABLE 10

| Compound | 1H NMR(CDCl3, 200 Mz) |
|---|---|
| 1-3 | δ = 8.30 (1H, d), 8.13 (1H, d), 7.67-7.50 (6H, m) |
| 1-4 | δ = 8.30 (1H, d), 7.75 (1H, s), 8.13 (1H, d), 7.67-7.50 (6H, m) |
| 1-7 | δ = 8.21 (1H, s), 7.86-7.83 (2H, m), 7.75-7.68 (4H, m), 7.49-7.41 (4H, m) |
| 1-10 | δ = 8.30 (1H, d), 8.13 (1H, d), 7.86 (1H, s), 7.67-7.43 (8H, m) |
| 1-14 | δ = 7.92-7.91 (4H, m), 7.83 (2H, m), 7.75 (2H, d), 7.67 (1H, s), 7.49-7.41 (3H, m) |
| 1-17 | δ = 8.00 (1H, s), 7.82 (1H, s), 7.67-7.43 (8H, m) |
| 1-23 | δ = 8.30 (1H, d), 8.13 (1H, d), 7.67-7.50 (6H, m) |
| 1-26 | δ = 8.11-8.09 (2H, m), 7.92-7.91 (4H, m), 7.83 (1H, s), 7.75 (2H, d), 7.67 (1H, |
| | s), 7.49-7.41 (3H, m) |
| 1-35 | δ = 8.21 (1H, s), 8.11 (1H, s), 7.83 (1H, s), 7.75-7.41 (9H, m) |
| 1-38 | δ = 8.00 (1H, s), 7.83 (1H, s), 7.67-7.43 (8H, m) |
| 1-43 | δ = 8.30 (1H, s), 7.67-7.50 (6H, m) |
| 1-44 | δ = 8.30 (1H, s), 7.67-7.50 (7H, m) |
| 1-46 | δ = 7.92-7.91 (4H, m), 7.77-7.75 (3H, m), 7.67 (1H, s), 7.49-7.41 (4H, m) |
| 1-51 | δ = 7.77 (1H, s), 7.67-7.43 (8H, m) |
| 1-54 | δ = 7.92-7.91 (4H, m), 7.77-7.75 (3H, m), 7.67 (1H, s), 7.49-7.41 (4H, m) |
| 1-56 | δ = 8.21 (1H, s), 8.1 (1H, s), 7.81-7.60 (5H, m), 7.49-7.41 (4H, m) |
| 1-62 | δ = 8.03 (1H, s), 7.81-7.77 (2H, m), 7.62-7.50 (7H, m) |
| 1-68 | δ = 8.21 (1H, s), 7.87 (1H, s), 7.81-7.75 (5H, m), 7.50-7.41 (5H, m) |
| 1-70 | δ = 8.11 (1H, s), 8.00 (1H, s), 7.86 (1H, s), 7.67-7.43 (8H, m) |
| 1-72 | δ = 7.86 (1H, s), 7.62-7.50 (6H, m) |
| 1-75 | δ = 8.21 (1H, s), 7.75-7.41 (9H, m) |
| 1-78 | δ = 8.00 (1H, s), 7.67-7.43 (8H, m) |
| 1-83 | δ = 8.00 (1H, s), 7.67-7.50 (6H, m) |
| 1-85 | δ = 8.00 (1H, s), 7.92-7.91 (4H, m), 7.84-7.75 (5H, m), 7.52-7.41 (4H, m) |
| 1-86 | δ = 7.92-7.91 (4H, m), 7.77-7.75 (3H, m), 7.67 (1H, s), 7.49-7.41 (4H, m) |
| 1-87 | δ = 8.21 (1H, s), 7.98 (1H, s), 7.75-7.60 (4H, m), 7.49-7.41 (5H, m) |
| 1-90 | δ = 8.00 (1H, s), 7.86-7.83 (2H, m), 7.67-7.43 (8H, m) |
| 1-93 | δ = 8.00 (1H, s), 7.92-7.91 (4H, m), 7.84 (1H, s), 7.75 (2H, d), 7.49-7.41 (3H, m) |
| 1-94 | δ = 7.92-7.91 (4H, m), 7.77-7.75 (3H, m), 7.67 (1H, s), 7.49-7.41 (4H, m) |
| 1-96 | δ = 8.21 (1H, s), 7.81-7.41 (10H, m) |
| 1-97 | δ = 7.81 (1H, s), 7.71-7.43 (9H, m) |
| 1-103 | δ = 8.00 (1H, s), 7.82 (1H, s), 7.67-7.50 (6H, m) |
| 1-104 | δ = 7.83 (1H, s), 7.67-7.50 (7H, m) |
| 1-106 | δ = 8.11-8.09 (2H, m), 7.92-7.91 (4H, m), 7.75 (2H, d), 7.67 (1H, s), 7.58 (1H, |
| | s), 7.49-7.41 (3H, m) |
| 1-107 | δ = 8.21-8.19 (2H, m), 7.75-7.41 (10H, m) |
| 1-110 | δ = 8.11 (1H, s), 8.00 (1H, s), 7.86 (1H, s), 7.67-7.43 (7H, m) |
| 1-114 | δ = 8.11 (1H, s), 7.92-7.91 (4H, m), 7.75 (2H, d), 7.67 (1H, s), 7.58 (1H, s), |
| | 7.52-7.41 (4H, m) |
| 1-118 | δ = 8.00 (1H, s), 7.67-7.43 (8H, m) |
| 1-122 | δ = 8.31 (1H, s), 7.84-7.81 (2H, m), 7.62-7.50 (7H, m) |
| 1-128 | δ = 8.21 (1H, s), 7.97 (1H, s), 7.75-7.41 (11H, m) |
| 1-129 | δ = 8.31 (1H, s), 7.84-7.43 (12H, m) |
| 1-134 | δ = 7.97-7.91 (5H, m), 7.77-7.75 (3H, m), 7.67 (1H, s), 7.49-7.41 (4H, m) |
| 1-136 | δ = 8.21 (1H, s), 7.97 (1H, s), 7.81-7.41 (11H, m) |
| 1-143 | δ = 8.30 (1H, s), 8.00-7.97 (2H, m), 7.82 (1H, s), 7.67-7.50 (6H, m) |
| 1-146 | δ = 8.30 (1H, s), 8.11-8.09 (2H, m), 7.92-7.91 (4H, m), 7.75 (2H, d), 7.67 (1H, |
| | s), 7.58-7.41 (4H, m) |
| 1-147 | δ = 8.30 (1H, s), 8.21-8.19 (2H, m), 7.97 (1H, s), 7.67-7.41 (10H, m) |
| 1-150 | δ = 8.11 (1H, s), 8.00-7.97 (2H, m), 7.86 (1H, s), 7.75 (1H, s), 7.67-7.43 (7H, m) |
| 1-155 | δ = 8.21 (1H, s), 8.11 (1H, s), 7.97 (1H, s), 7.68-7.41 (11H, m) |

TABLE 11

| Compound | 1H NMR(CDCl3, 200 Mz) |
|---|---|
| 2-9 | δ = 8.55 (1H, d), 8.36 (2H, m), 8.19 (1H, d), 7.94~8.03 (4H, m), 7.74~7.82 |
| | (5H, m), 7.16~7.61 (15H, m) |
| 2-10 | δ = 8.55 (1H, d), 8.36 (2H, m), 7.89~8.03 (6H, m), 7.74~7.76 (8H, m), 7.61 |
| | (1H, s), 7.25~7.50 (13H, m), 7.16 (1H, t) |
| 2-11 | δ = 8.55 (1H, d), 8.36~8.38 (3H, m), 8.19 (1H, d), 7.94~8.03 (4H, m), |
| | 7.73~7.82 (5H, m), 7.35~7.61 (11H, m), 7.16~7.25 (3H, m) |
| 2-13 | δ = 8.36 (2H, m), 7.96~8.03 (3H, m), 7.75~7.88 (9H, m), 7.41~7.50 (9H, m), |
| | 7.25 (6H, m) |
| 2-14 | δ = 8.36 (2H, m), 7.73~8.03 (14H, m), 7.61 (2H, d), 7.41~7.50 (9H, m), 7.25 |
| | (2H, d) |
| 2-16 | δ = 8.36 (2H, m), 7.94~8.03 (4H, m), 7.73~7.82 (9H, m), 7.41~7.61 (12H, |
| | m), 7.25 (6H, m) |
| 2-21 | δ = 8.45 (1H, d), 8.36 (4H, m), 8.17~8.24 (3H, m), 8.03 (1H, d), 7.76~7.93 |
| | (6H, m), 7.49~7.56 (8H, m) |
| 2-32 | δ = 8.55 (1H, d), 8.36 (2H, m), 8.19~8.24 (3H, m), 7.89~7.96 (5H, m), 7.75 |
| | (2H, d), 7.35~7.63 (11H, m), 7.16~7.25 (4H, m) |
| 2-55 | δ = 8.55 (1H, d), 8.36 (2H, m), 8.19 (1H, d), 7.74~8.08 (8H, m), 7.50~7.61 |
| | (8H, m), 7.31~7.39 (4H, m), 7.16~7.20 (2H, m) |
| 2-56 | δ = 8.55 (1H, d), 8.36 (2H, m), 8.19 (1H, d), 7.76~8.08 (8H, m), 7.50~7.58 |
| | (8H, m), 7.16~7.39 (6H, m) |
| 2-9 | δ = 8.55 (1H, d), 8.36 (2H, m), 8.19 (1H, d), 7.94~8.03 (4H, m), 7.74~7.82 |
| | (5H, m), 7.16~7.61 (15H, m) |
| 2-10 | δ = 8.55 (1H, d), 8.36 (2H, m), 7.89~8.03 (6H, m), 7.74~7.76 (8H, m), 7.61 |
| | (1H, s), 7.25~7.50 (13H, m), 7.16 (1H, t) |
| 2-11 | δ = 8.55 (1H, d), 8.36~8.38 (3H, m), 8.19 (1H, d), 7.94~8.03 (4H, m), |
| | 7.73~7.82 (5H, m), 7.35~7.61 (11H, m), 7.16~7.25 (3H, m) |
| 2-13 | δ = 8.36 (2H, m), 7.96~8.03 (3H, m), 7.75~7.88 (9H, m), 7.41~7.50 (9H, m), |
| | 7.25 (6H, m) |
| 2-14 | δ = 8.36 (2H, m), 7.73~8.03 (14H, m), 7.61 (2H, d), 7.41~7.50 (9H, m), 7.25 |
| | (2H, d) |
| 2-16 | δ = 8.36 (2H, m), 7.94~8.03 (4H, m), 7.73~7.82 (9H, m), 7.41~7.61 (12H, |
| | m), 7.25 (6H, m) |
| 2-21 | δ = 8.45 (1H, d), 8.36 (4H, m), 8.17~8.24 (3H, m), 8.03 (1H, d), 7.76~7.93 |
| | (6H, m), 7.49~7.56 (8H, m) |
| 2-32 | δ = 8.55 (1H, d), 8.36 (2H, m), 8.19~8.24 (3H, m), 7.89~7.96 (5H, m), 7.75 |
| | (2H, d), 7.35~7.63 (11H, m), 7.16~7.25 (4H, m) |
| 2-55 | δ = 8.55 (1H, d), 8.36 (2H, m), 8.19 (1H, d), 7.74~8.08 (8H, m), 7.50~7.61 |
| | (8H, m), 7.31~7.39 (4H, m), 7.16~7.20 (2H, m) |
| 2-56 | δ = 8.55 (1H, d), 8.36 (2H, m), 8.19 (1H, d), 7.76~8.08 (8H, m), 7.50~7.58 |
| | (8H, m), 7.16~7.39 (6H, m) |
| 2-62 | δ = 8.55 (1H, d), 8.36 (4H, m), 8.19 (1H, d), 7.94 (1H, d), 7.58~7.50 (14H, |
| | m), 7.40~7.35 (2H, m), 7.16~7.20 (2H, m) |
| 2-65 | δ = 8.55 (1H, d), 8.36 (4H, m), 8.19 (1H, d), 7.94~7.91 (5H, m), 7.62~7.50 |
| | (14H, m), 7.40~7.35 (2H, m), 7.16~7.20 (2H, m) |
| 2-67 | δ = 8.55 (1H, d), 8.36 (4H, m), 7.98~7.94 (2H, m), 7.84 (1H, d), 7.54~7.50 |
| | (8H, m), 7.39~7.31 (3H, m), 7.16 (1H, t) |
| 2-68 | δ = 8.55 (1H, d), 8.45 (1H, d), 8.36 (4H, m), 7.96~7.93 (3H, m), 7.56~7.49 |
| | (8H, m), 7.35~7.31 (2H, m), 7.16 (1H, t) |
| 2-73 | δ = 8.55 (1H, d), 8.36~8.30 (5H, m), 8.19~8.13 (2H, m), 7.99~7.89 (4H, m), |
| | 7.77 (1H, d), 7.62~7.50 (13H, m), 7.35 (1H, t), 7.20~7.16 (2H, m) |
| 2-75 | δ = 8.55 (1H, d), 8.36~8.30 (5H, m), 8.19~8.13 (2H, m), 7.99~7.89 (8H, m), |
| | 7.77 (1H, d), 7.62~7.50 (13H, m), 7.35 (1H, t), 7.20~7.16 (2H, m) |

### Experimental Example 1

### (1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1″-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by using a compound described in the following Table 12 as a host and tris(2-phenylpyridine)iridium (Ir(ppy)₃) as a green phosphorescent dopant to dope the host with Ir(ppy)₃ in an amount of 7%. Thereafter, BC P as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (AI) negative electrode was deposited to have a thickness of 1200 Å on the electron injection layer to form a negative electrode, thereby manufacturing an organic electroluminescence device.

Meanwhile, all the organic compounds required for manufacturing an organic electroluminescence device were subjected to vacuum sublimed purification under 10⁻⁸ to 10⁻⁶ torr for each material, and used for the manufacture of the organic electroluminescence device.

Compounds [A] to [H] used in Comparative Examples 1 to 8 were as follows.

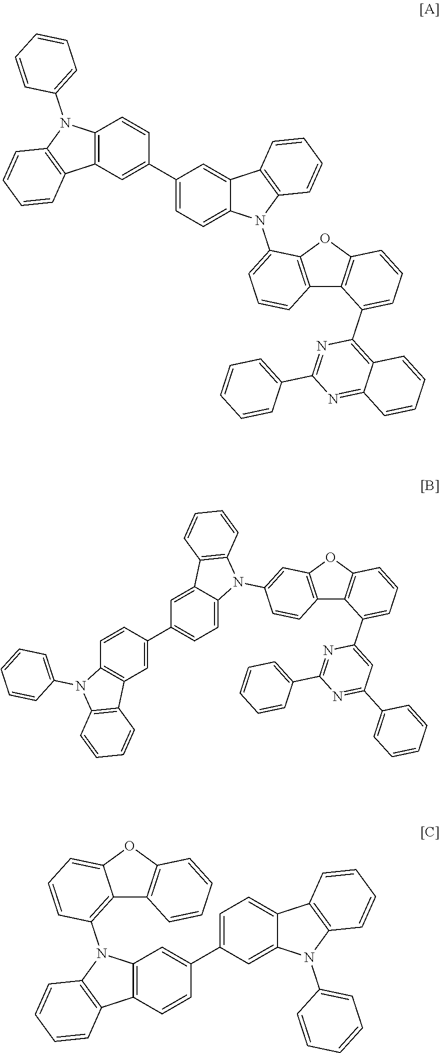 [SMILES: c1ccc(-c2nc(-c3cccc4oc5c(-n6c7ccccc7c7cc(-c8ccc9c(c8)c8ccccc8n9-c8ccccc8)ccc76)cccc5c34)c3ccccc3n2)cc1 ; c1ccc(-c2cc(-c3cccc4oc5cc(-n6c7ccccc7c7cc(-c8ccc9c(c8)c8ccccc8n9-c8ccccc8)ccc76)ccc5c34)nc(-c3ccccc3)n2)cc1 ; c1ccc(-n2c3ccccc3c3ccc(-c4ccc5c6ccccc6n(-c6cccc7oc8ccccc8c67)c5c4)cc32)cc1]

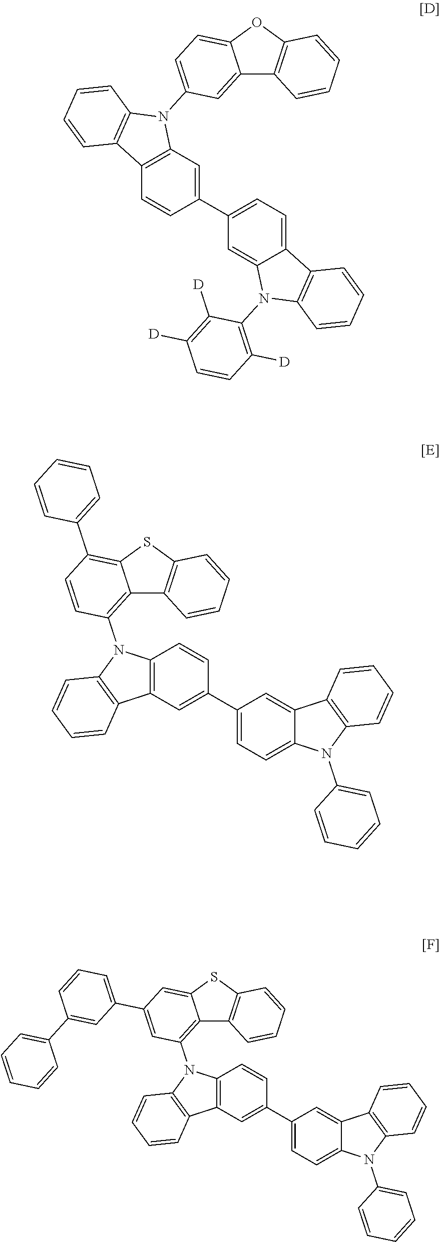 [SMILES: [2H]c1ccc([2H])c(-n2c3ccccc3c3ccc(-c4ccc5c6ccccc6n(-c6ccc7oc8ccccc8c7c6)c5c4)cc32)c1[2H] ; c1ccc(-c2ccc(-n3c4ccccc4c4cc(-c5ccc6c(c5)c5ccccc5n6-c5ccccc5)ccc43)c3c2sc2ccccc23)cc1 ; c1ccc(-c2cccc(-c3cc(-n4c5ccccc5c5cc(-c6ccc7c(c6)c6ccccc6n7-c6ccccc6)ccc54)c4c(c3)sc3ccccc34)c2)cc1]

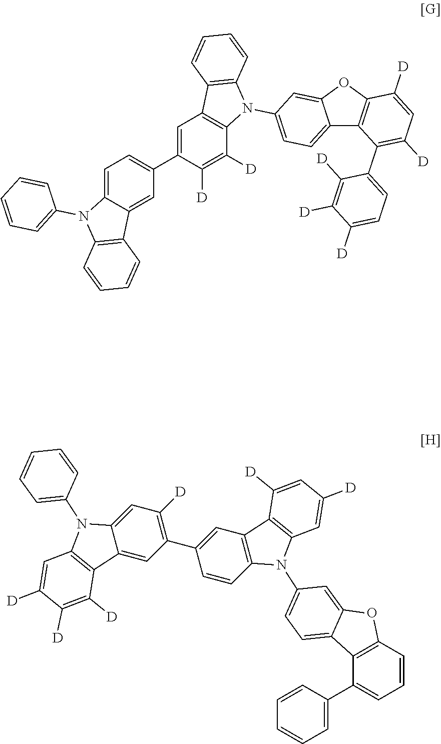 [SMILES: [2H]c1ccc(-c2c([2H])cc([2H])c3oc4cc(-n5c6ccccc6c6cc(-c7ccc8c(c7)c7ccccc7n8-c7ccccc7)c([2H])c([2H])c65)ccc4c23)c([2H])c1[2H] ; [2H]c1cc([2H])c2c3cc(-c4cc5c6c([2H])c([2H])c([2H])cc6n(-c6ccccc6)c5cc4[2H])ccc3n(-c3ccc4c(c3)oc3cccc(-c5ccccc5)c34)c2c1]

### (2) Driving Voltage and Light Emitting Efficiency of Organic ElIectroluminescence Device

For the organic electroluminescence device manufactured as described above, electroluminescence (EL) characteristics were measured by M7000 manufactured by Mc Science Inc., and based on the measurement result thereof, T₉₀ was measured by a service life measurement device (M6000) manufactured by Mc Science Inc., when the reference luminance was 6,000 cd/m². The measurement results are shown in the following Table 12.

TABLE 12

| | | Driving | | | Service |
|---|---|---|---|---|---|
| | | voltage | Efficiency | Color | life |
| | Compound | (V) | (cd/A) | coordinate | (T90) |
| Comparative | [A] | 6.20 | 40.3 | Green | 63 |
| Example 1 | | | | | |
| Comparative | [B] | 6.11 | 41.6 | Green | 60 |
| Example 2 | | | | | |
| Comparative | [C] | 5.97 | 42.1 | Green | 61 |
| Example 3 | | | | | |
| Comparative | [D] | 5.96 | 41.7 | Green | 62 |
| Example 4 | | | | | |
| Comparative | [E] | 5.87 | 42.9 | Green | 66 |
| Example 5 | | | | | |
| Comparative | [F] | 5.76 | 43.3 | Green | 65 |
| Example 6 | | | | | |
| Comparative | [G] | 5.53 | 44.7 | Green | 69 |
| Example 7 | | | | | |
| Comparative | [H] | 5.50 | 45.3 | Green | 72 |
| Example 8 | | | | | |
| Example 1 | 1-3 | 3.11 | 99.6 | Green | 152 |
| Example 2 | 1-4 | 3.29 | 100.8 | Green | 149 |
| Example 3 | 1-7 | 3.30 | 109.4 | Green | 155 |
| Example 4 | 1-10 | 3.17 | 110.4 | Green | 153 |
| Example 5 | 1-14 | 3.26 | 102.5 | Green | 148 |
| Example 6 | 1-17 | 3.32 | 98.7 | Green | 145 |
| Example 7 | 1-23 | 3.70 | 96.5 | Green | 148 |
| Example 8 | 1-26 | 3.68 | 92.7 | Green | 143 |
| Example 9 | 1-35 | 3.59 | 93.8 | Green | 144 |
| Example 10 | 1-38 | 3.62 | 90.4 | Green | 141 |
| Example 11 | 1-43 | 4.19 | 71.3 | Green | 130 |
| Example 12 | 1-44 | 4.13 | 70.6 | Green | 134 |
| Example 13 | 1-46 | 4.06 | 75.1 | Green | 131 |
| Example 14 | 1-51 | 4.26 | 73.2 | Green | 135 |
| Example 15 | 1-54 | 4.38 | 74.8 | Green | 132 |
| Example 16 | 1-56 | 4.42 | 65.9 | Green | 128 |
| Example 17 | 1-62 | 4.66 | 63.5 | Green | 120 |
| Example 18 | 1-68 | 4.58 | 61.2 | Green | 126 |
| Example 19 | 1-70 | 4.69 | 63.9 | Green | 121 |
| Example 20 | 1-72 | 4.72 | 60.8 | Green | 125 |
| Example 21 | 1-75 | 4.65 | 62.3 | Green | 120 |
| Example 22 | 1-78 | 5.03 | 61.5 | Green | 122 |
| Example 23 | 1-83 | 2.41 | 125.4 | Green | 168 |
| Example 24 | 1-85 | 2.32 | 123.7 | Green | 165 |
| Example 25 | 1-86 | 2.29 | 124.6 | Green | 169 |
| Example 26 | 1-87 | 2.32 | 125.3 | Green | 172 |
| Example 27 | 1-90 | 2.38 | 121.7 | Green | 160 |
| Example 28 | 1-93 | 2.16 | 122.8 | Green | 161 |
| Example 29 | 1-94 | 2.23 | 123.1 | Green | 169 |
| Example 30 | 1-96 | 2.05 | 124.6 | Green | 170 |
| Example 31 | 1-97 | 2.11 | 120.4 | Green | 163 |
| Example 32 | 1-103 | 3.16 | 110.9 | Green | 151 |
| Example 33 | 1-104 | 2.72 | 112.7 | Green | 154 |
| Example 34 | 1-106 | 2.98 | 111.2 | Green | 159 |
| Example 35 | 1-107 | 3.20 | 109.4 | Green | 162 |
| Example 36 | 1-110 | 3.19 | 118.6 | Green | 155 |
| Example 37 | 1-114 | 3.02 | 110.6 | Green | 150 |
| Example 38 | 1-118 | 3.13 | 113.5 | Green | 152 |
| Example 39 | 1-122 | 3.78 | 95.1 | Green | 100 |
| Example 40 | 1-128 | 3.51 | 94.8 | Green | 105 |
| Example 41 | 1-129 | 3.74 | 93.2 | Green | 110 |
| Example 42 | 1-134 | 3.67 | 90.4 | Green | 116 |
| Example 43 | 1-136 | 3.55 | 95.4 | Green | 120 |
| Example 44 | 1-143 | 4.06 | 85.5 | Green | 95 |
| Example 45 | 1-146 | 4.15 | 86.7 | Green | 91 |
| Example 46 | 1-147 | 4.19 | 83.2 | Green | 94 |
| Example 47 | 1-150 | 4.16 | 82.4 | Green | 89 |
| Example 48 | 1-155 | 4.21 | 80.5 | Green | 93 |

According to Table 12 above, it was confirmed that the heterocyclic compound of Chemical Formula 1 of the present application, in which Ar2 is bonded to positions 1 to 4 of dibenzofuran and dibenzothiophene bonded to the bis-carbazole core of Chemical Formula 1 of the present application, has reduced bond rotation due to the increased steric hindrance between the biscarbazole core and the substituent, and has enhanced stability of the molecular structure due to a decrease in vibrational energy of the molecule, compared to Comparative Examples [D], [E], and [F], so that the heterocyclic compound of Chemical Formula 1 of the present application has better service life characteristics than when Comparative Examples [D], [E], and [F] were used in a device.

Further, it was confirmed that Comparative Example [C], in which deuterium was not included in the structure of Chemical Formula 1 of the present application, was inferior in terms of service life to the case where the heterocyclic compound of the present invention was used.

In addition, it was confirmed that when the deuterium substitution rate is less than 60%, as in Comparative Examples [G] and [H], this effect is negligible, so that the performance is similar to that of a compound with a substitution rate of 0%.

Furthermore, it was confirmed that in Comparative Examples [A] and [B], the electron withdrawing group attracts electrons in the molecule, so that when Comparative Examples [A] and [B] were used in a device, the hole characteristics are insufficient, resulting in poor driving voltage and service life.

In particular, in the biscarbazole core of Chemical Formula 1 of the present application, positions 1 and 3 of the carbazole are more likely to have unshared electron pairs than positions 2 and 4 of the carbazole due to a resonance structure, and carry a negative charge.

Therefore, it was confirmed that since a biscarbazole core in which positions 3 of carbazole are bonded to each other and a biscarbazole core in which position 1 of carbazole is bonded to position 3 of carbazole can transfer and share the unshared electron pair at position 3 of carbazole to the adjacent carbazole in the molecule, the biscarbazole cores have relatively smoother electron transport between intramolecular P-orbitals than in the case of a biscarbazole core in which position 3 of carbazole is bonded to position 2 of carbazole and a biscarbazole core in which position 3 of carbazole is bonded to position 4 of carbazole, and thus, have excellent driving and efficiency.

### Experimental Example 2

### (1) Manufacture of Organic Light Emitting Device

An organic electroluminescence device was manufactured in the same manner as in the above-described Experimental Example 1, except that as a host material for a light emitting layer, a compound described in the following Table 13 was used. In the following Table 13, the ratios mean weight ratios.

### (2) Driving Voltage and Light Emitting Efficiency of Organic Electroluminescence Device

For the organic electroluminescence device manufactured as described above, electroluminescence (EL) characteristics were measured by M7000 manufactured by Mc Science Inc., and based on the measurement result thereof, T₉₀ was measured by a service life measurement device (M6000) manufactured by Mc Science Inc., when the reference luminance was 6,000 cd/m². The measurement results are shown in the following Table 13.

TABLE 13

| | Light | | | | | |
|---|---|---|---|---|---|---|
| | emitting | | Driving | Effi- | Color | Service |
| | layer | | voltage | ciency | coordi- | life |
| | compound | Ratio | (V) | (cd/A) | nate | (T90) |
| Comparative | [A]:2-56 | 1:1 | 4.87 | 72.5 | Green | 90 |
| Example 9 | | | | | | |
| Comparative | | 2:1 | 4.92 | 71.6 | Green | 92 |
| Example 10 | | | | | | |
| Comparative | | 3:1 | 4.99 | 70.4 | Green | 95 |
| Example 11 | | | | | | |
| Comparative | [B]:2-78 | 1:1 | 4.72 | 73.9 | Green | 93 |
| Example 12 | | | | | | |
| Comparative | | 2:1 | 4.77 | 73.2 | Green | 96 |
| Example 13 | | | | | | |
| Comparative | | 3:1 | 4.82 | 72.5 | Green | 99 |
| Example 14 | | | | | | |
| Comparative | [C]:2-64 | 1:1 | 4.69 | 76.9 | Green | 95 |
| Example 15 | | | | | | |
| Comparative | | 2:1 | 4.72 | 76.4 | Green | 99 |
| Example 16 | | | | | | |
| Comparative | | 3:1 | 4.75 | 75.3 | Green | 103 |
| Example 17 | | | | | | |
| Comparative | [D]:2-75 | 1:1 | 4.63 | 78.2 | Green | 97 |
| Example 18 | | | | | | |
| Comparative | | 2:1 | 4.69 | 77.3 | Green | 105 |
| Example 19 | | | | | | |
| Comparative | | 3:1 | 4.71 | 76.1 | Green | 109 |
| Example 20 | | | | | | |
| Comparative | [E]:2-79 | 1:1 | 4.43 | 82.6 | Green | 100 |
| Example 21 | | | | | | |
| Comparative | | 2:1 | 4.52 | 81.7 | Green | 107 |
| Example 22 | | | | | | |
| Comparative | | 3:1 | 4.68 | 81.1 | Green | 110 |
| Example 23 | | | | | | |
| Comparative | [F]:2-65 | 1:1 | 4.36 | 83.0 | Green | 95 |
| Example 24 | | | | | | |
| Comparative | | 2:1 | 4.41 | 82.4 | Green | 102 |
| Example 25 | | | | | | |
| Comparative | | 3:1 | 4.44 | 81.7 | Green | 109 |
| Example 26 | | | | | | |
| Comparative | [G]:2-76 | 1:1 | 4.02 | 84.7 | Green | 108 |
| Example 27 | | | | | | |
| Comparative | | 2:1 | 4.11 | 83.5 | Green | 112 |
| Example 28 | | | | | | |
| Comparative | | 3:1 | 4.23 | 83.1 | Green | 115 |
| Example 29 | | | | | | |
| Comparative | [H]:2-56 | 1:1 | 4.16 | 85.0 | Green | 103 |
| Example 30 | | | | | | |
| Comparative | | 2:1 | 4.23 | 84.2 | Green | 105 |
| Example 31 | | | | | | |
| Comparative | | 3:1 | 4.38 | 83.4 | Green | 110 |
| Example 32 | | | | | | |
| Example 49 | 1-3:2-79 | 1:1 | 2.13 | 132.5 | Green | 173 |
| Example 50 | | 2:1 | 2.18 | 131.4 | Green | 175 |
| Example 51 | | 3:1 | 2.21 | 130.2 | Green | 181 |
| Example 52 | 1-4:2-64 | 1:1 | 2.05 | 135.4 | Green | 182 |
| Example 53 | | 2:1 | 2.16 | 134.2 | Green | 188 |
| Example 54 | | 3:1 | 2.23 | 133.7 | Green | 191 |
| Example 55 | 1-7:2-75 | 1:1 | 2.22 | 130.5 | Green | 174 |
| Example 56 | | 2:1 | 2.34 | 129.4 | Green | 177 |
| Example 57 | | 3:1 | 2.41 | 128.7 | Green | 180 |
| Example 58 | 1-10:2-56 | 1:1 | 2.08 | 134.4 | Green | 185 |
| Example 59 | | 2:1 | 2.17 | 132.6 | Green | 189 |
| Example 60 | | 3:1 | 2.25 | 131.5 | Green | 192 |
| Example 61 | 1-14:2-68 | 1:1 | 2.16 | 132.8 | Green | 176 |
| Example 62 | | 2:1 | 2.24 | 131.5 | Green | 180 |
| Example 63 | | 3:1 | 2.38 | 130.4 | Green | 183 |
| Example 64 | 1-17:2-32 | 1:1 | 2.05 | 135.4 | Green | 180 |
| Example 65 | | 2:1 | 2.11 | 134.2 | Green | 183 |
| Example 66 | | 3:1 | 2.18 | 133.1 | Green | 190 |
| Example 67 | 1-23:2-76 | 1:1 | 3.16 | 124.8 | Green | 173 |
| Example 68 | | 2:1 | 3.20 | 123.1 | Green | 177 |
| Example 69 | | 3:1 | 3.25 | 122.6 | Green | 179 |
| Example 70 | 1-26:2-73 | 1:1 | 2.89 | 125.4 | Green | 181 |
| Example 71 | | 2:1 | 2.93 | 124.7 | Green | 183 |
| Example 72 | | 3:1 | 2.99 | 123.1 | Green | 188 |
| Example 73 | 1-35:2-79 | 1:1 | 3.05 | 123.8 | Green | 171 |
| Example 74 | | 2:1 | 3.16 | 122.6 | Green | 174 |
| Example 75 | | 3:1 | 3.19 | 121.4 | Green | 175 |
| Example 76 | 1-38:2-68 | 1:1 | 3.10 | 122.8 | Green | 177 |
| Example 77 | | 2:1 | 3.15 | 121.6 | Green | 180 |
| Example 78 | | 3:1 | 3.22 | 120.4 | Green | 183 |
| Example 79 | 1-43:2-73 | 1:1 | 3.48 | 95.2 | Green | 162 |
| Example 80 | | 2:1 | 3.50 | 94.6 | Green | 166 |
| Example 81 | | 3:1 | 3.52 | 93.2 | Green | 170 |
| Example 82 | 1-44:2-75 | 1:1 | 3.64 | 94.4 | Green | 161 |
| Example 83 | | 2:1 | 3.66 | 93.6 | Green | 165 |
| Example 84 | | 3:1 | 3.72 | 92.1 | Green | 169 |
| Example 85 | 1-46:2-72 | 1:1 | 3.51 | 93.3 | Green | 158 |
| Example 86 | | 2:1 | 3.55 | 92.6 | Green | 162 |
| Example 87 | | 3:1 | 3.59 | 91.1 | Green | 166 |
| Example 88 | 1-51:2-64 | 1:1 | 3.54 | 93.8 | Green | 163 |
| Example 89 | | 2:1 | 3.58 | 92.1 | Green | 166 |
| Example 90 | | 3:1 | 3.60 | 91.6 | Green | 169 |
| Example 91 | 1-54:2-56 | 1:1 | 3.52 | 94.5 | Green | 160 |
| Example 92 | | 2:1 | 3.59 | 93.9 | Green | 163 |
| Example 93 | | 3:1 | 3.62 | 92.1 | Green | 164 |
| Example 94 | 1-56:2-21 | 1:1 | 3.50 | 95.4 | Green | 166 |
| Example 95 | | 2:1 | 3.55 | 94.6 | Green | 169 |
| Example 96 | | 3:1 | 3.62 | 93.2 | Green | 172 |
| Example 97 | 1-62:2-73 | 1:1 | 3.94 | 83.7 | Green | 152 |
| Example 98 | | 2:1 | 3.99 | 83.3 | Green | 155 |
| Example 99 | | 3:1 | 4.02 | 82.6 | Green | 159 |
| Example | 1-68:2-62 | 1:1 | 3.87 | 82.9 | Green | 150 |
| 100 | | | | | | |
| Example | | 2:1 | 3.94 | 81.5 | Green | 153 |
| 101 | | | | | | |
| Example | | 3:1 | 4.02 | 80.4 | Green | 156 |
| 102 | | | | | | |
| Example | 1-70:2-64 | 1:1 | 3.99 | 84.6 | Green | 151 |
| 103 | | | | | | |
| Example | | 2:1 | 4.05 | 83.2 | Green | 153 |
| 104 | | | | | | |
| Example | | 3:1 | 4.12 | 82.6 | Green | 155 |
| 105 | | | | | | |
| Example | 1-72:2-76 | 1:1 | 4.08 | 85.1 | Green | 152 |
| 106 | | | | | | |
| Example | | 2:1 | 4.11 | 84.6 | Green | 159 |
| 107 | | | | | | |
| Example | | 3:1 | 4.19 | 83.2 | Green | 162 |
| 108 | | | | | | |
| Example | 1-75:2-55 | 1:1 | 4.13 | 81.5 | Green | 152 |
| 109 | | | | | | |
| Example | | 2:1 | 4.19 | 80.4 | Green | 157 |
| 110 | | | | | | |
| Example | | 3:1 | 4.20 | 79.9 | Green | 161 |
| 111 | | | | | | |
| Example | 1-78:2-56 | 1:1 | 4.15 | 81.8 | Green | 150 |
| 112 | | | | | | |
| Example | | 2:1 | 4.18 | 81.2 | Green | 152 |
| 113 | | | | | | |
| Example | | 3:1 | 4.20 | 80.5 | Green | 155 |
| 114 | | | | | | |
| Example | 1-83:2-62 | 1:1 | 1.05 | 153.2 | Green | 216 |
| 115 | | | | | | |
| Example | | 2:1 | 1.12 | 152.6 | Green | 219 |
| 116 | | | | | | |
| Example | | 3:1 | 1.22 | 151.7 | Green | 223 |
| 117 | | | | | | |
| Example | 1-85:2-56 | 1:1 | 1.13 | 154.6 | Green | 210 |
| 118 | | | | | | |
| Example | | 2:1 | 1.15 | 153.7 | Green | 212 |
| 119 | | | | | | |
| Example | | 3:1 | 1.23 | 152.1 | Green | 219 |
| 120 | | | | | | |
| Example | 1-86:2-75 | 1:1 | 1.01 | 155.4 | Green | 205 |
| 121 | | | | | | |
| Example | | 2:1 | 1.09 | 154.3 | Green | 213 |
| 122 | | | | | | |
| Example | | 3:1 | 1.11 | 153.7 | Green | 219 |
| 123 | | | | | | |
| Example | 1-87:2-73 | 1:1 | 1.07 | 154.9 | Green | 198 |
| 124 | | | | | | |
| Example | | 2:1 | 1.12 | 153.2 | Green | 201 |
| 125 | | | | | | |
| Example | | 3:1 | 1.15 | 152.7 | Green | 206 |
| 126 | | | | | | |
| Example | 1-90:2-21 | 1:1 | 1.12 | 155.1 | Green | 205 |
| 127 | | | | | | |
| Example | | 2:1 | 1.18 | 154.1 | Green | 216 |
| 128 | | | | | | |
| Example | | 3:1 | 1.23 | 153.6 | Green | 219 |
| 129 | | | | | | |
| Example | 1-93:2-32 | 1:1 | 1.15 | 153.8 | Green | 208 |
| 130 | | | | | | |
| Example | | 2:1 | 1.18 | 152.6 | Green | 215 |
| 131 | | | | | | |
| Example | | 3:1 | 1.23 | 151.1 | Green | 220 |
| 132 | | | | | | |
| Example | 1-94:2-56 | 1:1 | 1.13 | 153.7 | Green | 215 |
| 133 | | | | | | |
| Example | | 2:1 | 1.15 | 152.8 | Green | 219 |
| 134 | | | | | | |
| Example | | 3:1 | 1.19 | 152.2 | Green | 222 |
| 135 | | | | | | |
| Example | 1-96:2-64 | 1:1 | 1.06 | 154.6 | Green | 211 |
| 136 | | | | | | |
| Example | | 2:1 | 1.13 | 153.7 | Green | 216 |
| 137 | | | | | | |
| Example | | 3:1 | 1.19 | 152.6 | Green | 220 |
| 138 | | | | | | |
| Example | 1-97:2-79 | 1:1 | 1.05 | 155.2 | Green | 219 |
| 139 | | | | | | |
| Example | | 2:1 | 1.12 | 154.8 | Green | 221 |
| 140 | | | | | | |
| Example | | 3:1 | 1.15 | 154.2 | Green | 225 |
| 141 | | | | | | |
| Example | 1-103:2-73 | 1:1 | 2.05 | 146.8 | Green | 192 |
| 142 | | | | | | |
| Example | | 2:1 | 2.16 | 145.6 | Green | 195 |
| 143 | | | | | | |
| Example | | 3:1 | 2.19 | 144.2 | Green | 199 |
| 144 | | | | | | |
| Example | 1-104:2-68 | 1:1 | 2.01 | 149.4 | Green | 190 |
| 145 | | | | | | |
| Example | | 2:1 | 2.11 | 148.2 | Green | 193 |
| 146 | | | | | | |
| Example | | 3:1 | 2.16 | 147.1 | Green | 196 |
| 147 | | | | | | |
| Example | 1-106:2-75 | 1:1 | 2.13 | 148.8 | Green | 193 |
| 148 | | | | | | |
| Example | | 2:1 | 2.19 | 147.2 | Green | 195 |
| 149 | | | | | | |
| Example | | 3:1 | 2.24 | 146.5 | Green | 198 |
| 150 | | | | | | |
| Example | 1-107:2-62 | 1:1 | 2.16 | 145.2 | Green | 196 |
| 151 | | | | | | |
| Example | | 2:1 | 2.23 | 144.9 | Green | 199 |
| 152 | | | | | | |
| Example | | 3:1 | 2.25 | 143.1 | Green | 203 |
| 153 | | | | | | |
| Example | 1-110:2-65 | 1:1 | 2.11 | 146.5 | Green | 194 |
| 154 | | | | | | |
| Example | | 2:1 | 2.23 | 145.2 | Green | 205 |
| 155 | | | | | | |
| Example | | 3:1 | 2.26 | 144.3 | Green | 207 |
| 156 | | | | | | |
| Example | 1-114:2-76 | 1:1 | 2.05 | 143.8 | Green | 191 |
| 157 | | | | | | |
| Example | | 2:1 | 2.09 | 142.6 | Green | 195 |
| 158 | | | | | | |
| Example | | 3:1 | 2.12 | 141.2 | Green | 198 |
| 159 | | | | | | |
| Example | 1-118:2-73 | 1:1 | 2.15 | 142.1 | Green | 190 |
| 160 | | | | | | |
| Example | | 2:1 | 2.19 | 141.6 | Green | 192 |
| 161 | | | | | | |
| Example | | 3:1 | 2.20 | 140.4 | Green | 195 |
| 162 | | | | | | |
| Example | 1-122:2-71 | 1:1 | 3.09 | 115.2 | Green | 138 |
| 163 | | | | | | |
| Example | | 2:1 | 3.12 | 114.8 | Green | 141 |
| 164 | | | | | | |
| Example | | 3:1 | 3.18 | 114.1 | Green | 143 |
| 165 | | | | | | |
| Example | 1-128:2-32 | 1:1 | 3.13 | 113.6 | Green | 144 |
| 166 | | | | | | |
| Example | | 2:1 | 3.15 | 112.7 | Green | 148 |
| 167 | | | | | | |
| Example | | 3:1 | 3.19 | 111.5 | Green | 152 |
| 168 | | | | | | |
| Example | 1-129:2-55 | 1:1 | 3.05 | 115.0 | Green | 140 |
| 169 | | | | | | |
| Example | | 2:1 | 3.10 | 114.2 | Green | 142 |
| 170 | | | | | | |
| Example | | 3:1 | 3.16 | 113.8 | Green | 149 |
| 171 | | | | | | |
| Example | 1-134:2-56 | 1:1 | 3.04 | 113.6 | Green | 143 |
| 172 | | | | | | |
| Example | | 2:1 | 3.09 | 112.5 | Green | 146 |
| 173 | | | | | | |
| Example | | 3:1 | 3.12 | 111.9 | Green | 150 |
| 174 | | | | | | |
| Example | 1-136:2-64 | 1:1 | 3.05 | 114.2 | Green | 140 |
| 175 | | | | | | |
| Example | | 2:1 | 3.11 | 114.0 | Green | 142 |
| 176 | | | | | | |
| Example | | 3:1 | 3.15 | 113.2 | Green | 145 |
| 177 | | | | | | |
| Example | 1-143:2-79 | 1:1 | 3.52 | 105.2 | Green | 132 |
| 178 | | | | | | |
| Example | | 2:1 | 3.55 | 104.9 | Green | 136 |
| 179 | | | | | | |
| Example | | 3:1 | 3.61 | 103.2 | Green | 139 |
| 180 | | | | | | |
| Example | 1-146:2-78 | 1:1 | 3.64 | 110.5 | Green | 130 |
| 181 | | | | | | |
| Example | | 2:1 | 3.66 | 109.4 | Green | 133 |
| 182 | | | | | | |
| Example | | 3:1 | 3.70 | 108.2 | Green | 139 |
| 183 | | | | | | |
| Example | 1-147:2-21 | 1:1 | 3.59 | 107.6 | Green | 132 |
| 184 | | | | | | |
| Example | | 2:1 | 3.61 | 106.4 | Green | 135 |
| 185 | | | | | | |
| Example | | 3:1 | 3.68 | 105.8 | Green | 139 |
| 186 | | | | | | |
| Example | 1-150:2-56 | 1:1 | 3.62 | 110.6 | Green | 131 |
| 187 | | | | | | |
| Example | | 2:1 | 3.64 | 109.7 | Green | 138 |
| 188 | | | | | | |
| Example | | 3:1 | 3.69 | 106.7 | Green | 140 |
| 189 | | | | | | |
| Example | | 1:1 | 3.58 | 102.2 | Green | 133 |
| 190 | | | | | | |
| Example | 1-155:2-73 | 2:1 | 3.61 | 101.6 | Green | 139 |
| 191 | | | | | | |
| Example | | 3:1 | 3.70 | 100.4 | Green | 141 |
| 192 | | | | | | |

The heterocyclic compound represented by Chemical Formula 1 of the present invention allows a biscarbazole core to be bonded to Ar2-bonded dibenzofuran or Ar2-bonded dibenzothiophene as a substituent to expand the aromaticity and resonance of the core and allows more P-orbital electrons to move smoothly, thereby increasing intramolecular electron transport.

According to Table 13, when Chemical Formula 1, which has abundant electrons in the molecule, is used in combination with Chemical Formula 2, which is relatively electron-poor, intermolecular electron transport occurs very smoothly, resulting in excellent characteristics in terms of driving voltage, efficiency and service life.

In particular, when Comparative Example Compounds [A] and [B] including an electron withdrawing group are used in combination with Chemical Formula 2, the movement of holes and electrons is not balanced because Chemical Formula 2 also includes an electron withdrawing group, so that it was confirmed that driving and efficiency significantly deteriorated.

Furthermore, it was confirmed that even the case where Comparative Example Compounds C to H, which do not satisfy the deuterium substitution rate according to Chemical Formula 1 of the present application, are used in combination with the compound of Chemical Formula 2 was inferior in terms of driving voltage, efficiency, and service life to the case where the heterocyclic compound represented by Chemical Formula 1 of the present application and the compound represented by Chemical Formula 2 of the present application are used in combination.

1. A heterocyclic compound represented by the following Chemical Formula 1: 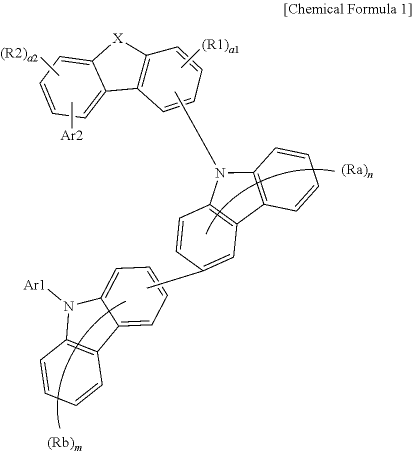
wherein, in Chemical Formula 1,
X is O or S,
Ar1 is a substituted or unsubstituted C6 to C60 aryl group,
Ar2 is a C6 to C60 aryl group substituted with at least one deuterium,
R1, R2, Ra, and Rb are the same as or different from each other, and are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group,
a1 is an integer from 0 to 3, and when a1 is 2 or higher, substituents in the parenthesis are the same as or different from each other,
a2 is an integer from 0 to 3, and when a2 is 2 or higher, substituents in the parenthesis are the same as or different from each other,
m is an integer from 0 to 7, and when m is 2 or higher, substituents in the parenthesis are the same as or different from each other,
n is an integer from 0 to 7, and when n is 2 or higher, substituents in the parenthesis are the same as or different from each other, and
a deuterium substitution rate of 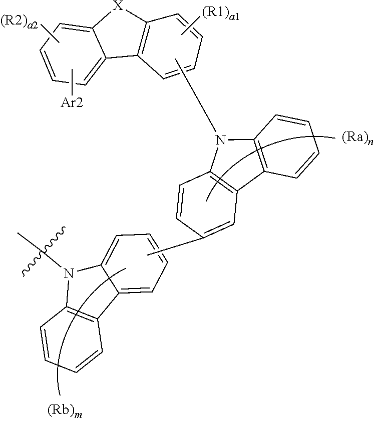
of Chemical Formula 1 is 60% to 100%.

2. The heterocyclic compound of claim 1, wherein Chemical Formula 1 is represented by the following Structural Formulae A to C: 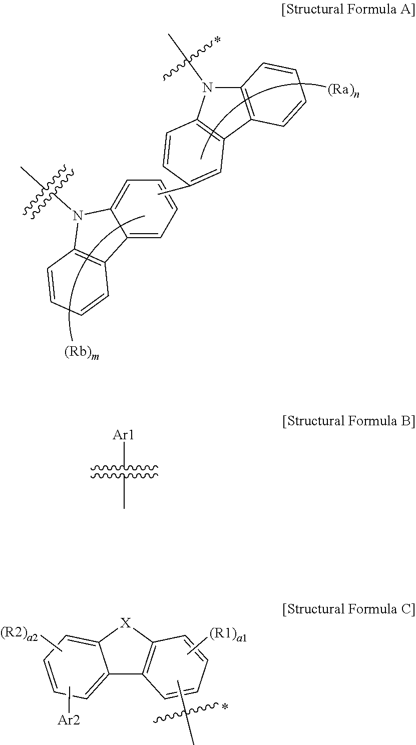
in Structural Formulae A to C,
the definitions of X, Ar1, Ar2, R1, R2, Ra, Rb, a1, a2, m, and n are the same as the definitions in Chemical Formula 1,
the deuterium substitution rate of a structure in which Structural Formula A and Structural Formula C are combined is 60% to 100%, and
the 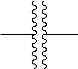
is a position where Structural Formula A and Structural Formula B are linked, and the 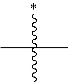
is a position where Structural Formula A and Structural Formula C are linked.

3. The heterocyclic compound of claim 2, wherein a deuterium substitution rate of Structural Formula B is 0%.

4. The heterocyclic compound of claim 2, wherein a deuterium substitution rate of a structure in which Structural Formula A and Structural Formula C are combined is 80% to 100%.

5. The heterocyclic compound of claim 1, wherein Ar1 is a C6 to C20 aryl group.

6. The heterocyclic compound of claim 1, wherein Ar2 is a C6 to C20 aryl group substituted with at least one deuterium.

7. The heterocyclic compound of claim 1, wherein R1, R2, Ra, and Rb are the same as or different from each other, and are each independently hydrogen; or deuterium.

8. The heterocyclic compound of claim 1, wherein Chemical Formula 1 is represented by any one of the following compounds:

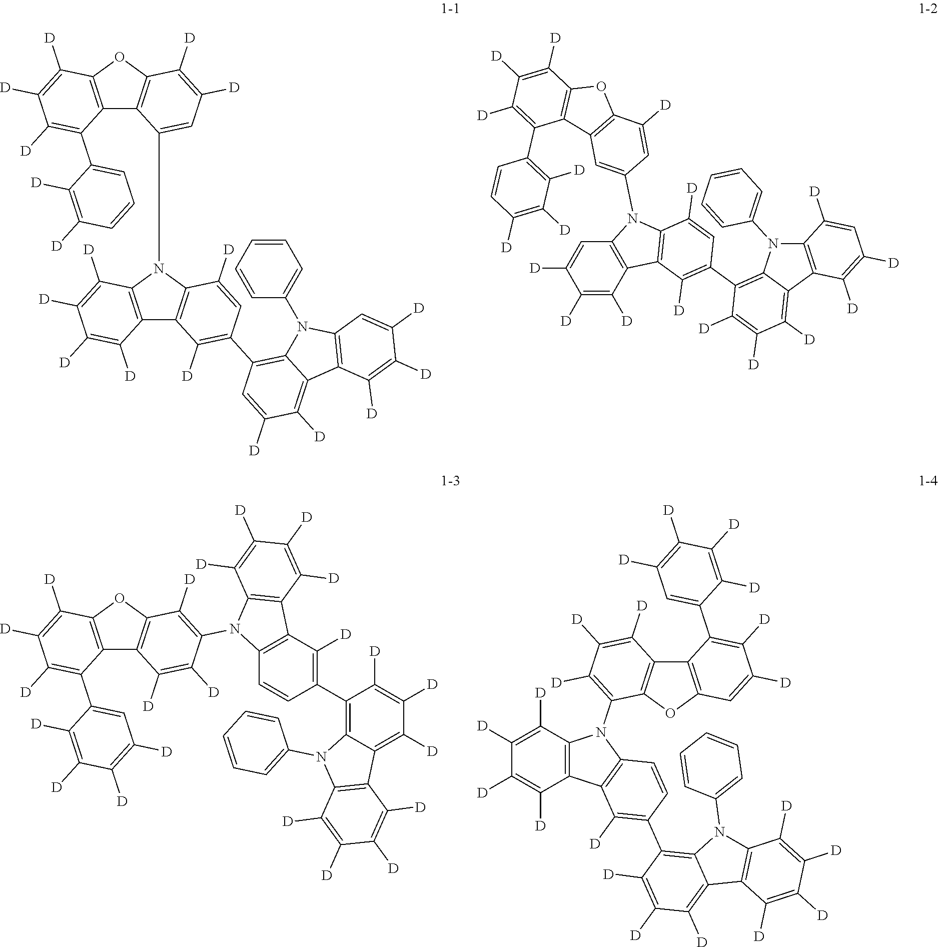

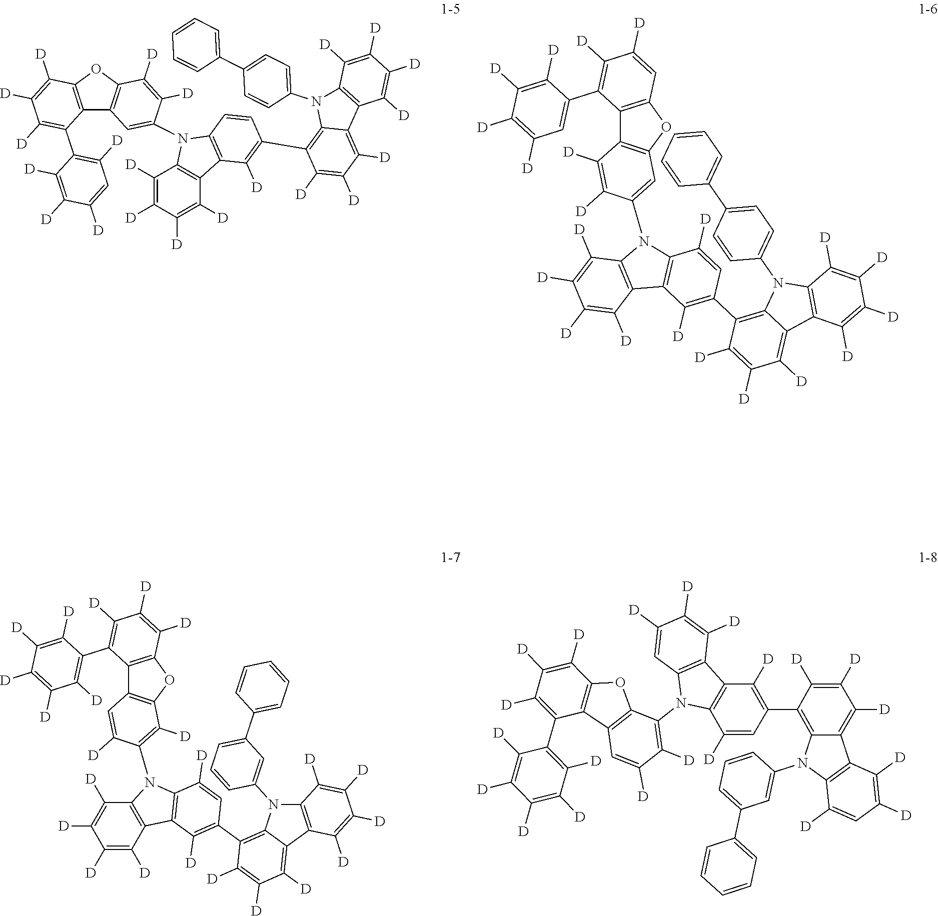

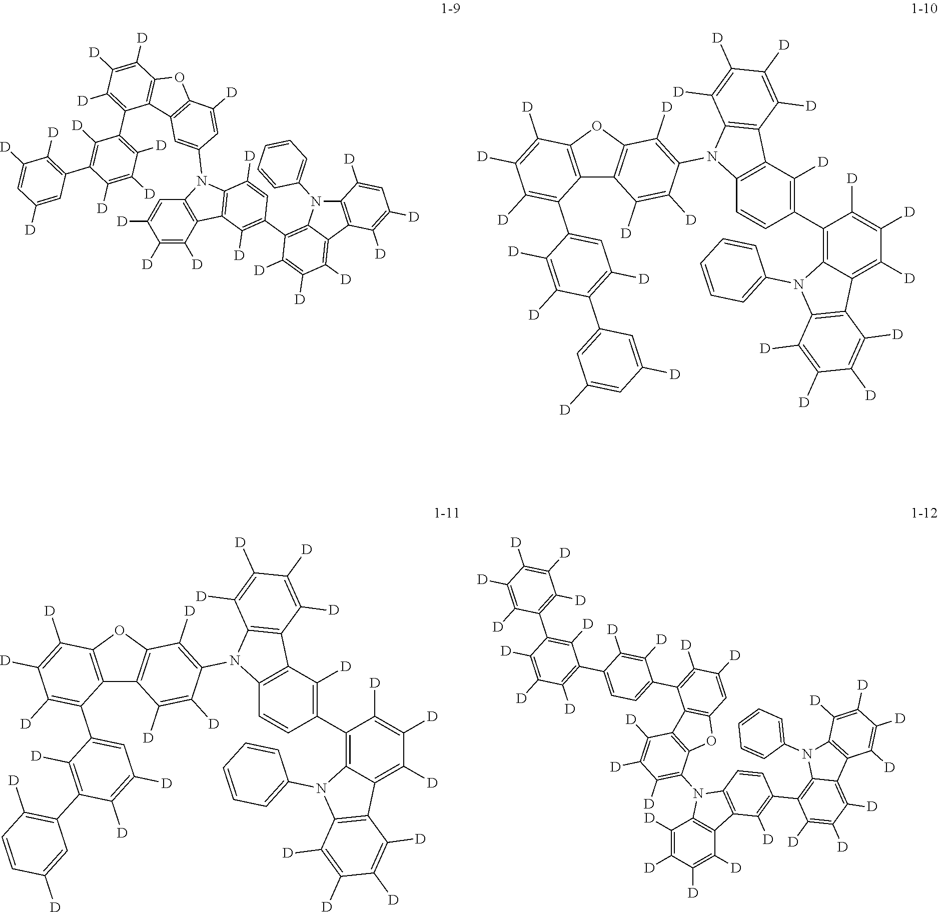

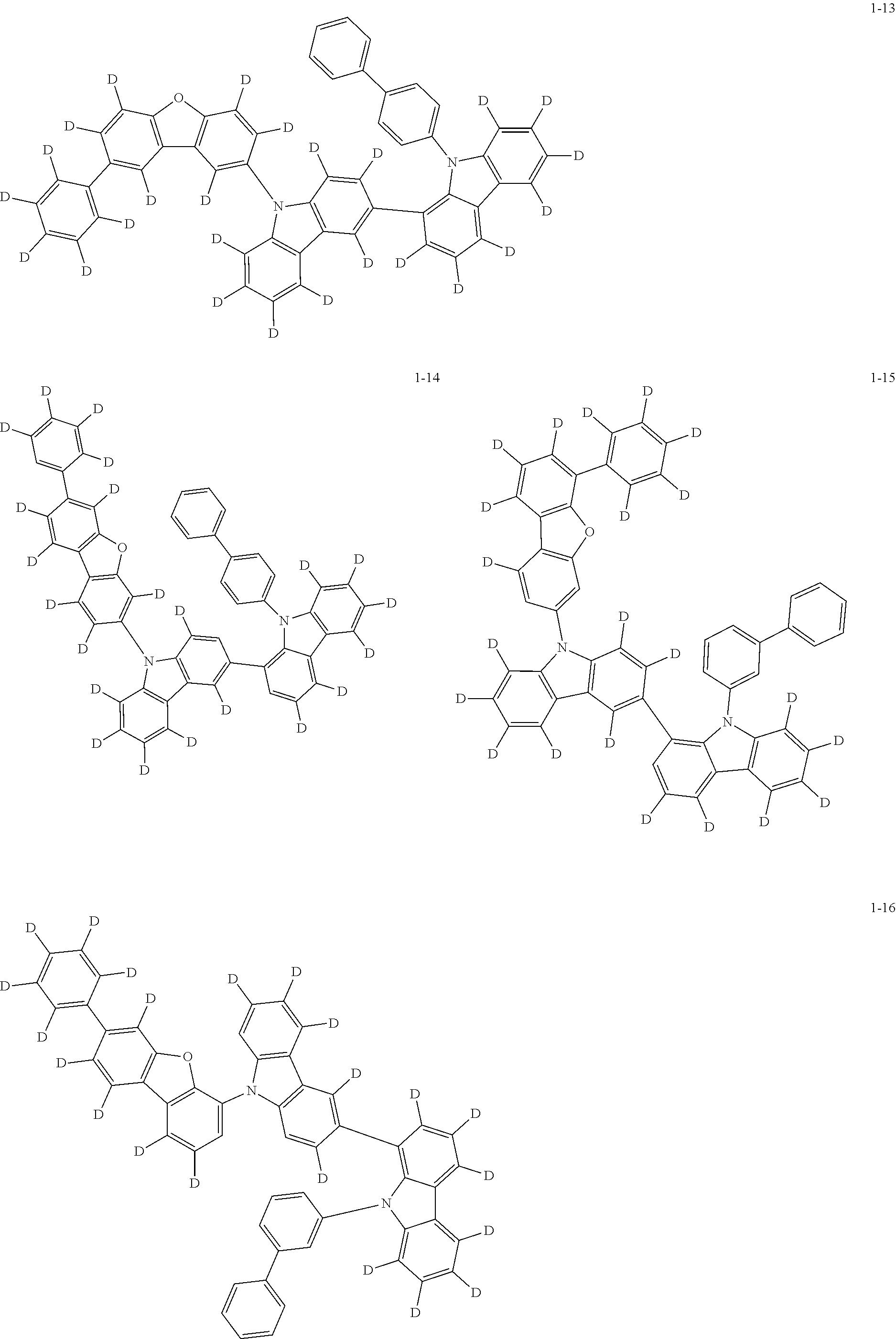

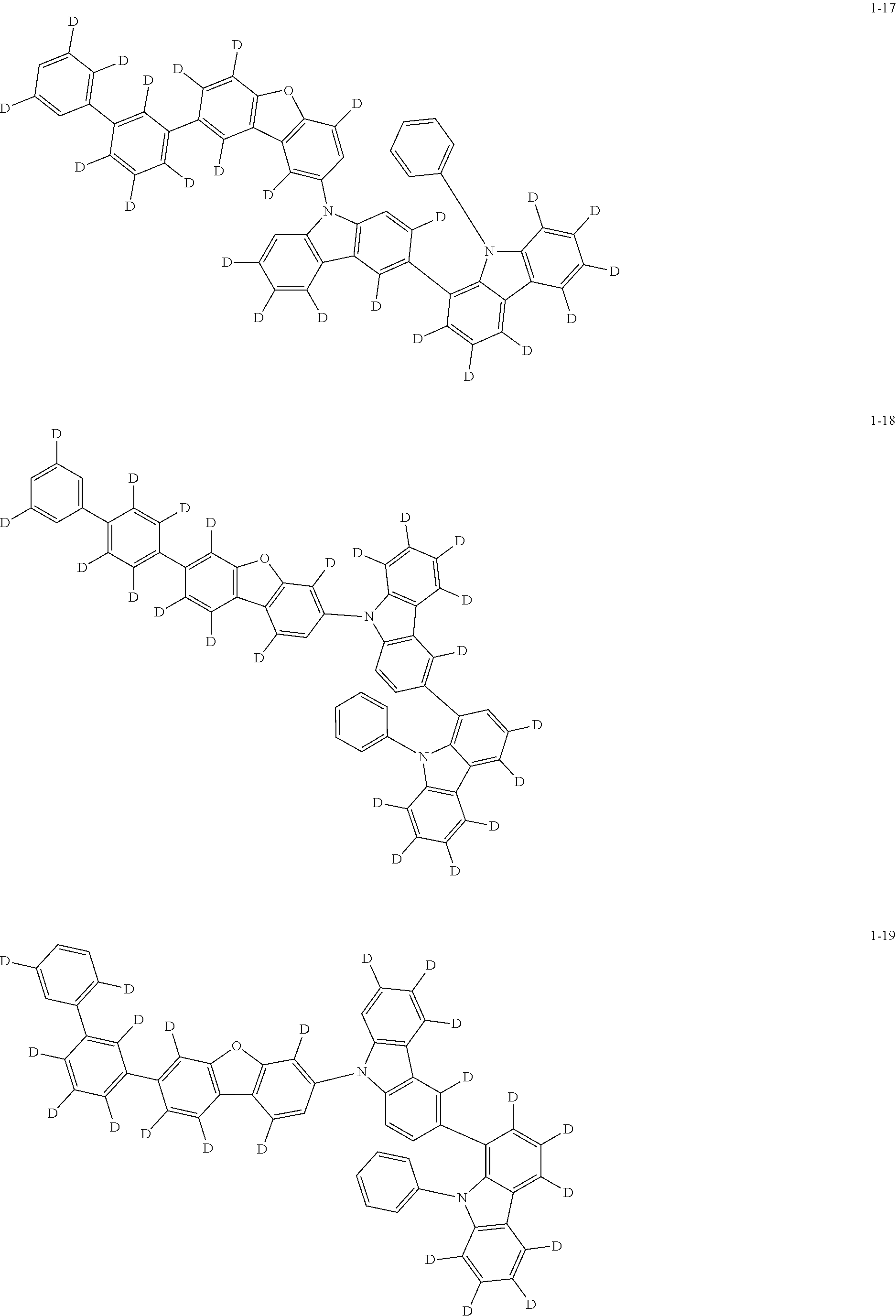

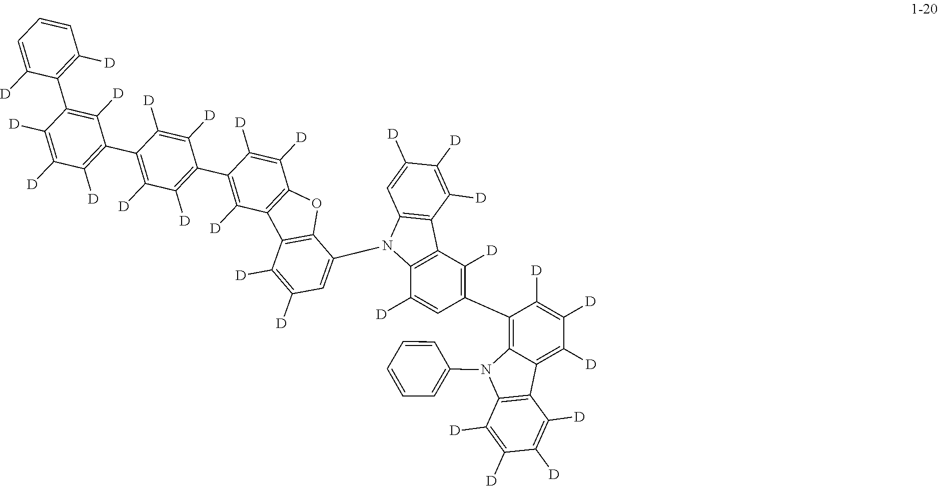 [SMILES: [2H]c1cc2c(c([2H])c1[2H])c1c([2H])c(-c3c([2H])c([2H])c([2H])c4c5c([2H])c([2H])c([2H])c([2H])c5n(-c5ccccc5)c34)cc([2H])c1n2-c1cc([2H])c([2H])c2c1oc1c([2H])c([2H])c(-c3c([2H])c([2H])c(-c4c([2H])c([2H])c([2H])c(-c5c([2H])cccc5[2H])c4[2H])c([2H])c3[2H])c([2H])c12]

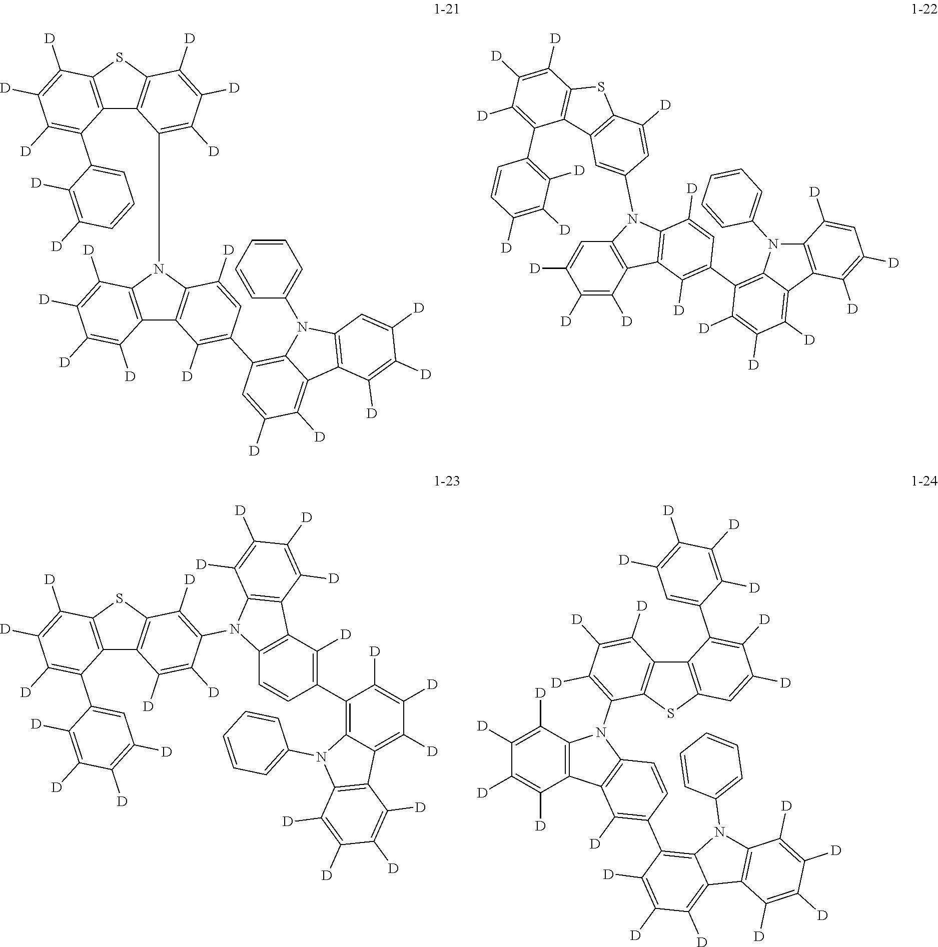

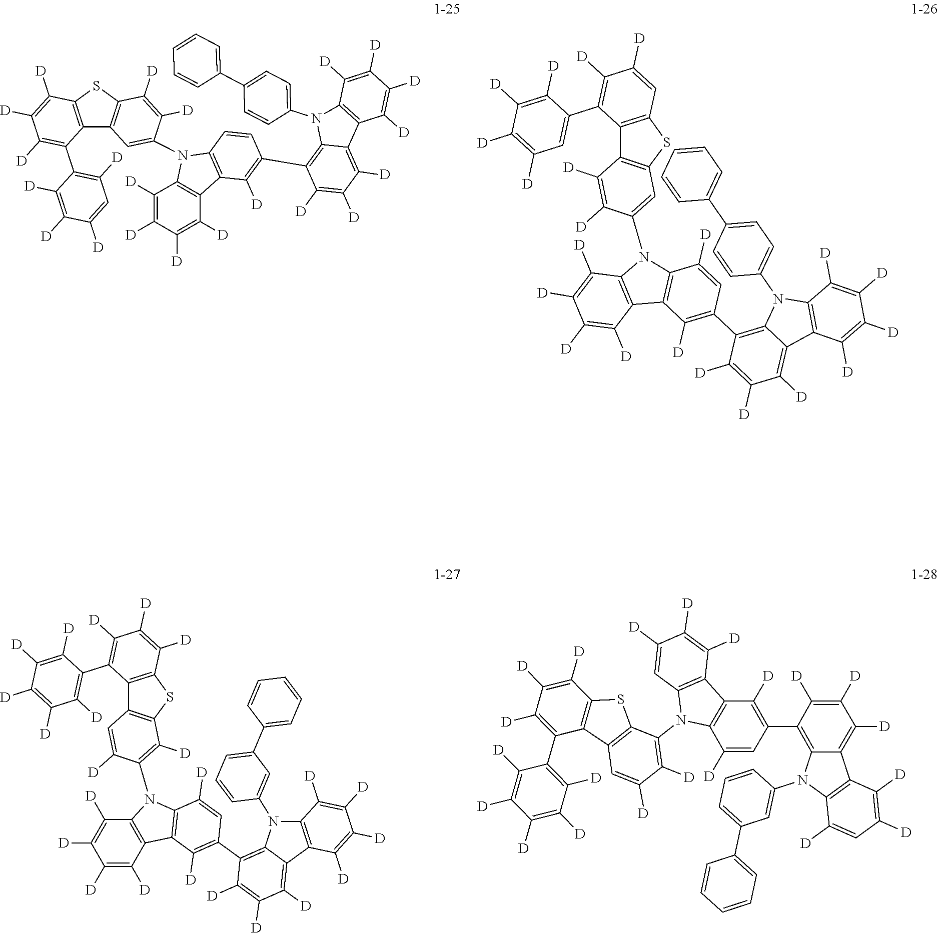

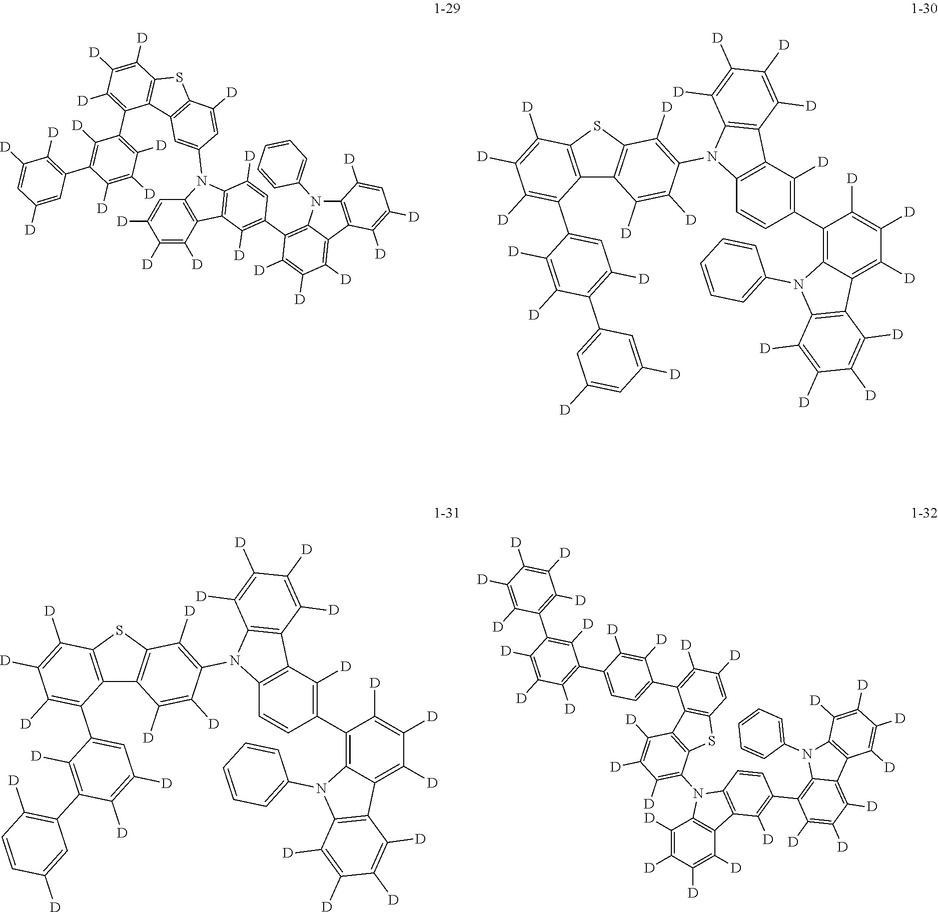

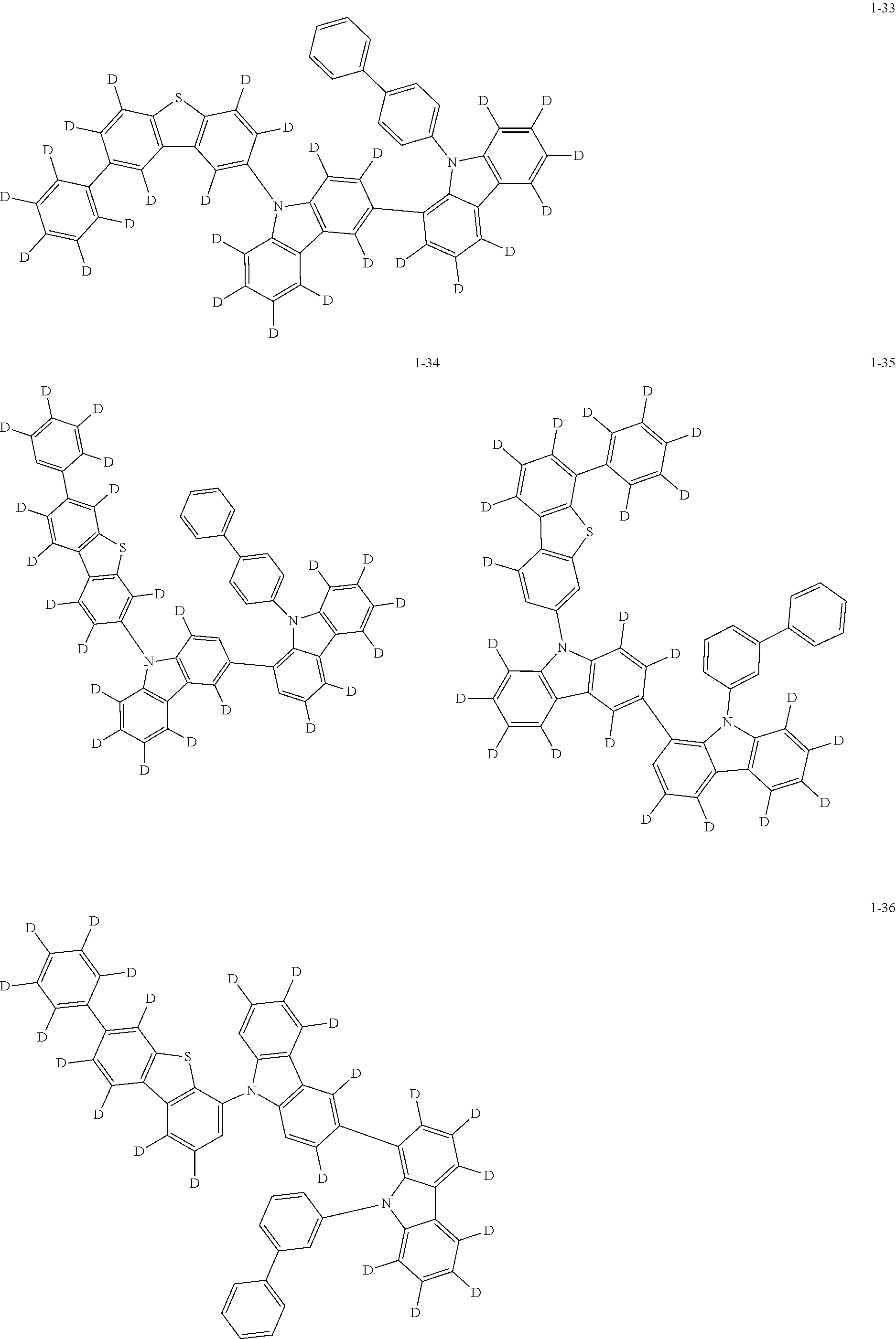

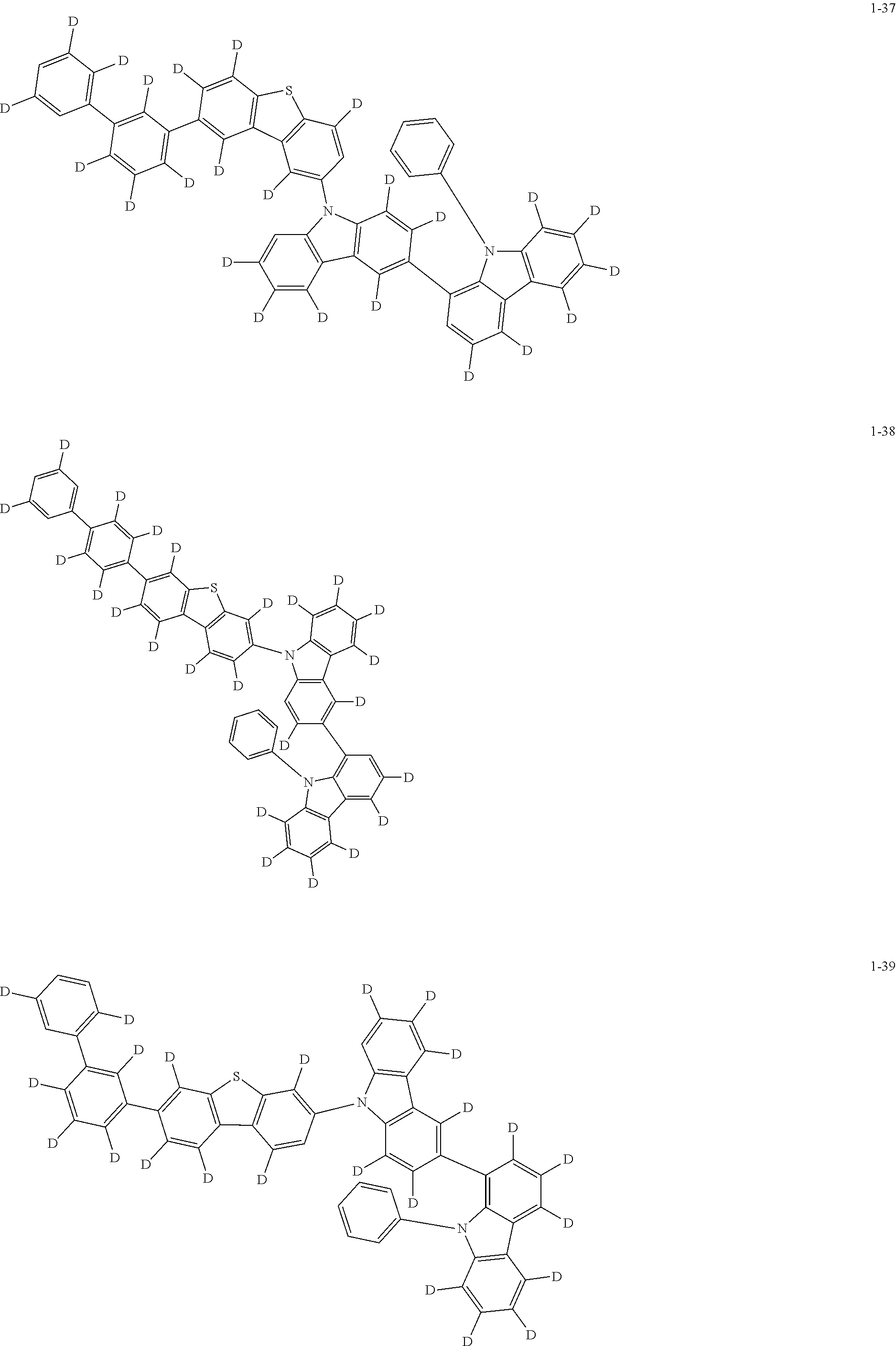

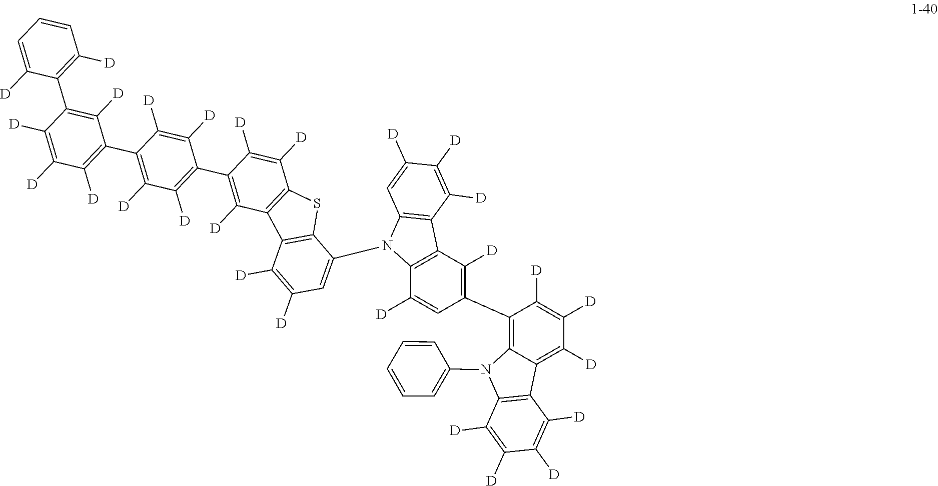 [SMILES: [2H]c1cc2c(c([2H])c1[2H])c1c([2H])c(-c3c([2H])c([2H])c([2H])c4c5c([2H])c([2H])c([2H])c([2H])c5n(-c5ccccc5)c34)cc([2H])c1n2-c1cc([2H])c([2H])c2c1sc1c([2H])c([2H])c(-c3c([2H])c([2H])c(-c4c([2H])c([2H])c([2H])c(-c5c([2H])cccc5[2H])c4[2H])c([2H])c3[2H])c([2H])c12]

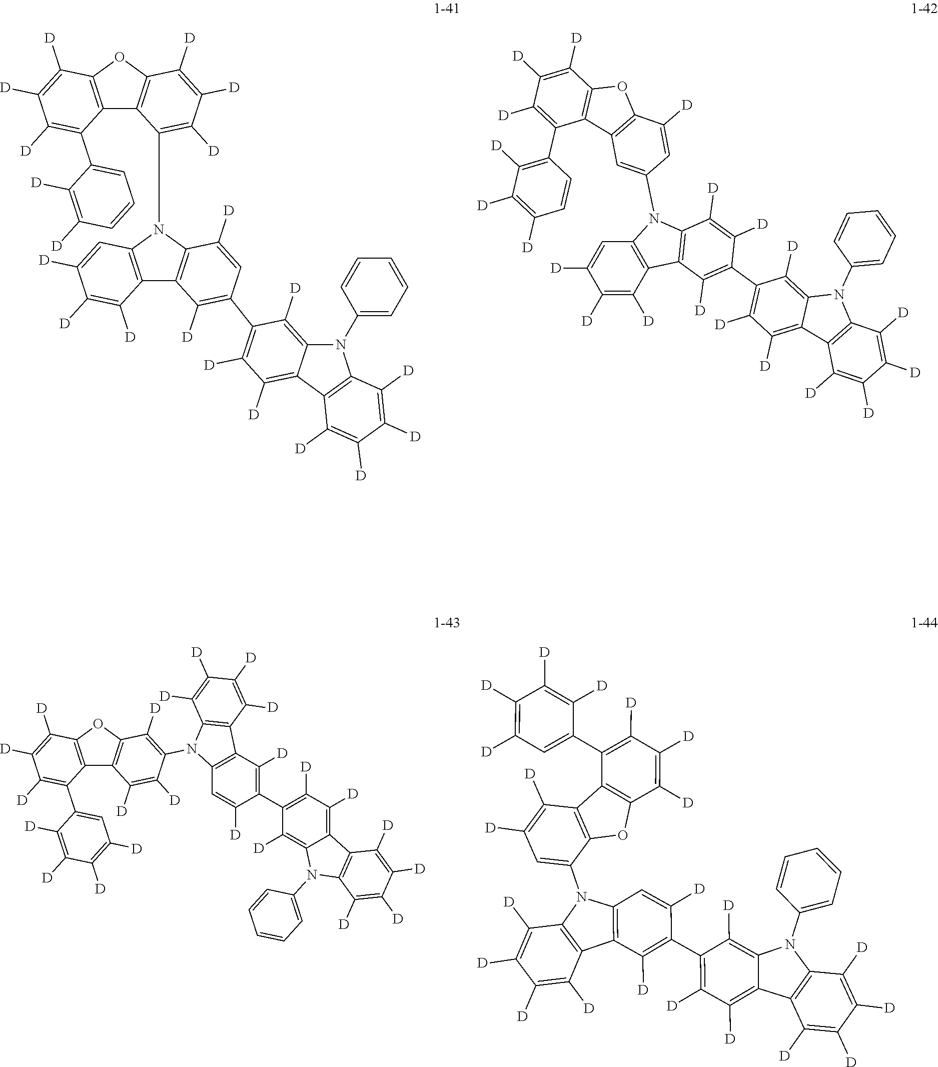

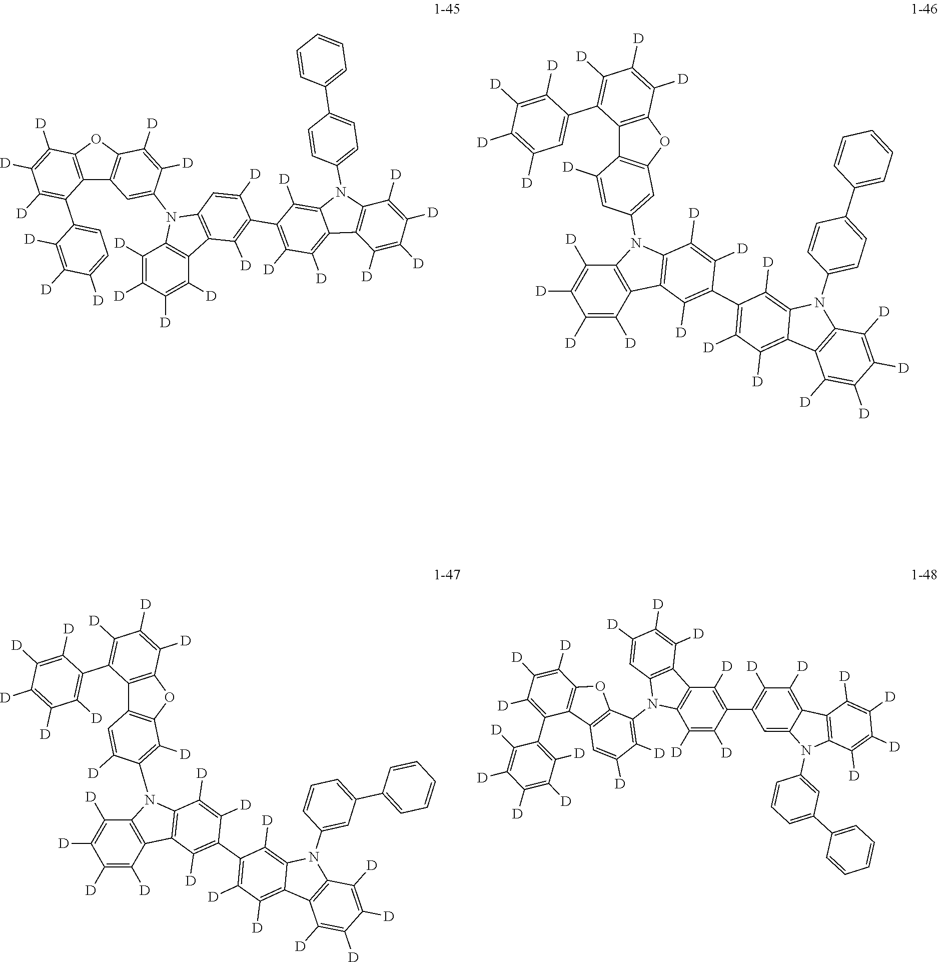

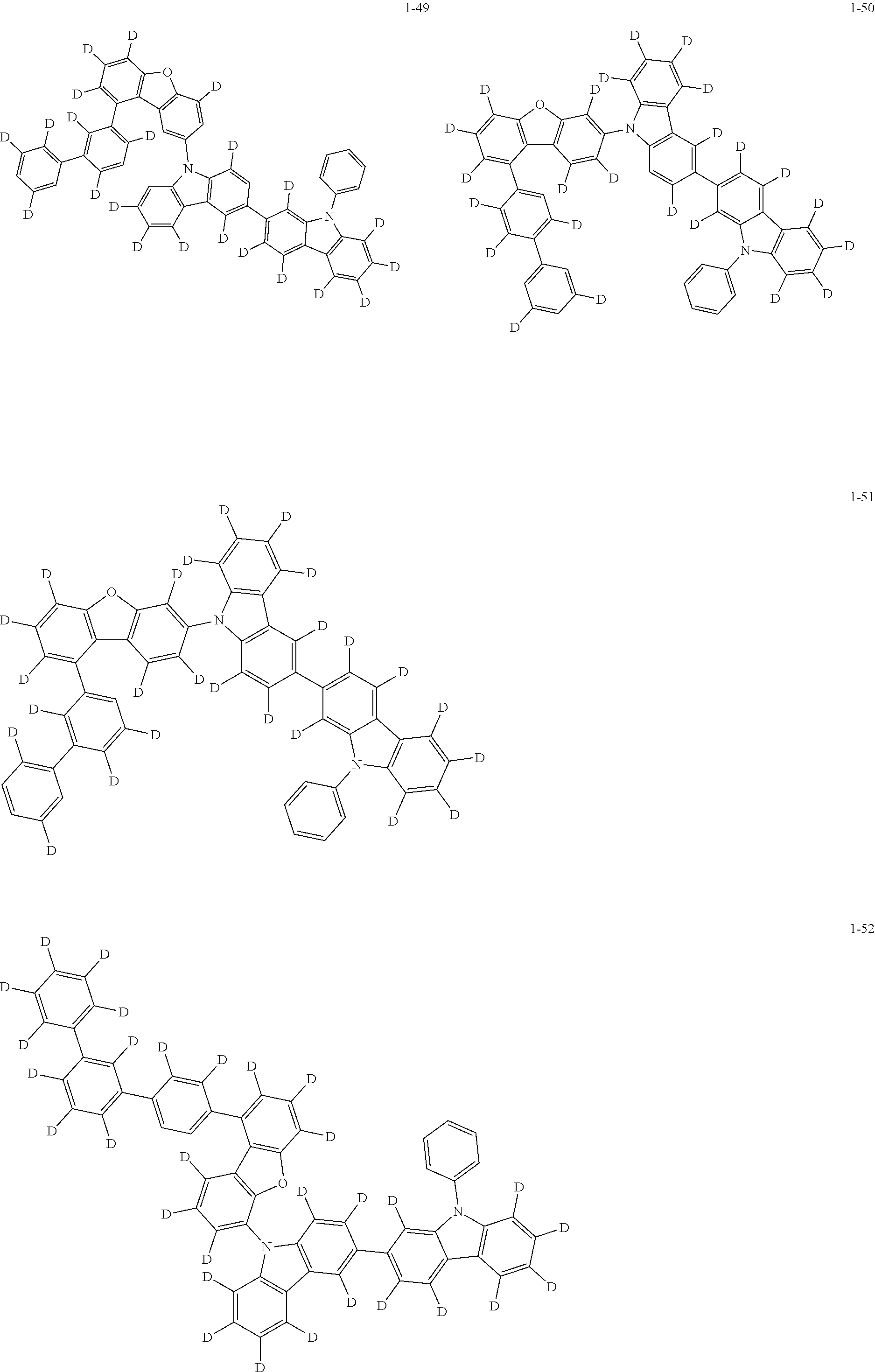

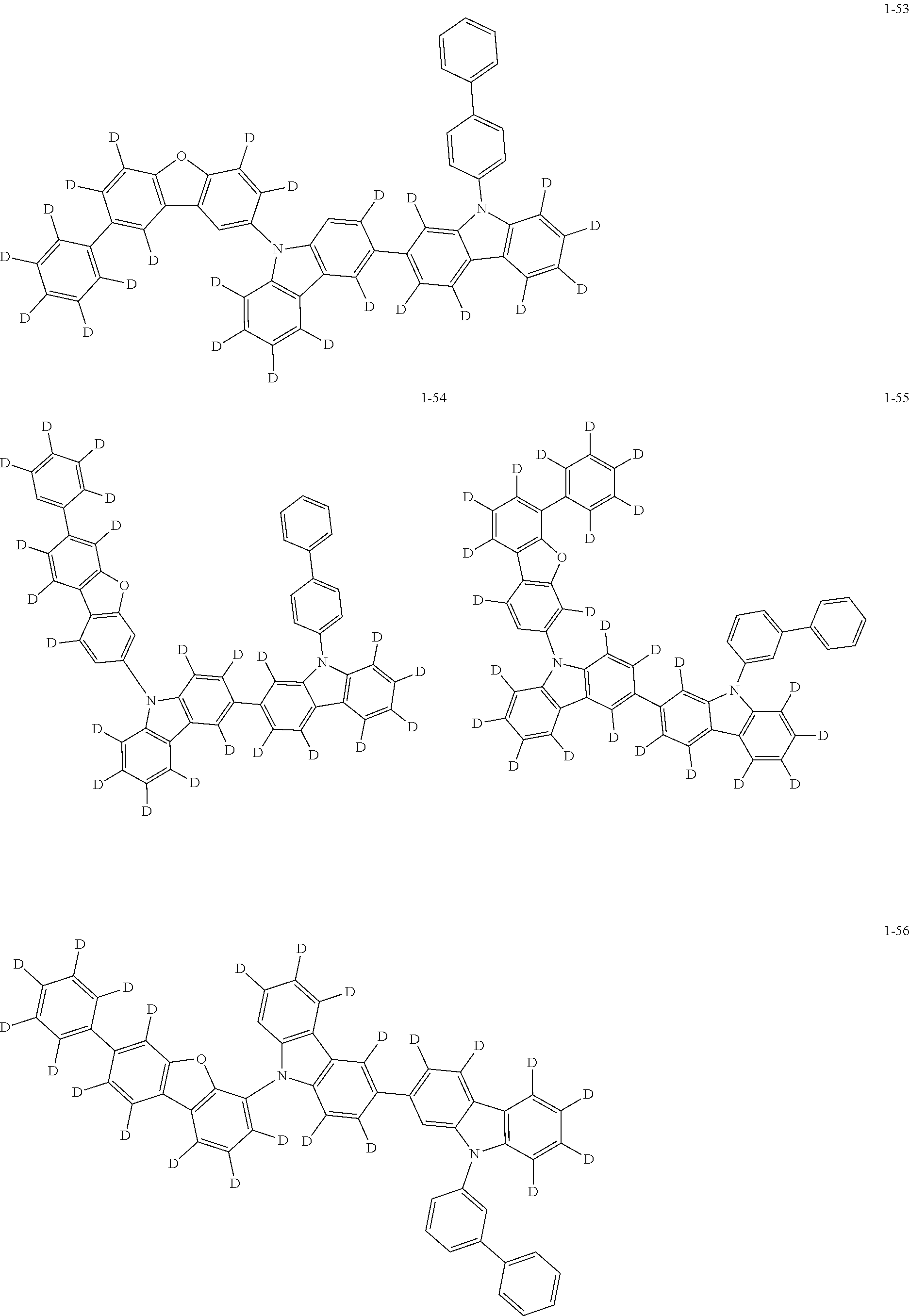

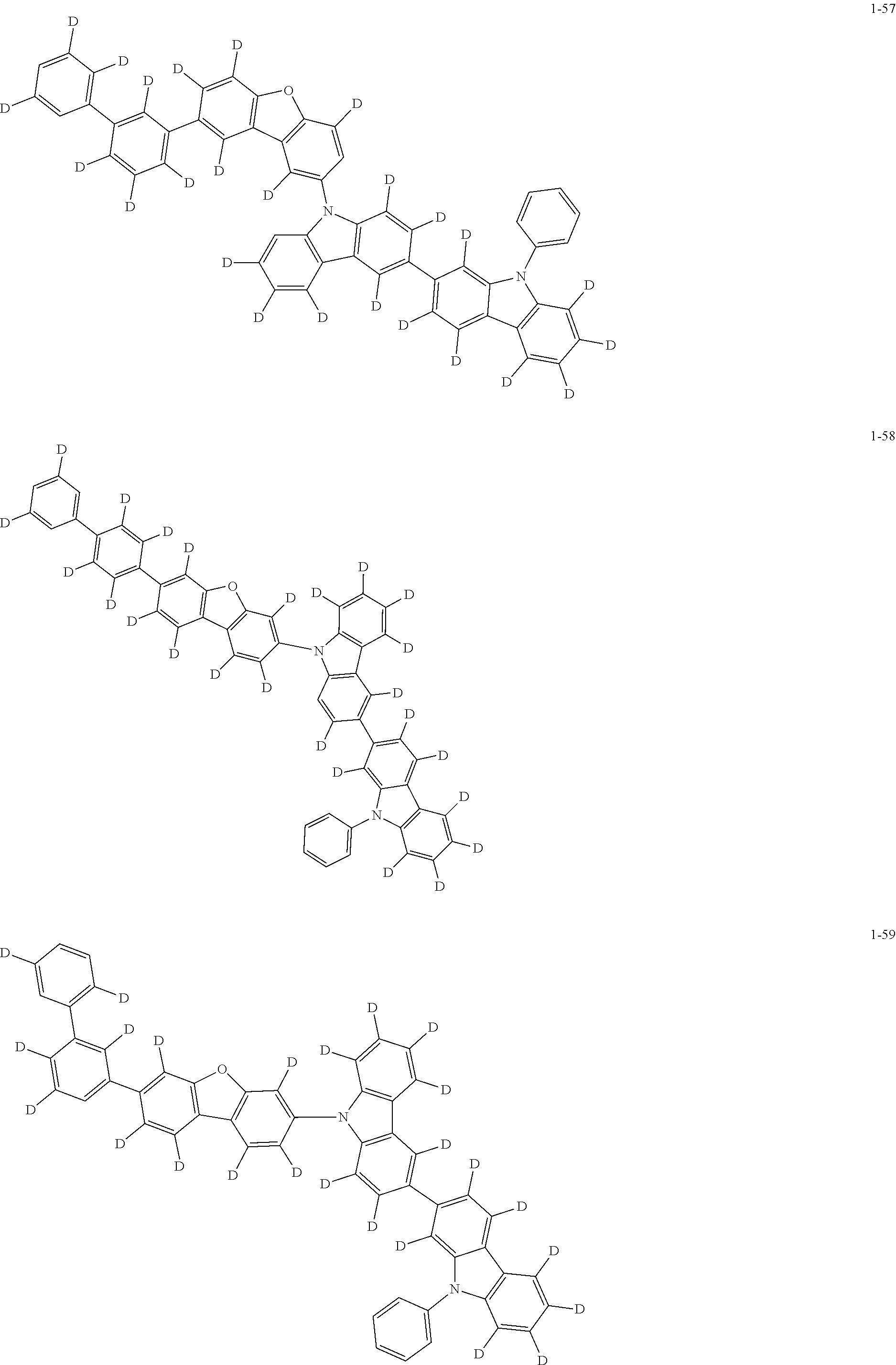

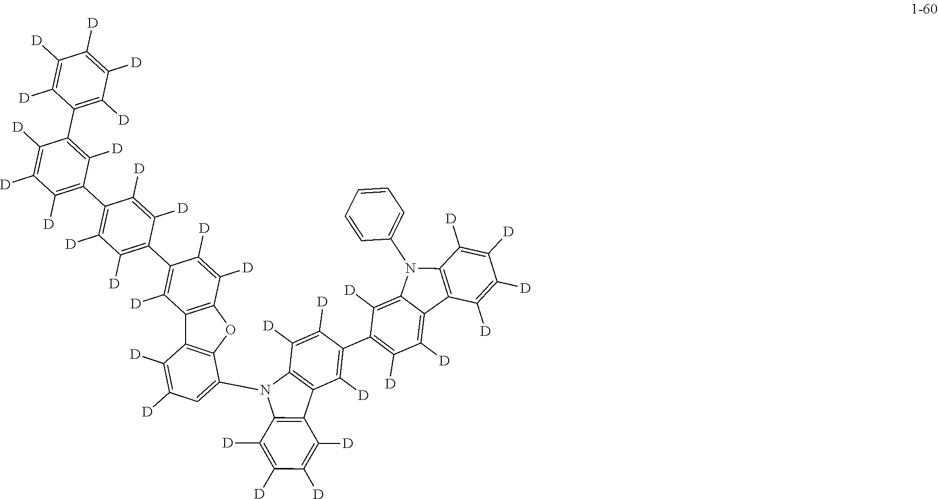 [SMILES: [2H]c1cc(-n2c3c([2H])c([2H])c([2H])c([2H])c3c3c([2H])c(-c4c([2H])c([2H])c5c6c([2H])c([2H])c([2H])c([2H])c6n(-c6ccccc6)c5c4[2H])c([2H])c([2H])c32)c2oc3c([2H])c([2H])c(-c4c([2H])c([2H])c(-c5c([2H])c([2H])c([2H])c(-c6c([2H])c([2H])c([2H])c([2H])c6[2H])c5[2H])c([2H])c4[2H])c([2H])c3c2c1[2H]]

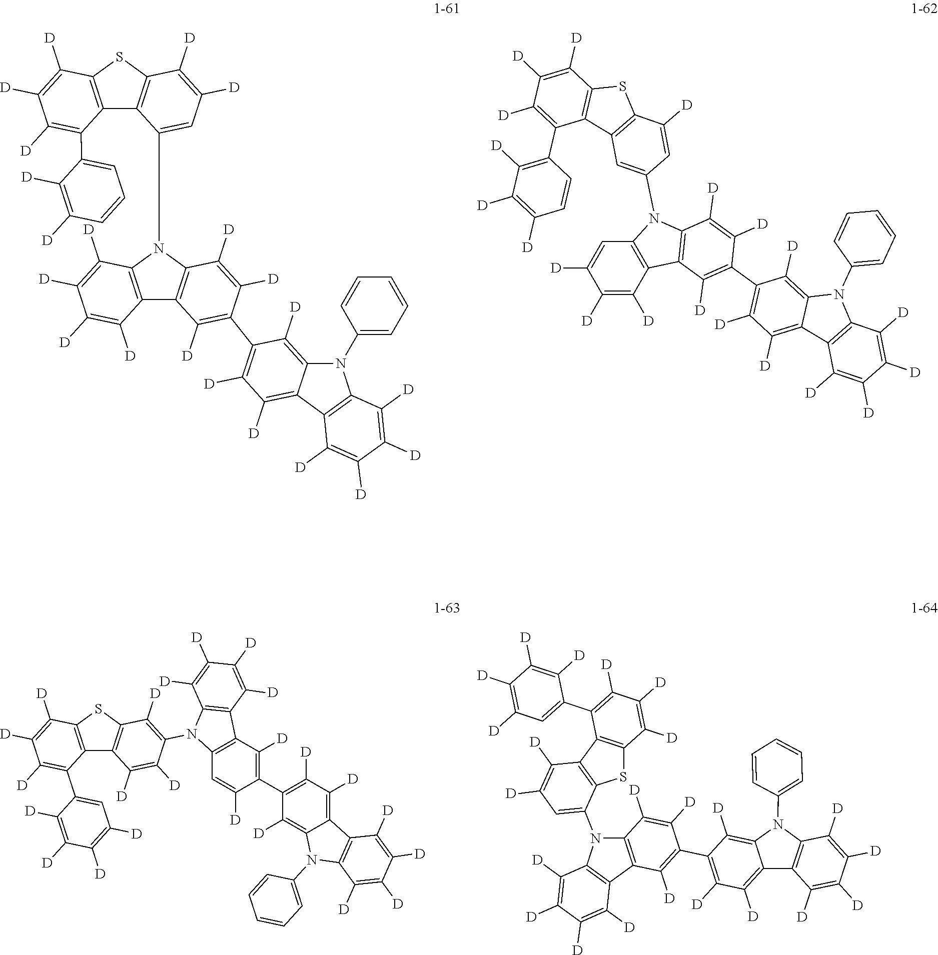

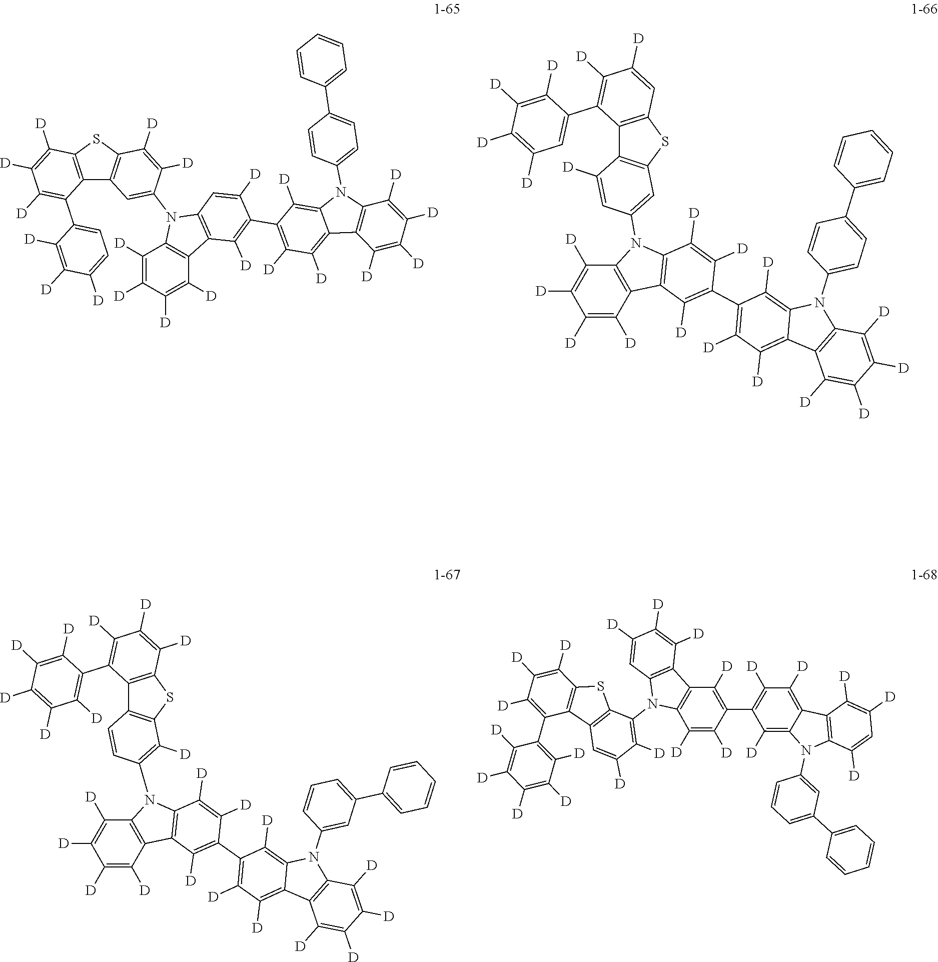

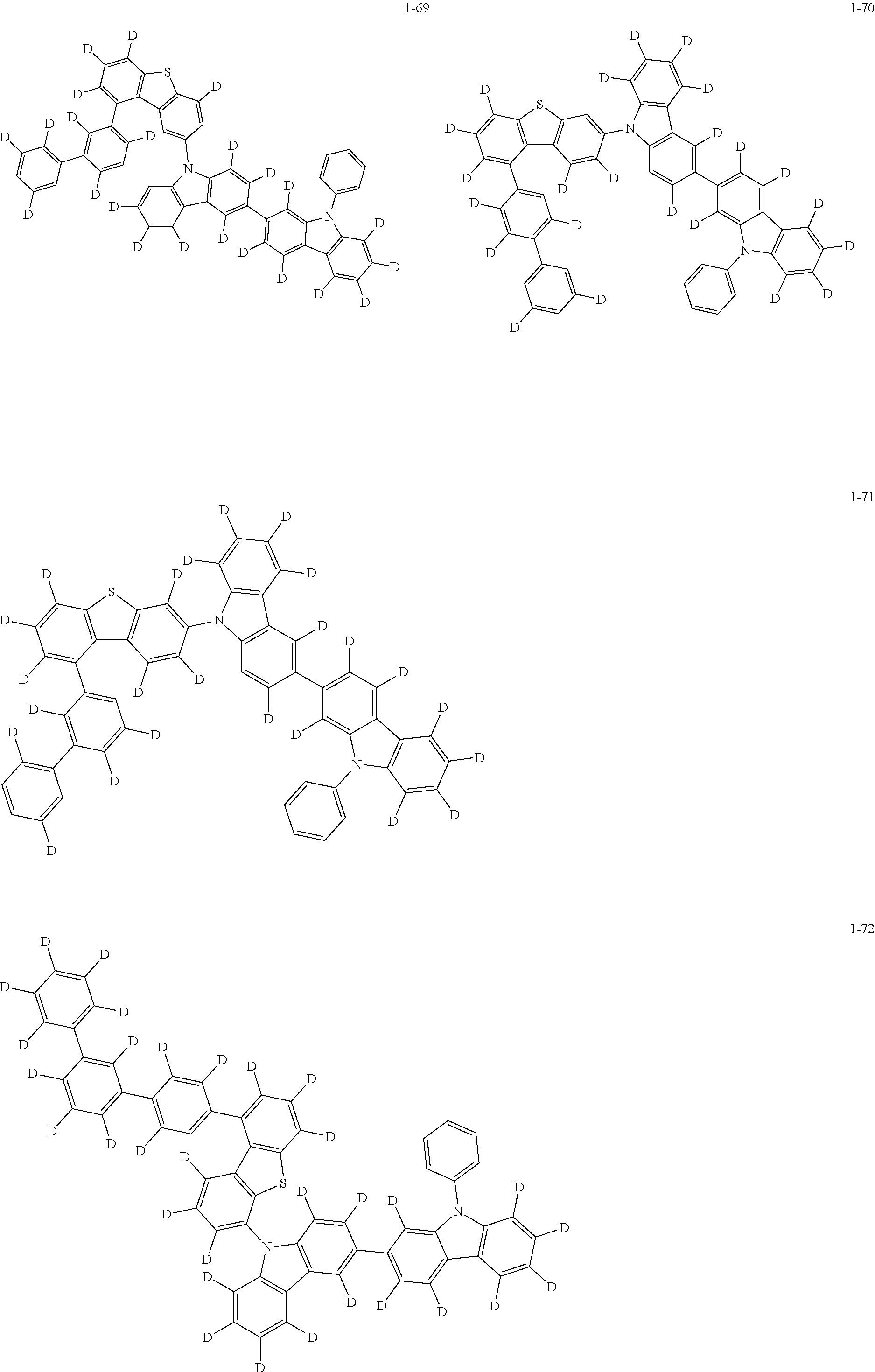

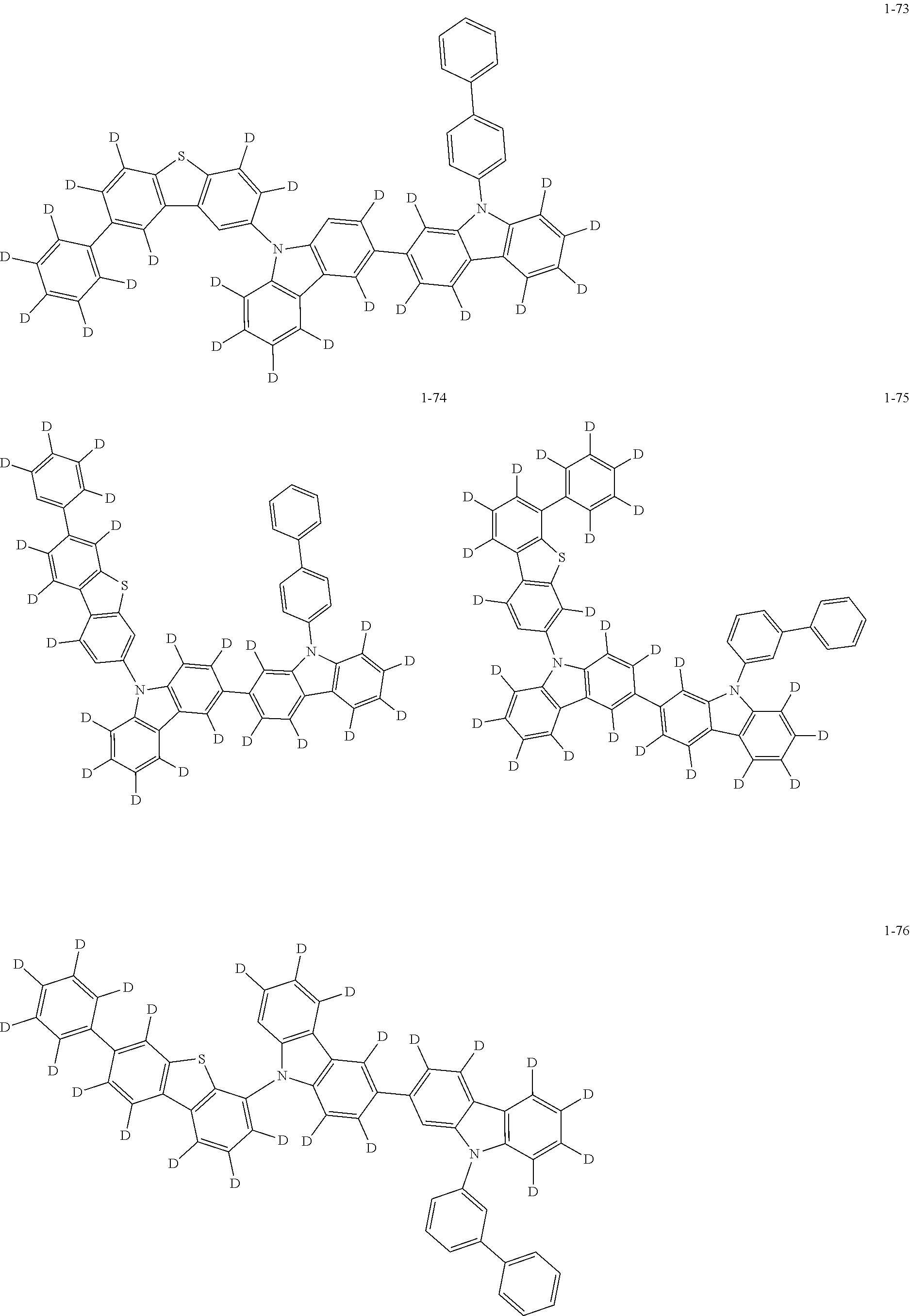

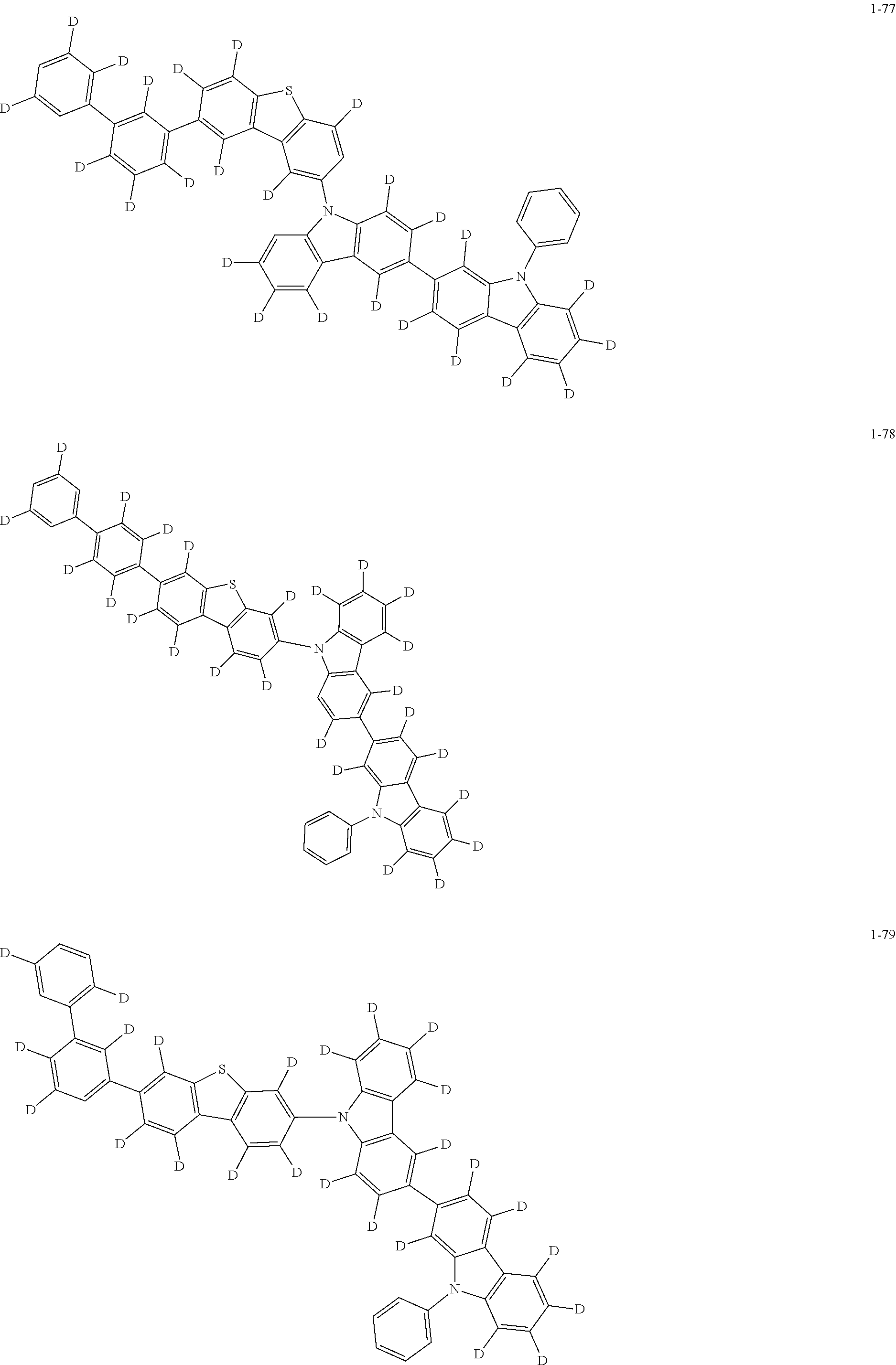

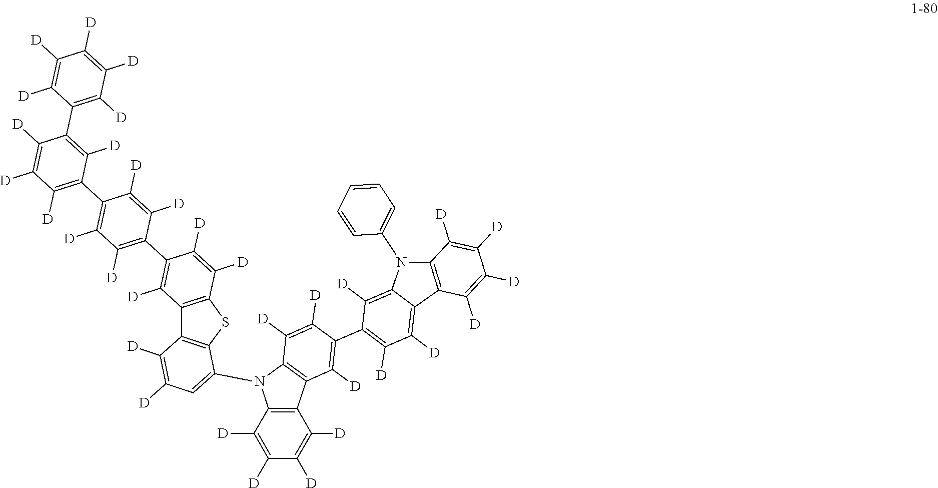 [SMILES: [2H]c1cc(-n2c3c([2H])c([2H])c([2H])c([2H])c3c3c([2H])c(-c4c([2H])c([2H])c5c6c([2H])c([2H])c([2H])c([2H])c6n(-c6ccccc6)c5c4[2H])c([2H])c([2H])c32)c2sc3c([2H])c([2H])c(-c4c([2H])c([2H])c(-c5c([2H])c([2H])c([2H])c(-c6c([2H])c([2H])c([2H])c([2H])c6[2H])c5[2H])c([2H])c4[2H])c([2H])c3c2c1[2H]]

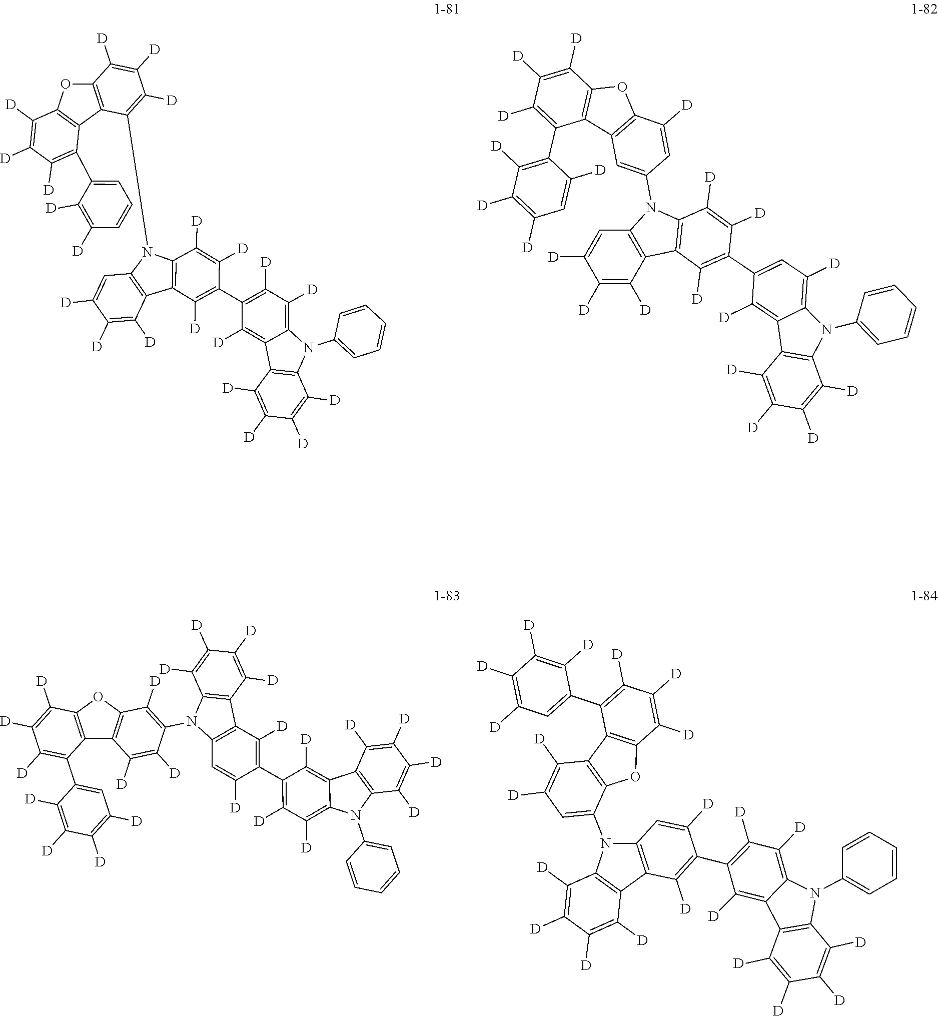

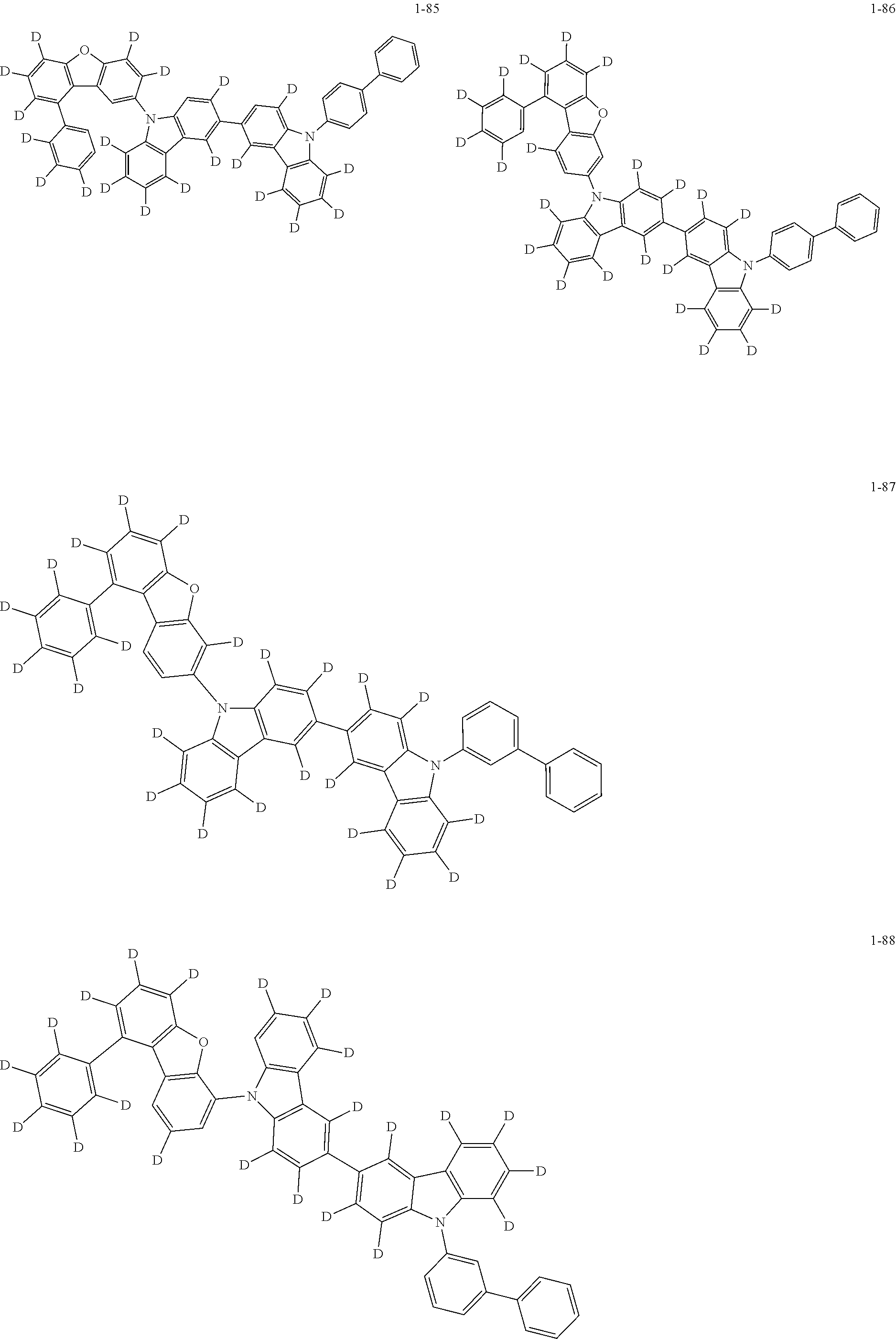

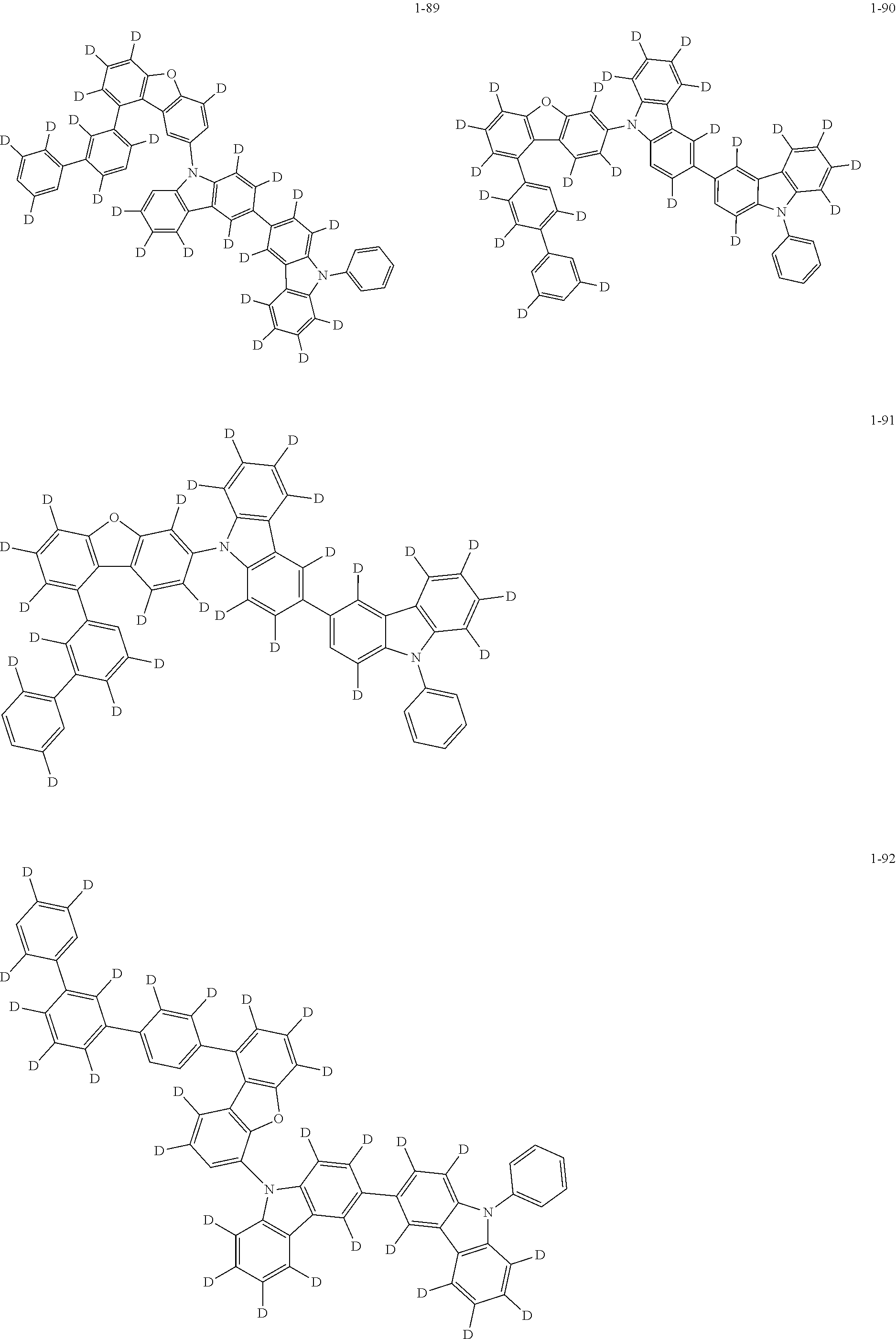

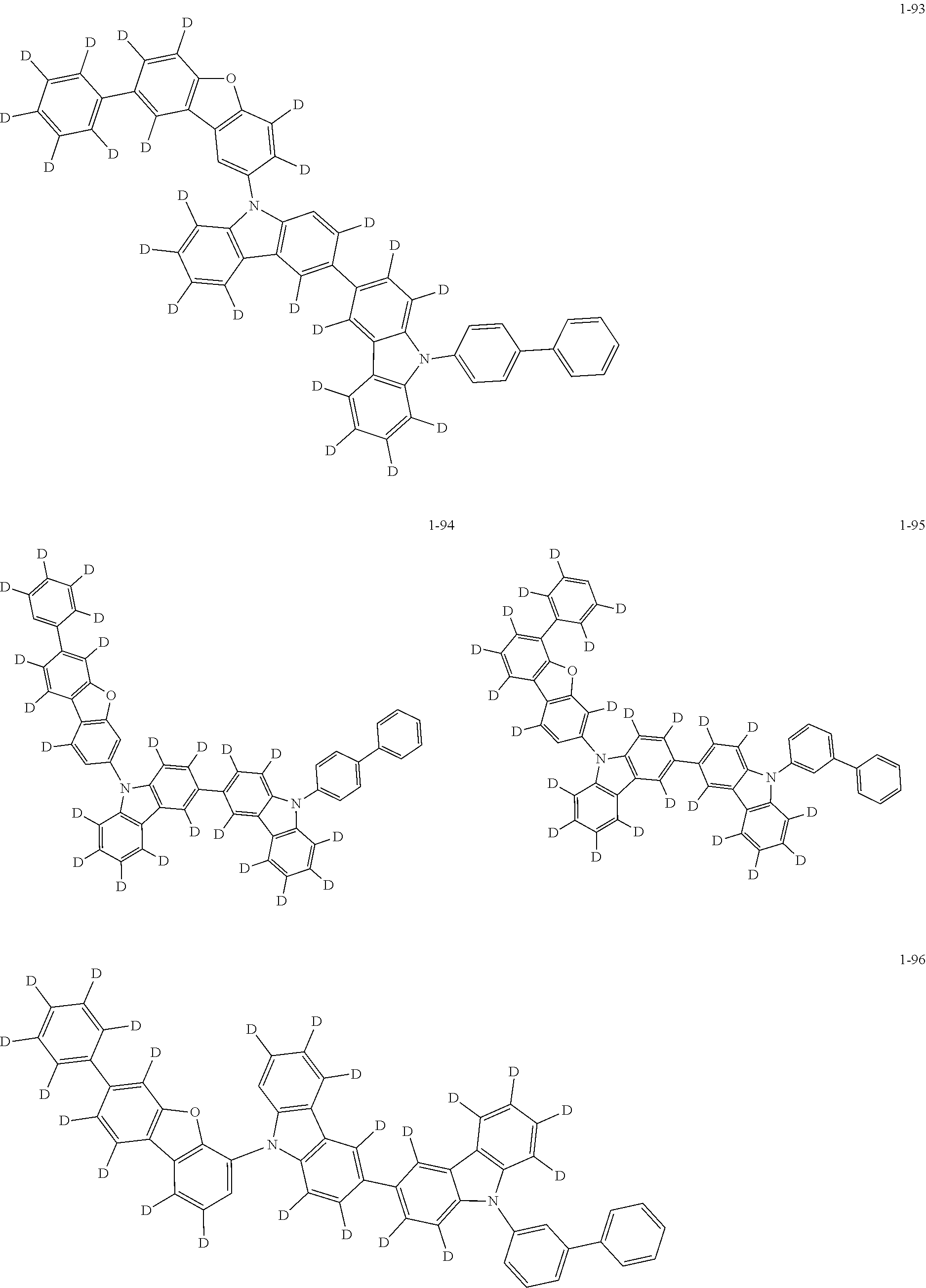

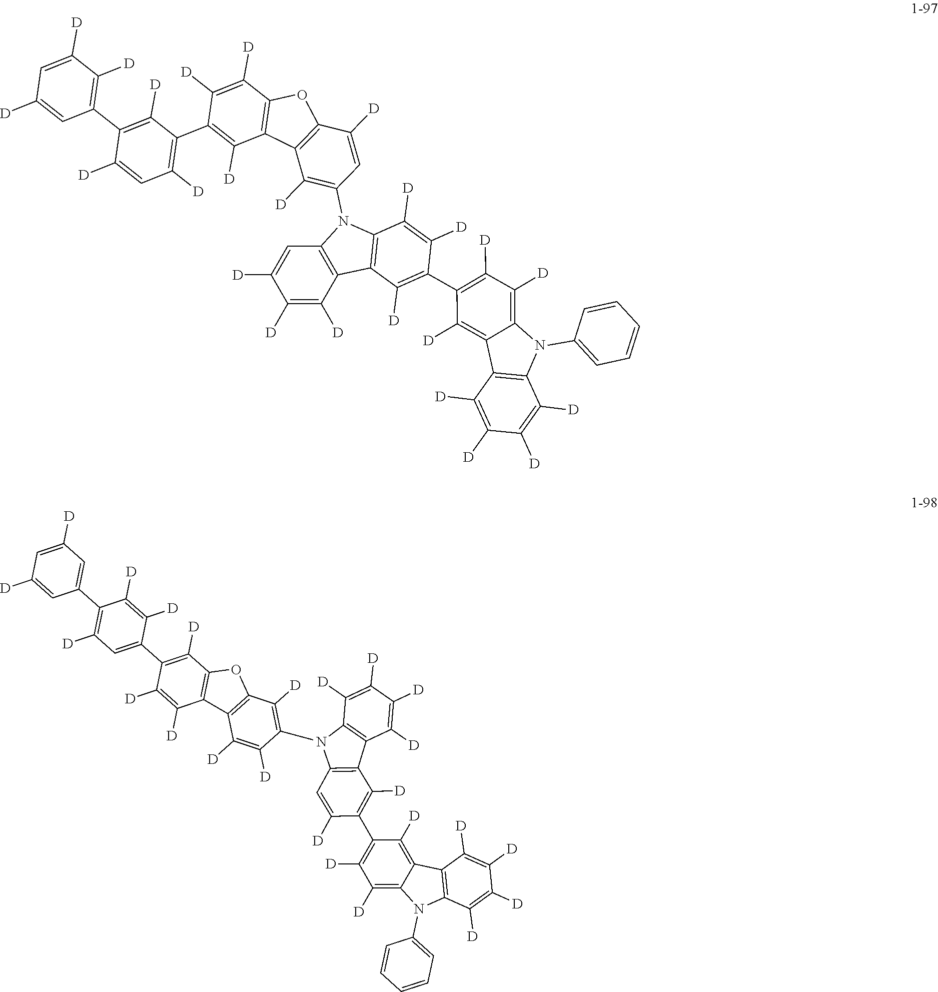 [SMILES: [2H]c1cc([2H])c([2H])c(-c2c([2H])cc([2H])c(-c3c([2H])c([2H])c4oc5c([2H])cc(-n6c7cc([2H])c([2H])c([2H])c7c7c([2H])c(-c8c([2H])c([2H])c9c(c8[2H])c8c([2H])c([2H])c([2H])c([2H])c8n9-c8ccccc8)c([2H])c([2H])c76)c([2H])c5c4c3[2H])c2[2H])c1 ; [2H]c1cc([2H])cc(-c2c([2H])cc(-c3c([2H])c([2H])c4c(oc5c([2H])c(-n6c7cc([2H])c(-c8c([2H])c([2H])c9c(c8[2H])c8c([2H])c([2H])c([2H])c([2H])c8n9-c8ccccc8)c([2H])c7c7c([2H])c([2H])c([2H])c([2H])c76)c([2H])c([2H])c54)c3[2H])c([2H])c2[2H])c1]

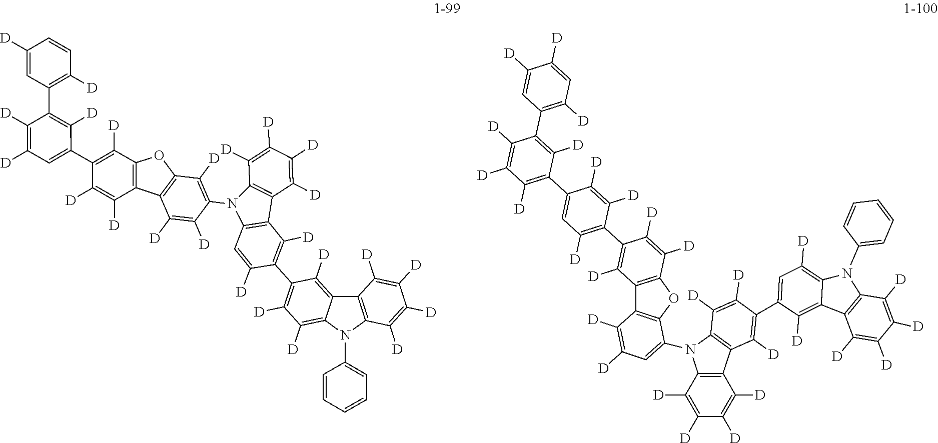

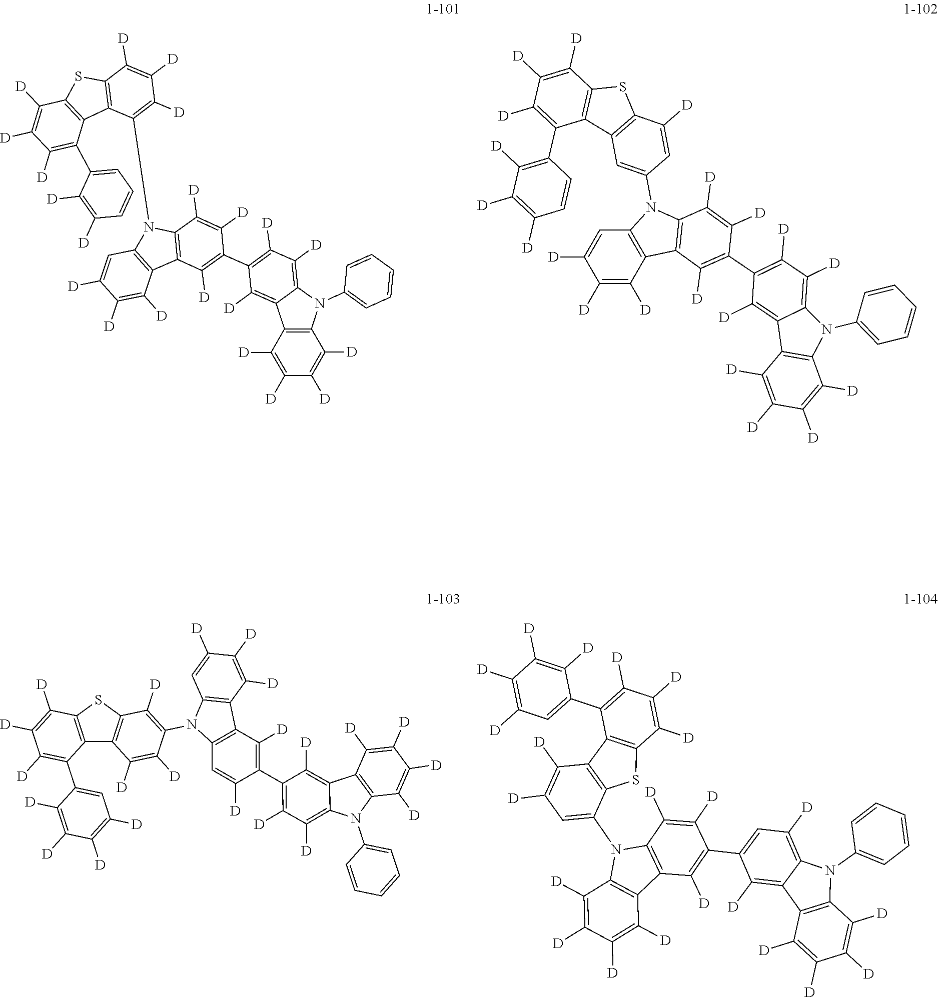

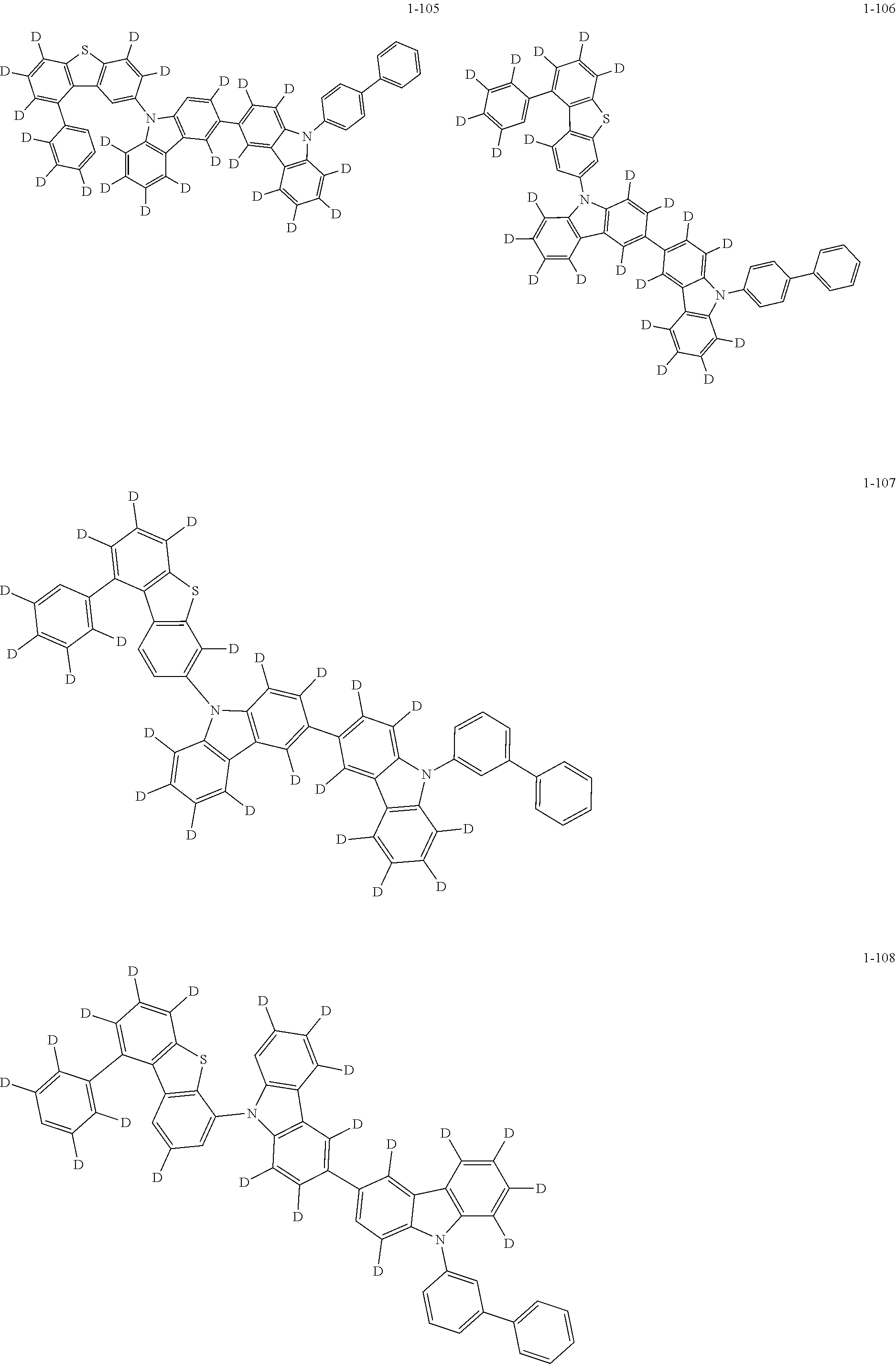

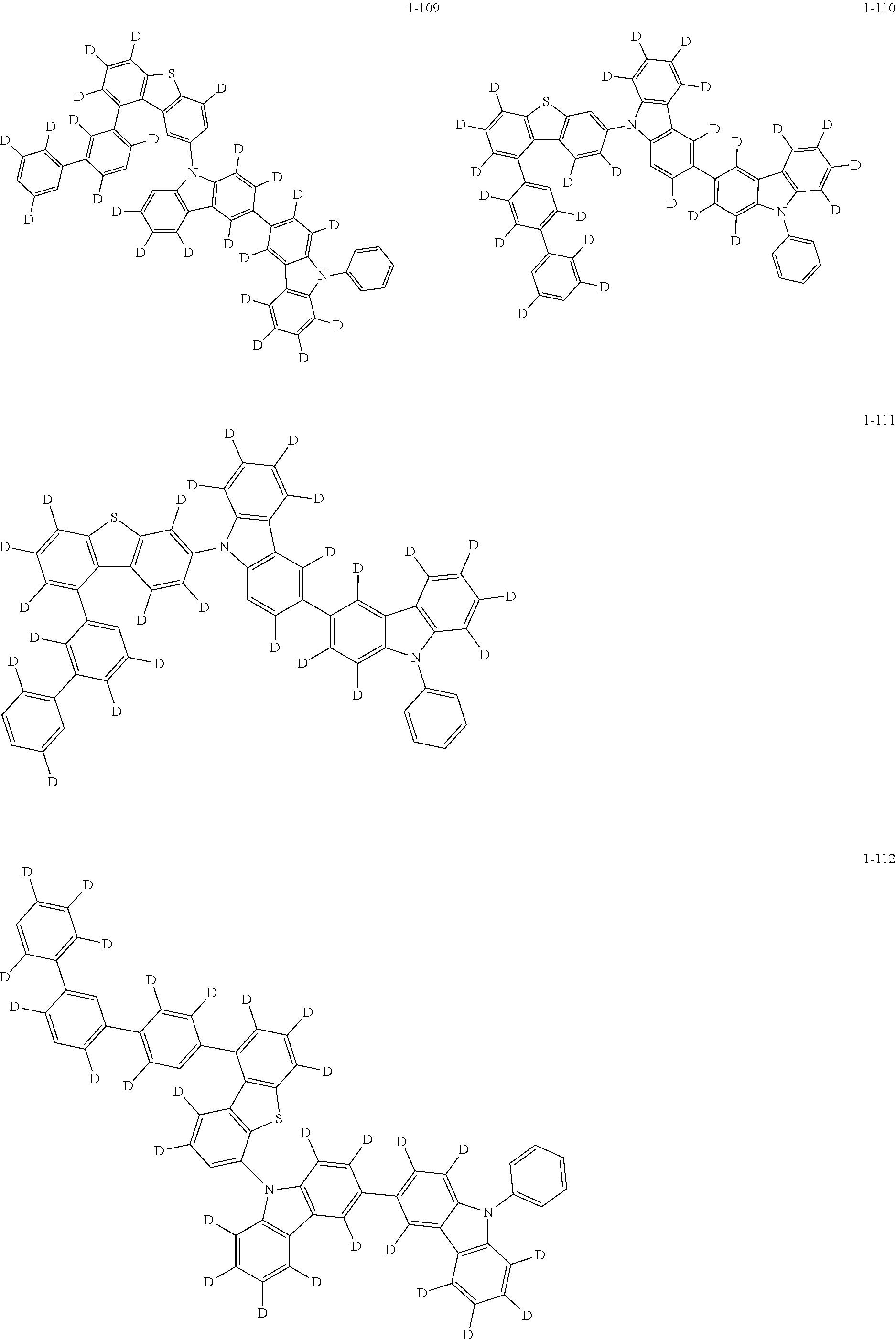

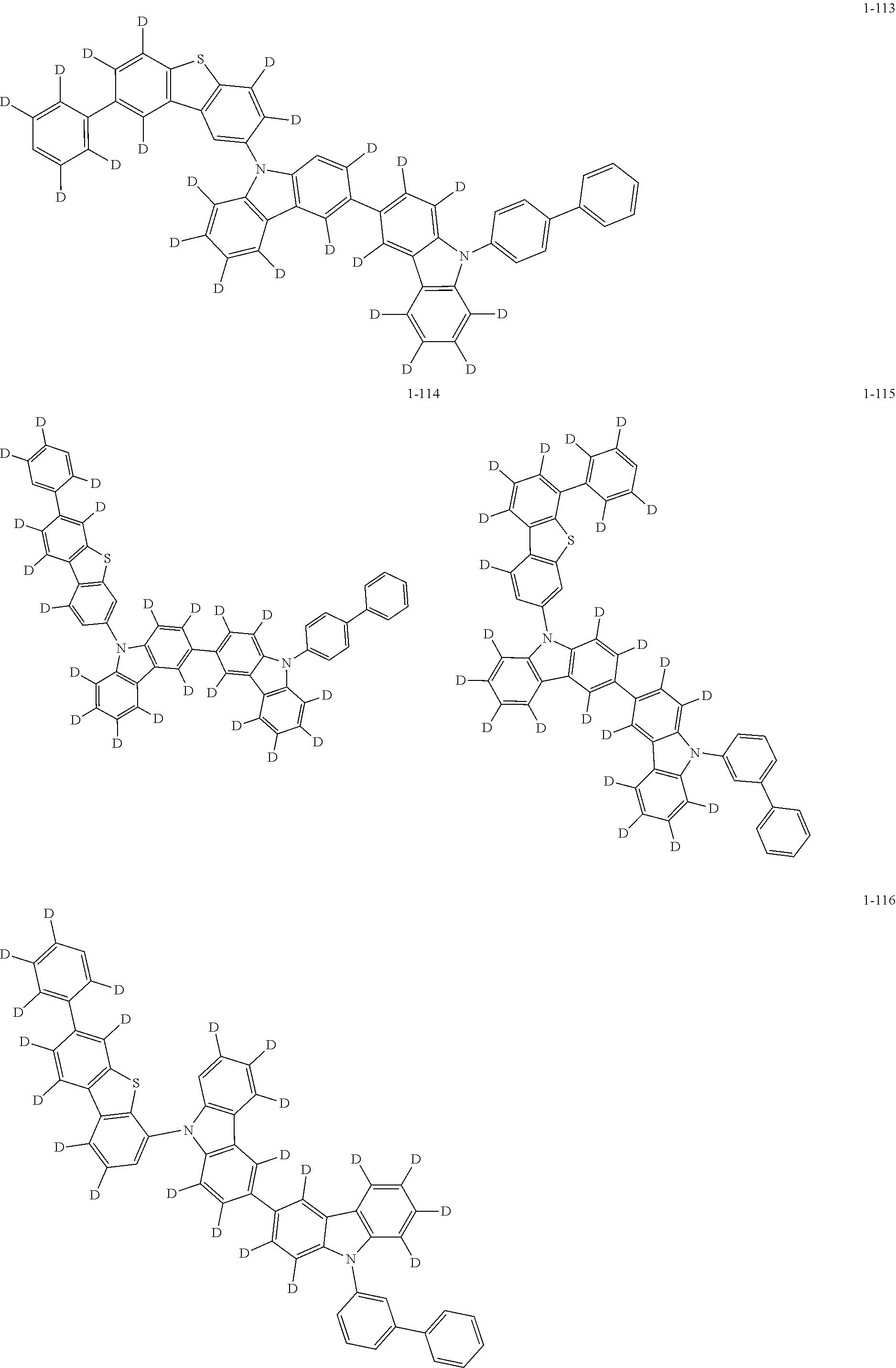

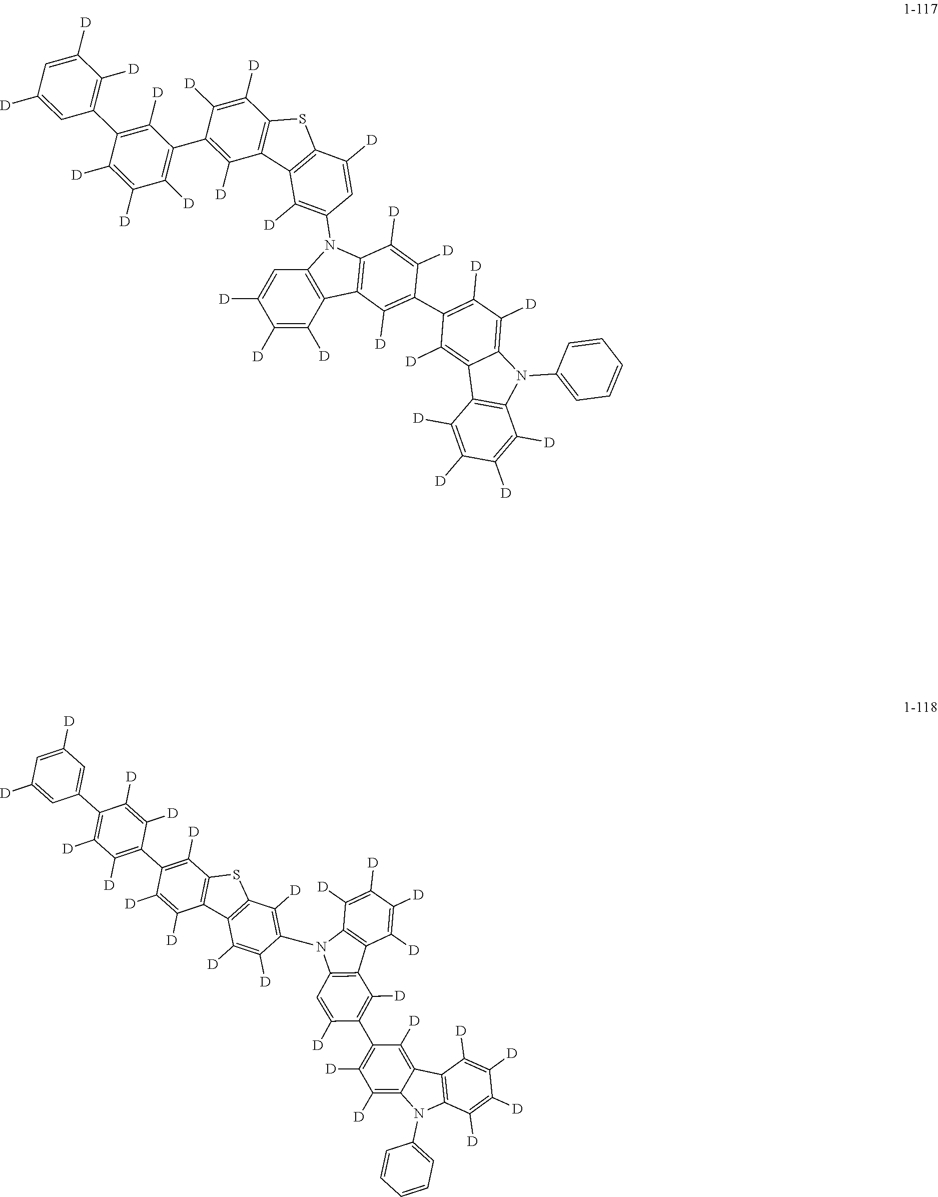 [SMILES: [2H]c1cc([2H])c([2H])c(-c2c([2H])c([2H])c([2H])c(-c3c([2H])c([2H])c4sc5c([2H])cc(-n6c7cc([2H])c([2H])c([2H])c7c7c([2H])c(-c8c([2H])c([2H])c9c(c8[2H])c8c([2H])c([2H])c([2H])c([2H])c8n9-c8ccccc8)c([2H])c([2H])c76)c([2H])c5c4c3[2H])c2[2H])c1 ; [2H]c1cc([2H])cc(-c2c([2H])c([2H])c(-c3c([2H])c([2H])c4c(sc5c([2H])c(-n6c7cc([2H])c(-c8c([2H])c([2H])c9c(c8[2H])c8c([2H])c([2H])c([2H])c([2H])c8n9-c8ccccc8)c([2H])c7c7c([2H])c([2H])c([2H])c([2H])c76)c([2H])c([2H])c54)c3[2H])c([2H])c2[2H])c1]

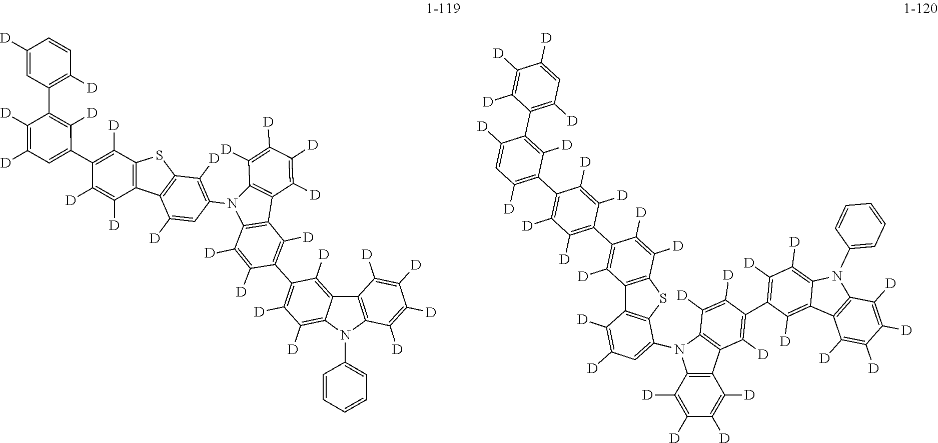

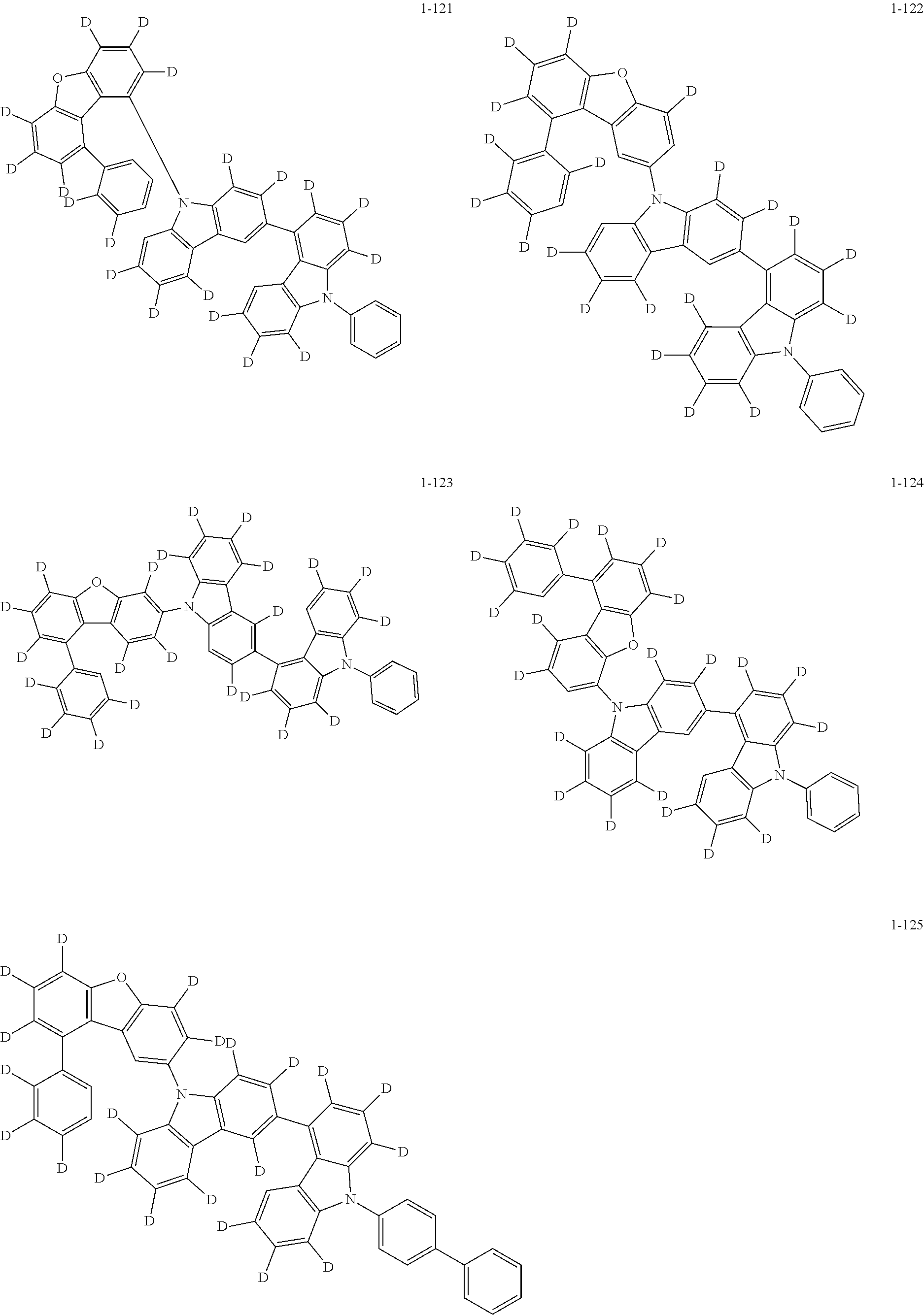

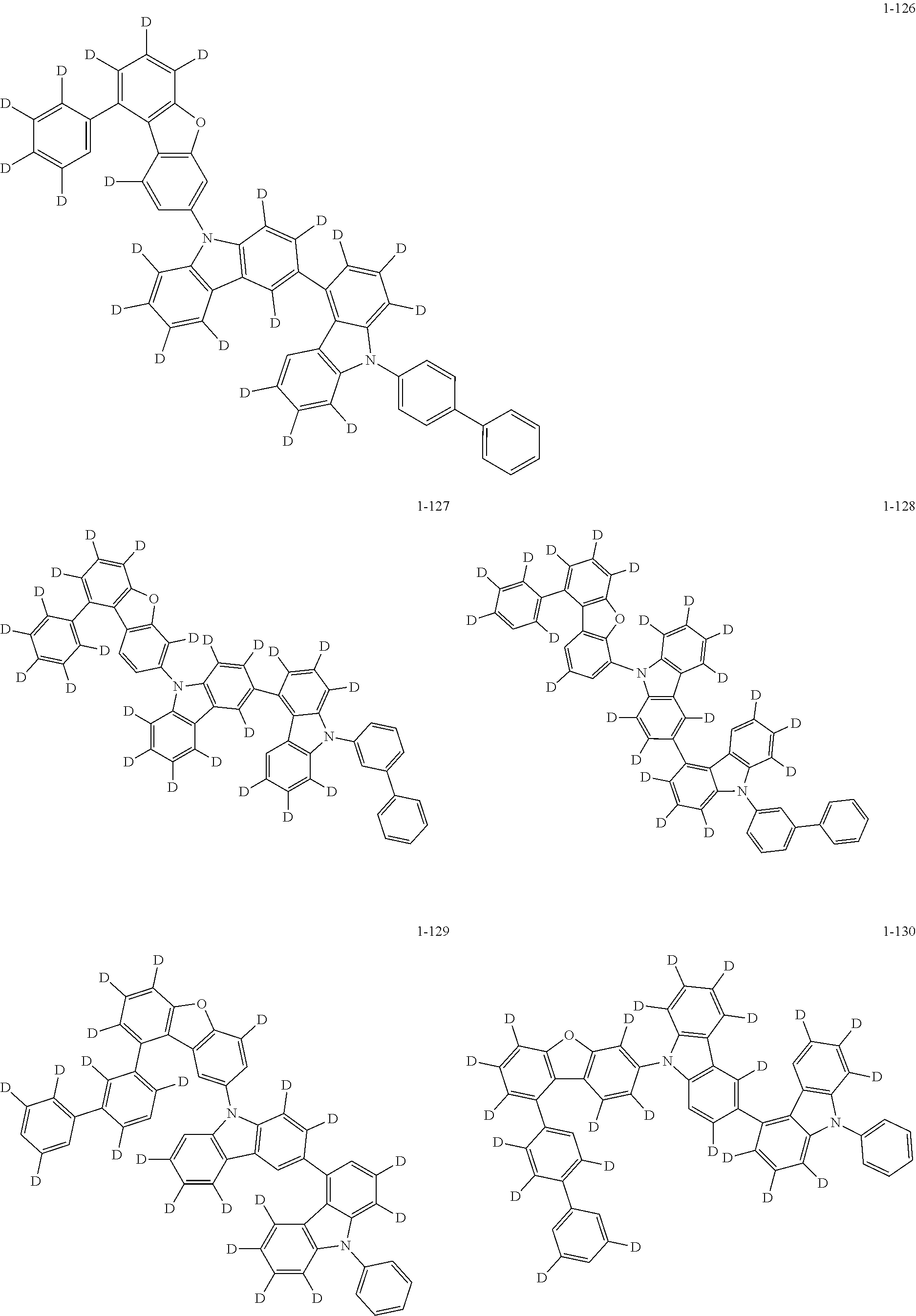

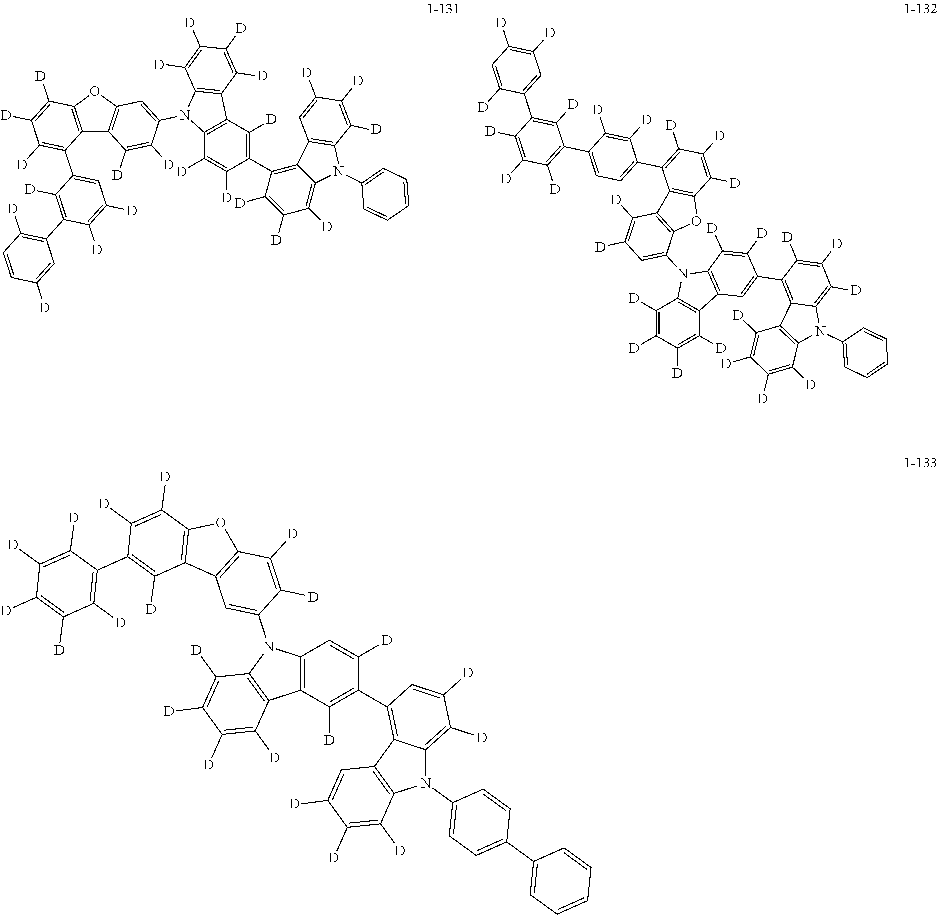

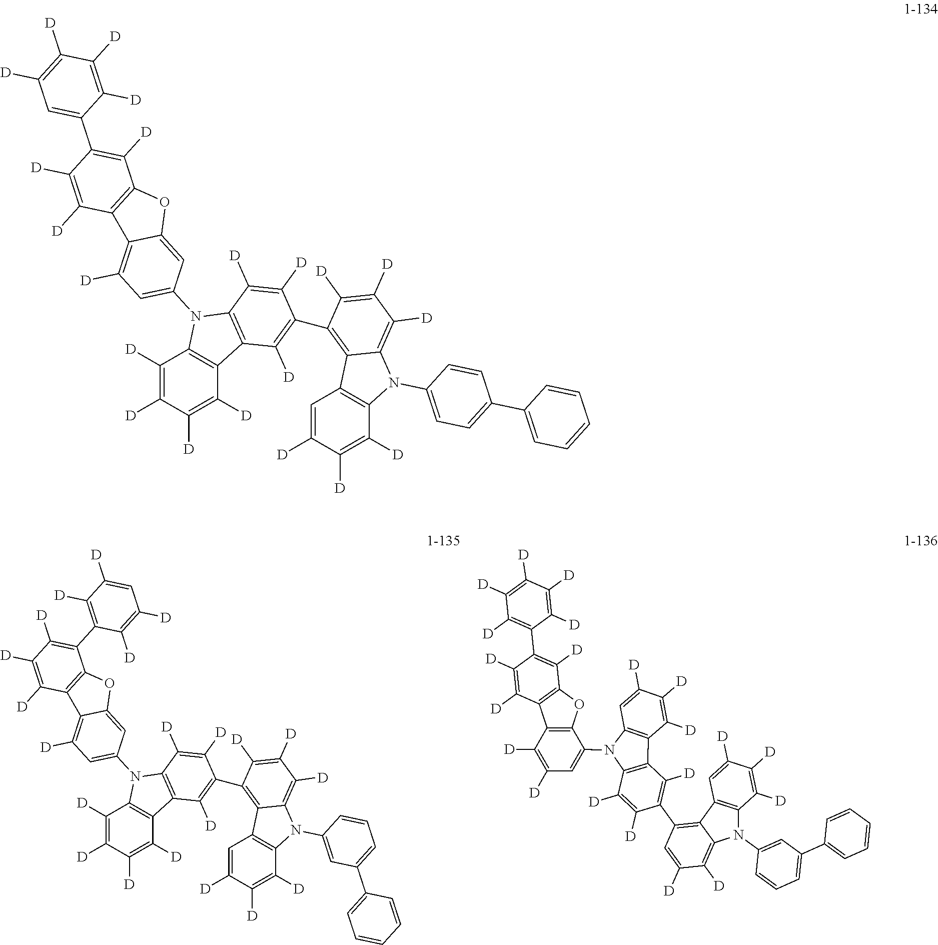

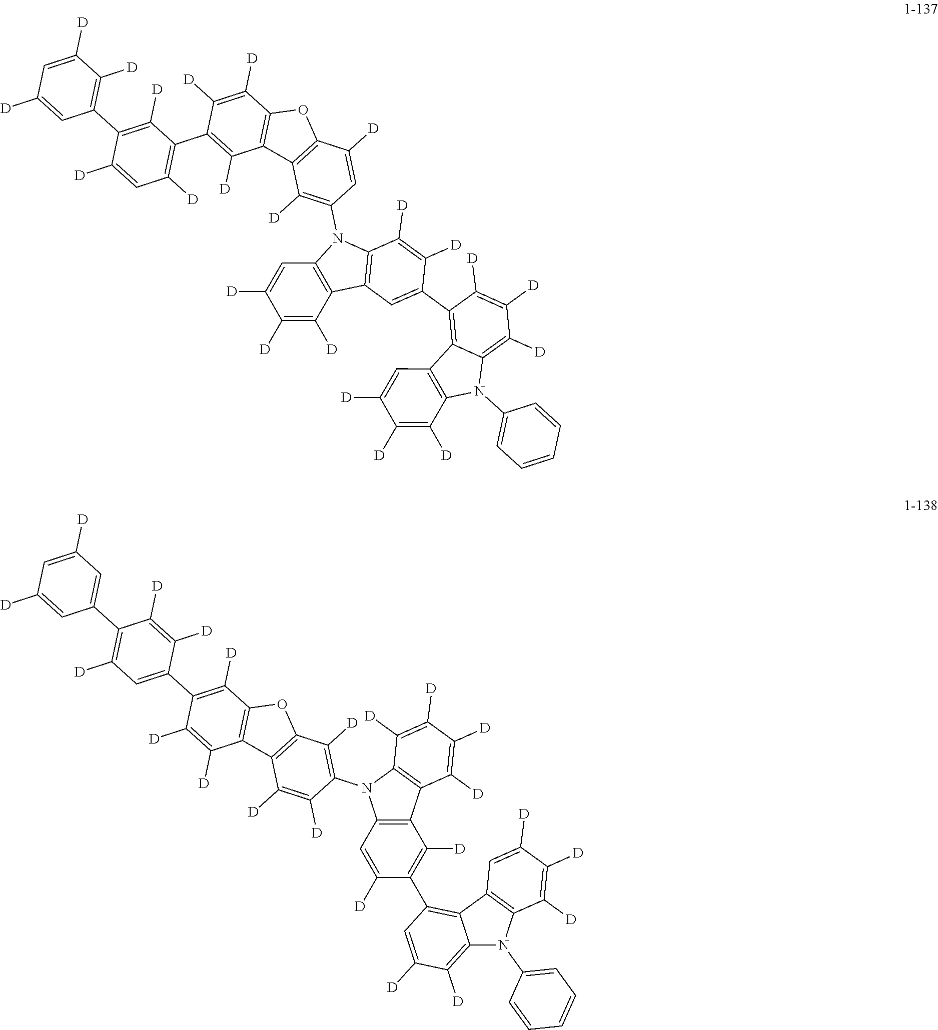 [SMILES: [2H]c1cc([2H])c([2H])c(-c2c([2H])cc([2H])c(-c3c([2H])c([2H])c4oc5c([2H])cc(-n6c7cc([2H])c([2H])c([2H])c7c7cc(-c8c([2H])c([2H])c([2H])c9c8c8cc([2H])c([2H])c([2H])c8n9-c8ccccc8)c([2H])c([2H])c76)c([2H])c5c4c3[2H])c2[2H])c1 ; [2H]c1cc([2H])cc(-c2c([2H])cc(-c3c([2H])c([2H])c4c(oc5c([2H])c(-n6c7cc([2H])c(-c8cc([2H])c([2H])c9c8c8cc([2H])c([2H])c([2H])c8n9-c8ccccc8)c([2H])c7c7c([2H])c([2H])c([2H])c([2H])c76)c([2H])c([2H])c54)c3[2H])c([2H])c2[2H])c1]

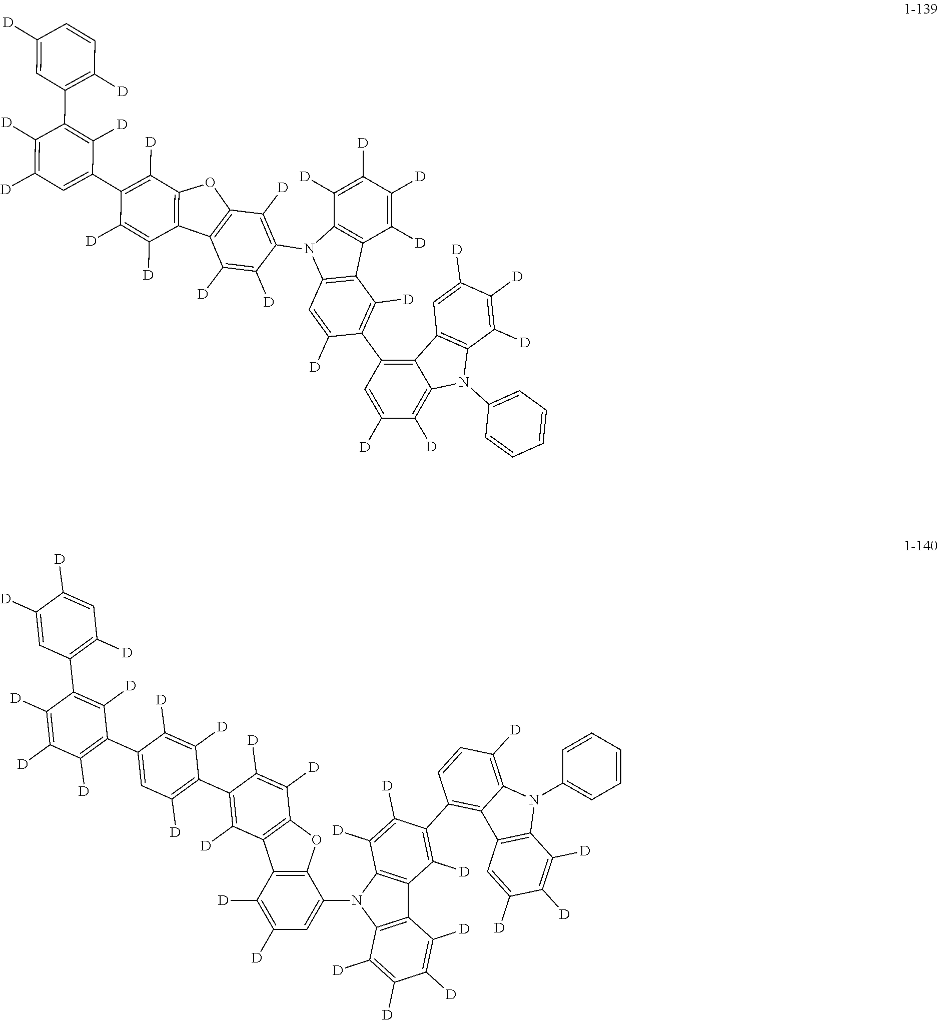 [SMILES: [2H]c1ccc([2H])c(-c2c([2H])c([2H])cc(-c3c([2H])c([2H])c4c(oc5c([2H])c(-n6c7cc([2H])c(-c8cc([2H])c([2H])c9c8c8cc([2H])c([2H])c([2H])c8n9-c8ccccc8)c([2H])c7c7c([2H])c([2H])c([2H])c([2H])c76)c([2H])c([2H])c54)c3[2H])c2[2H])c1 ; [2H]c1cc([2H])c(-c2c([2H])c([2H])c([2H])c(-c3cc([2H])c(-c4c([2H])c([2H])c5oc6c(-n7c8c([2H])c([2H])c([2H])c([2H])c8c8c([2H])c(-c9ccc([2H])c%10c9c9cc([2H])c([2H])c([2H])c9n%10-c9ccccc9)c([2H])c([2H])c87)cc([2H])c([2H])c6c5c4[2H])c([2H])c3[2H])c2[2H])cc1[2H]]

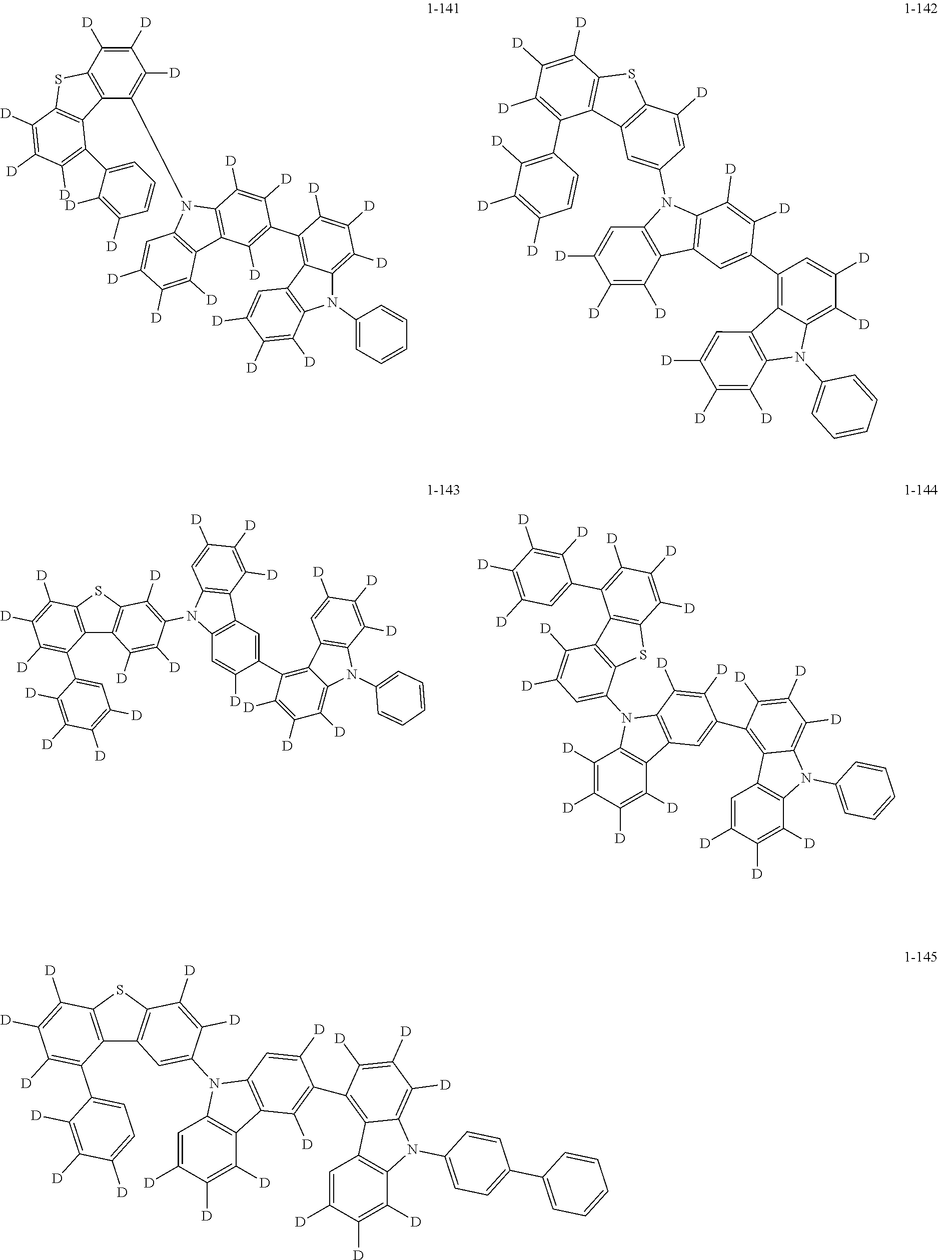

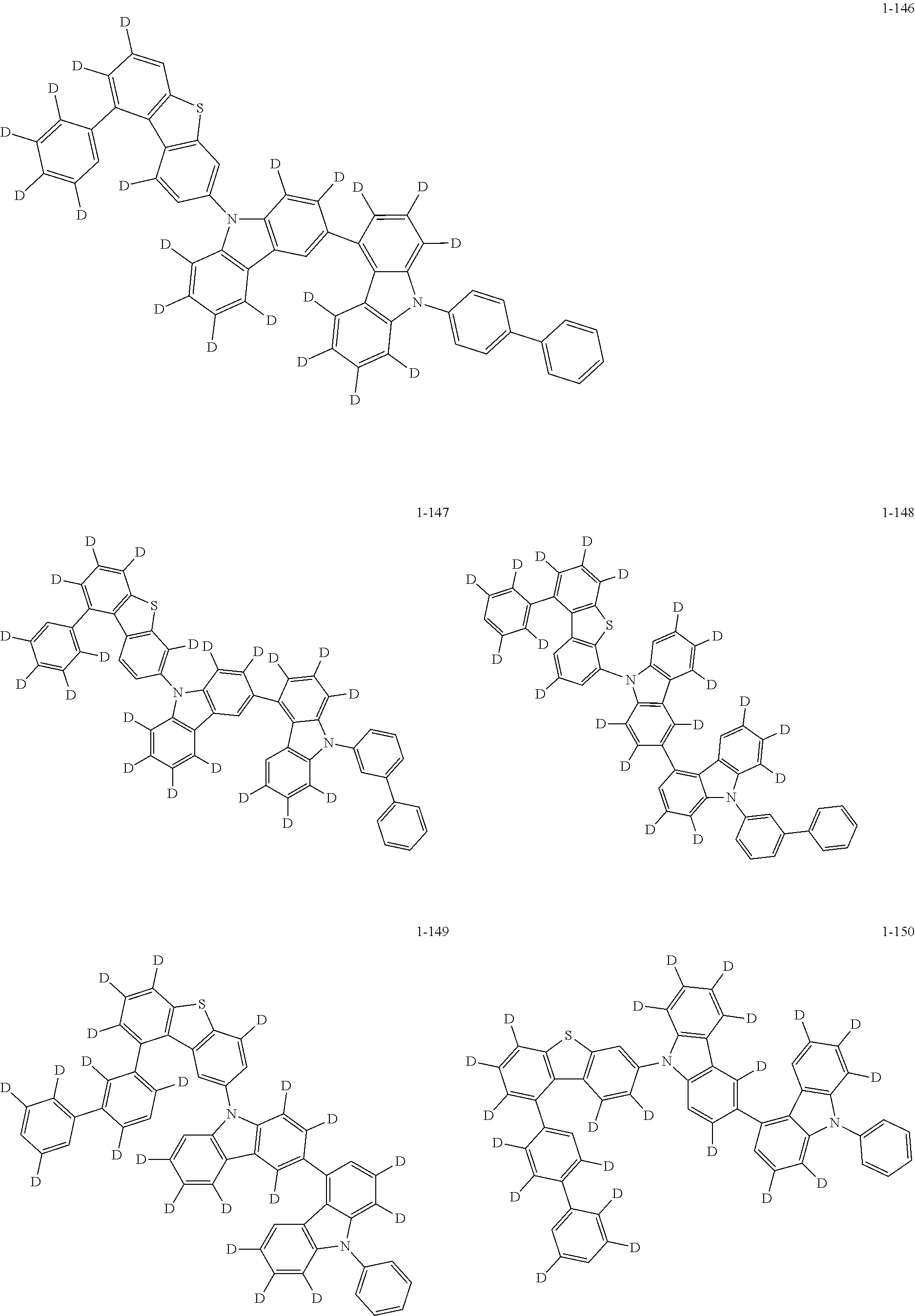

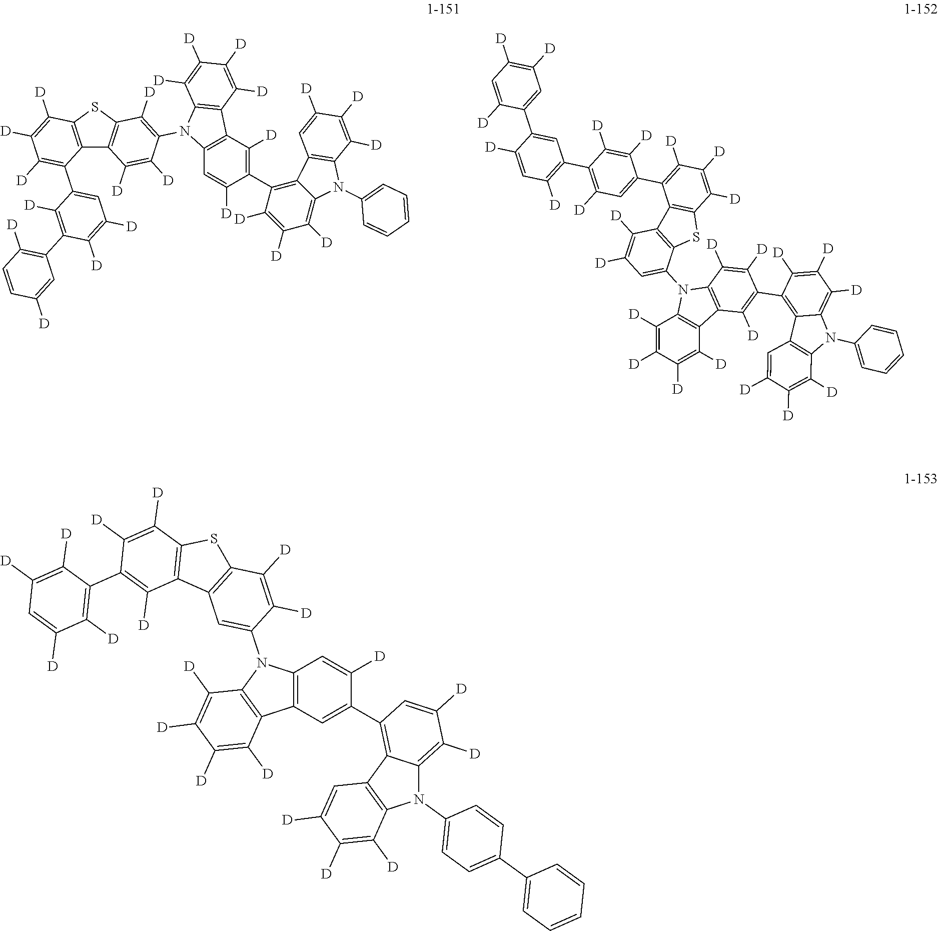

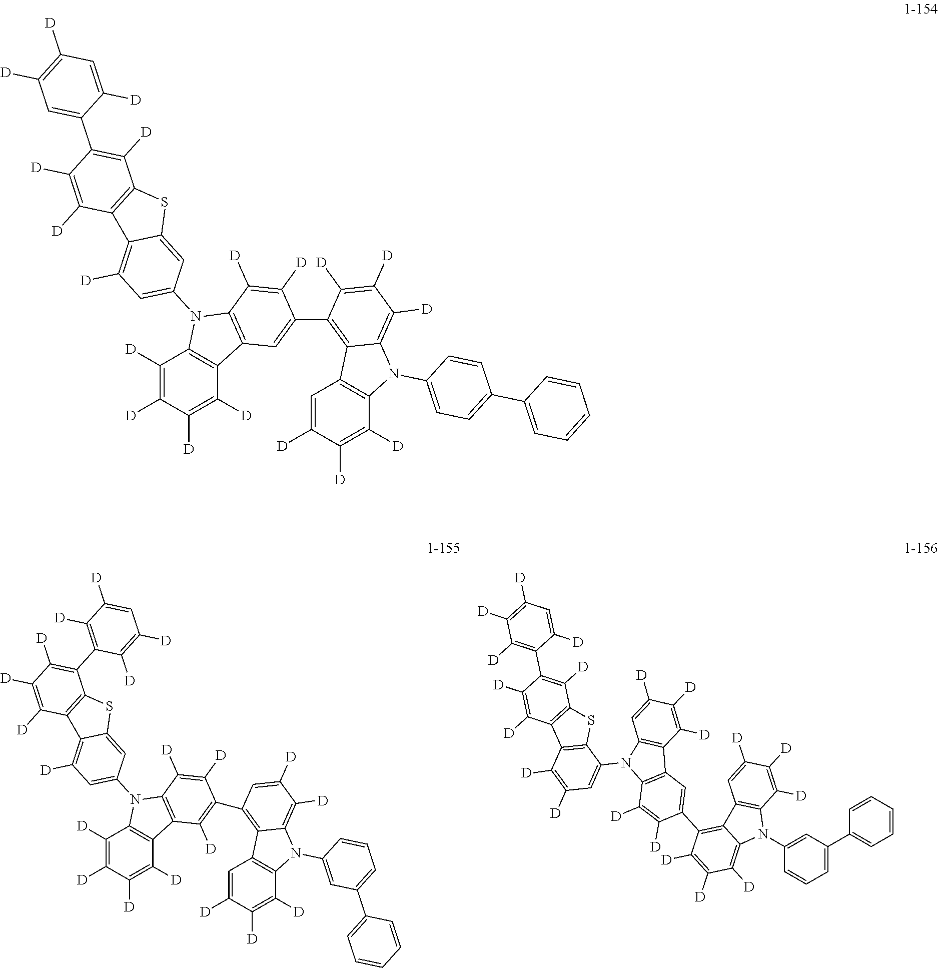

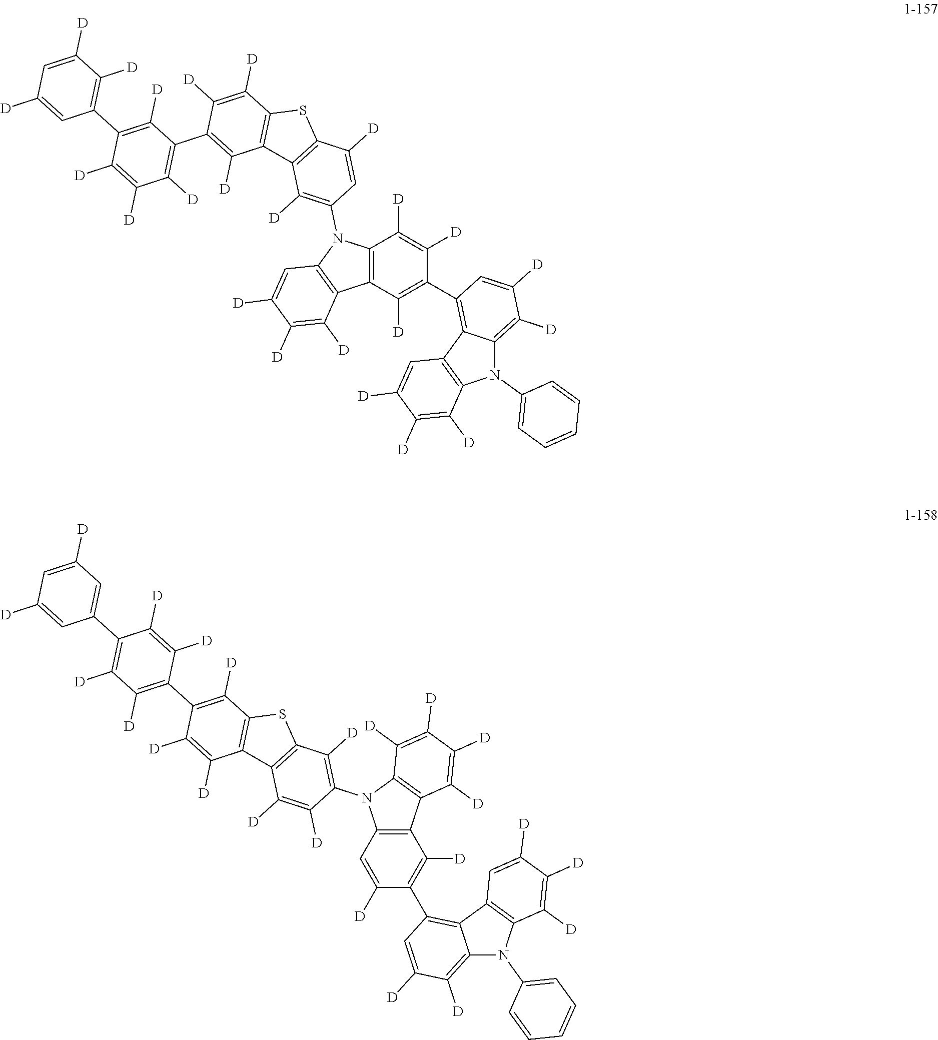 [SMILES: [2H]c1cc([2H])c([2H])c(-c2c([2H])c([2H])c([2H])c(-c3c([2H])c([2H])c4sc5c([2H])cc(-n6c7cc([2H])c([2H])c([2H])c7c7c([2H])c(-c8cc([2H])c([2H])c9c8c8cc([2H])c([2H])c([2H])c8n9-c8ccccc8)c([2H])c([2H])c76)c([2H])c5c4c3[2H])c2[2H])c1 ; [2H]c1cc([2H])cc(-c2c([2H])c([2H])c(-c3c([2H])c([2H])c4c(sc5c([2H])c(-n6c7cc([2H])c(-c8cc([2H])c([2H])c9c8c8cc([2H])c([2H])c([2H])c8n9-c8ccccc8)c([2H])c7c7c([2H])c([2H])c([2H])c([2H])c76)c([2H])c([2H])c54)c3[2H])c([2H])c2[2H])c1]

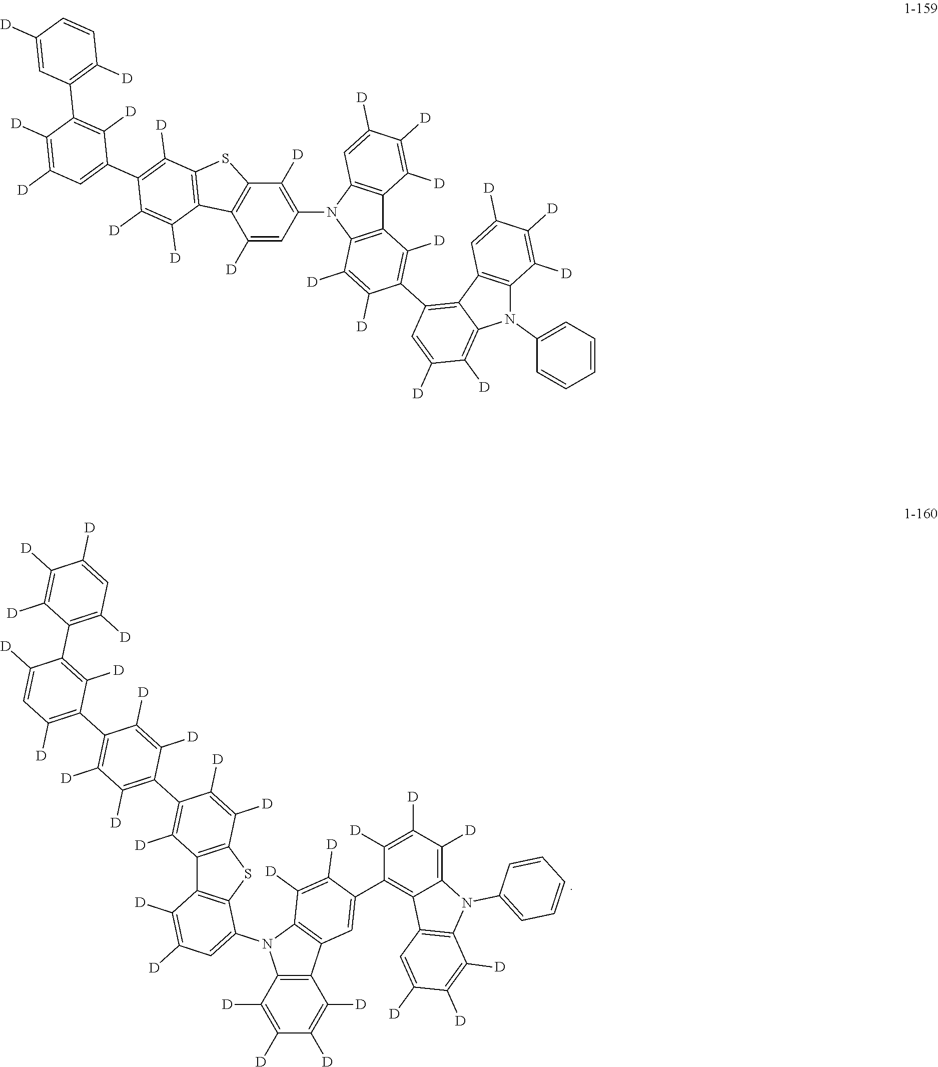 [SMILES: [2H]c1ccc([2H])c(-c2c([2H])c([2H])cc(-c3c([2H])c([2H])c4c(sc5c([2H])c(-n6c7cc([2H])c([2H])c([2H])c7c7c([2H])c(-c8cc([2H])c([2H])c9c8c8cc([2H])c([2H])c([2H])c8n9-c8ccccc8)c([2H])c([2H])c76)cc([2H])c54)c3[2H])c2[2H])c1 ; [2H]c1cc([2H])c(-c2c([2H])cc([2H])c(-c3c([2H])c([2H])c(-c4c([2H])c([2H])c5sc6c(-n7c8c([2H])c([2H])c(-c9c([2H])c([2H])c([2H])c%10c9c9cc([2H])c([2H])c([2H])c9n%10-c9ccccc9)cc8c8c([2H])c([2H])c([2H])c([2H])c87)cc([2H])c([2H])c6c5c4[2H])c([2H])c3[2H])c2[2H])c([2H])c1[2H]]

9. An organic light emitting device comprising:
a first electrode;
a second electrode provided to face the first electrode; and
an organic material layer having one or more layers provided between the first electrode and the second electrode,
wherein one or more layers of the organic material layer comprise the heterocyclic compound of claim 1.

10. The organic light emitting device of claim 9, wherein the organic material layer further comprises a heterocyclic compound represented by the following Chemical Formula 2: 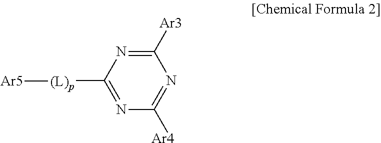
wherein, in Chemical Formula 2,
L is a direct bond; a substituted or unsubstituted C6 to C60 arylene group; or a substituted or unsubstituted C2 to C60 heteroarylene group,
p is an integer from 0 to 4, and when p is 2 or higher, substituents in the parenthesis are the same as or different from each other, and
Ar3 to Ar5 are the same as or different from each other, and are each independently a substituted or unsubstituted C6 to C60 aryl group; or a substituted or unsubstituted C2 to C60 heteroaryl group.

11. The organic light emitting device of claim 10, wherein when both Ar3 and Ar4 are a substituted or unsubstituted C6 to C60 aryl group, L is a substituted or unsubstituted C2 to C60 heteroarylene group, or Ar5 is a substituted or unsubstituted C2 to C60 heteroaryl group.

12. The organic light emitting device of claim 10, wherein a deuterium content of the heterocyclic compound represented by Chemical Formula 2 is 0% to 100%.

13. The organic light emitting device of claim 10, wherein Chemical Formula 2 is represented by any one of the following compounds:

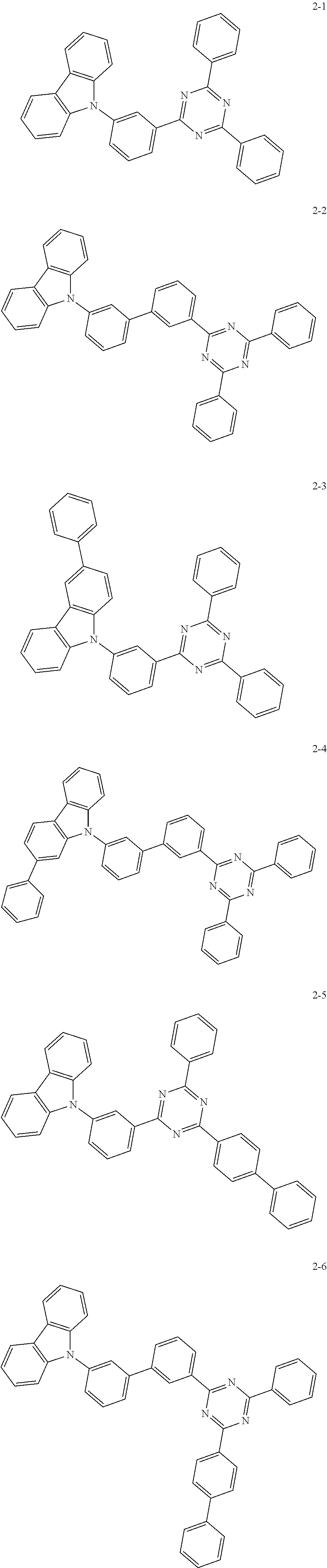 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cccc(-c4cccc(-n5c6ccccc6c6ccccc65)c4)c3)n2)cc1 ; c1ccc(-c2ccc3c(c2)c2ccccc2n3-c2cccc(-c3nc(-c4ccccc4)nc(-c4ccccc4)n3)c2)cc1 ; c1ccc(-c2ccc3c4ccccc4n(-c4cccc(-c5cccc(-c6nc(-c7ccccc7)nc(-c7ccccc7)n6)c5)c4)c3c2)cc1 ; c1ccc(-c2ccc(-c3nc(-c4ccccc4)nc(-c4cccc(-n5c6ccccc6c6ccccc65)c4)n3)cc2)cc1 ; c1ccc(-c2ccc(-c3nc(-c4ccccc4)nc(-c4cccc(-c5cccc(-n6c7ccccc7c7ccccc76)c5)c4)n3)cc2)cc1]

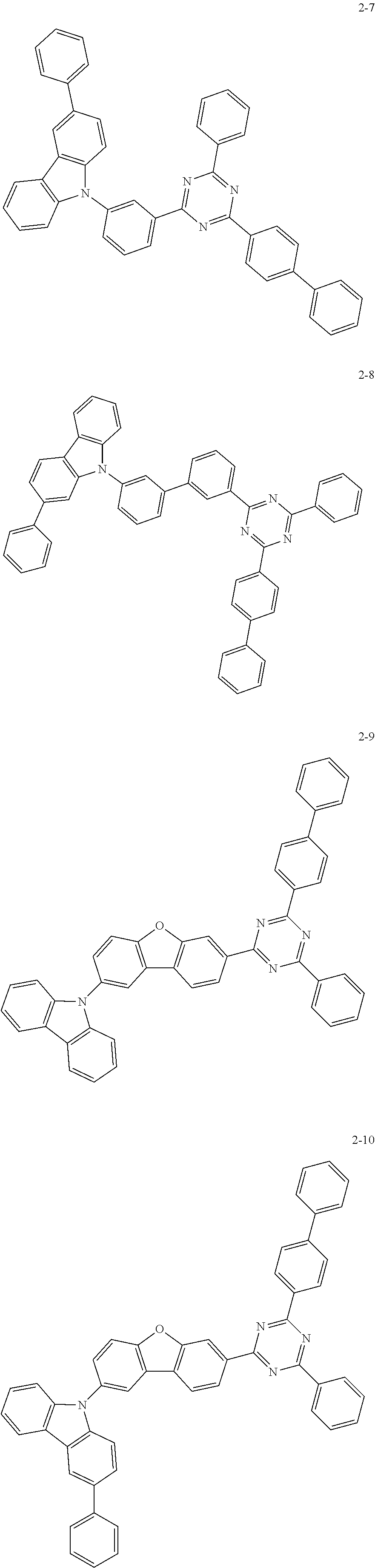 [SMILES: c1ccc(-c2ccc(-c3nc(-c4ccccc4)nc(-c4cccc(-n5c6ccccc6c6cc(-c7ccccc7)ccc65)c4)n3)cc2)cc1 ; c1ccc(-c2ccc(-c3nc(-c4ccccc4)nc(-c4cccc(-c5cccc(-n6c7ccccc7c7ccc(-c8ccccc8)cc76)c5)c4)n3)cc2)cc1 ; c1ccc(-c2ccc(-c3nc(-c4ccccc4)nc(-c4ccc5c(c4)oc4ccc(-n6c7ccccc7c7ccccc76)cc45)n3)cc2)cc1 ; c1ccc(-c2ccc(-c3nc(-c4ccccc4)nc(-c4ccc5c(c4)oc4ccc(-n6c7ccccc7c7cc(-c8ccccc8)ccc76)cc45)n3)cc2)cc1]

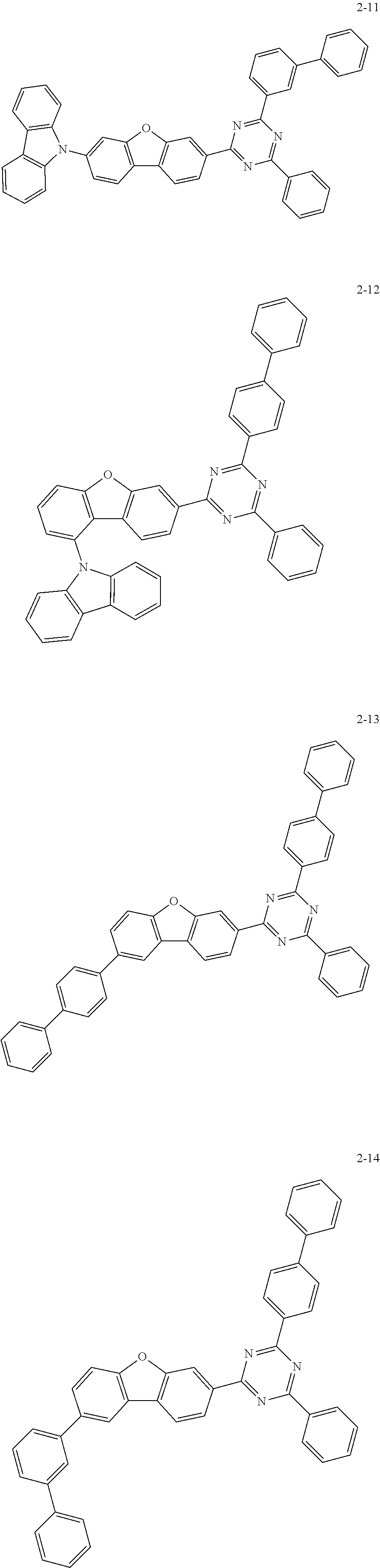 [SMILES: c1ccc(-c2cccc(-c3nc(-c4ccccc4)nc(-c4ccc5c(c4)oc4cc(-n6c7ccccc7c7ccccc76)ccc45)n3)c2)cc1 ; c1ccc(-c2ccc(-c3nc(-c4ccccc4)nc(-c4ccc5c(c4)oc4cccc(-n6c7ccccc7c7ccccc76)c45)n3)cc2)cc1 ; c1ccc(-c2ccc(-c3ccc4oc5cc(-c6nc(-c7ccccc7)nc(-c7ccc(-c8ccccc8)cc7)n6)ccc5c4c3)cc2)cc1 ; c1ccc(-c2ccc(-c3nc(-c4ccccc4)nc(-c4ccc5c(c4)oc4ccc(-c6cccc(-c7ccccc7)c6)cc45)n3)cc2)cc1]

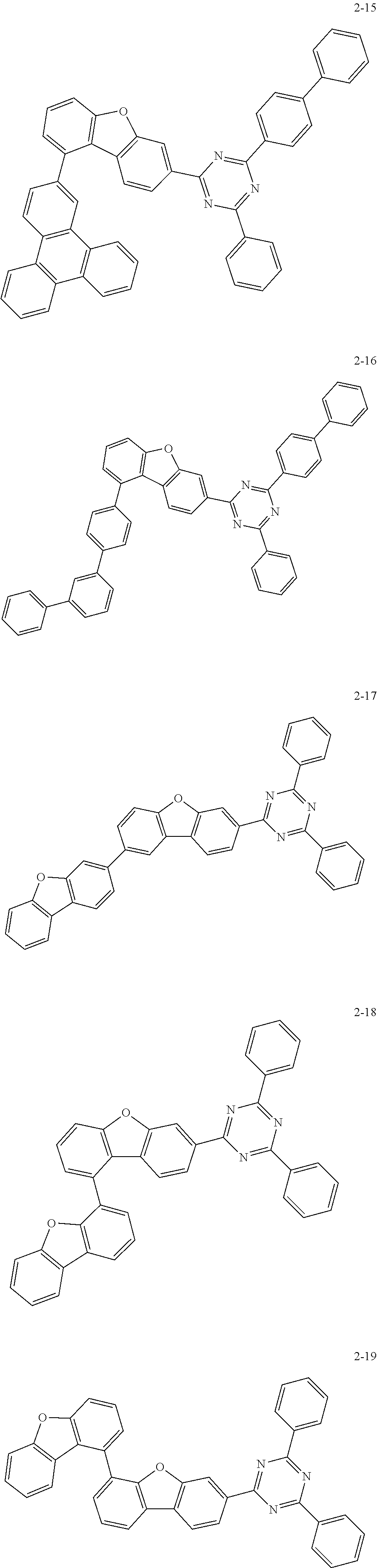 [SMILES: c1ccc(-c2ccc(-c3nc(-c4ccccc4)nc(-c4ccc5c(c4)oc4cccc(-c6ccc7c8ccccc8c8ccccc8c7c6)c45)n3)cc2)cc1 ; c1ccc(-c2ccc(-c3nc(-c4ccccc4)nc(-c4ccc5c(c4)oc4cccc(-c6ccc(-c7cccc(-c8ccccc8)c7)cc6)c45)n3)cc2)cc1 ; c1ccc(-c2nc(-c3ccccc3)nc(-c3ccc4c(c3)oc3ccc(-c5ccc6c(c5)oc5ccccc56)cc34)n2)cc1 ; c1ccc(-c2nc(-c3ccccc3)nc(-c3ccc4c(c3)oc3cccc(-c5cccc6c5oc5ccccc56)c34)n2)cc1 ; c1ccc(-c2nc(-c3ccccc3)nc(-c3ccc4c(c3)oc3c(-c5cccc6oc7ccccc7c56)cccc34)n2)cc1]

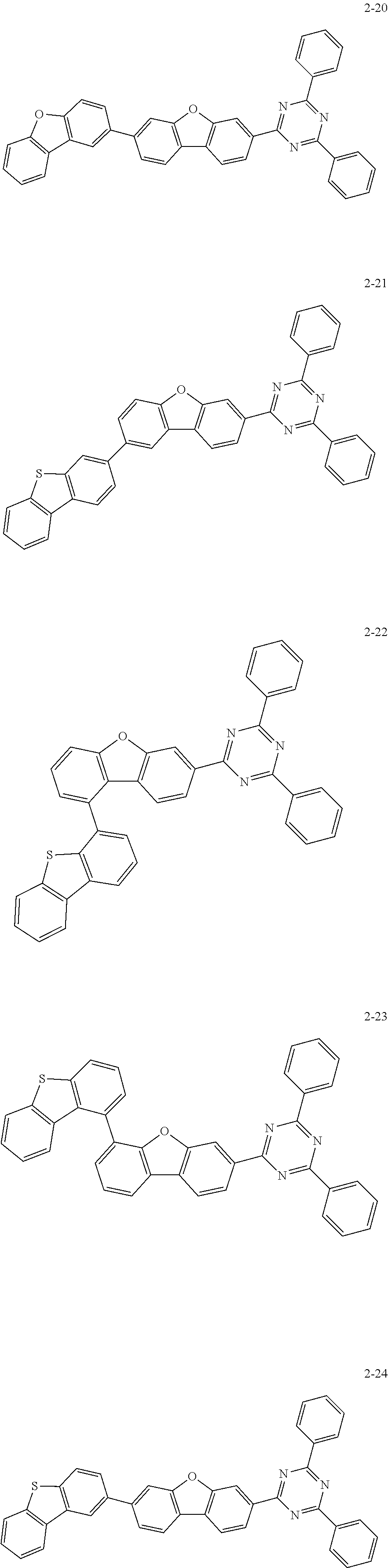 [SMILES: c1ccc(-c2nc(-c3ccccc3)nc(-c3ccc4c(c3)oc3cc(-c5ccc6oc7ccccc7c6c5)ccc34)n2)cc1 ; c1ccc(-c2nc(-c3ccccc3)nc(-c3ccc4c(c3)oc3ccc(-c5ccc6c(c5)sc5ccccc56)cc34)n2)cc1 ; c1ccc(-c2nc(-c3ccccc3)nc(-c3ccc4c(c3)oc3cccc(-c5cccc6c5sc5ccccc56)c34)n2)cc1 ; c1ccc(-c2nc(-c3ccccc3)nc(-c3ccc4c(c3)oc3c(-c5cccc6sc7ccccc7c56)cccc34)n2)cc1 ; c1ccc(-c2nc(-c3ccccc3)nc(-c3ccc4c(c3)oc3cc(-c5ccc6sc7ccccc7c6c5)ccc34)n2)cc1]

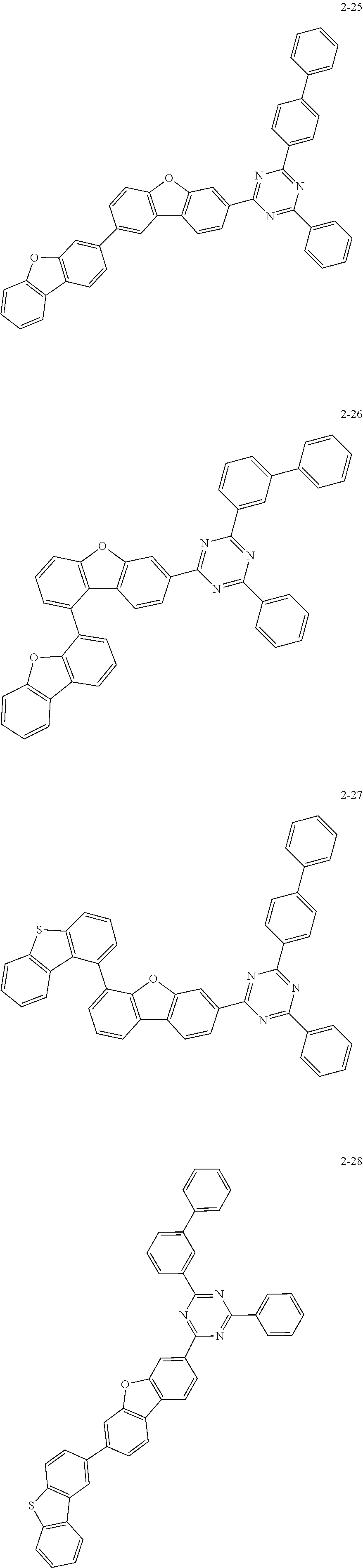 [SMILES: c1ccc(-c2ccc(-c3nc(-c4ccccc4)nc(-c4ccc5c(c4)oc4ccc(-c6ccc7c(c6)oc6ccccc67)cc45)n3)cc2)cc1 ; c1ccc(-c2cccc(-c3nc(-c4ccccc4)nc(-c4ccc5c(c4)oc4cccc(-c6cccc7c6oc6ccccc67)c45)n3)c2)cc1 ; c1ccc(-c2ccc(-c3nc(-c4ccccc4)nc(-c4ccc5c(c4)oc4c(-c6cccc7sc8ccccc8c67)cccc45)n3)cc2)cc1 ; c1ccc(-c2cccc(-c3nc(-c4ccccc4)nc(-c4ccc5c(c4)oc4cc(-c6ccc7sc8ccccc8c7c6)ccc45)n3)c2)cc1]

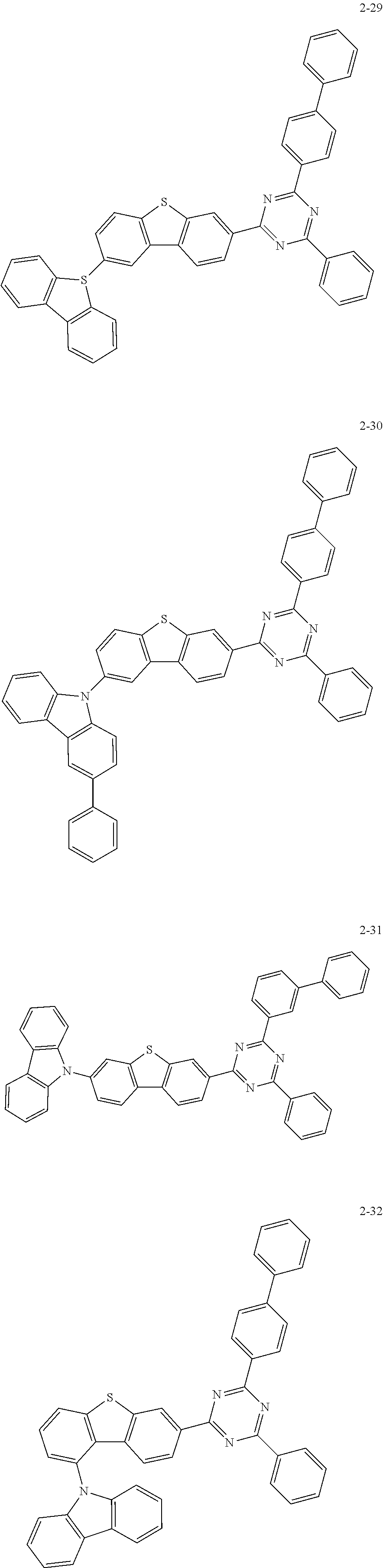

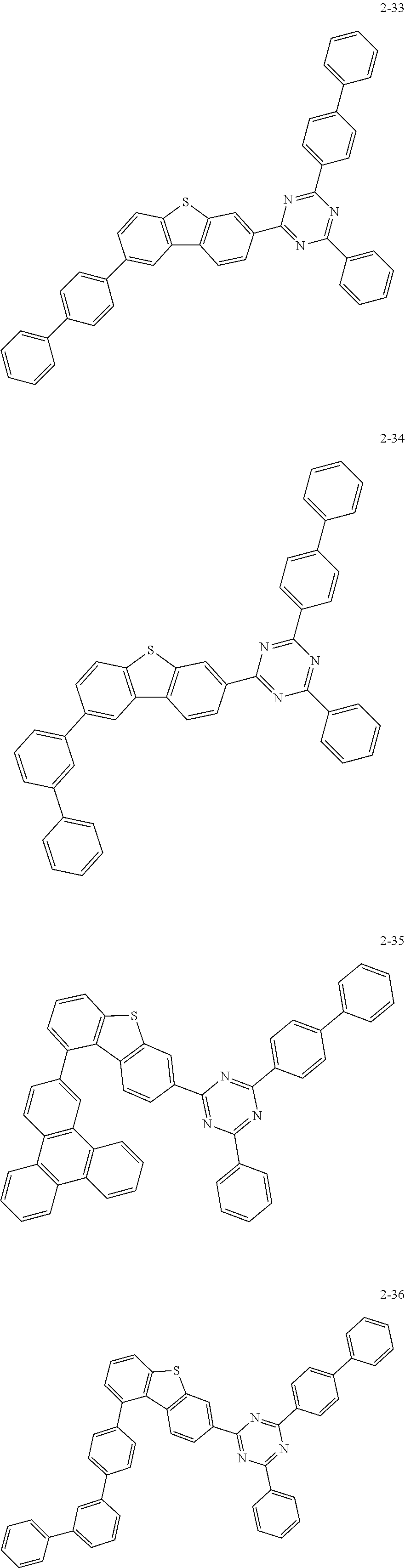 [SMILES: c1ccc(-c2ccc(-c3ccc4sc5cc(-c6nc(-c7ccccc7)nc(-c7ccc(-c8ccccc8)cc7)n6)ccc5c4c3)cc2)cc1 ; c1ccc(-c2ccc(-c3nc(-c4ccccc4)nc(-c4ccc5c(c4)sc4ccc(-c6cccc(-c7ccccc7)c6)cc45)n3)cc2)cc1 ; c1ccc(-c2ccc(-c3nc(-c4ccccc4)nc(-c4ccc5c(c4)sc4cccc(-c6ccc7c8ccccc8c8ccccc8c7c6)c45)n3)cc2)cc1 ; c1ccc(-c2ccc(-c3nc(-c4ccccc4)nc(-c4ccc5c(c4)sc4cccc(-c6ccc(-c7cccc(-c8ccccc8)c7)cc6)c45)n3)cc2)cc1]

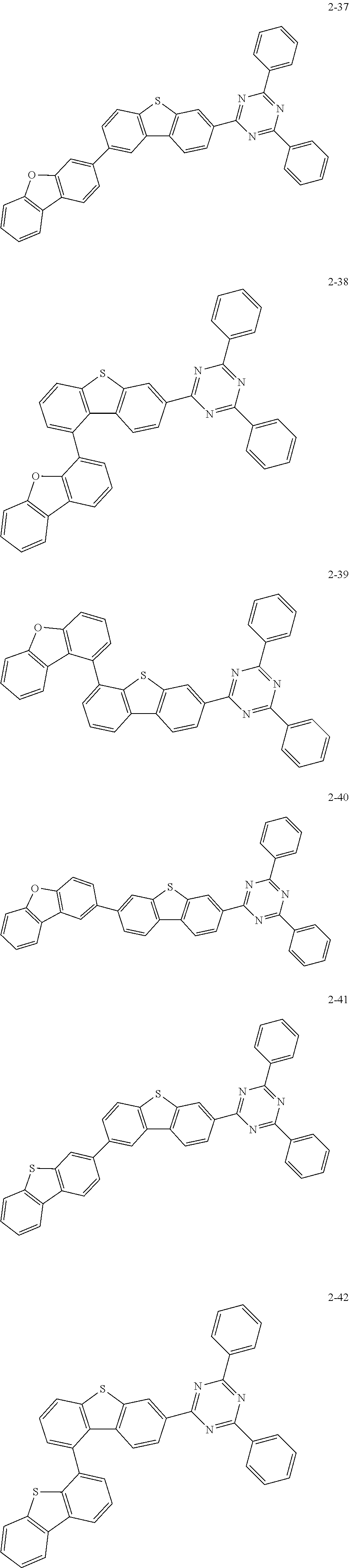 [SMILES: c1ccc(-c2nc(-c3ccccc3)nc(-c3ccc4c(c3)sc3ccc(-c5ccc6c(c5)oc5ccccc56)cc34)n2)cc1 ; c1ccc(-c2nc(-c3ccccc3)nc(-c3ccc4c(c3)sc3cccc(-c5cccc6c5oc5ccccc56)c34)n2)cc1 ; c1ccc(-c2nc(-c3ccccc3)nc(-c3ccc4c(c3)sc3c(-c5cccc6oc7ccccc7c56)cccc34)n2)cc1 ; c1ccc(-c2nc(-c3ccccc3)nc(-c3ccc4c(c3)sc3cc(-c5ccc6oc7ccccc7c6c5)ccc34)n2)cc1 ; c1ccc(-c2nc(-c3ccccc3)nc(-c3ccc4c(c3)sc3ccc(-c5ccc6c(c5)sc5ccccc56)cc34)n2)cc1 ; c1ccc(-c2nc(-c3ccccc3)nc(-c3ccc4c(c3)sc3cccc(-c5cccc6c5sc5ccccc56)c34)n2)cc1]

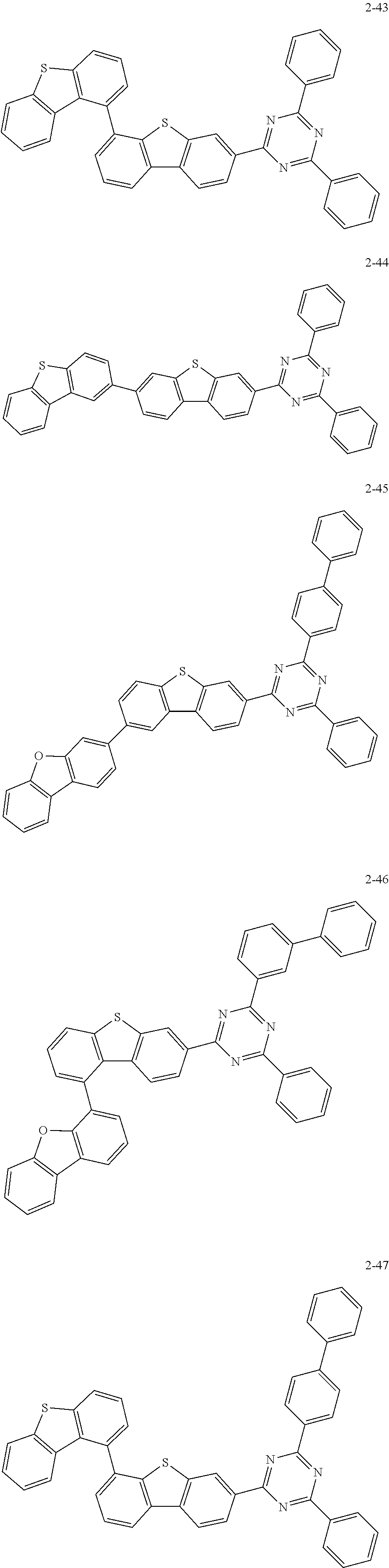 [SMILES: c1ccc(-c2nc(-c3ccccc3)nc(-c3ccc4c(c3)sc3c(-c5cccc6sc7ccccc7c56)cccc34)n2)cc1 ; c1ccc(-c2nc(-c3ccccc3)nc(-c3ccc4c(c3)sc3cc(-c5ccc6sc7ccccc7c6c5)ccc34)n2)cc1 ; c1ccc(-c2ccc(-c3nc(-c4ccccc4)nc(-c4ccc5c(c4)sc4ccc(-c6ccc7c(c6)oc6ccccc67)cc45)n3)cc2)cc1 ; c1ccc(-c2cccc(-c3nc(-c4ccccc4)nc(-c4ccc5c(c4)sc4cccc(-c6cccc7c6oc6ccccc67)c45)n3)c2)cc1 ; c1ccc(-c2ccc(-c3nc(-c4ccccc4)nc(-c4ccc5c(c4)sc4c(-c6cccc7sc8ccccc8c67)cccc45)n3)cc2)cc1]

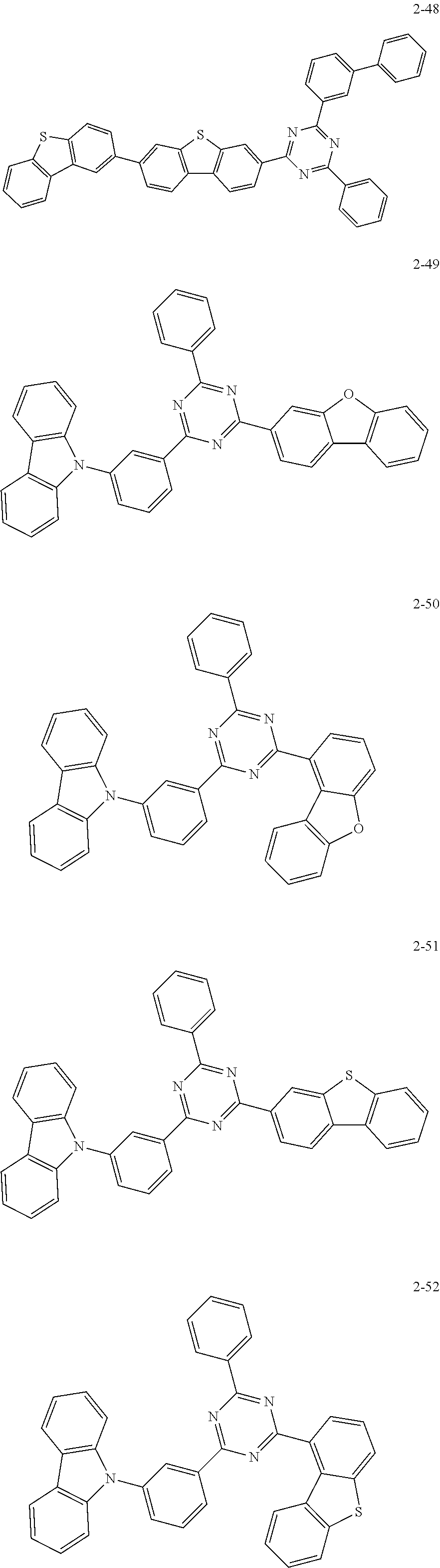 [SMILES: c1ccc(-c2cccc(-c3nc(-c4ccccc4)nc(-c4ccc5c(c4)sc4cc(-c6ccc7sc8ccccc8c7c6)ccc45)n3)c2)cc1 ; c1ccc(-c2nc(-c3cccc(-n4c5ccccc5c5ccccc54)c3)nc(-c3ccc4c(c3)oc3ccccc34)n2)cc1 ; c1ccc(-c2nc(-c3cccc(-n4c5ccccc5c5ccccc54)c3)nc(-c3cccc4oc5ccccc5c34)n2)cc1 ; c1ccc(-c2nc(-c3cccc(-n4c5ccccc5c5ccccc54)c3)nc(-c3ccc4c(c3)sc3ccccc34)n2)cc1 ; c1ccc(-c2nc(-c3cccc(-n4c5ccccc5c5ccccc54)c3)nc(-c3cccc4sc5ccccc5c34)n2)cc1]

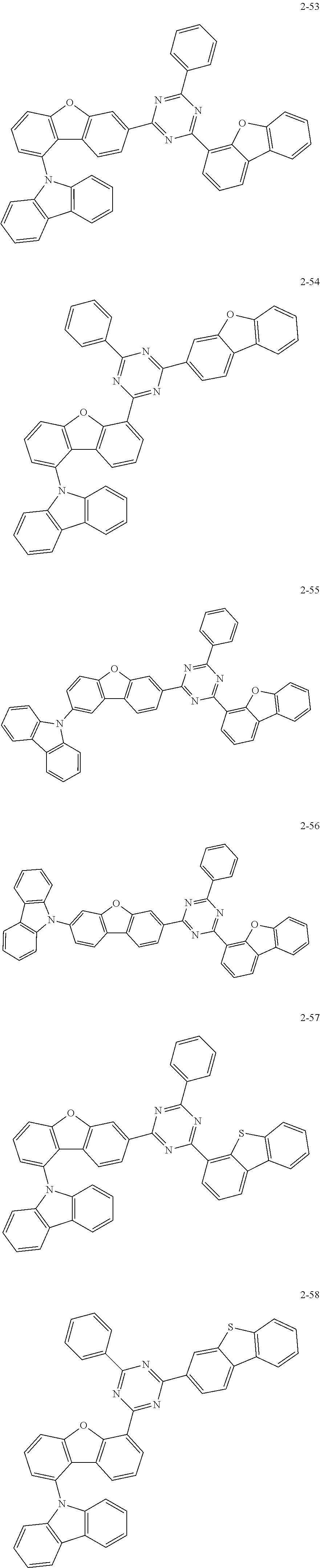

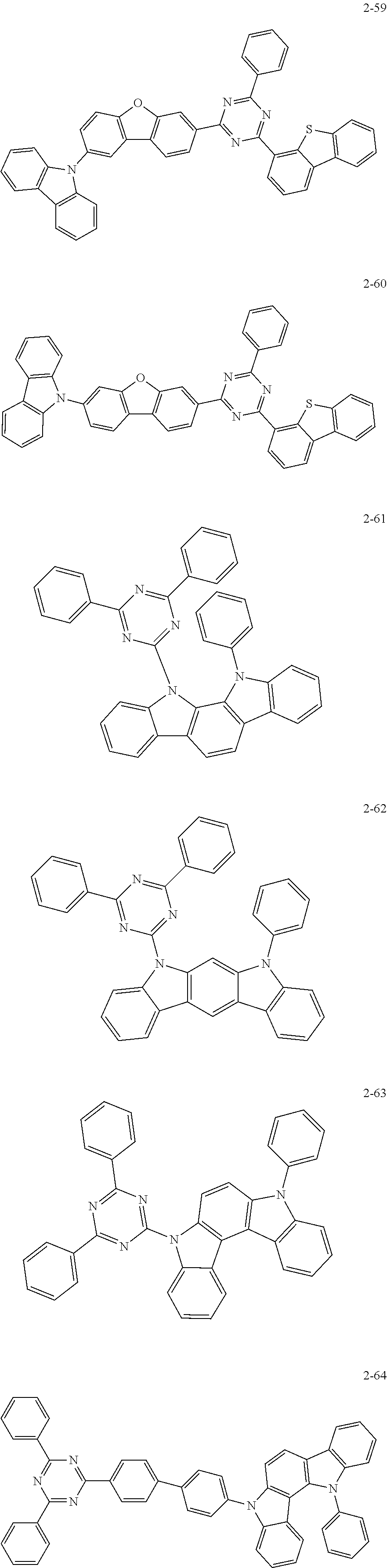

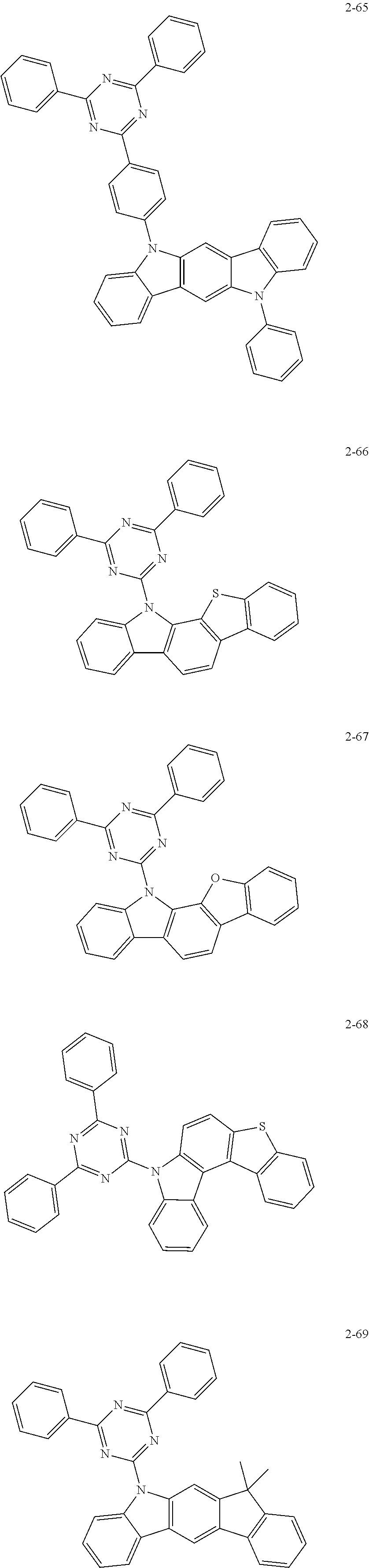 [SMILES: c1ccc(-c2nc(-c3ccccc3)nc(-c3ccc(-n4c5ccccc5c5cc6c(cc54)c4ccccc4n6-c4ccccc4)cc3)n2)cc1 ; c1ccc(-c2nc(-c3ccccc3)nc(-n3c4ccccc4c4ccc5c6ccccc6sc5c43)n2)cc1 ; c1ccc(-c2nc(-c3ccccc3)nc(-n3c4ccccc4c4ccc5c6ccccc6oc5c43)n2)cc1 ; c1ccc(-c2nc(-c3ccccc3)nc(-n3c4ccccc4c4c5c(ccc43)sc3ccccc35)n2)cc1 ; CC1(C)c2ccccc2-c2cc3c4ccccc4n(-c4nc(-c5ccccc5)nc(-c5ccccc5)n4)c3cc21]

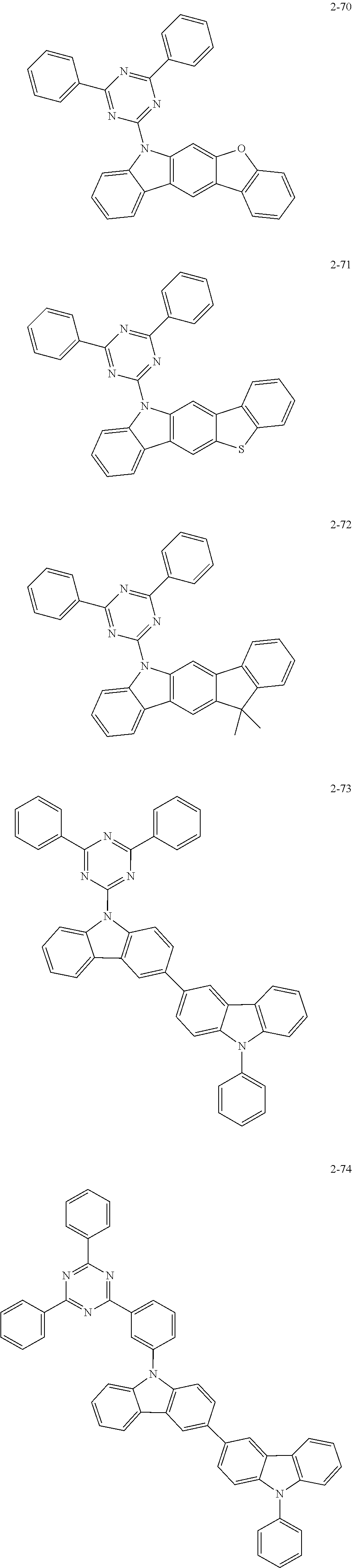 [SMILES: c1ccc(-c2nc(-c3ccccc3)nc(-n3c4ccccc4c4cc5c(cc43)oc3ccccc35)n2)cc1 ; c1ccc(-c2nc(-c3ccccc3)nc(-n3c4ccccc4c4cc5sc6ccccc6c5cc43)n2)cc1 ; CC1(C)c2ccccc2-c2cc3c(cc21)c1ccccc1n3-c1nc(-c2ccccc2)nc(-c2ccccc2)n1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

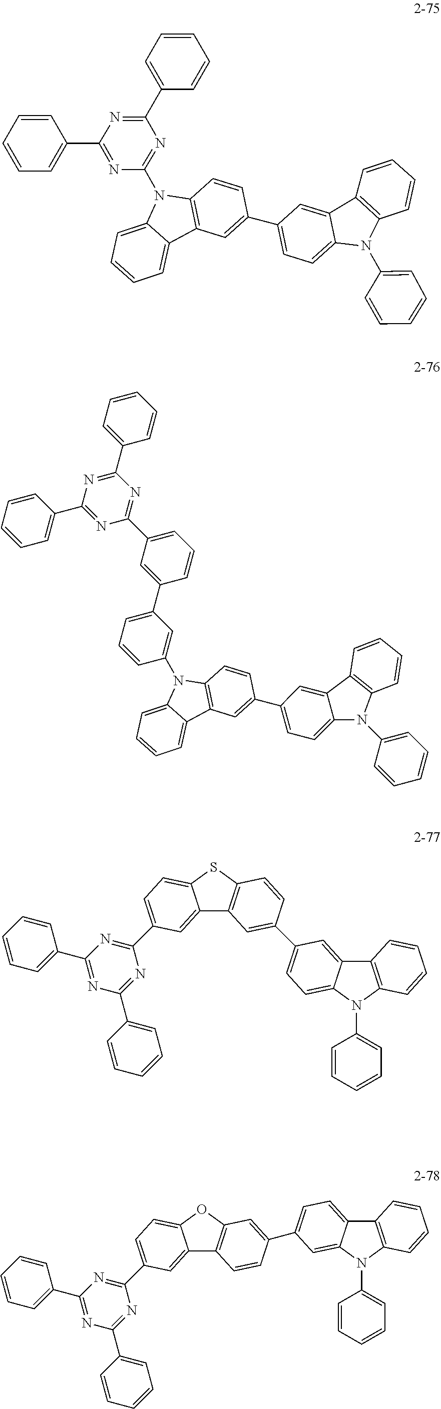 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3cccc(-c4cccc(-n5c6ccccc6c6cc(-c7ccc8c(c7)c7ccccc7n8-c7ccccc7)ccc65)c4)c3)n2)cc1 ; c1ccc(-c2nc(-c3ccccc3)nc(-c3ccc4sc5ccc(-c6ccc7c(c6)c6ccccc6n7-c6ccccc6)cc5c4c3)n2)cc1 ; c1ccc(-c2nc(-c3ccccc3)nc(-c3ccc4oc5cc(-c6ccc7c8ccccc8n(-c8ccccc8)c7c6)ccc5c4c3)n2)cc1]

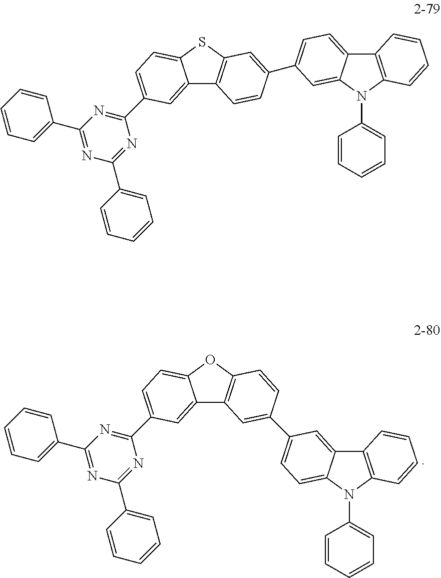 [SMILES: c1ccc(-c2nc(-c3ccccc3)nc(-c3ccc4sc5cc(-c6ccc7c8ccccc8n(-c8ccccc8)c7c6)ccc5c4c3)n2)cc1 ; c1ccc(-c2nc(-c3ccccc3)nc(-c3ccc4oc5ccc(-c6ccc7c(c6)c6ccccc6n7-c6ccccc6)cc5c4c3)n2)cc1]

14. The organic light emitting device of claim 9, wherein the organic material layer comprises a light emitting layer, and the light emitting layer comprises the heterocyclic compound represented by Chemical Formula 1.

15. The organic light emitting device of claim 9, wherein the organic material layer comprises a light emitting layer, the light emitting layer comprises a host material, and the host material comprises the heterocyclic compound represented by Chemical Formula 1.

16. The organic light emitting device of claim 9, further comprising one or two or more layers selected from the group consisting of a light emitting layer, a hole injection layer, a hole transport layer, an electron blocking layer, a hole blocking layer, an electron transport layer and an electron injection layer.

17. A composition for an organic material layer of an organic light emitting device, comprising the heterocyclic compound represented by Chemical Formula 1 according to claim 1; and a compound represented by the following Chemical Formula 2: 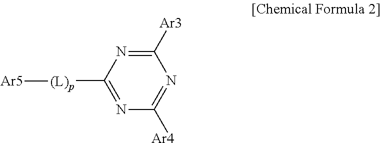
wherein, in Chemical Formula 2,
L is a direct bond; a substituted or unsubstituted C6 to C60 arylene group; or a substituted or unsubstituted C2 to C60 heteroarylene group,
p is an integer from 0 to 4, and when p is 2 or higher, substituents in the parenthesis are the same as or different from each other, and
Ar3 to Ar5 are the same as or different from each other, and are each independently a substituted or unsubstituted C6 to C60 aryl group; or a substituted or unsubstituted C2 to C60 heteroaryl group.

18. The composition of claim 17, wherein a weight ratio of the heterocyclic compound represented by Chemical Formula 1:the heterocyclic compound represented by Chemical Formula 2 in the composition is 1:10 to 10:1.
